(19) United States
(12) Patent Application Publication
(10) Pub. No.: US 2006/0141462 A1
(43) Pub. Date: Jun. 29, 2006

(54) Human type II diabetes gene-slit-3 located on chromosome 5q35

(76) Inventors: Inga Reynisdottir, Reykjavik (IS); Jeffrey R. Gulcher, Lake Barrington, IL (US); Struan F. Grant, Reykjavik (IS); Gudmar Thorleifsson, Reykjavik (IS)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/533,365

(22) PCT Filed: Oct. 31, 2003
(86) PCT No.: PCT/US03/34801

§ 371 Date: Nov. 9, 2005

Related U.S. Application Data
(60) Provisional application No. 60/423,541, filed on Nov. 1, 2002.

(51) Int. Cl. C12Q 1/68

(52) U.S. Cl. 435/6

(57) ABSTRACT

Association of Type II diabetes and a locus on chromosome 5q35 is disclosed. In particular, the gene SLIT-3 with this locus is shown by linkage analysis to be a susceptibility gene for Type II diabetes. Pathway targeting for drug delivery and diagnosis applications in identifying those have Type II diabetes or at risk of developing Type II diabetes, in particular those that are non-obese are described.

### RELATED APPLICATION

This application claims the benefit of U.S. Provisional Application No. 60/423,541, filed on Nov. 1, 2002. The entire teachings of the above application are incorporated herein by reference.

### BACKGROUND OF THE INVENTION

Diabetes mellitus, a metabolic disease in which carbohydrate utilization is reduced and lipid and protein utilization is enhanced, is caused by an absolute or relative deficiency of insulin. In the more severe cases, diabetes is characterized by chronic hyperglycemia, glycosuria, water and electrolyte loss, ketoacidosis and coma. Long term complications include development of neuropathy, retinopathy, nephropathy, generalized degenerative changes in large and small blood vessels and increased susceptibility to infection. The most common form of diabetes is Type II, non-insulin-dependent diabetes which is characterized by hyperglycemia due to impaired insulin secretion and insulin resistance in target tissues. Both genetic and environmental factors contribute to the disease. For example, obesity plays a major role in the development of the disease. Type II diabetes is often a mild form of diabetes mellitus of gradual onset.

The health implications of Type II diabetes are enormous. In 1995, there were 135 million adults with diabetes worldwide. It is estimated that close to 300 million will have diabetes in the year 2025. (King H., et al., Diabetes Care, 21(9): 1414-1431 (1998)). The prevalence of Type II diabetes in the adult population in Iceland is 2.5% (Vilbergsson, S., et al., Diabet. Med., 14(6): 491-498 (1997)), which comprises approximately 5,000 people over the age of 34 who have the disease.

### SUMMARY OF THE INVENTION

As described herein, a locus on chromosome 5q35 has been demonstrated which plays a major role in Type II diabetes. The locus, referred to as the Type II diabetes locus, comprises a nucleic acid that encodes, SLIT-3.

The present invention relates to genes located within the Type II diabetes-related locus, particularly nucleic acids comprising the SLIT-3 gene, and the amino acids encoded by these nucleic acids. The invention further relates to pathway targeting for drug delivery and diagnosis in identifying those who have Type II diabetes and those at risk of developing Type II diabetes. Also, described are a haplotype and SNPs that can be used to identify individuals with Type II diabetes or at risk of developing Type II diabetes, particularly in those that are non-obese. As a consequence, intervention can be prescribed to these individuals before symptoms of the disease present, e.g., dietary changes, exercise and/or medication. Identification of genes in the Type II diabetes locus can pave the way for a better understanding of the disease process, which in turn can lead to improved diagnostics and therapeutics.

The present invention pertains to methods of diagnosing a susceptibility to Type II diabetes in an individual, comprising detecting a polymorphism in a SLIT-3 nucleic acid, wherein the presence of the polymorphism in the nucleic acid is indicative of a susceptibility to Type II diabetes. The invention additionally pertains to methods of diagnosing Type II diabetes in an individual, comprising detecting a polymorphism in a SLIT-3 nucleic acid, wherein the presence of the polymorphism in the nucleic acid is indicative of Type II diabetes. In one embodiment, in diagnosing Type II diabetes or susceptibility to Type U diabetes by detecting the presence of a polymorphism in a SLIT-3 nucleic acid, the presence of the polymorphism in the SLIT-3 nucleic acid can be indicated, for example, by the presence of one or more of the polymorphisms indicated FIG. 11.

In other embodiments, the invention relates to methods of diagnosing a susceptibility to Type II diabetes in an individual, comprising detecting an alteration in the expression or composition of a polypeptide encoded by a SLIT-3 nucleic acid in a test sample, in comparison with the expression or composition of a polypeptide encoded by a SLIT-3 nucleic acid in a control sample, wherein the presence of an alteration in expression or composition of the polypeptide in the test sample is indicative of a susceptibility to Type II diabetes. The invention additionally relates to a method of diagnosing Type II diabetes in an individual, comprising detecting an alteration in the expression or composition of a polypeptide encoded by a SLIT-3 nucleic acid in a test sample, in comparison with the expression or composition of a polypeptide encoded by SLIT-3 nucleic acid in a control sample, wherein the presence of an alteration in expression or composition of the polypeptide in the test sample is indicative of Type II diabetes.

The invention also relates to an isolated nucleic acid molecule comprising a SLIT-3 nucleic acid, wherein the SLIT-3 nucleic acid comprises one or more nucleotide sequence(s) selected from the group of nucleic acid sequences as shown in FIG. 10 and the complements of the group of nucleic acid sequences as shown in FIG. 10. In certain embodiments, the nucleotide sequence contains one or more polymorphism(s), such as those shown in FIG. 11. In another embodiment, the invention relates to an isolated nucleic acid molecule which hybridizes under high stringency conditions to a nucleotide sequence selected from the group of nucleic acid sequences as shown in FIG. 10 and the complements of the group of nucleic acid sequences as shown in FIG. 10. In certain embodiments, wherein the nucleotide sequence contains one or more polymorphism(s), such as those shown in FIG. 11.

Also contemplated by the invention, is a method for assaying for the presence of a first nucleic acid molecule in a sample, comprising contacting said sample with a second nucleic acid molecule, where the second nucleic acid molecule comprises a nucleic acid sequence selected from the group of nucleic acid sequences shown in FIG. 10 and the complements of the nucleic acid sequences shown in FIG. 10, wherein the nucleic acid sequence hybridizes to the first nucleic acid under high stringency conditions. In certain embodiments, the second nucleic acid molecule contains one or more polymorphism(s), such as those shown in FIG. 11.

The invention also relates to a vector comprising an isolated nucleic acid molecule of the invention (e.g., a sequence as shown in FIG. 10 or the complement of a sequence as shown in FIG. 10) operably linked to a regulatory sequence, as well as to a recombinant host cell comprising the vector. The invention also provides a method for producing a polypeptide encoded by an isolated nucleic acid molecule having a polymorphism, comprising culturing the recombinant host cell under conditions suitable for expression of the nucleic acid molecule.

Also contemplated by the invention is a method of assaying for the presence of a polypeptide encoded by an isolated nucleic acid molecule of the invention in a sample, the method comprising contacting the sample with an antibody that specifically binds to the encoded polypeptide.

The invention further pertains to a method of identifying an agent that alters expression of a SLIT-3 nucleic acid, comprising: contacting a solution containing a nucleic acid comprising the promoter region of the SLIT-3 gene operably linked to a reporter gene, with an agent to be tested; assessing the level of expression of the reporter gene in the presence of the agent; and comparing the level of expression of the reporter gene in the presence of the agent with a level of expression of the reporter gene in the absence of the agent; wherein if the level of expression of the reporter gene in the presence of the agent differs, by an amount that is statistically significant, from the level of expression in the absence of the agent, then the agent is an agent that alters expression of the SLIT-3 gene or nucleic acid. An agent identified by this method is also contemplated.

The invention additionally comprises a method of identifying an agent that alters expression of a SLIT-3 nucleic acid, comprising contacting a solution containing a nucleic acid of the invention or a derivative or fragment thereof, with an agent to be tested; comparing expression of the nucleic acid, derivative or fragment in the presence of the agent with expression of the nucleic acid, derivative or fragment in the absence of the agent; wherein if expression of the nucleic acid, derivative or fragment in the presence of the agent differs, by an amount that is statistically significant, from the expression in the absence of the agent, then the agent is an agent that alters expression of the SLIT-3 nucleic acid. In certain embodiments, the expression of the nucleic acid, derivative or fragment in the presence of the agent comprises expression of one or more splicing variants(s) that differ in kind or in quantity from the expression of one or more splicing variant(s) the absence of the agent. Agents identified by this method are also contemplated.

Representative agents that alter expression of a SLIT-3 nucleic acid contemplated by the invention include, for example, antisense nucleic acids to a SLIT-3 gene or nucleic acid; a SLIT-3 gene or nucleic acid; a SLIT-3 polypeptide; a SLIT-3 gene or nucleic acid receptor; a SLIT-3 binding agent; a peptidomimetic; a fusion protein; a prodrug thereof; an antibody; and a ribozyme. A method of altering expression of a SLIT-3 nucleic acid, comprising contacting a cell containing a nucleic acid with such an agent is also contemplated.

The invention further pertains to a method of identifying a polypeptide which interacts with a SLIT-3 polypeptide (e.g., a SLIT-3 polypeptide encoded by a nucleic acid comprising one or more polymorphism(s) indicated in FIG. 11), comprising employing a yeast two-hybrid system using a first vector which comprises a nucleic acid encoding a DNA binding domain and a SLIT-3 polypeptide, splicing variant, or a fragment or derivative thereof, and a second vector which comprises a nucleic acid encoding a transcription activation domain and a nucleic acid encoding a test polypeptide. If transcriptional activation occurs in the yeast two-hybrid system, the test polypeptide is a polypeptide, which interacts with a SLIT-3 polypeptide.

In certain methods of the invention, a Type II diabetes therapeutic agent is used. The Type II diabetes therapeutic agent can be an agent that alters (e.g., enhances or inhibits) SLIT-3 polypeptide activity and/or SLIT-3 nucleic acid expression, as described herein (e.g., a nucleic acid agonist or antagonist). In another embodiment, a Type II diabetes therapeutic agent is an agent that alters (e.g., enhances or inhibits) polypeptide activity and/or nucleic acid expression of a member of the Robo family (e.g., robo 1, robo 2 or rig-1).

Type II diabetes therapeutic agents can alter polypeptide activity or nucleic acid expression of a SLIT-3 nucleic acid or member of the Robo family by a variety of means, such as, for example, by providing additional polypeptide or upregulating the transcription or translation of the nucleic acid encoding the SLIT-3 polypeptide or a polypeptide that is a member of the Robo family; by altering posttranslational processing of the polypeptide; by altering transcription of splicing variants; or by interfering with polypeptide activity (e.g., by binding to the polypeptide, or by binding to another polypeptide that interacts with SLIT-3 or a member of the Robo family, such as a SLIT-3 binding agent as described herein or some other binding agent of a member of the Robo family), by altering (e.g., downregulating) the expression, transcription or translation of a nucleic acid encoding SLIT-3 or the member of the Robo family, by altering activity of a polypeptide member of the Robo family; or by altering interaction among SLIT-3 and one or more members of the Robo family. In another embodiment, agents include those that alter metabolism or activity of a Robo family polypeptide (e.g., robo 1, Robo 2 or rig-1), such as Robo family agonists or antagonists, as well as agents that alter activity of a Robo family receptor.

In a further embodiment, the invention relates to Type II diabetes therapeutic agent, such as an agent selected from the group consisting of: a SLIT-3 nucleic acid or fragment or derivative thereof; a Robo family nucleic acid or fragment or derivative thereof; a polypeptide encoded by a SLIT-3 nucleic acid (e.g., encoded by a SLIT-3 nucleic acid having one or more polymorphism(s) such as those set forth in FIG. 11); a polypeptide encoded by a Robo family gene or nucleic acid; a SLIT-3 receptor; a Robo family receptor, a SLIT-3 binding agent; a Robo family binding agent, such as a robo 1 binding agent, a robo 2 binding agent and a rig-1 binding agent; a peptidomimetic; a fusion protein; a prodrug, an antibody; an agent that alters SLIT-3 gene or nucleic acid expression; an agent that alters a Robo family member nucleic acid expression; an agent that alters activity of a polypeptide encoded by a SLIT-3 gene; an agent that alters activity of a polypeptide encoded by a Robo family gene or nucleic acid; an agent that alters posttranscriptional processing of a polypeptide encoded by a SLIT-3 gene or nucleic acid; an agent that alters posttranscriptional processing of a polypeptide encoded by a member of the Robo family gene or nucleic acid; an agent that alters interaction of a SLIT-3 polypeptide with a SLIT-3 binding agent; an agent that alters interaction of a Robo family polypeptide with a Robo family binding agent, an agent that alters interaction of a SLIT-3 polypeptide with a Robo family member; an agent that alters transcription of splicing variants encoded by a SLIT-3 gene or nucleic acid; an agent that alters transcription of splicing variants encoded by a Robo family member gene or nucleic acid ;and ribozymes. The invention also relates to pharmaceutical compositions comprising at least one Type II diabetes therapeutic agent as described herein.

The invention also pertains to a method of treating a disease or condition associated with a SLIT-3 polypeptide (e.g., Type II diabetes) or with members of the Robo family (such as, robo 1, robo 2 and rig-1) in an individual, comprising administering a Type II diabetes therapeutic agent to the individual, in a therapeutically effective amount. In certain embodiments, the Type II diabetes therapeutic agent is a SLIT-3 agonist or an agonist of a member of the Robo family; in other embodiments, the Type II diabetes therapeutic agent is a SLIT-3 antagonist or an antagonist of a member of the Robo family. The invention additionally pertains to use of a Type II diabetes therapeutic agent as described herein, for the manufacture of a medicament for use in the treatment of Type II diabetes, such as by the methods described herein.

A transgenic animal comprising a nucleic acid selected from the group consisting of: an exogenous SLIT-3 gene or nucleic acid and a nucleic acid encoding a SLIT-3 polypeptide, is further contemplated by the invention.

In yet another embodiment, the invention relates to a method for assaying a sample for the presence of a SLIT-3 nucleic acid: comprising contacting the sample with a nucleic acid comprising a contiguous nucleotide sequence which is at least partially complementary to a part of the sequence of said SLIT-3 nucleic acid under conditions appropriate for hybridization, and assessing whether hybridization has occurred between a SLIT-3 nucleic acid and said nucleic acid comprising a contiguous nucleotide sequence which is at least partially complementary to a part of the sequence of said SLIT-3 nucleic acid; wherein if hybridization has occurred, a SLIT-3 nucleic acid is present in sample. In certain embodiments, the contiguous nucleotide sequence is completely complementary to a part of the sequence of said SLIT-3 nucleic acid. If desired, amplification of at least part of said SLIT-3 nucleic acid can be performed.

In certain other embodiments, the contiguous nucleotide sequence is 100 or fewer nucleotides in length and is either at least 80% identical to a contiguous sequence of nucleotides in one of the nucleic acid sequences as shown in FIG. 10, at least 80% identical to the complement of a contiguous sequence of nucleotides in one of the nucleic acid sequences as shown in FIG. 10, or capable of selectively hybridizing to said SLIT-3 nucleic acid.

In other embodiments, the invention relates to a reagent for assaying a sample for the presence of a SLIT-3 gene or nucleic acid, the reagent comprising a contiguous nucleotide sequence which is at least partially complementary to a part of the nucleic acid sequence of said SLIT-3 gene (nucleic acid) or the reagent is completely complementary to a part of the nucleic acid sequence of said SLIT-3 gene or nucleic acid. Also contemplated by the invention is a reagent kit, e.g., for assaying a sample for the presence of a SLIT-3 nucleic acid, comprising (e.g., in separate containers) one or more labeled nucleic acids comprising a contiguous nucleotide sequence which is at least partially complementary to a part of the nucleic acid sequence of the SLIT-3 nucleic acid, and reagents for detection of said label. In certain embodiments, the labeled nucleic acid comprises a contiguous nucleotide sequence that is completely complementary to a part of the nucleotide sequence of said SLIT-3 gene or nucleic acid. In other embodiments, the labeled nucleic acid can comprise a contiguous nucleotide sequence which is at least partially complementary to a part of the nucleotide sequence of said SLIT-3 gene or nucleic acid, and which is capable of acting as a primer for said SLIT-3 nucleic acid when maintained under conditions for primer extension.

The invention also provides for the use of a nucleic acid which is 100 or fewer nucleotides in length and which is either: a) at least 80% identical to a contiguous sequence of nucleotides in one of the nucleic acid sequences as shown in FIG. 10; b) at least 80% identical to the complement of a contiguous sequence of nucleotides in one of the nucleic acid sequences as shown in FIG. 10; or c) capable of selectively hybridizing to said SLIT-3 nucleic acid, for assaying a sample for the presence of a SLIT-3 nucleic acid.

In yet another embodiment, the use of a first nucleic acid which is 100 or fewer nucleotides in length and which is either: a) at least 80% identical to a contiguous sequence of nucleotides in one of the nucleic acid sequences as shown in FIG. 10; b) at least 80% identical to the complement of a contiguous sequence of nucleotides in one of the nucleic acid sequences as shown in FIG. 10; or c) capable of selectively hybridizing to said SLIT-3 nucleic acid; for assaying a sample for the presence of a SLIT-3 gene that has at least one nucleotide difference from the first nucleic acid (e.g., a SNP or marker as set forth in FIG. 11), such as for diagnosing a susceptibility to a disease or condition associated with a SLIT-3.

The invention also relates to a method of diagnosing a susceptibility to Type II diabetes in an individual, comprising determining the presence or absence in the individual of a certain haplotypes (combinations of genetic markers). In one aspect of the invention of diagnosising a susceptibility of the disease, methods are described comprising screening for one of the at-risk haplotypes in the SLIT3 gene that is more frequently present in an individual susceptible to Type II diabetes, compared to the frequency of its presence in the general population, wherein the presence of an at-risk haplotype is indicative of a susceptibility to Type II diabetes. An “at-risk haplotype” is intended to embrace one or a combination of haplotypes described herein over the SLIT3 gene that show high correlation to Type II diabetes. In one embodiment, the at-risk haplotype is characterized by the presence of at least one single nucleotide polymorphisms as described in FIG. 11. In one embodiment, a haplotype associated with Type II diabetes or a susceptibility to type II diabetes comprises one or more haplotypes identified in Table 2 (haplotypes identified as A1, A2, A3, A4, A5, A6, B1, B2, B3, B4 and B5) or Table 5 (haplotypes identified as C1, C2, C3, C4, and C5). In other embodiments, the at-risk haplotype comprises comprising one or more of the markers set forth in FIG. 11, at the 5q35 locus, wherein the presence of the haplotype is diagnostic of susceptibility to Type II diabetes. In another embodiment, the invention relates to a method of diagnosing a susceptibility to Type It diabetes in an individual, comprising determining the presence or absence in the individual of a haplotype comprising one or more of the following markers: one or more of the to markers in the haplotypes set forth in Table 2 and/or Table 5, and/or one or more of the makers set forth in Table 4, at the 5q35 locus. The presence or absence of the haplotype can be determined by various methods, including, for example, using enzymatic amplification of nucleic acid from the individual, electrophoretic analysis, restriction fragment length polymorphism analysis and/or sequence analysis.

The invention also relates to a method of diagnosing a susceptibility to Type II diabetes in an individual, comprising: obtaining a nucleic acid sample from said individual; and analyzing the nucleic acid sample for the presence or absence of a haplotype comprising one or more of the markers set forth in FIG. 11, at the 5q35 locus, wherein the presence of the haplotype is diagnostic for a susceptibility to Type II diabetes. In another embodiment, the invention relates to a method of diagnosing a susceptibility to Type II diabetes in an individual, comprising: obtaining a nucleic acid sample from said individual; and analyzing the nucleic acid sample for the presence or absence of a haplotype comprising one or more of the following markers: one or more markers set forth in the haplotypes set forth in Table 2 and/or Table 5, and/or one or more of the makers set forth in Table 4, at the 5q35 locus, wherein the presence of the haplotype is diagnostic for a susceptibility to Type II diabetes.

Also described herein is a method of diagnosing Type II diabetes or a susceptibility to Type II diabetes in an individual, comprising determining the presence or absence in the individual of a haplotype comprising one or more markers and/or single nucleotide polymorphisms as shown in FIG. 11 in the locus on chromosome 5q35, wherein the presence of the haplotype is diagnostic of Type II diabetes or a susceptibility to Type II diabetes.

A method for the diagnosis and identification or a susceptibility to Type II diabetes in an individual is also described, comprising: screening for an at-risk haplotype in the SLIT-3 nucleic acid that is more frequently present in an individual susceptible to Type It diabetes compared to an individual who is not susceptible to Type II diabetes wherein the at-risk haplotype increases the risk significantly. In certain embodiments, the significant increase is at least about 20% or the significant increase is identified as an odds ratio of at least about 1.2.

A major application of the current invention involves prediction of those at higher risk of developing a Type II diabetes. Diagnostic tests that define genetic factors contributing to Type II diabetes might be used together with or independent of the known clinical risk factors to define an individual's risk relative to the general population. Better means for identifying those individuals at risk for Type II diabetes should lead to better prophylactic and treatment regimens, including more aggressive management of the current clinical risk factors.

Another application of the current invention is the specific identification of a rate-limiting pathway involved in Type II diabetes. A disease gene with genetic variation that is significantly more common in diabetic patients as compared to controls represents a specifically validated causative step in the pathogenesis of Type II diabetes. That is, the uncertainty about whether a gene is causative or simply reactive to the disease process is eliminated. The protein encoded by the disease gene defines a rate-limiting molecular pathway involved in the biological process of Type II diabetes predisposition. The proteins encoded by such Type II genes or its interacting proteins in its molecular pathway may represent drug targets that may be selectively modulated by small molecule, protein, antibody, or nucleic acid therapies. Such specific information is greatly needed since the population affected with Type II diabetes is growing.

A third application of the current invention is its use to predict an individual's response to a particular drug, even drugs that do not act on SLIT3 or its pathway. It is a well-known phenomenon that in general, patients do not respond equally to the same drag. Much of the differences in drug response to a given drug is thought to be based on genetic and protein differences among individuals in certain genes and their corresponding pathways. Our invention defines the association of SLIT3 with Type II diabetes. Some current or future therapeutic agents may be able to affect this gene directly or indirectly and therefore, be effective in those patients whose Type II diabetes risk is in part determined by the SLIT3 genetic variation. On the other hand, those same drugs may be less effective or ineffective in those patients who do not have at risk variation in the SLIT3 gene. Therefore, SLIT3 variation or haplotypes may be used as a pharmacogenomic diagnostic to predict drug response and guide choice of therapeutic agent in a given individual.

### BRIEF DESCRIPTION OF THE DRAWINGS

The foregoing and other objects, features and advantages of the invention will be apparent from the following more particular description of preferred embodiments of the invention, as illustrated in the accompanying drawings.

FIGS. 1A-1O9 shows the SLIT-3 genomic DNA (SEQ ID NO: 1). This sequence is taken from NCBI Build33. The numbering in FIG. 1, as well as the “Start” and “End” numbers in all of the Figures, refer to the location in Chromosome is 5 in NCBI Build33. The numbering in FIG. 1 refers to the last base in the line immediately preceding the number; the numbers are in decreasing order because of the “reverse orientation” of the gene.

FIG. 2 is a series of graphs showing the results of a genome-wide scan using 906 microsatellite markers. Results are shown for three phenotypes: all type 2 diabetics (solid lines), obese diabetics (dotted lines) and non-obese diabetics (dashed lines). The multipoint allele-sharing LOD-score is on the vertical axis, and the centiMorgan distance from the P-terminus of the chromosome is on the horizontal axis.

FIG. 3 graphically shows the multipoint allele-sharing LOD-score of the locus on chromosome 5 after 38 microsatellite markers have been added to the framework set in a 40-cM interval, from 160 cM to 200 cM. Results are shown for the same three phenotypes as in FIG. 2; all II diabetics (solid line), non-obese (dashed line) and obese diabetics (dotted SNPs).

FIG. 4 graphically depicts single-marker and haplotype association within the 1-LOD-drop for 590 non-obese diabetics vs 477 unrelated population controls. The location of the markers/haplotypes is on the horizontal axis and the corresponding two-sided P-value on the vertical axis. All haplotypes with a P-value less than 0.01 are shown. The horizontal bars indicate the span of the corresponding haplotypes and the marker density is shown at the bottom of the figure. All locations refer to NCBI Build33 and the 1-LOD-drop spans from 167.64 to 171.28 Mb.

FIG. 5 schematically shows the locations of genes and markers in region A The microsatellites used in the locus-wide association study are shown as filled circles at the top. The filled boxes indicate the locations of exons, or clusters of exons, for SLIT3. Note that the orientation of the SLIT3 gene, 5′ to 3′, is from righ to left. The shaded boxes indicate the location and size of the neighboring genes, ODZ2, KIAA0869, RARS and PANK3, and the grey horizontal bars indicate the span of the six most significant microsatellite haplotypes in the region.

FIG. 6 graphically depicts the single-marker allelic association within SLIT3. a The exonic structure of SLIT3. b Location of all microsatellites (top) and SNPs (bottom) used in the association analysis. c Single-marker allelic association, with P-value<0.05, across SLIT3. The plot shows negative log P-values versus the physical location in megabases (NCBI33). The grey horizontal bar at the bottom indicates the span of the most significant microsatellite and SNP haplotoype C1. The same horizontal scale is used for a, b and c.

FIGS. 7A-Q shows the DNA sequence of microsatellites employed for the C05 locus wide association (including Build33 locations).

FIG. 8 shows the Build33 location of SLIT3 exons.

FIGS. 9A and B shows the Build33 location of SNPs found across SLIT3 after sequencing of the exons and flanking sequences.

FIGS. 10A-P2 shows the DNA sequence of the SNPs identified across SLIT3.

FIGS. 11A-C shows the Build33 location of all SNPs and microsatellites identified as polymorphic across SLIT3.

FIGS. 12A-F shows the DNA sequence of the microsatellites employed for the association studies across SLIT3 (including Build33 locations).

FIGS. 13A-C shows the names of the SNPs and microsatellites employed for the association analysis across SLIT3.

FIGS. 14A and B shows the amino acid sequence for the SLIT3 protein.

### DETAILED DESCRIPTION OF THE INVENTION

Extensive genealogical information for a population with population-based lists of patients with Type II diabetes has been combined with powerful gene sharing methods to map a locus on chromosome 5q35. Diabetics and their relatives were genotyped with a genome-wide marker set Due to the role obesity plays in the development of diabetes, the material was fractionated according to body mass index (BMI). Presented herein are results of a genome wide search of genes that cause Type II diabetes in Iceland.

Loci Associated with Diabetes

Evidence for genes causing the early onset monogenic form of diabetes have been previously identified. Mutations in six genes have been discovered that cause MODY, or maturity onset diabetes of the young. MODY1-MODY6 are due to mutations in HNF4a, glucokinase, HNF1a, IPF1, HNF1b and NEUROD1 (MODY1: Yamagata K, et al., Nature 384:458-460 (1996); MODY2: Froguel P, F et al. Nature 356: 162-164(1992); MODY3: Yamagata, K., et al., Nature 384: 455-458 (1996); MODY4: Yoshioka M., et al. Diabetes May;46(5):887-94 (1997) MODY5: Horikawa, Y., et al. Nat. Genet. 17: 384-3 85 (1997) MODY6: Kristinsson S. Y., et al., Diabetologia November;44(11):2098-103 (2001)).

One gene has been identified as a disease gene that contributes to the late-onset form of diabetes, the calpain 10 gene (CAPN10). CAPN10, was identified though a genome-wide screen of Mexican American sibpairs with diabetes (Horikawa, Y., et al., Nat. Genet. 26(2) 163-175(2000)). The risk allele has been shown to be associated with impaired regulation of glucose-induced secretion and decreased rate of insulin-stimulated glucose disposal (Lynn, S., et al., Diabetes, 51(1): 247-250 (2002); Sreenan, S. K., et al., Diabetes 50(9) 2013-2020 (2001) and Baier, L. J., et al., J. Clin. Invest. 106(7) R69-73 (2000)).

Many genome-wide screens in a variety of populations have been performed that have resulted in major loci for Diabetes. Loci are reported on chromosome 2q37 (Hanis, C. L., et al., Nat. Genet., 13(2):161-166 (1996)), chromosome 15q21 (Cox, et al., Nat. Genet. 21(2):213-215 (1999)), chromosome 10q26 (Duggirala, R., et al., Am. J. Hum. Genet., 68(5):1149-1164 (2001)), chromosome 3p (Ehm, M. G., et al., Am. J. Hum. Genet., 66(6):1871-1881 (2000)) in Mexican Americans, and chromosomes 1q21-23 and 11q23-q25 (Hanson R. L. et al., Am J. Hum Genet., 63(4):1130-1138 (1998)) in PIMA Indians. In the Caucasian population, linkages have been observed to chromosome 12q24 in Finns (Mahtani, et al., Nat. Genet., 14(1):90-4 (1994)), chromosome 1q21-q23 in Americans in Utah (Elbein, S. C., et al., Diabetes, 48(5):1175-1182 (1999)), chromosome 3q27-pter in French families (Vionnet, N., et al., Am. J. Hum. Genet. 67(6): 1470-80 (2000) and chromosome 18p11 in Scandinavians (Parker, A., et al., Diabetes, 50(3) 675-680 (2001)). A recent study reported a major locus in indigenous Australians on chromosome 2q24.3 (Busfield, F, et al., Am. J. Hum. Genet., 70(2): 349-357 (2002)). Many other studies have resulted in suggestive loci or have replicated these loci.

Association studies have been reported for Type II diabetes. Most of these studies show modest association to the disease in a group of people but do not account for the disease. Altshuler et al. reviewed the association work that has been done and concluded that association to only one of 16 genes revealed held up to scrutiny. Altshuler et al. confirmed that the Pro12A1a polymorphism in PPARg is associated with Type II diabetes. Until now, there have been no linkage studies in Type II diabetes linking the disease to chromosome 5q35.

SLIT-3

The invention described herein has linked Type II diabetes to a gene known as SLIT-3 (slit homolog 3 (Drosophila)). Drosophila SLIT is a secreted protein involved in midline patterning. In the Drosophila nervous system, SLIT is produced by midline glial cells and functions as a chemorepellant to prevent the recrossing of commissural axons (Kidd, T., et al., Cell, 96:785-794 (1999)). This is mediated by the Roundabout, or Robo, family of receptors, which contain five Ig domains, three fibronectin type III (FNIII) repeats, a single transmembrane domain, and an intracellular domain with a number of conserved cytoplasmic motifs (Kidd, T., et al., Cell, 92:205-215 (1998)). There are three vertebrate SLIT genes and three distinct Robo genes (robo1, robo2, rig-1) (Yuan, S. S., et al., Dev. Biol., 207:62-75 (1999); Brose, K., et al., Cell, 96:795-806 (1999)). At the vertebrate midline, it has been proposed that expression of SLITs and Robos controls the crossing axons in the spinal cord (Zou, Y., et al., Cell, 102:363-375 (2000)), retinal ganglion cell axons at the optic chiasm (Fricke, C., et al., Science, 292:507-510 (2001); Erskine, L., et al., J. Neurosci., 20:4975-4982 (2000); and Niclou, S. P., et al., J. Neurosci., 20:4962-4974 (2000)), and fibers of the corpus callosum (Shu, T., and L. J. Richards, J. Neurosci., 21:2749-2758 (2001)).

In addition to the Robo family of receptors, SLIT proteins have been demonstrated to be ligands for CDO in myogenic differentiation (Kang, J. S., et al., J. Cell Biol., 143:403-413 (1998)), DCC (a netrin receptor) in midline crossing (Stein, E and M. Tessier-Lavigne, Science, 291:1928-1938 (2001)) and glypican (expressed in motor neurons).

In situ hybridization studies in the developing mouse embryo have shown that SLIT-3 is expressed in the developing brain, eyes, ears, nose and limb buds (Yuan, S. S., et al., Dev. Biol., 207:62-75 (1999)). In addition, in situ hybridizations of rat brains (embryonic and adult) demonstrate that SLIT proteins have a role in both the developing and adult brain (Marillat, V., et al., J. Comp. Neurol., 442: 130-155 (2002)).

Itoh et al. cloned human SLIT-3 in 1998 (Itoh, A., et al., Brain Res. Mol. Brain Res., 62:175-186 (1998)). The mRNA size for SLIT-3 is 5.5 kb and 9.5 kb with the smaller transcript being predominant. The open reading frame (ORF) is 4569 bp and encodes a 1523 amino acid polypeptide. Northern blot analysis revealed expression in fetal lung and fetal kidney. In human adult tissues, SLIT-1 and SLIT-3 mRNAs are mainly expressed in the brain, spinal cord, and thyroid, respectively. SLIT-2 is also expressed weakly in the adrenal gland, thyroid, and trachea. SLIT-3 is expressed in the ovary, heart and small intestine (Itoh, A., et al., ibid.). Based on expression patterns of these proteins, it has been suggested that SLIT proteins have a role in the endocrine system as well as in the nervous system. SLIT-3 has been proposed to contribute to the morphogenesis of the endocrine system (Itoh, A., et al., ibid.). Expression in pancreas, liver, skeletal muscle, adipose tissue, small intestine and hypothalamus has been observed with PCR on tissue-specific cDNA (data not shown). PCR analysis of radiation hybrid panels mapped the SLIT-3 gene to chromosome 5q35 (Nakayama, M, et al., Genomics, 51:27-34 (1998)).

The predicted amino acid sequences of human SLIT-2 and SLIT-3 display the same domain structures and an approximately 60% similarity to SLIT-1. SLIT-1, SLIT-2 and SLIT-3 all comprise a putative signal peptide, four units of tandem arrays of leucine-rich repeats (LRR) bordering amino- and carboxy-terminal conserved flanking regions (LRR-NR, LRR-CR), two groups of EGF-like motif repeats, an Agrin-Laminin Perlecan-SLIT (ALPS) conserved domain, and a cysteine-rich (Cys-rich) carboxy-terminal domain. However, they have no putative transmembrane domains as predicted by hydrophobicity plots. As such, the SLIT proteins contain many binding domains and may interact with one or more proteins. Compared with the drosophila SLIT protein, the three human SLIT proteins share a number of EGF-like motifs and the repeat number of LRR1 and LRR3. The region containing four units of LRR is the most conserved element among the human SLIT proteins, and the number of amino acids that make up this region is completely conserved among the three proteins (Itoh, A., et al., ibid.).

It has been proposed that SLIT-3 has potentially unique functions not shared by other SLIT proteins (Little, M. H., et al., Am. J. Physiol. Cell. Physiol., 281: C485-495 (2001)). The cellular distribution and processing of mammalian SLIT-3 gene product has been characterized in kidney epithelial cells. SLIT-3, but not SLIT-2, is predominantly localized within the mitochondria. In confluent epithelial monolayers, SLIT-3 is also transported to the cell surface. However, there is no evidence of SLIT-3 proteolytic processing similar to that seen for SLIT-2. SLIT-3 contains an NH₂-terminal mitochondrial localization signal that can direct a reporter green fluorescent protein to the mitochondria. The equivalent region from SLIT-1 cannot elicit mitochondrial targeting. As such, it has been concluded the SLIT-3 protein is targeted and localized to two distinct sites within epithelial cells: the mitochondria, and, in more confluent cells, the cell surface. Targeting to both locations is driven by specific NH₂-terminal sequences.

Studies have shown a link between disruptions in mitochondrial functioning and Type II diabetes. Indeed, mitochondrial dysfunction in the β-cell is well described (Maechler, P., and C. B. Wollheim, Nature, 414:807-812 (2001)). Genetic disturbances in mitochondrial DNA (mtDNA) can lead to the development of a number of genetic disorders that present with a Type II diabetes phenotype. A mutation in the mitochondrial tRNA (Leu)(UUR) gene was described in a large pedigree with maternally transmitted Type II diabetes and deafness (van den Ouweland, J. M., et al., Nat. Genet., 1:368-371 (1992)). Decreases in mtDNA copy number have also been linked to the pathogenesis of diabetes. Although the contribution of variations in mtDNA to the development of Type II diabetes in unknown, a 50% decrease in mtDNA copy number in skeletal muscle of Type II diabetes has been observed (Antonetti, D. A., et al., J. Clin. Invest., 95:1383-1388 (1 995)). Reduced mtDNA content has also been reported in peripheral blood cells in such patients even before the onset of the disease (Lee, H. K., et al., Diabetes Res. Clin. Pract., 42:161-167 (1998)).

Described herein is the first known linkage study of Type II diabetes showing a connection to chromosome 5q35. Based on the linkage studies conducted, a direct relationship between Type II diabetes and the locus on chromosome 5q35, in particular the SLIT-3 gene, has been discovered.

### NUCLEIC ACIDS OF THE INVENTION

SLIT-3 Nucleic Acids, Portions and Variants

Accordingly, the invention pertains to isolated nucleic acid molecules comprising human SLIT-3 nucleic acid. The term, “SLIT-3 nucleic acid,” as used herein, refers to an isolated nucleic acid molecule encoding a SLIT-3 polypeptide (e.g., a SLIT-3 gene). The SLIT-3 nucleic acid molecules of the present invention can be RNA, for example, mRNA, or DNA, such as cDNA and genomic DNA. DNA molecules can be double-stranded or single-stranded; single stranded RNA or DNA can be either the coding, or sense, strand or the non-coding, or antisense strand. The nucleic acid molecule can include all or a portion of the coding sequence of the gene and can further comprise additional non-coding sequences such as introns and non-coding 3′ and 5′ sequences (including regulatory sequences, for example).

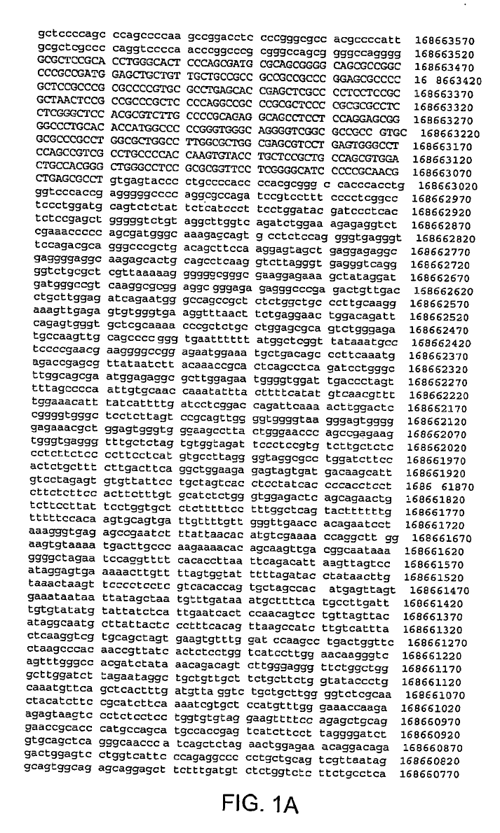

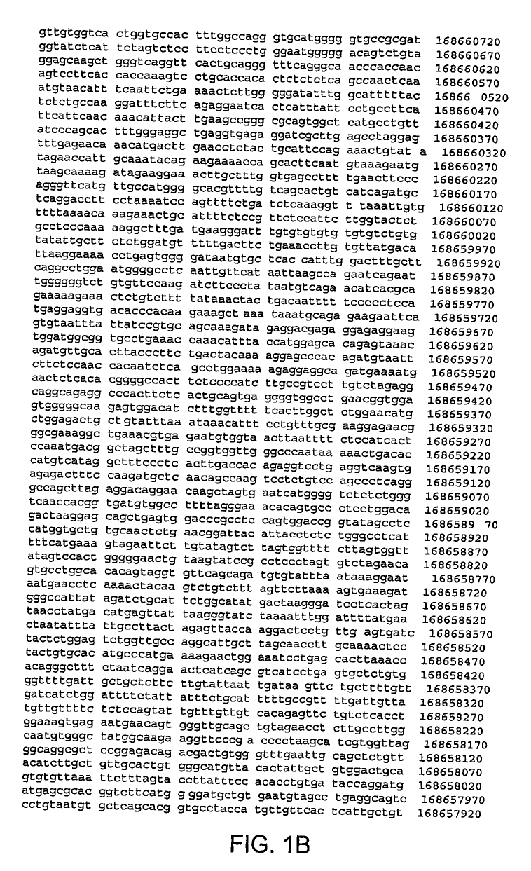

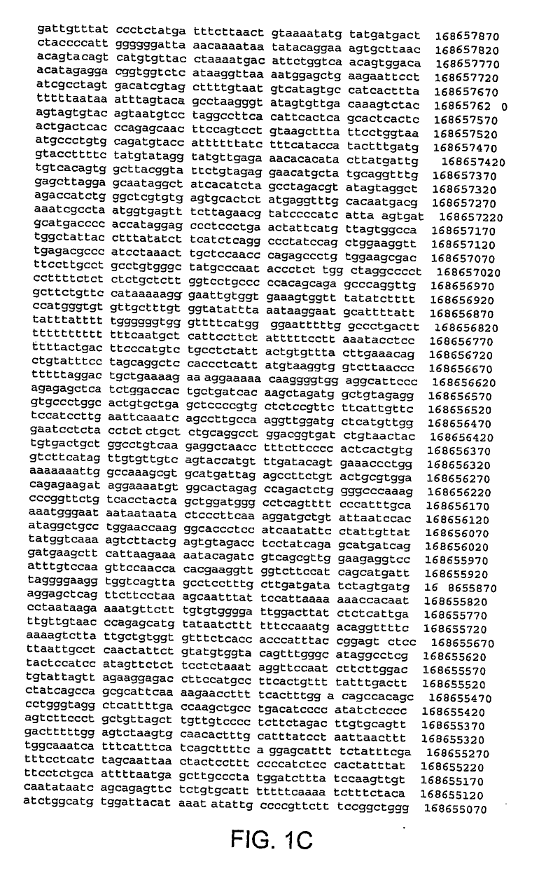

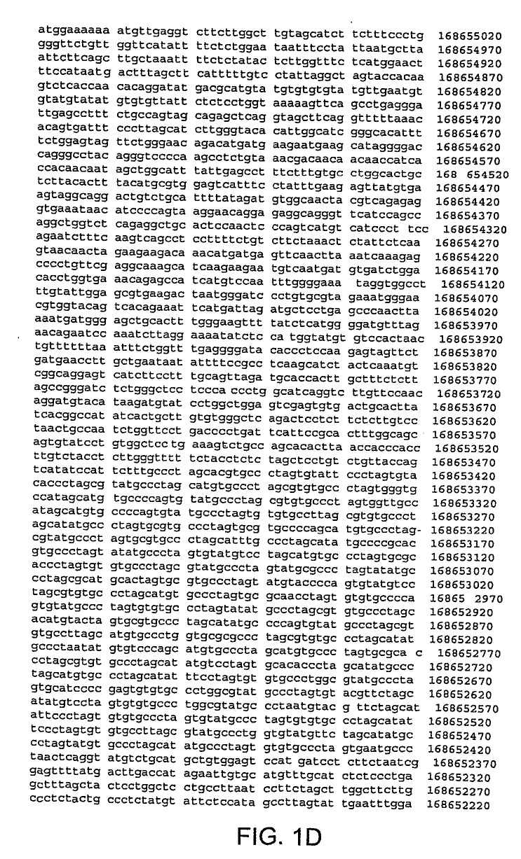

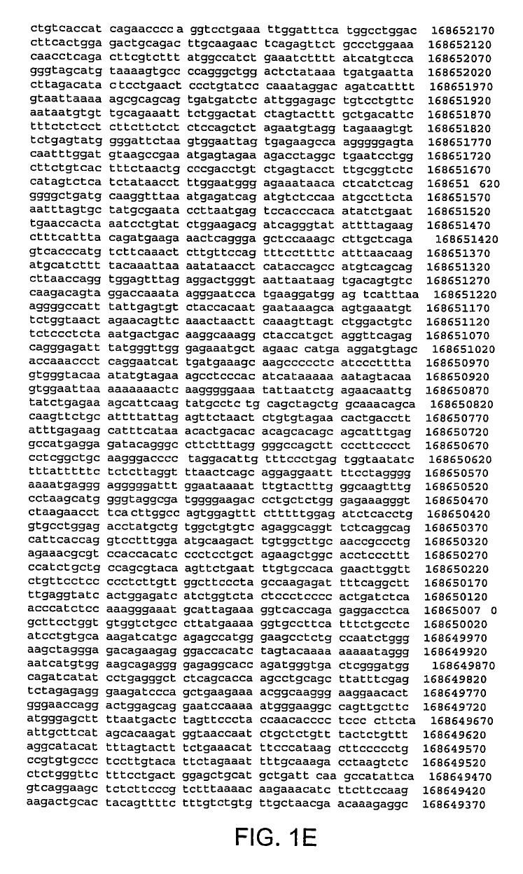

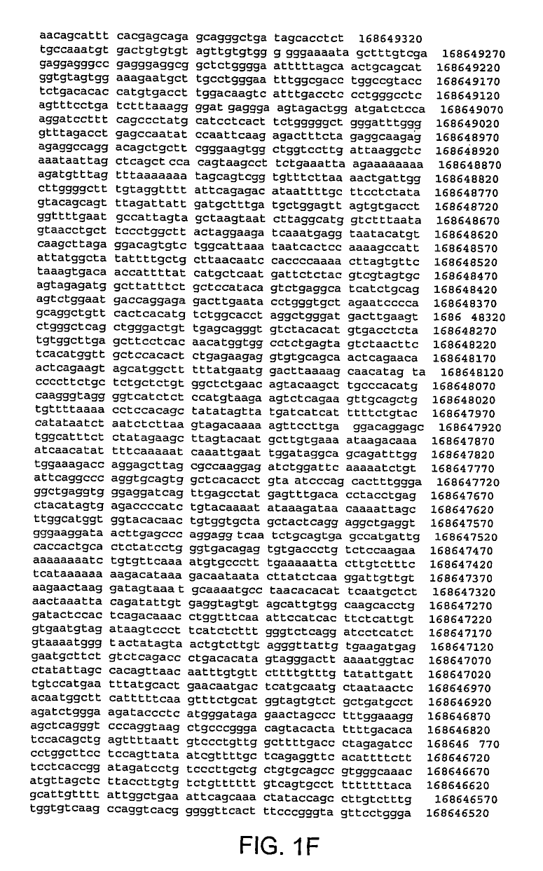

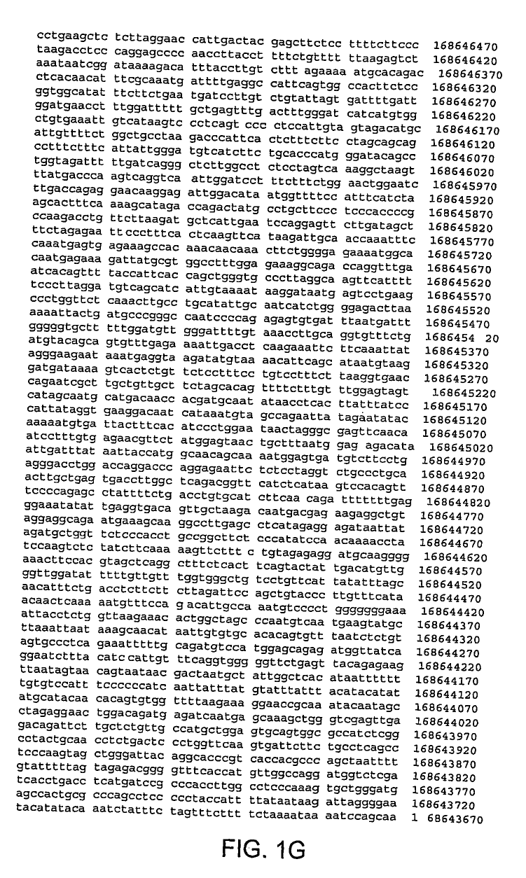

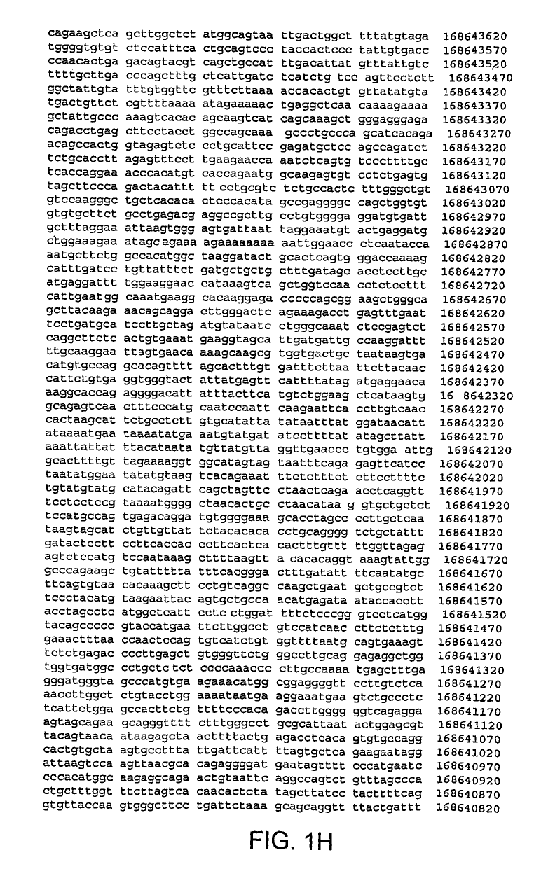

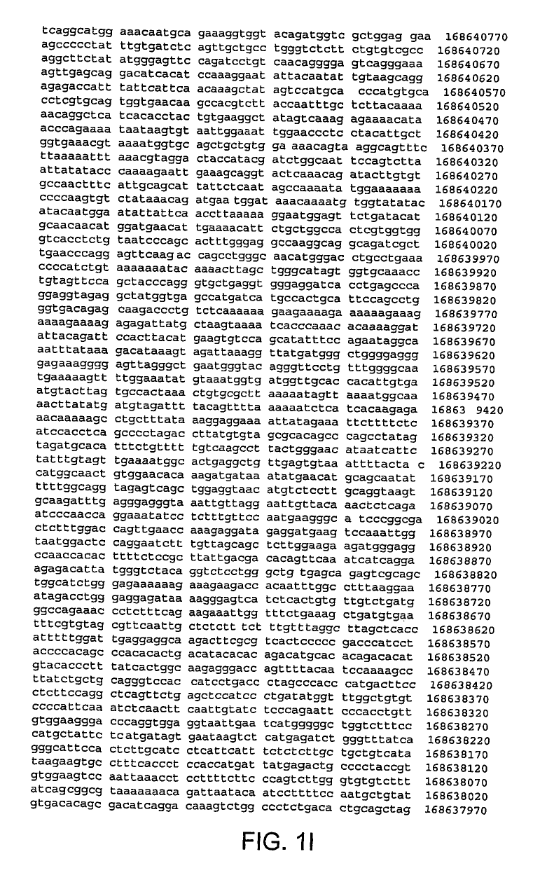

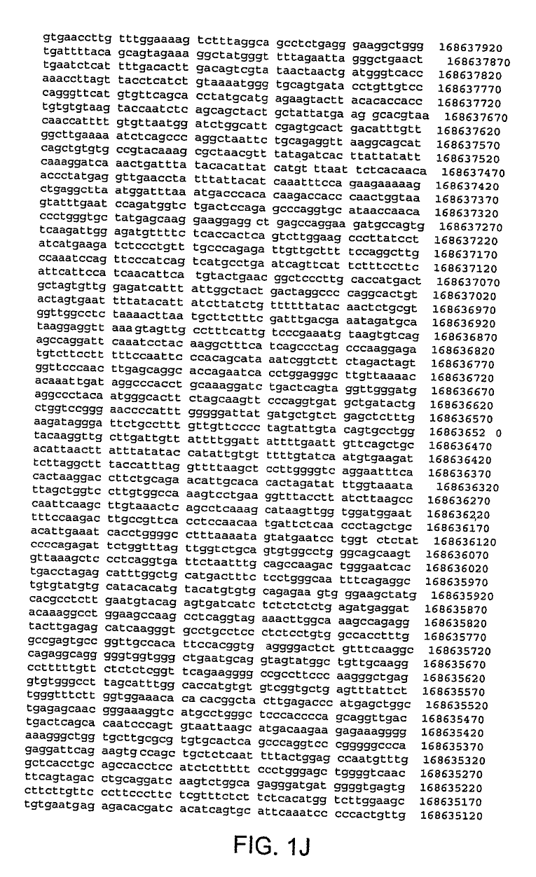

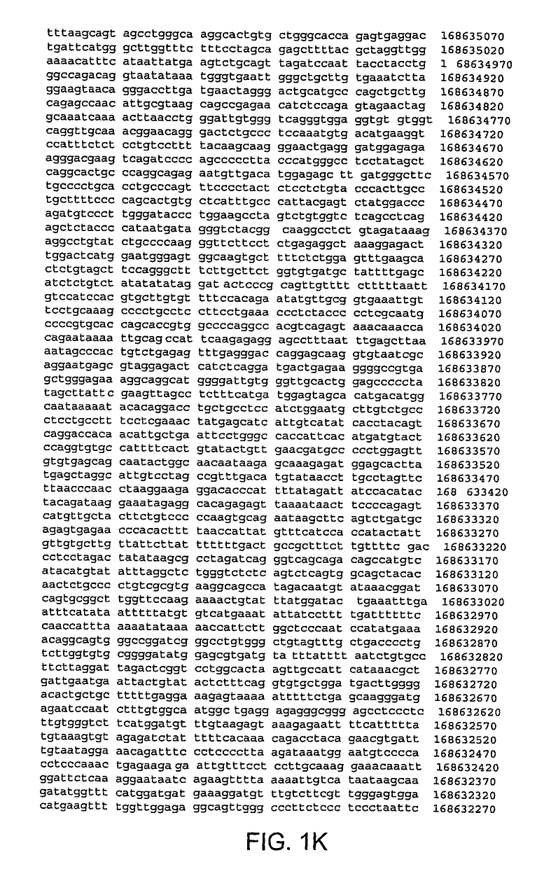

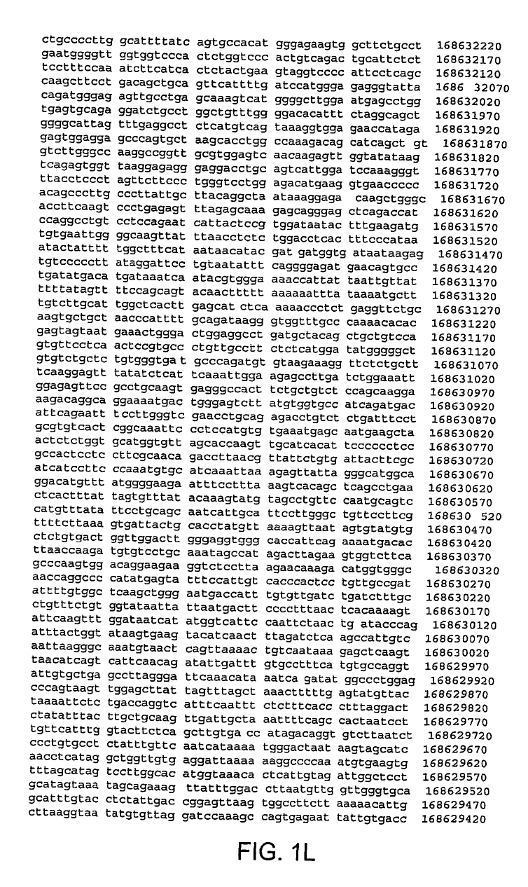

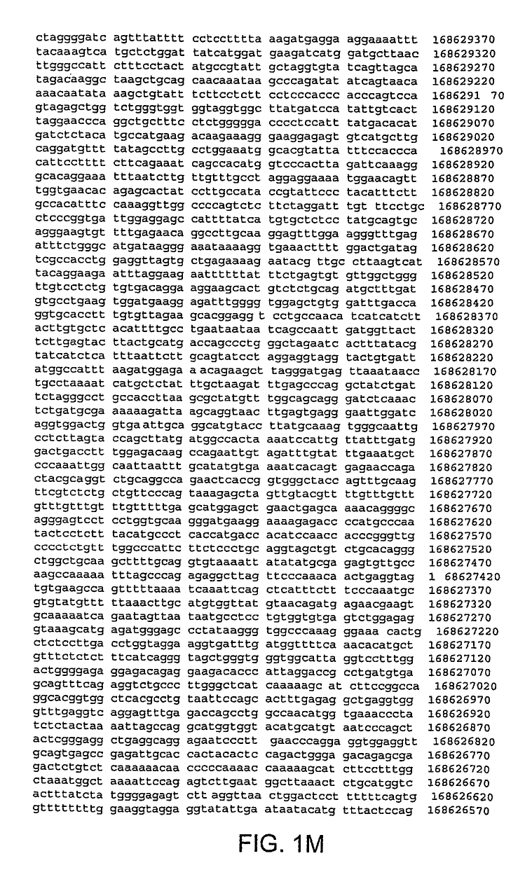

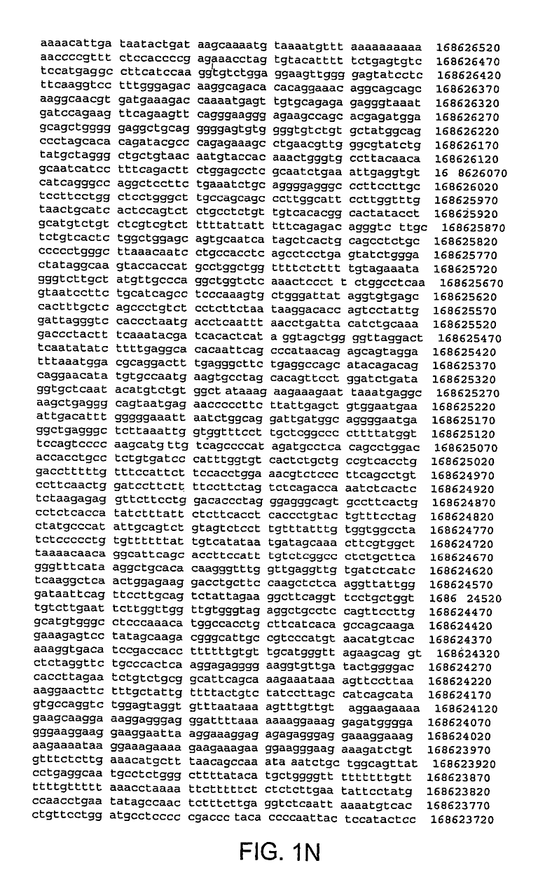

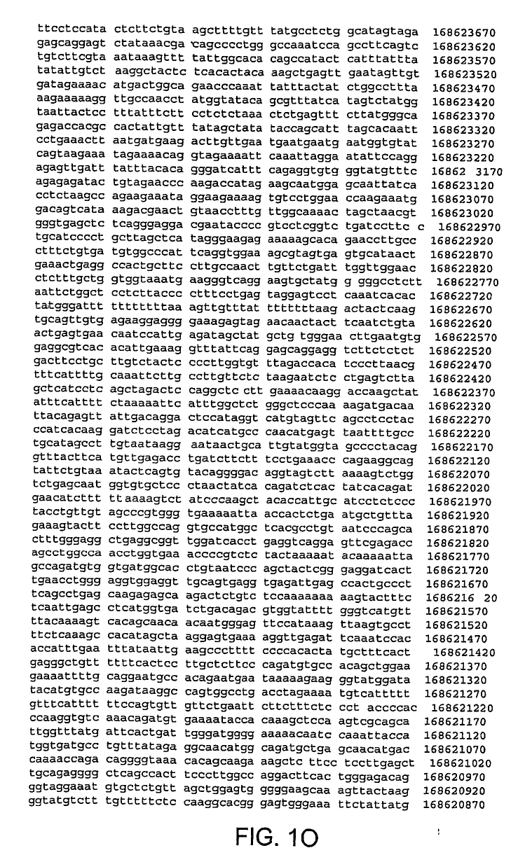

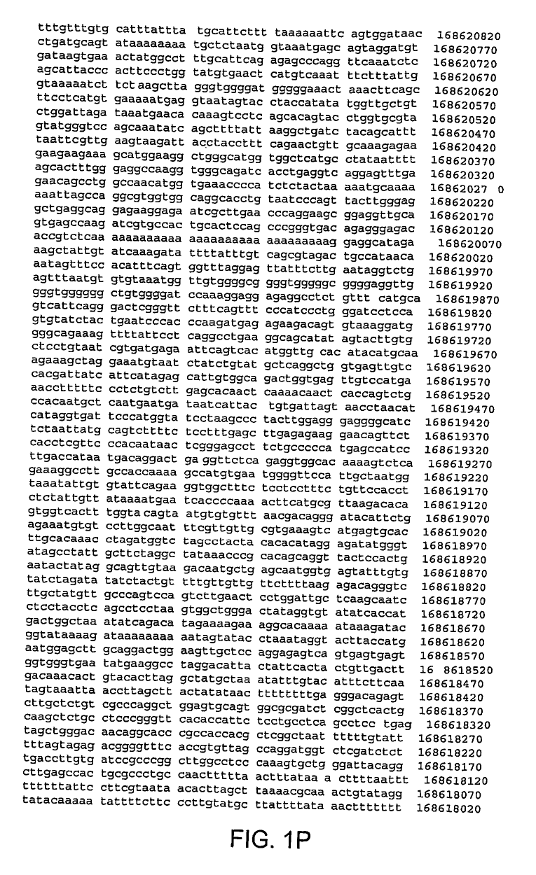

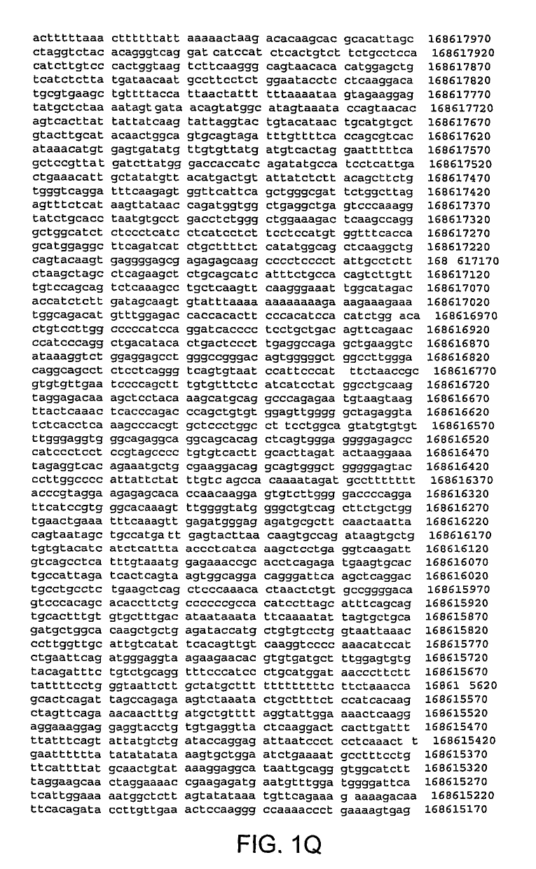

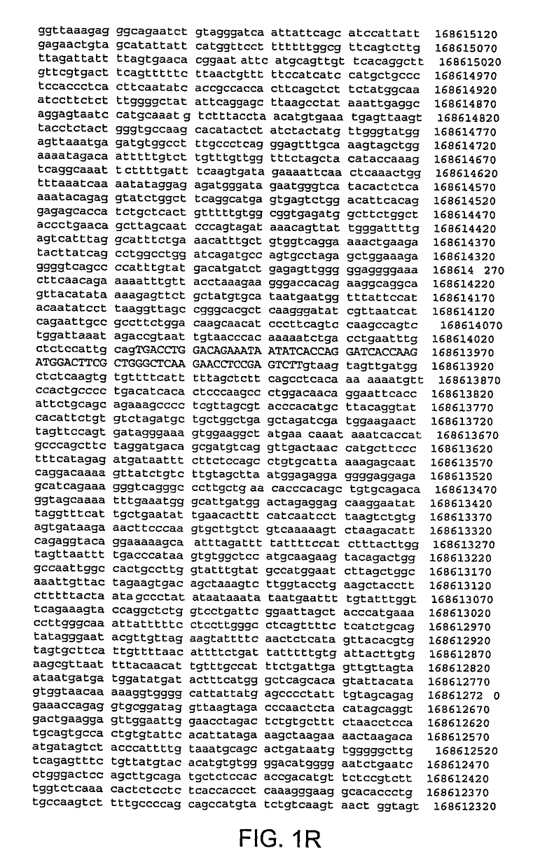

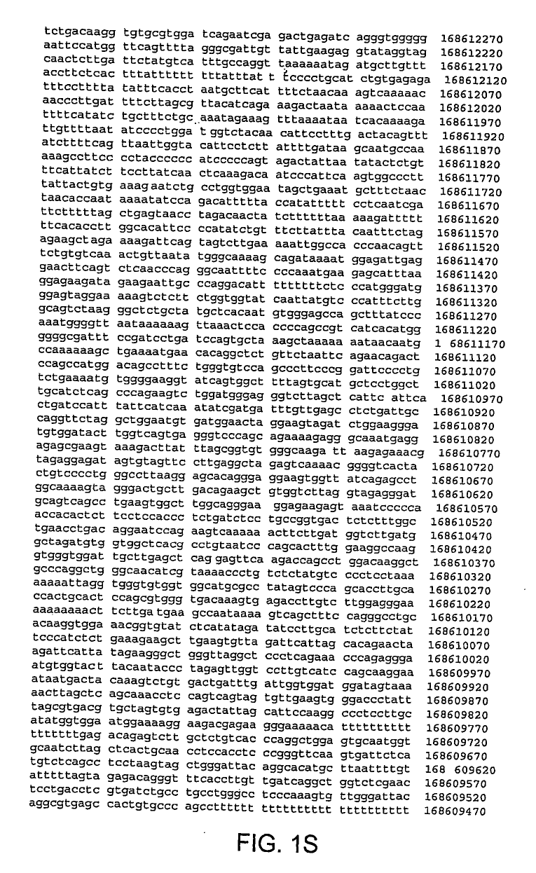

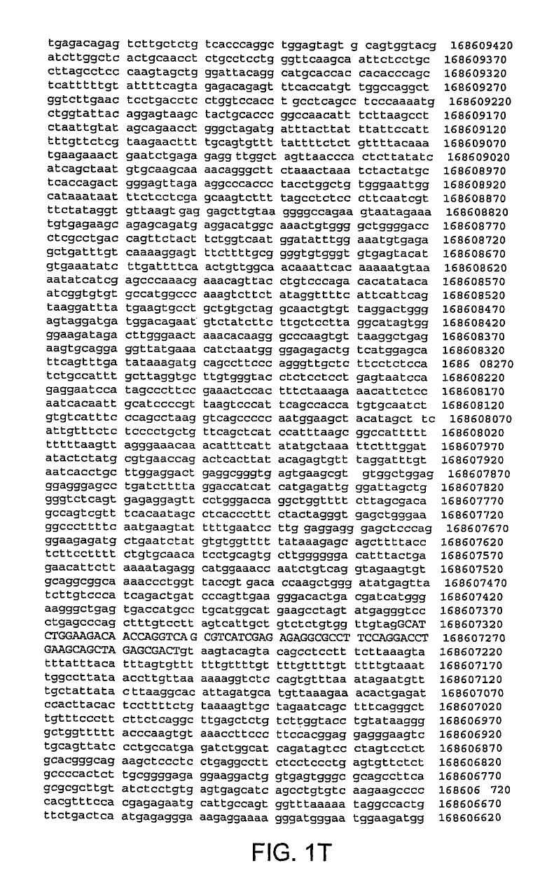

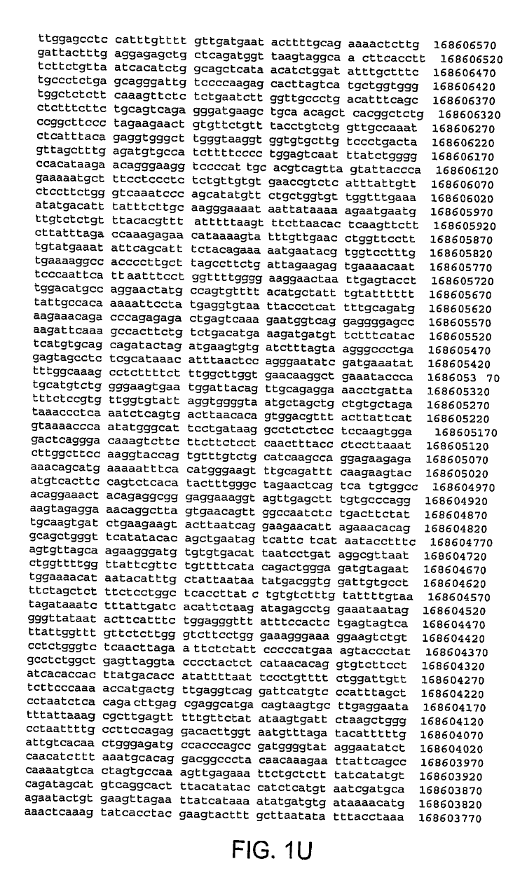

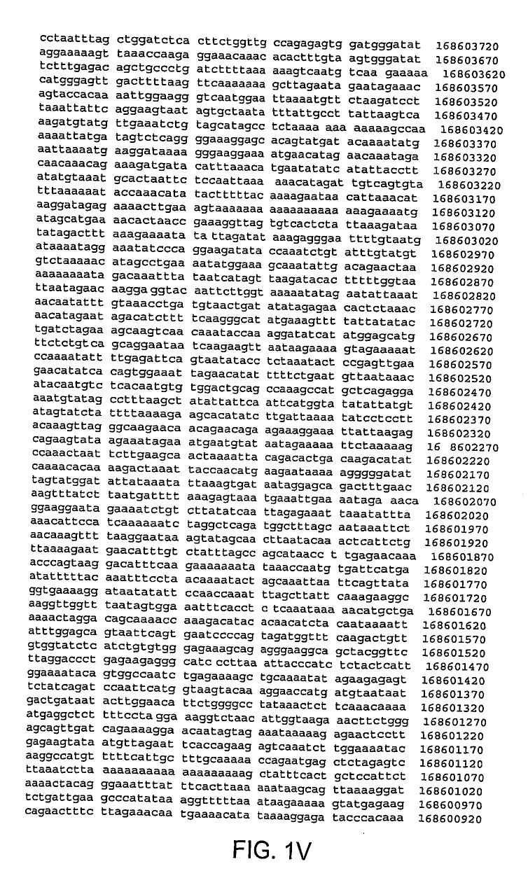

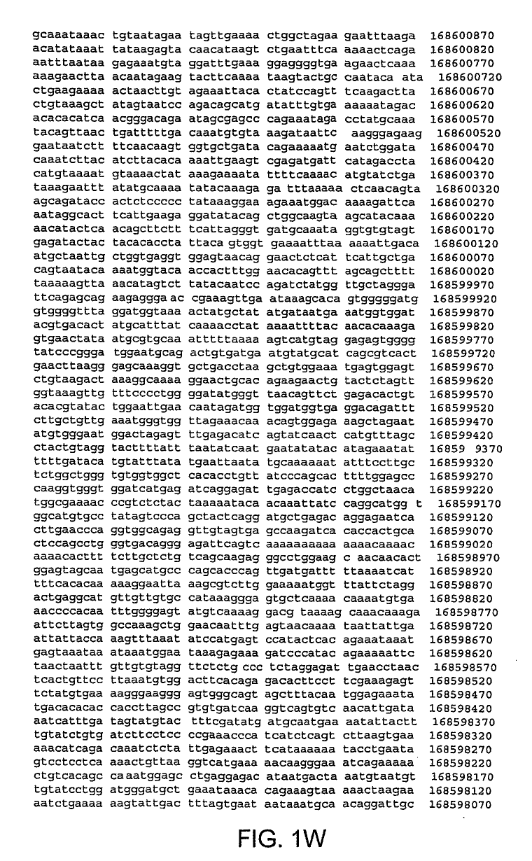

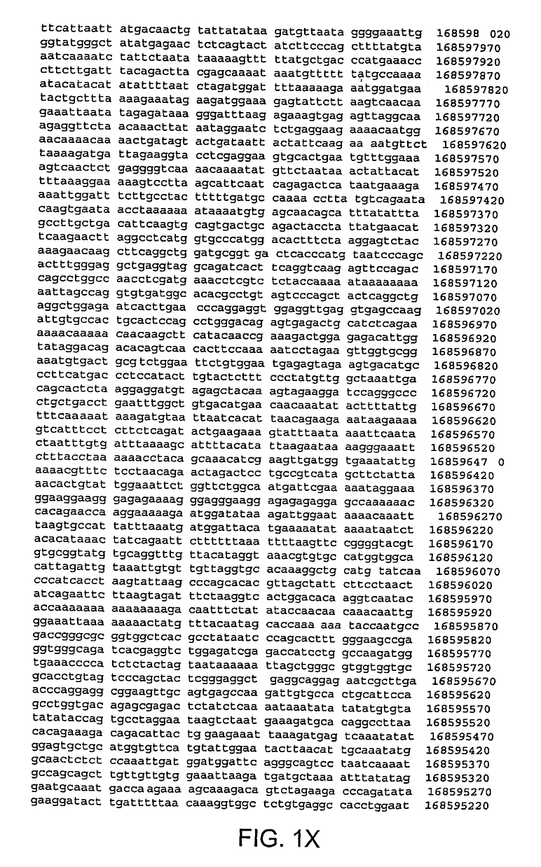

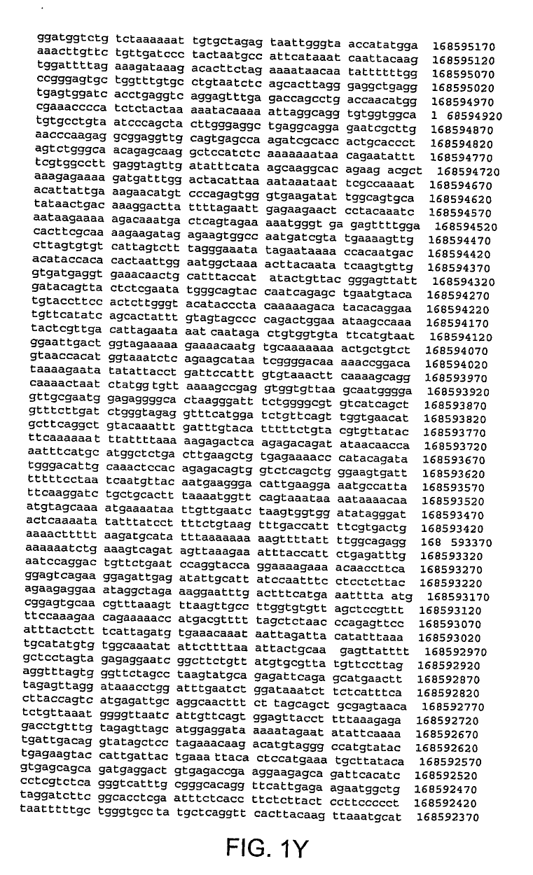

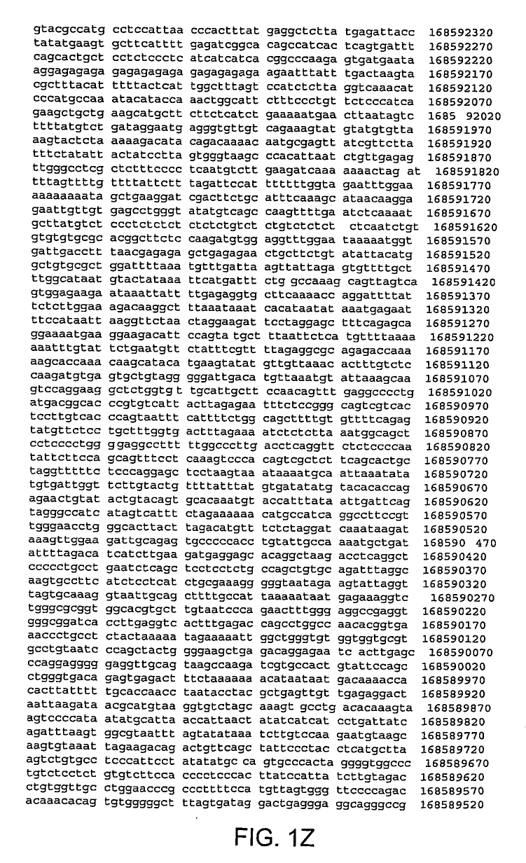

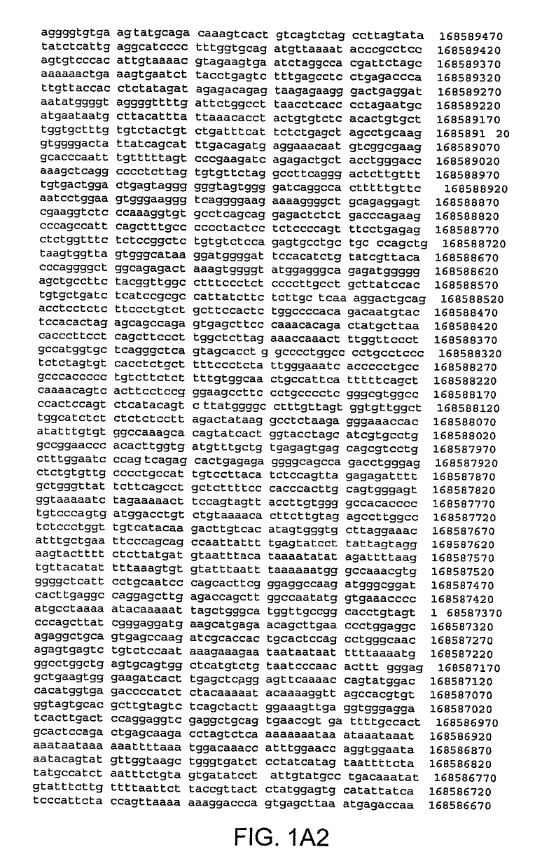

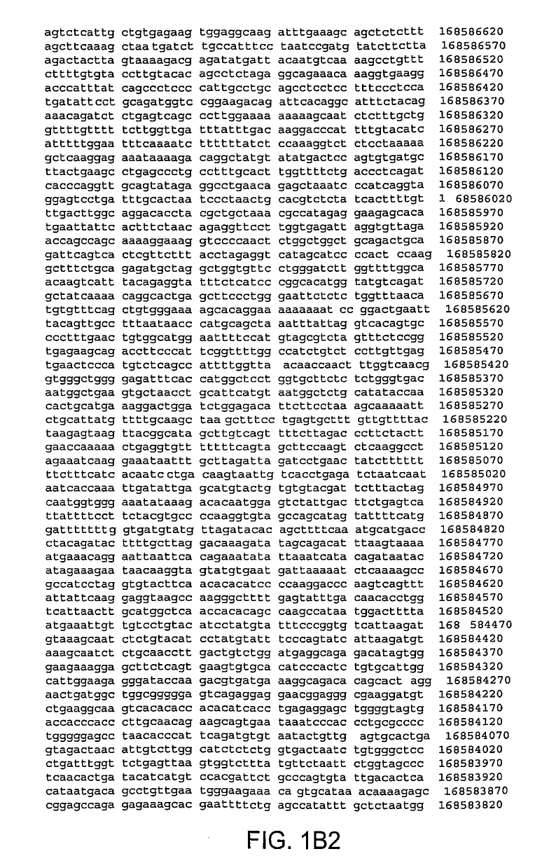

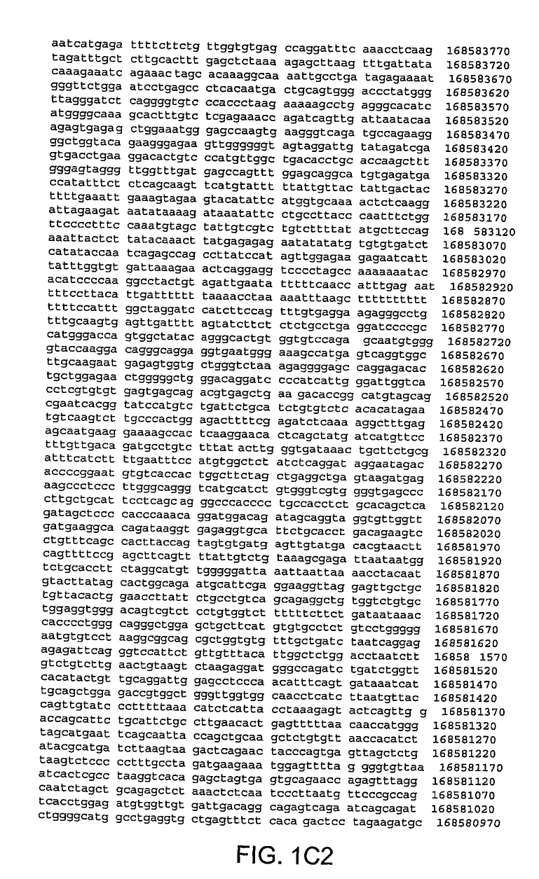

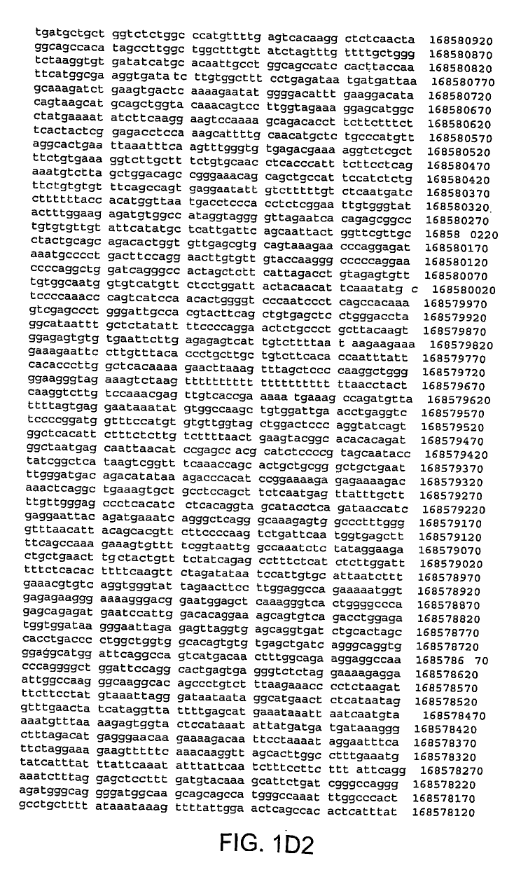

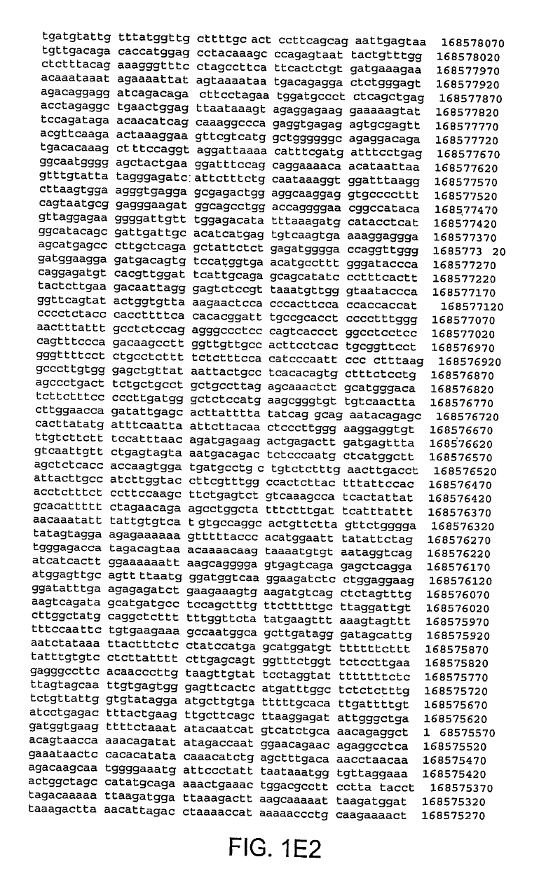

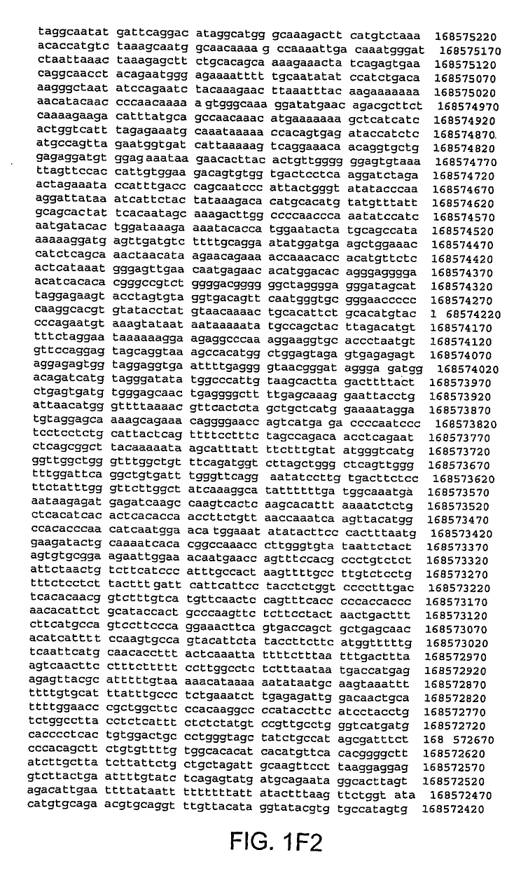

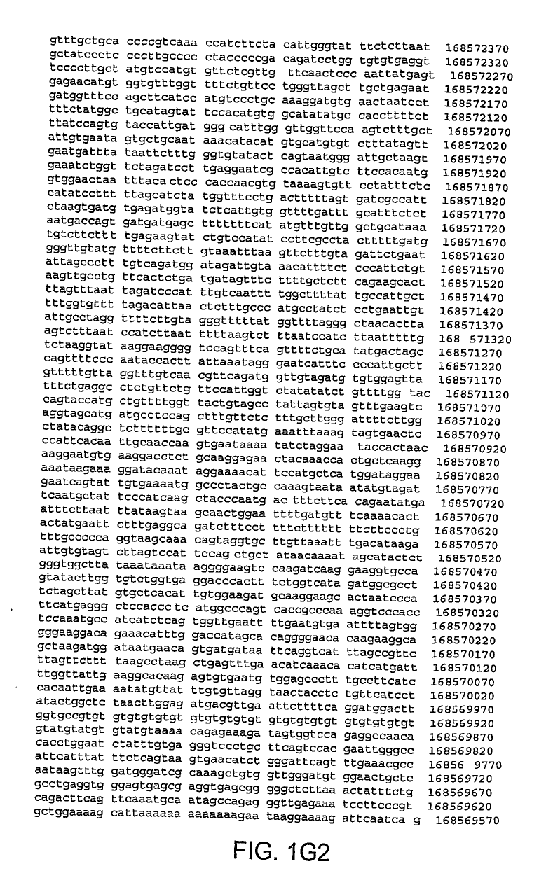

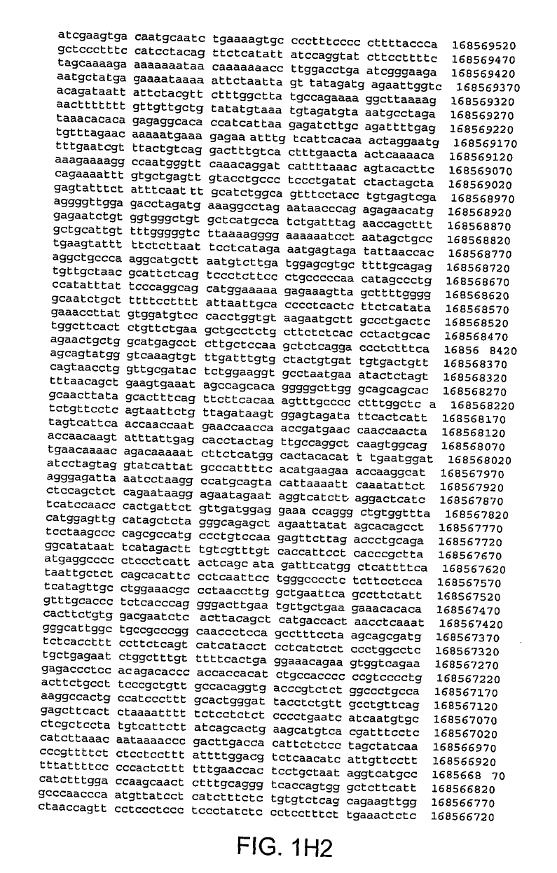

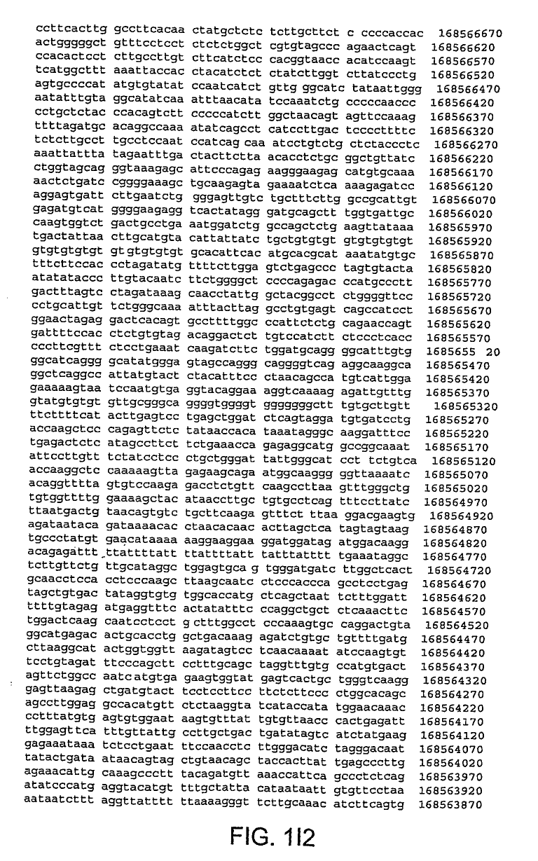

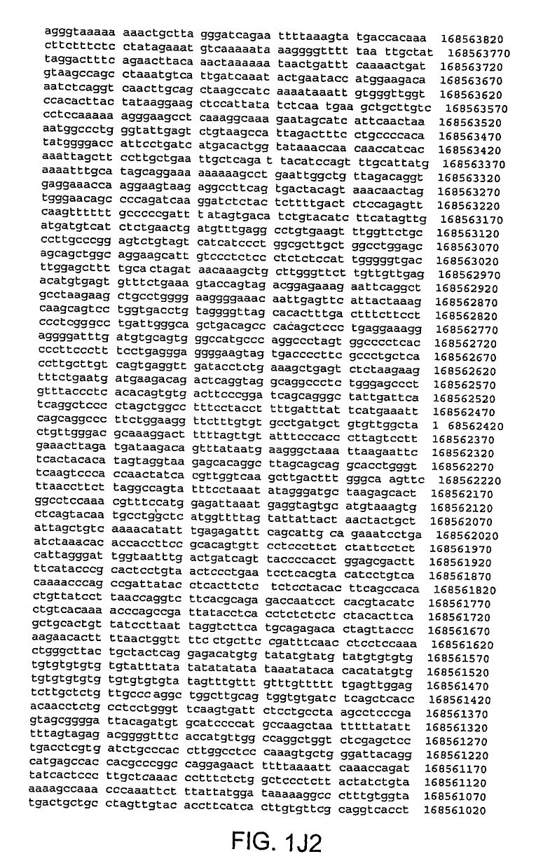

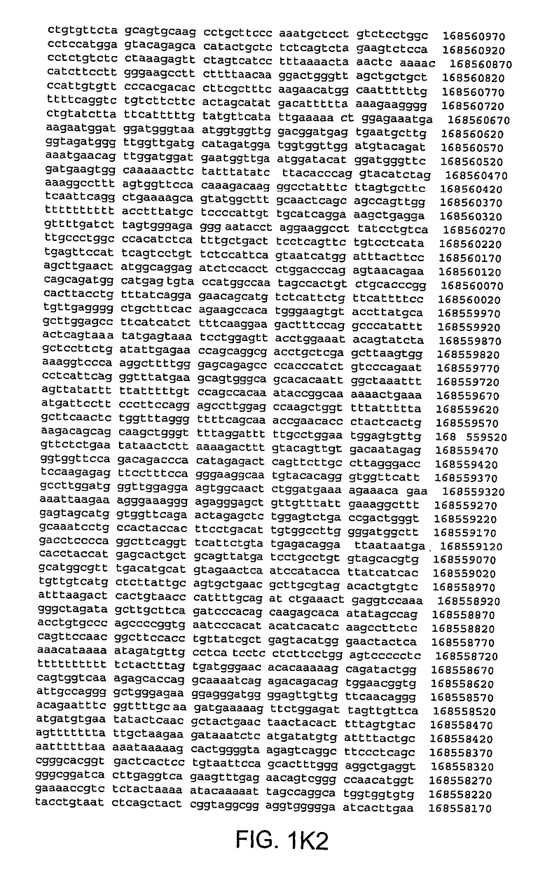

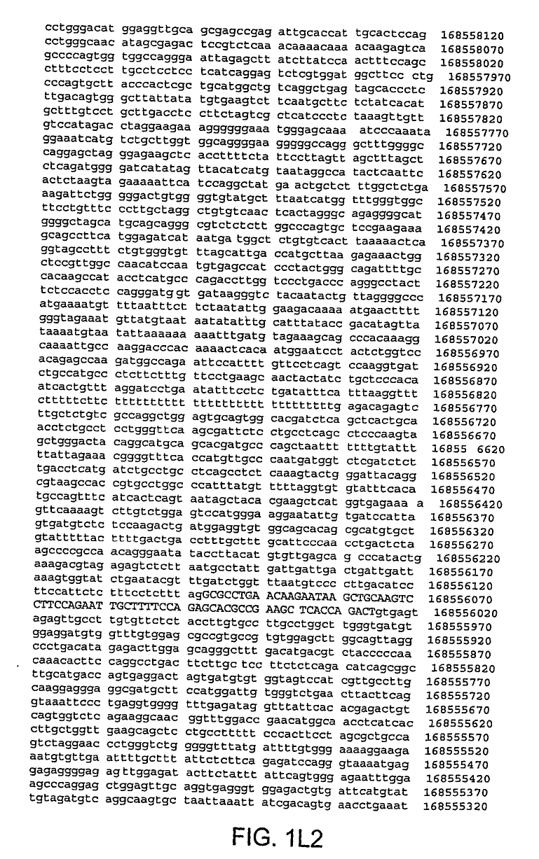

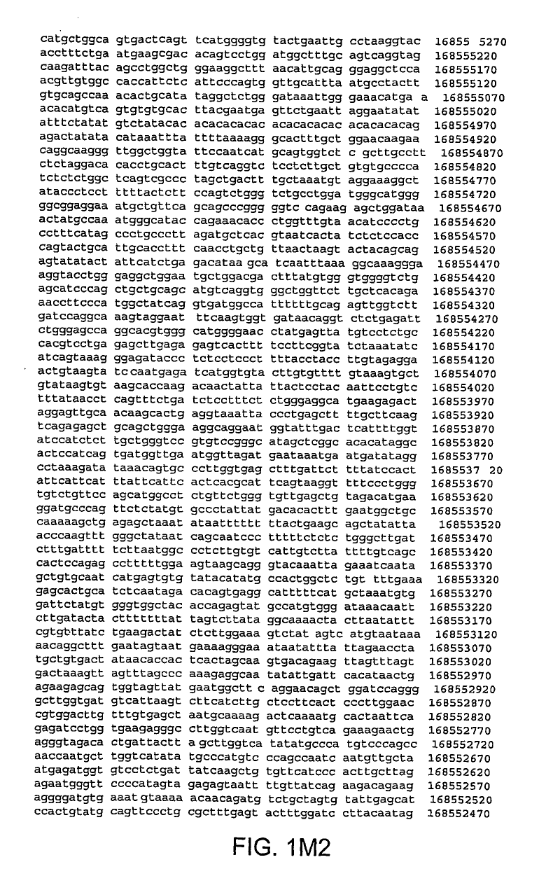

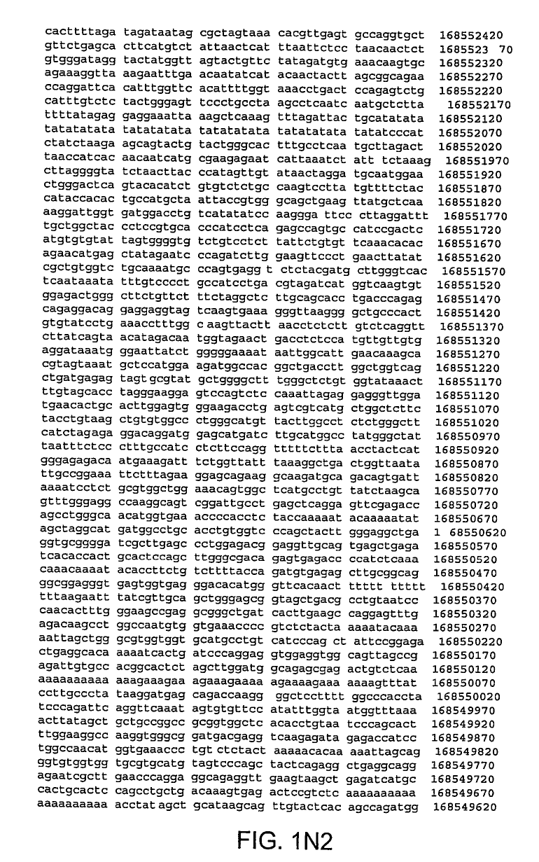

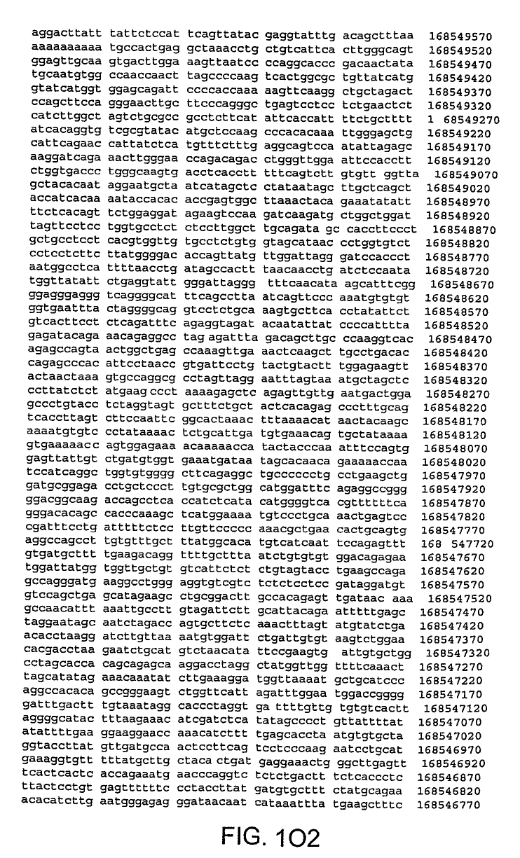

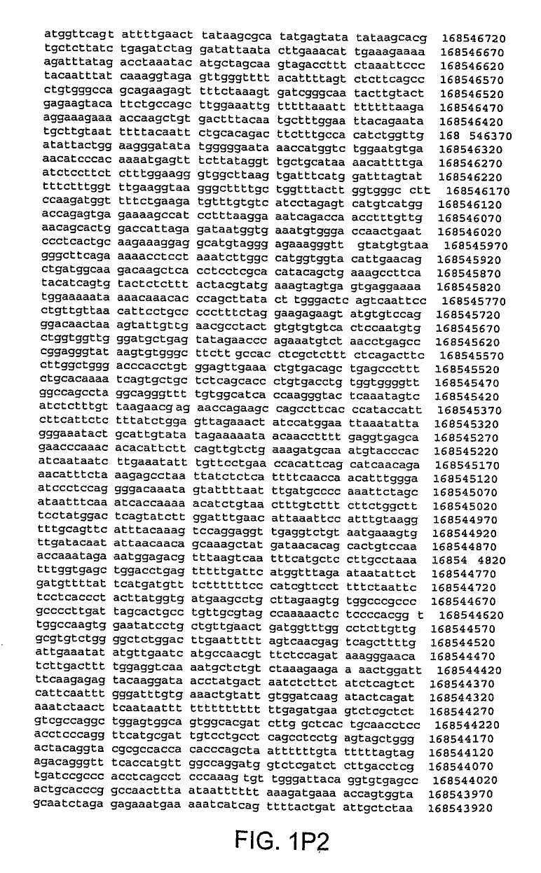

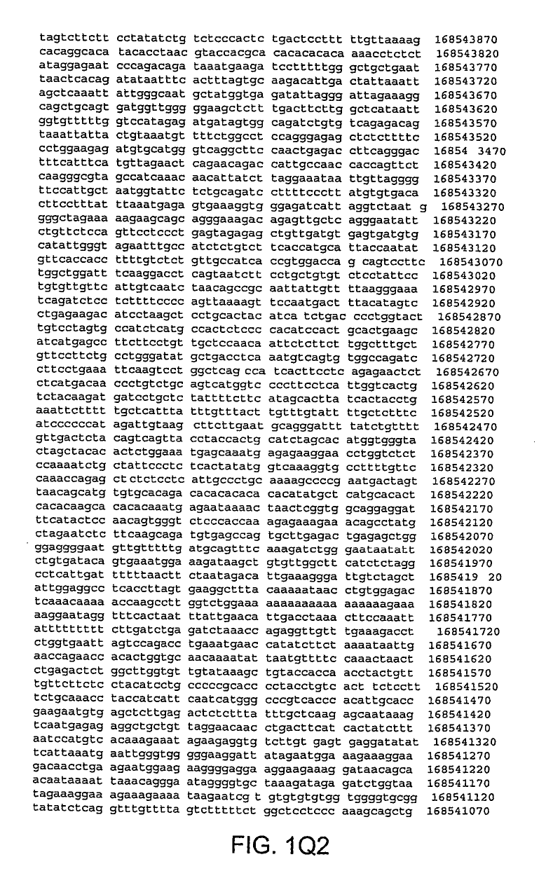

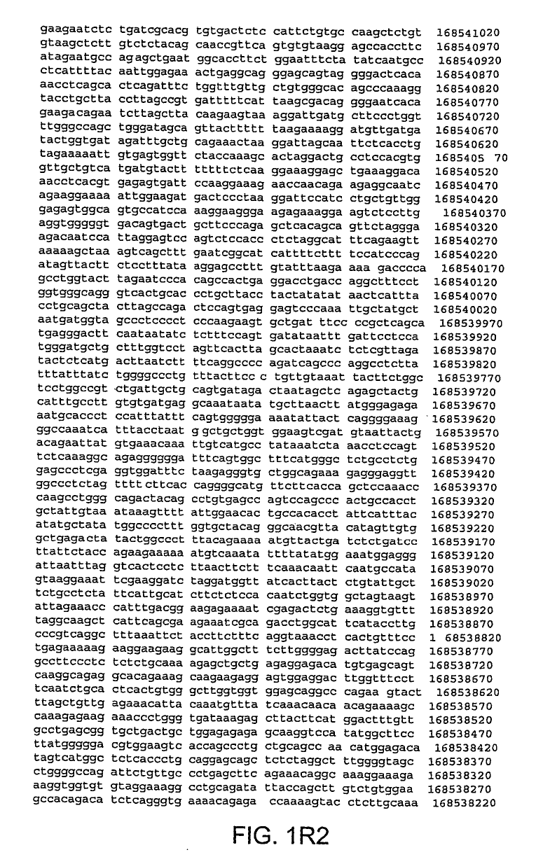

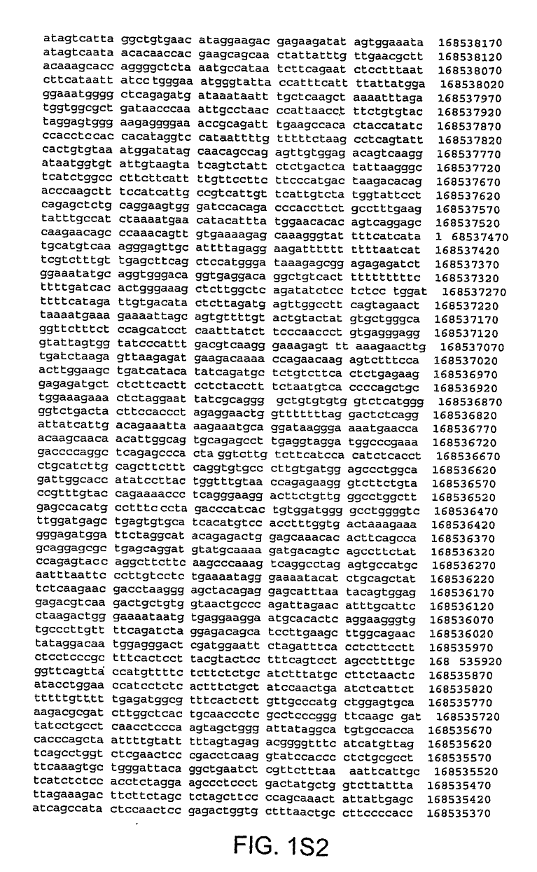

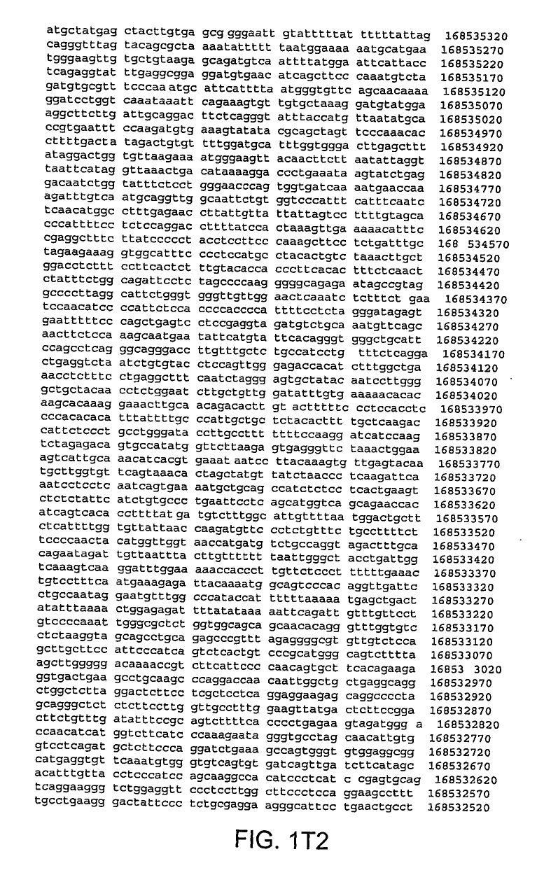

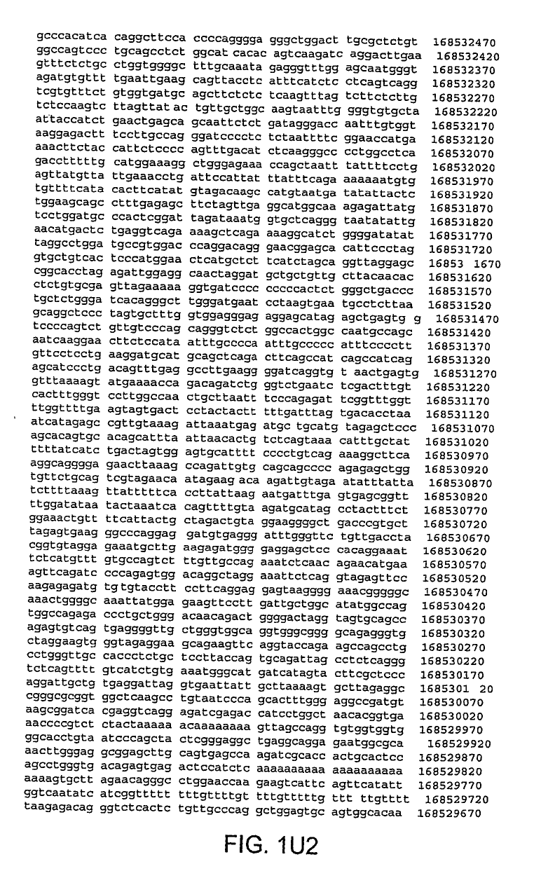

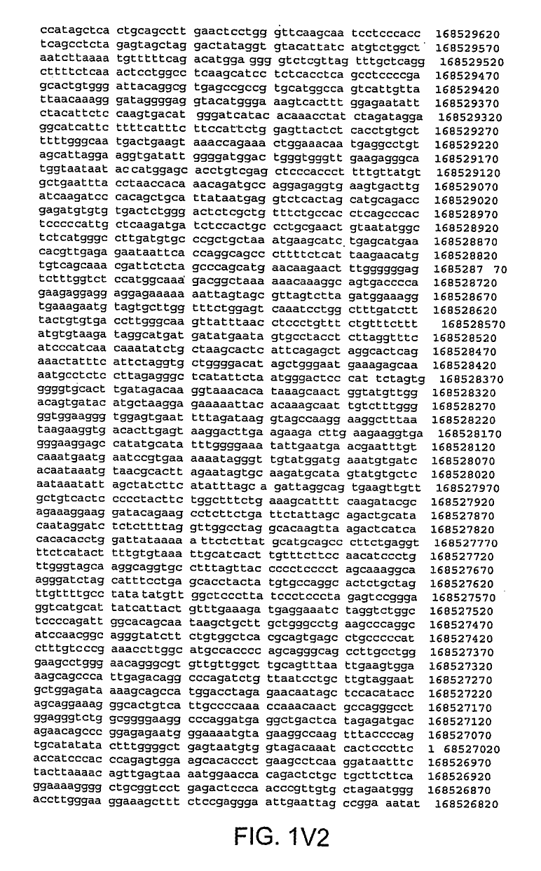

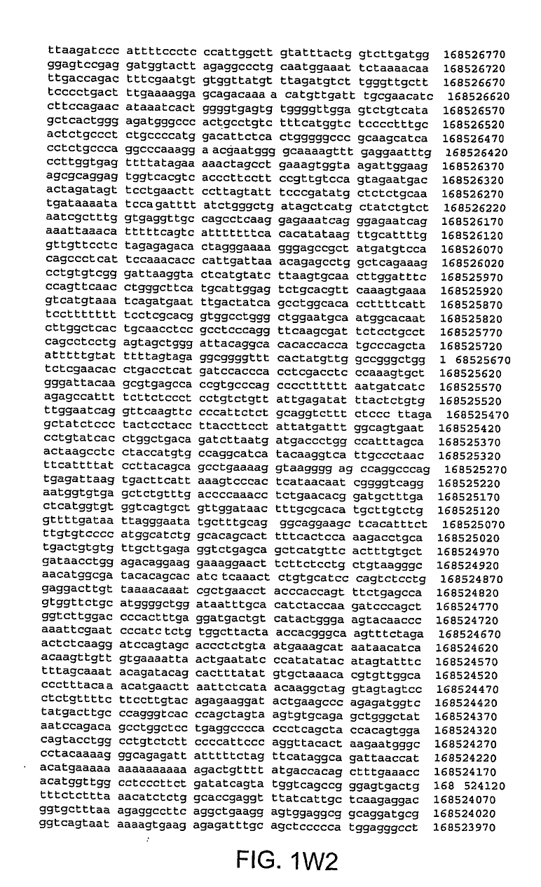

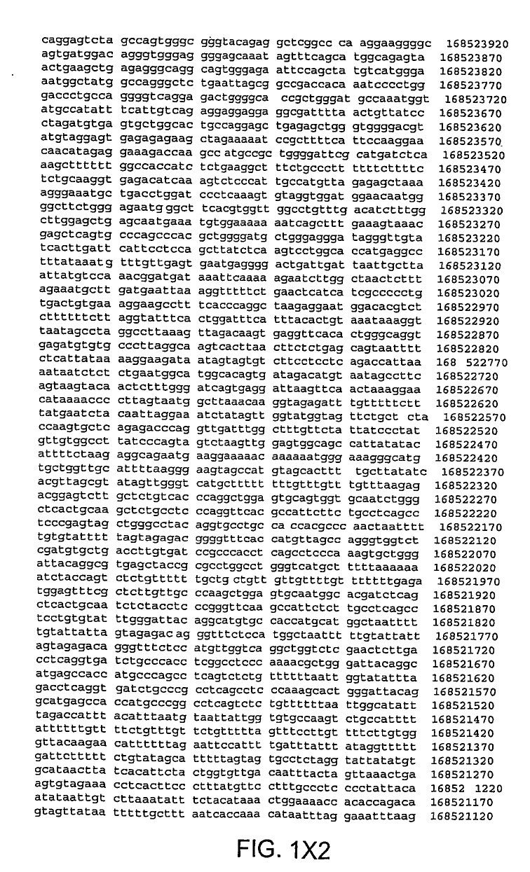

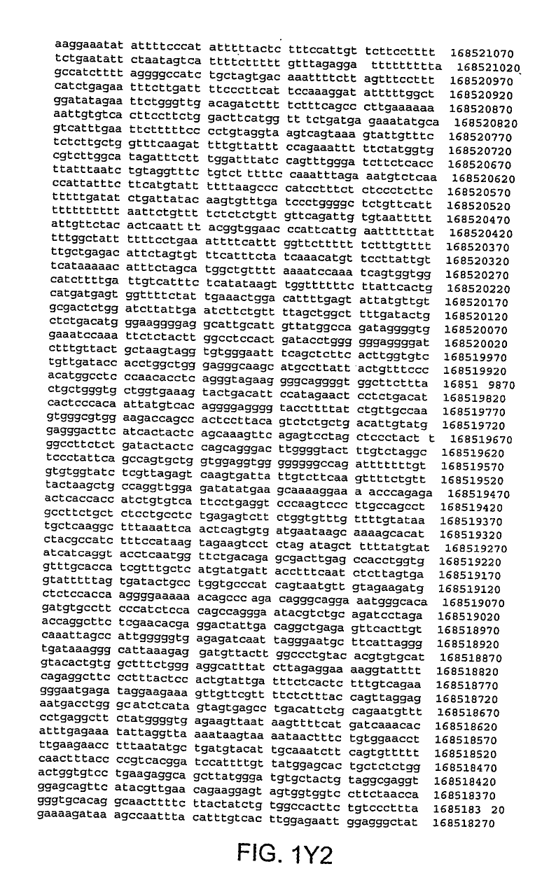

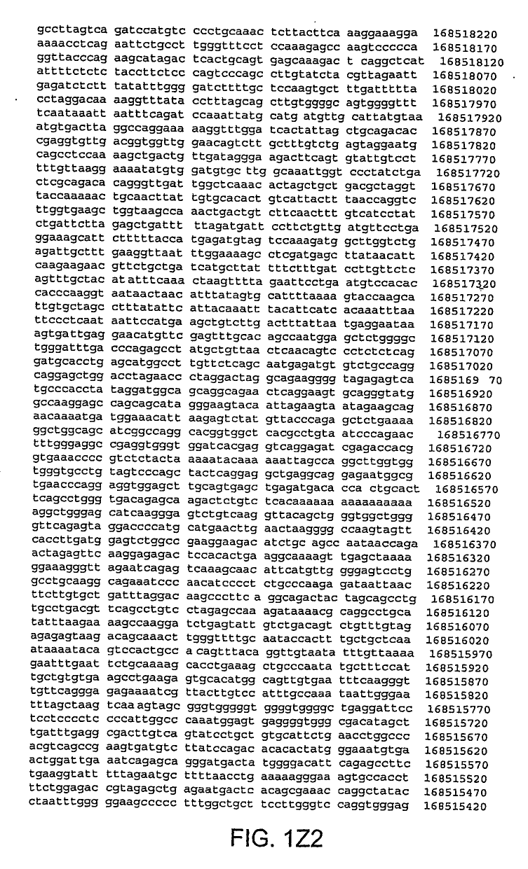

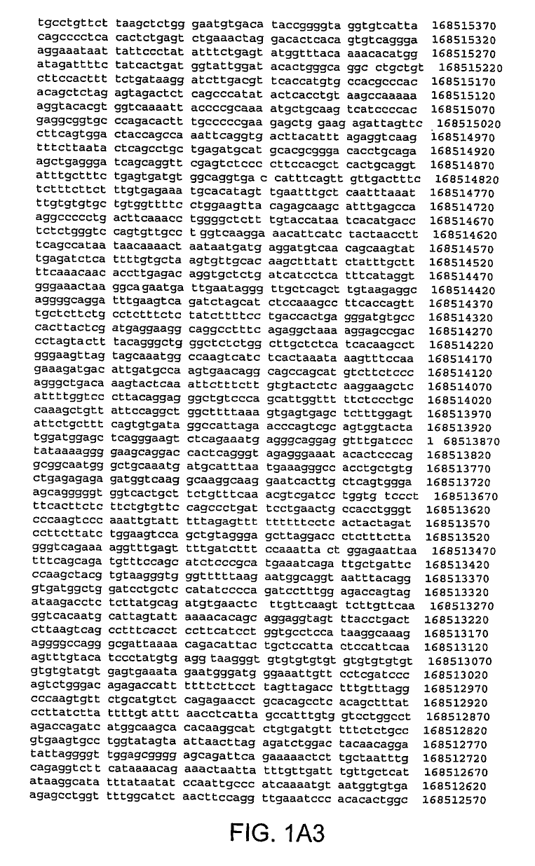

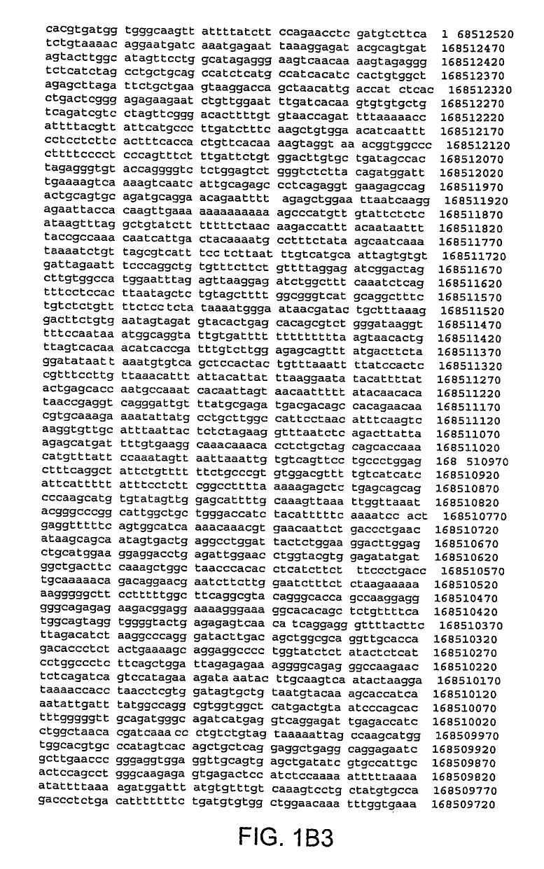

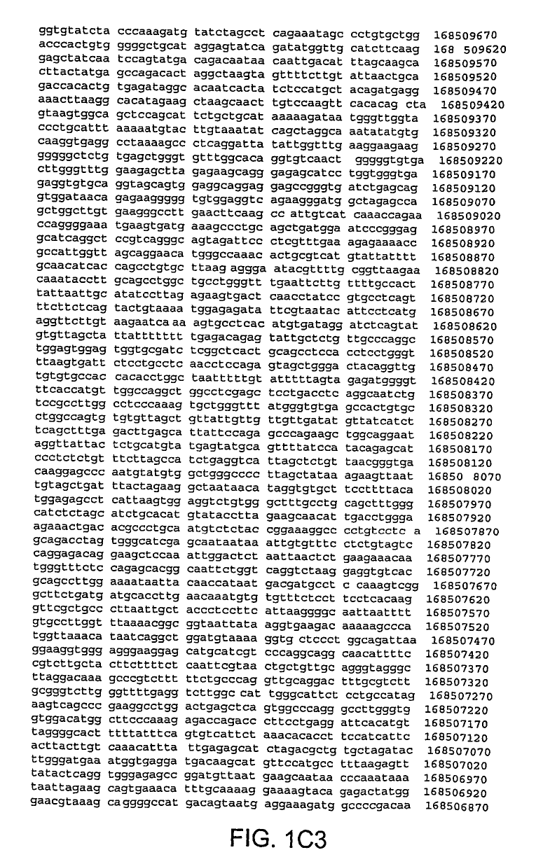

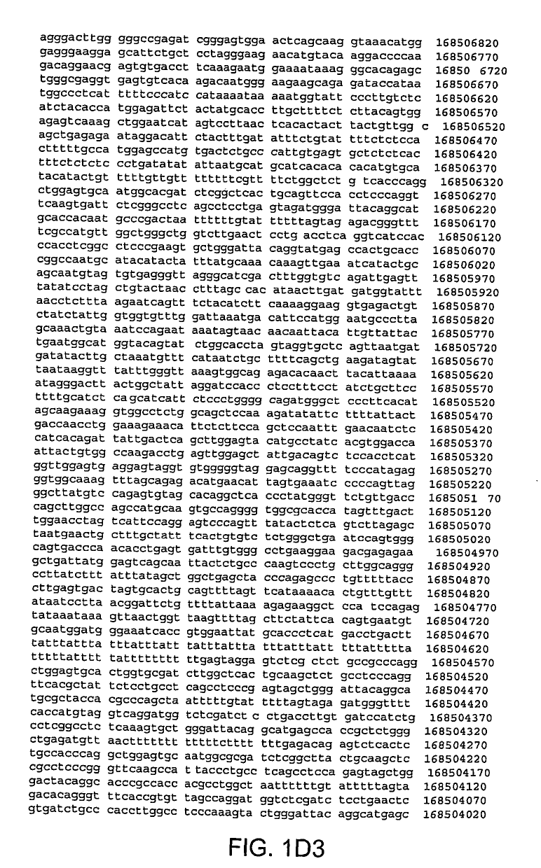

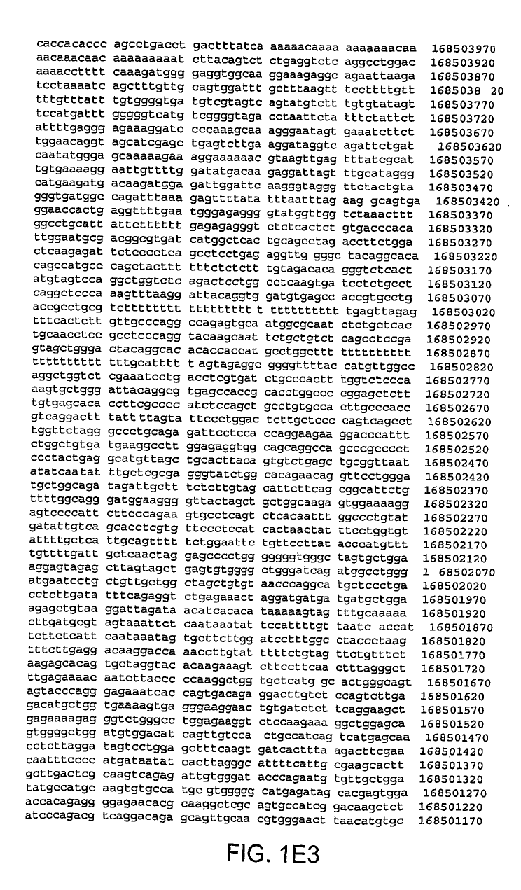

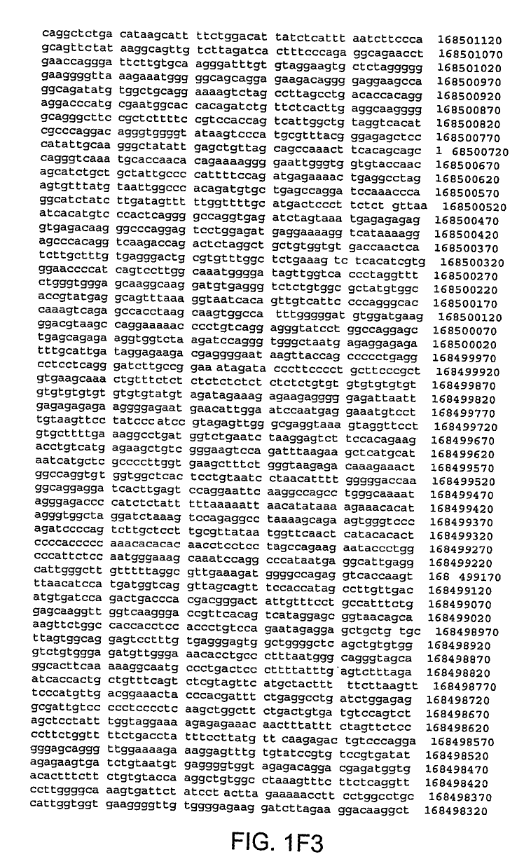

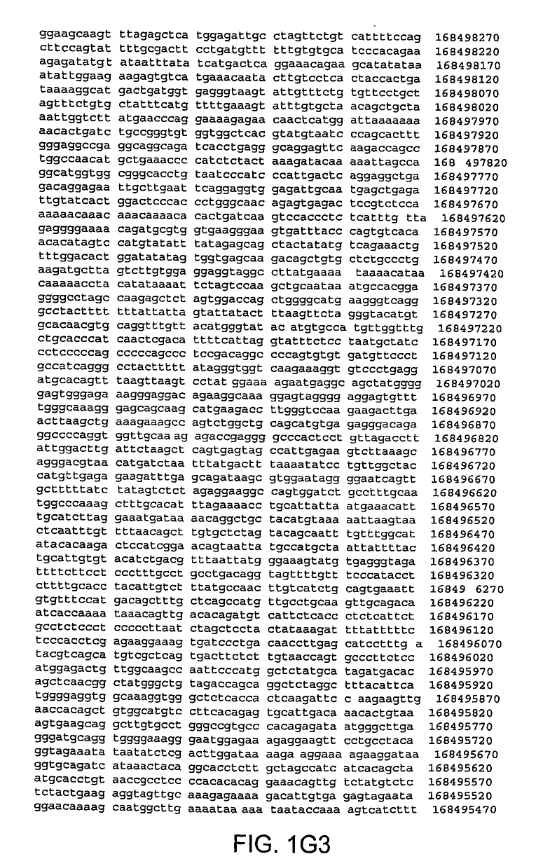

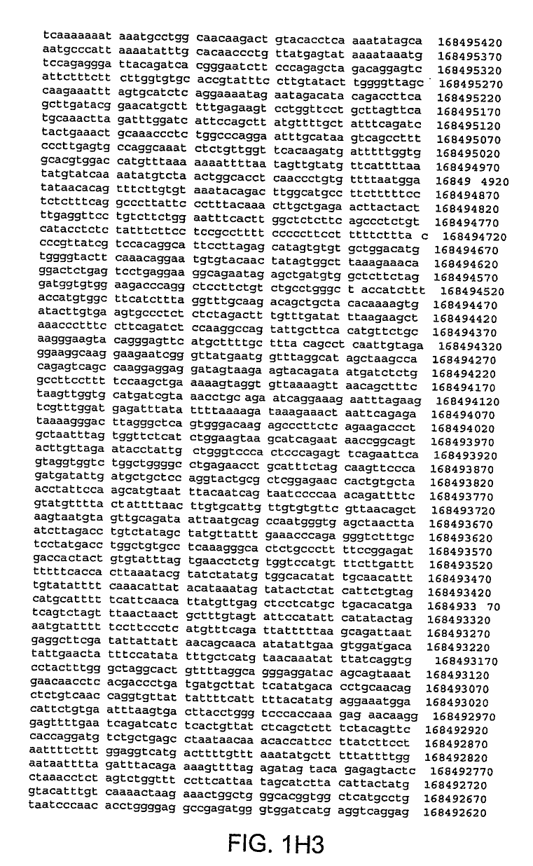

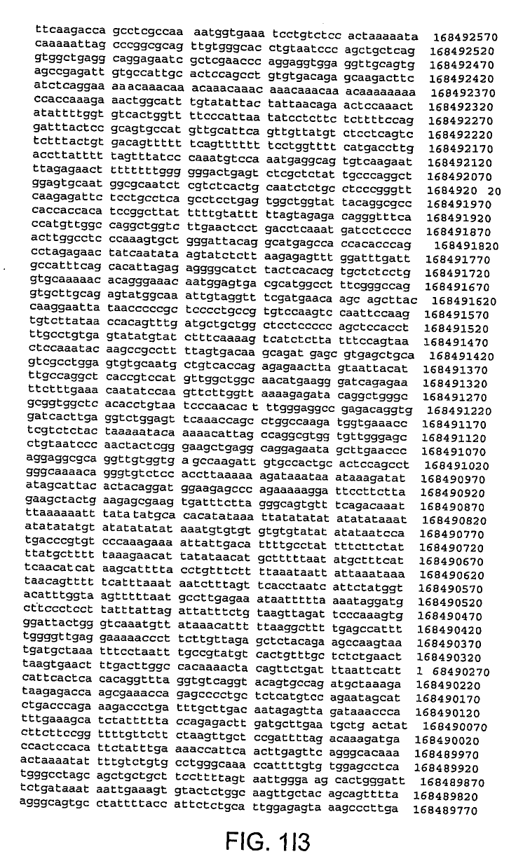

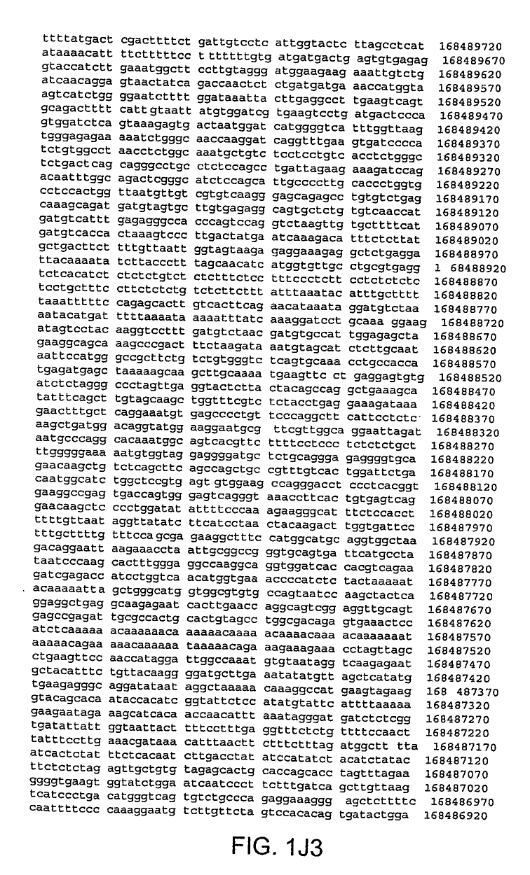

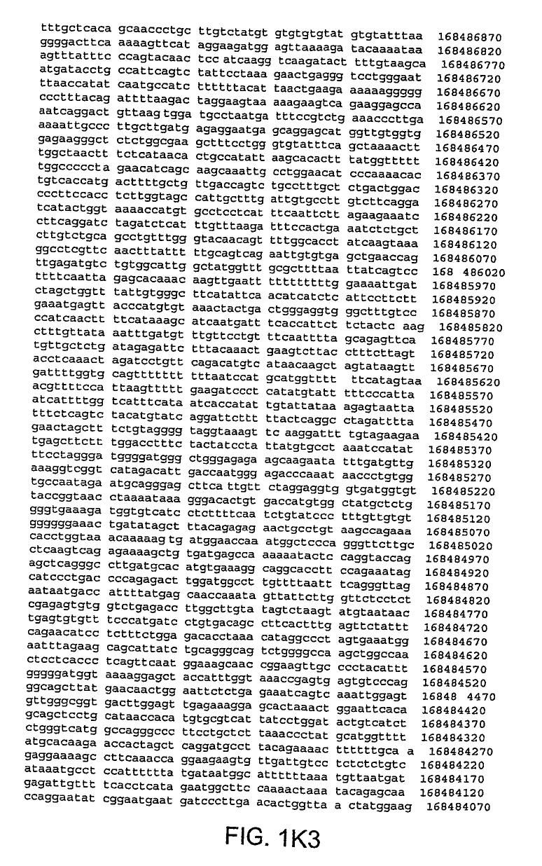

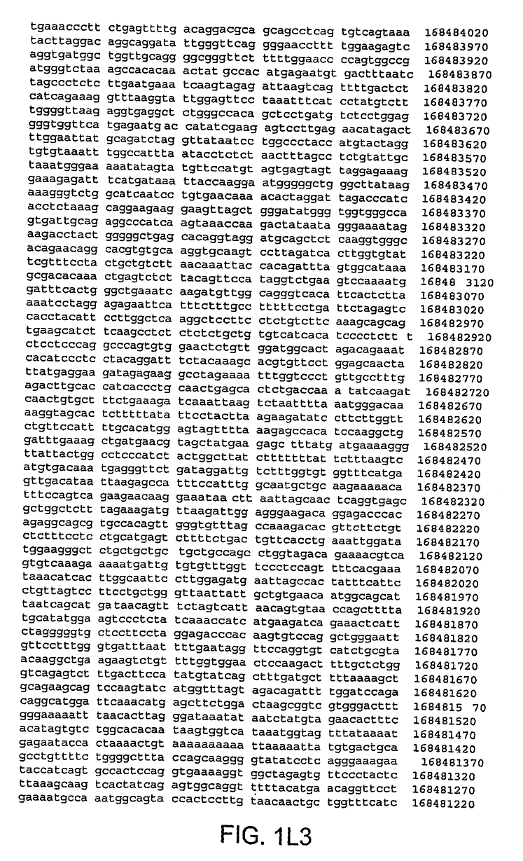

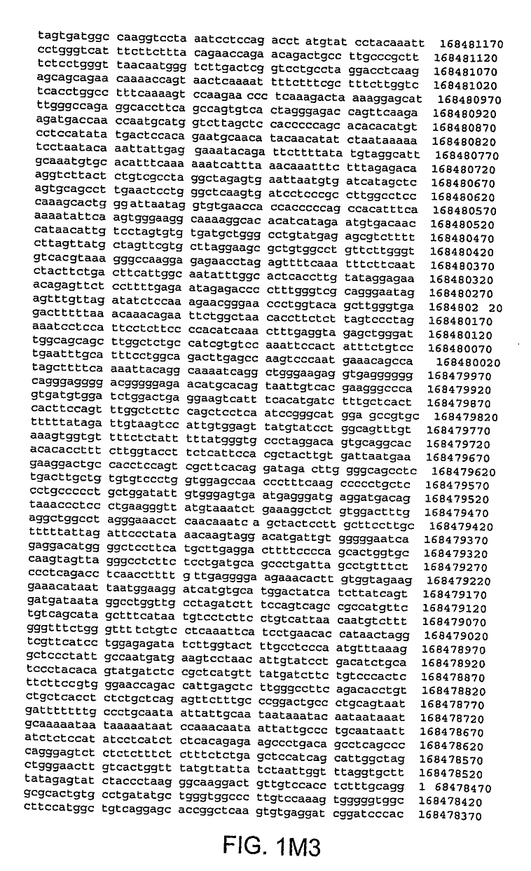

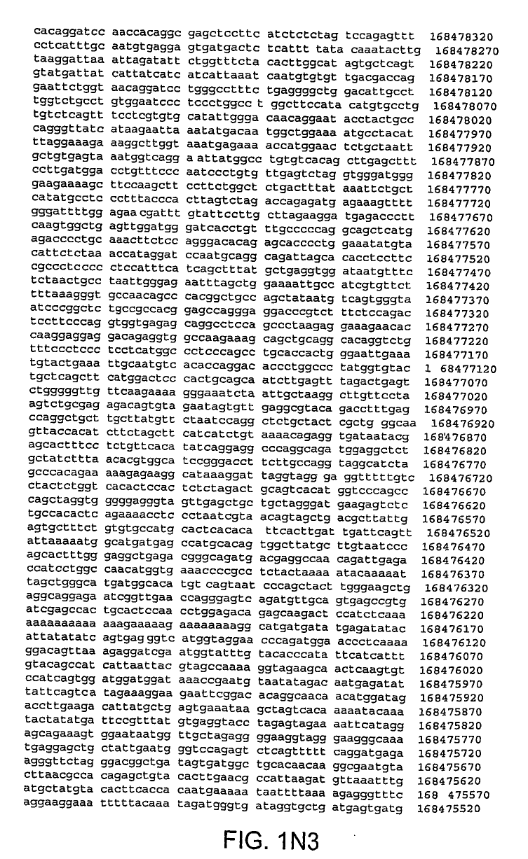

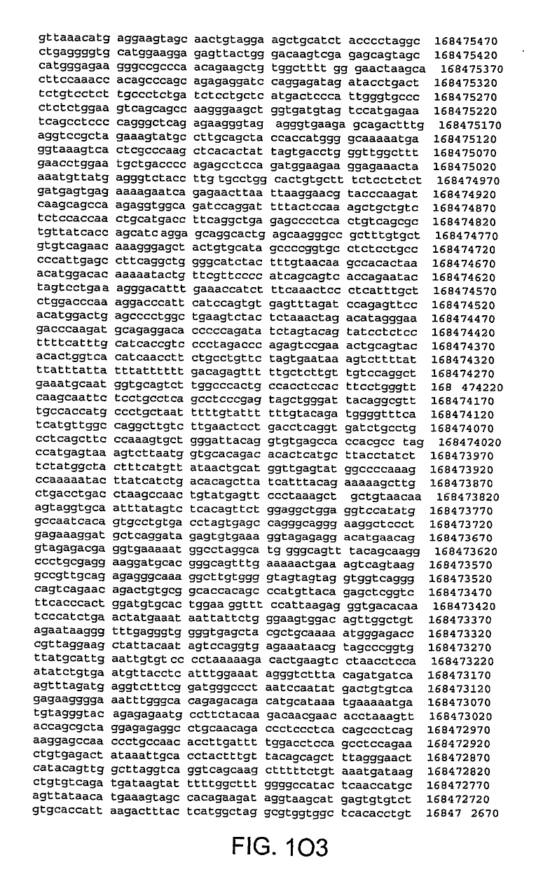

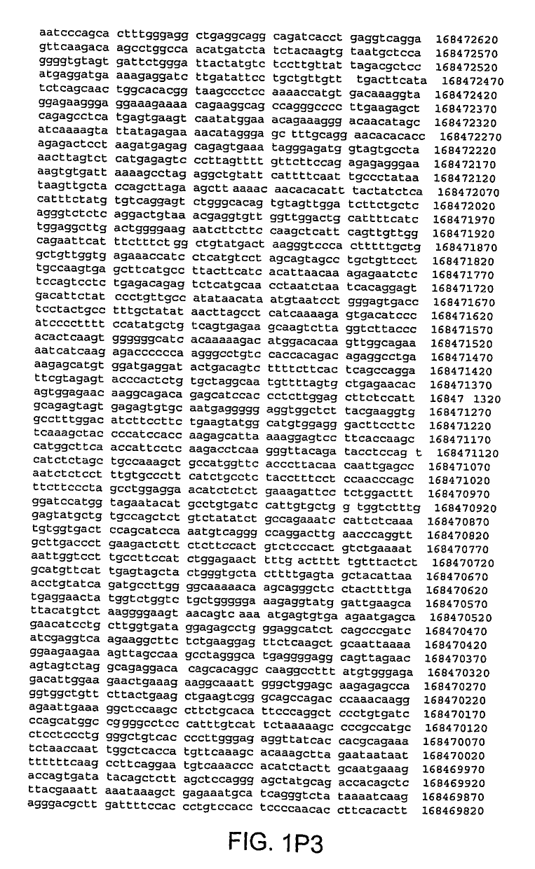

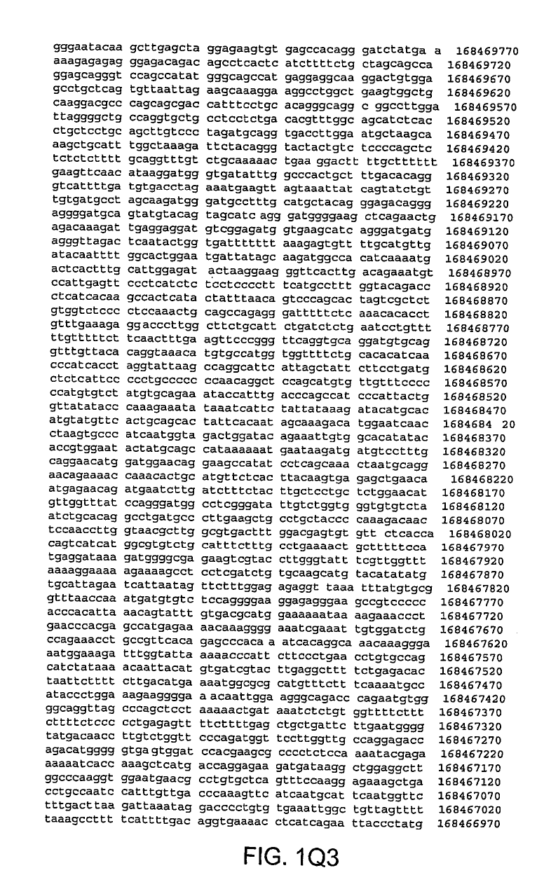

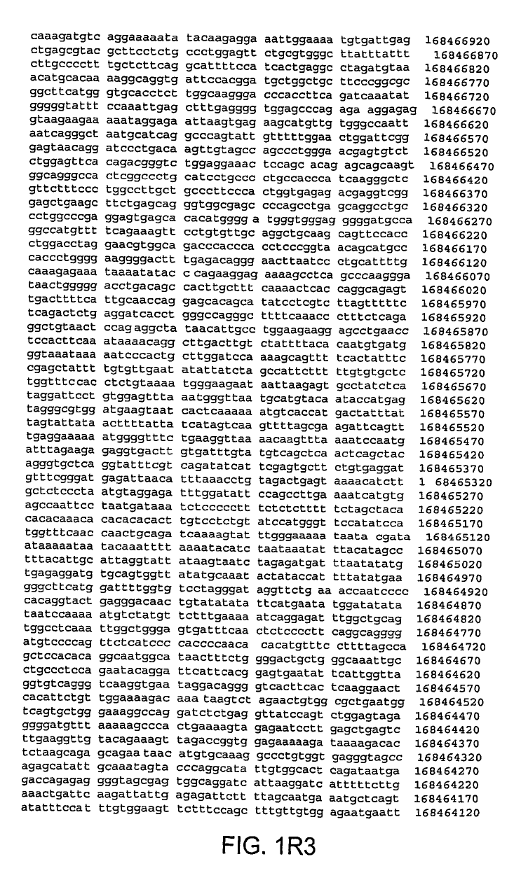

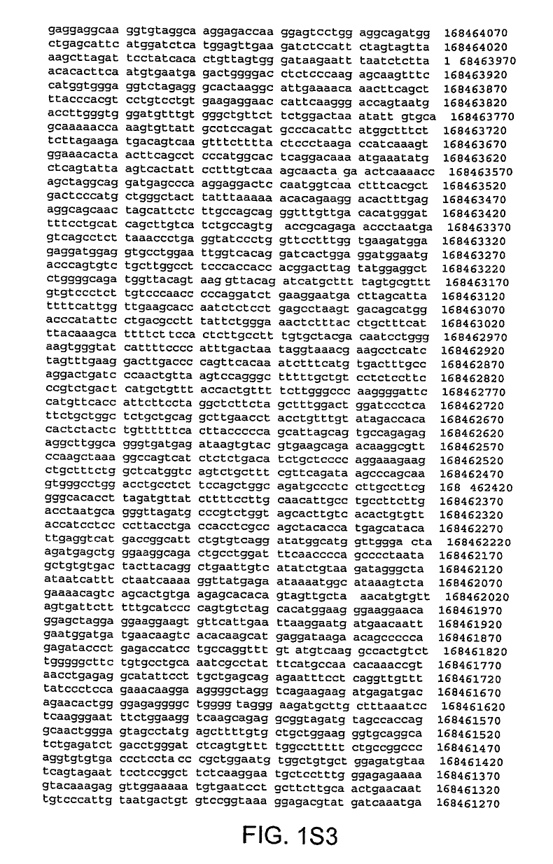

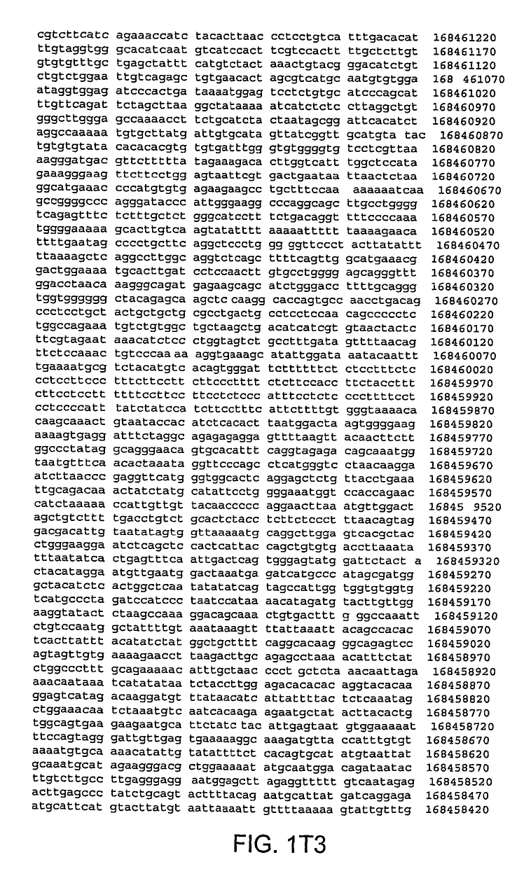

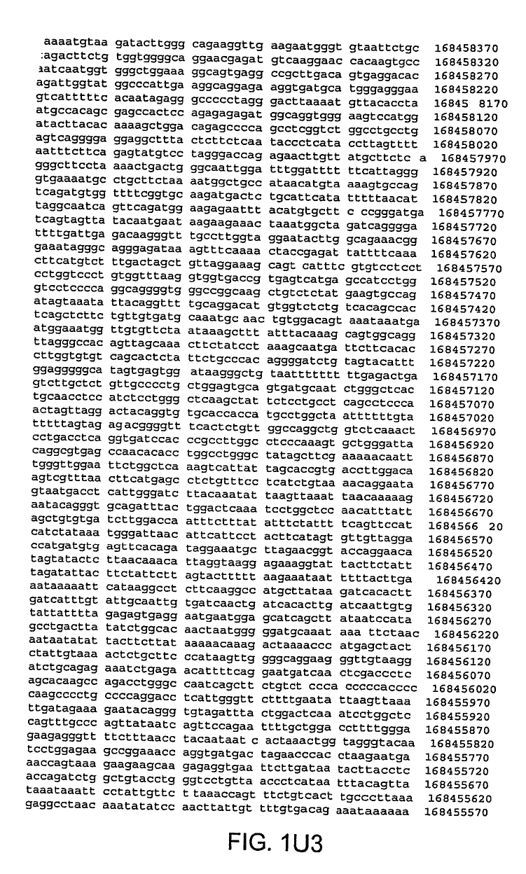

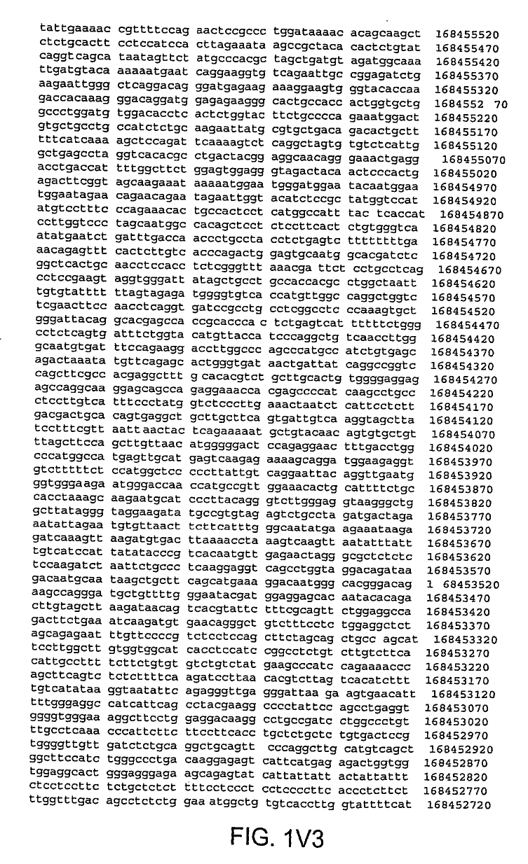

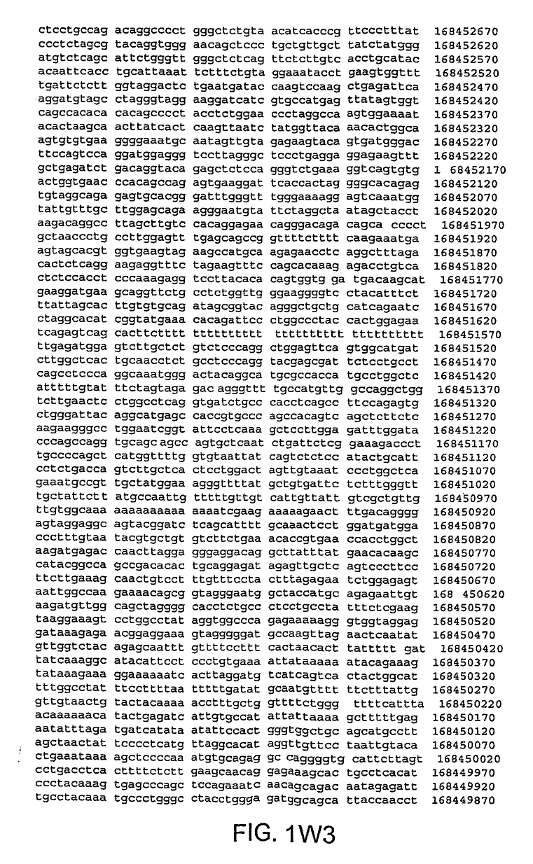

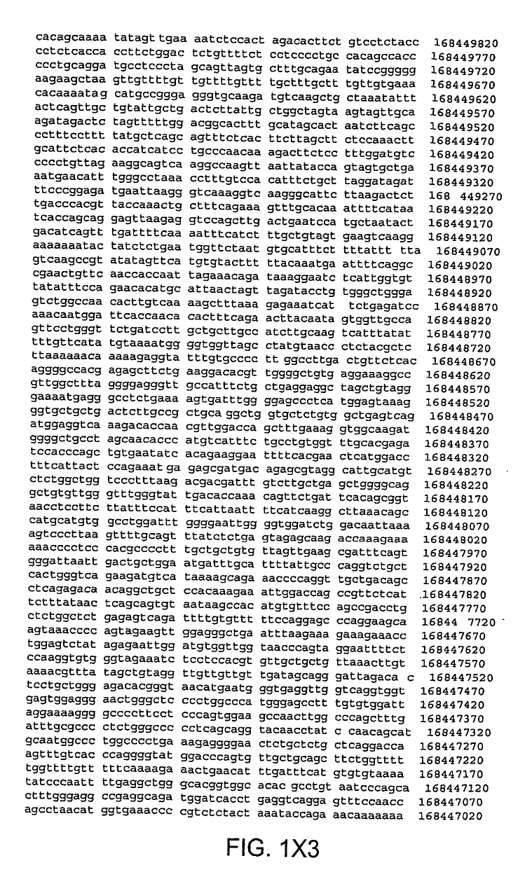

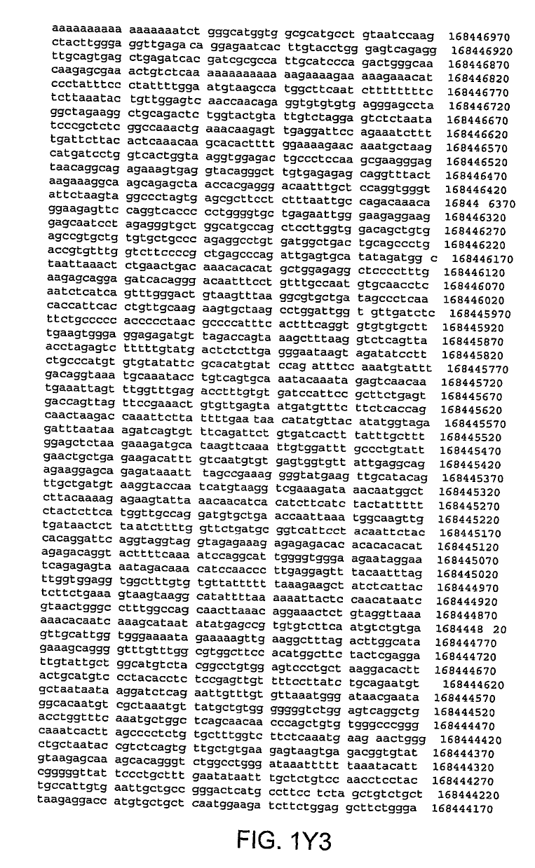

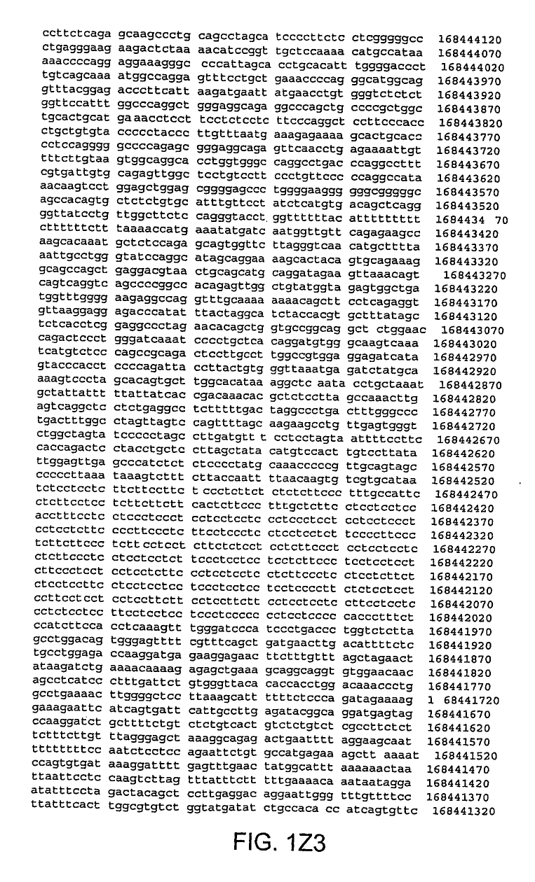

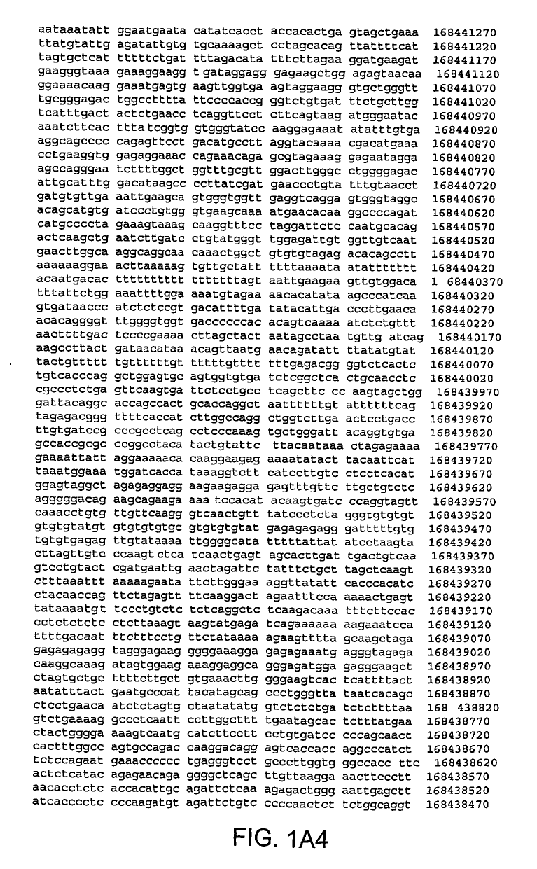

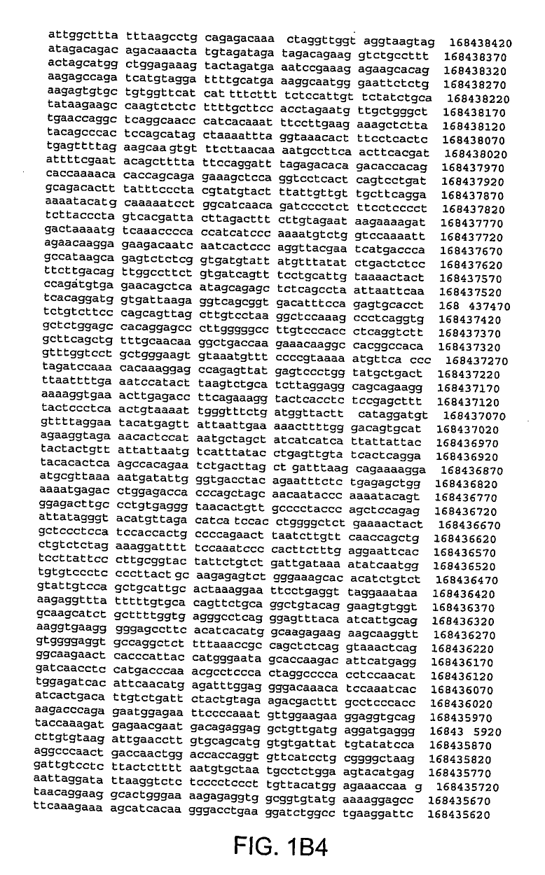

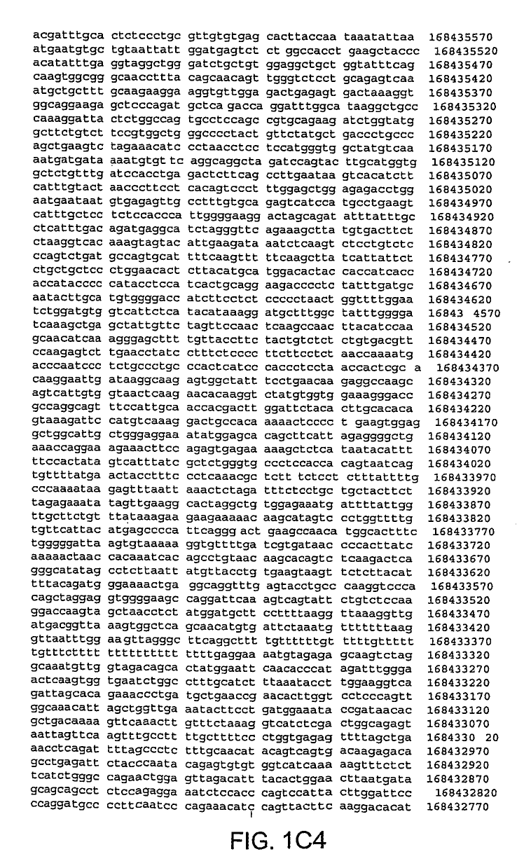

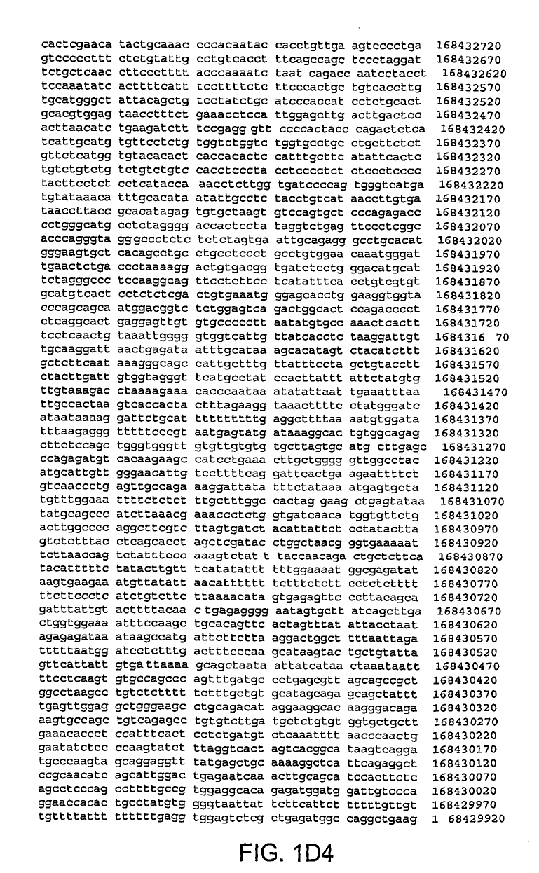

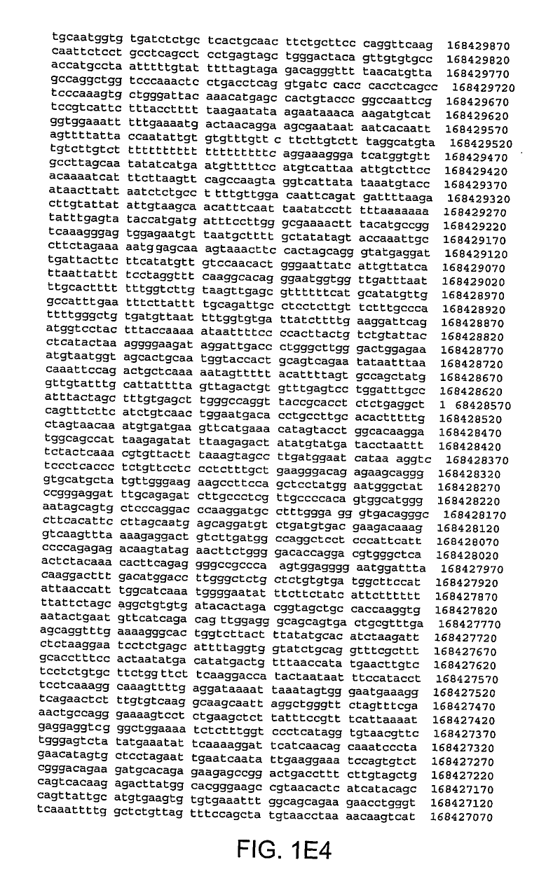

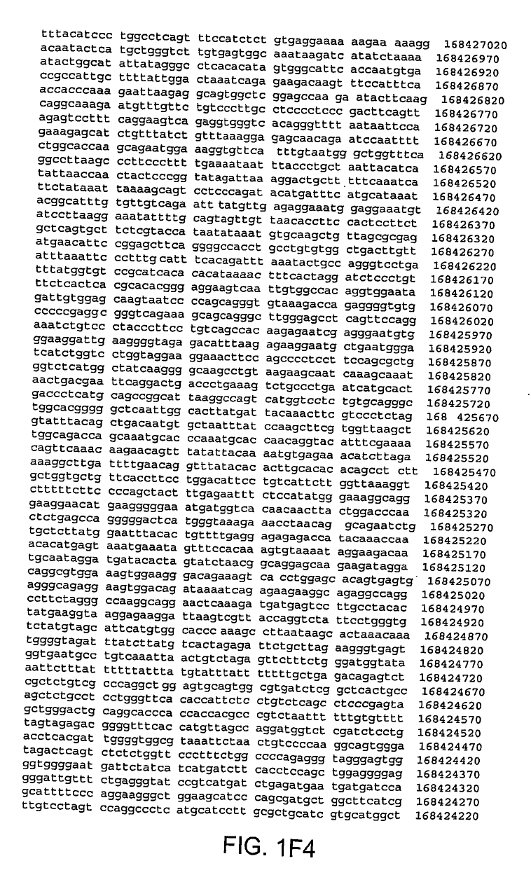

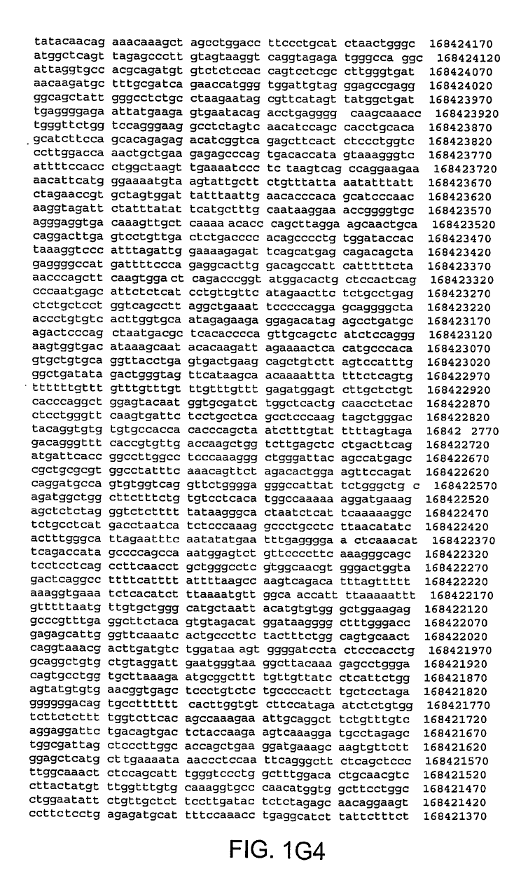

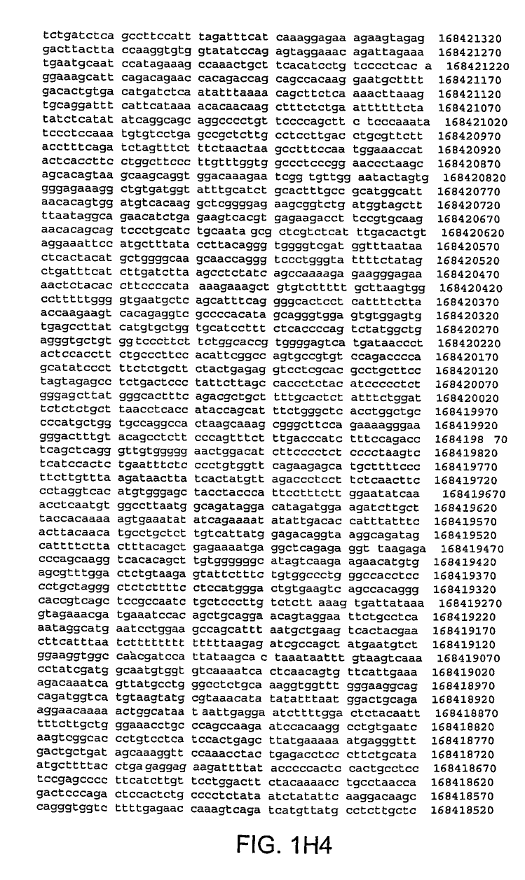

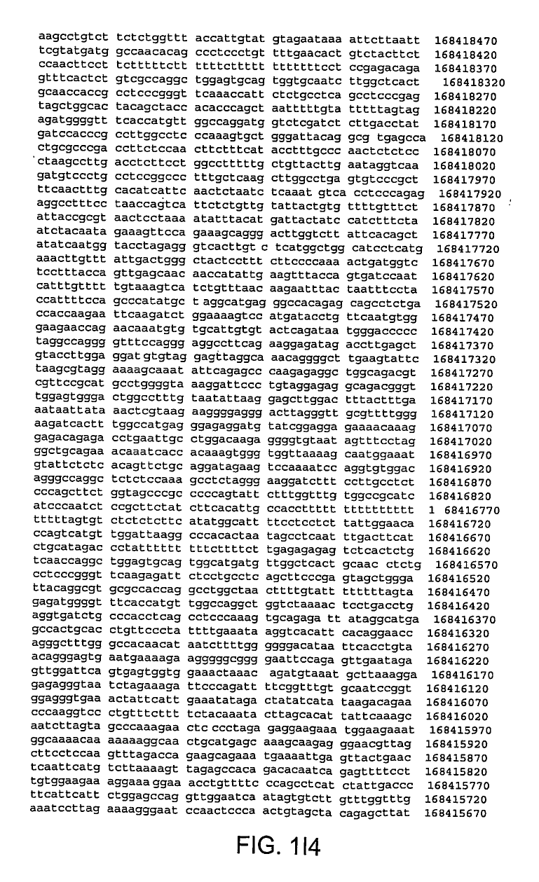

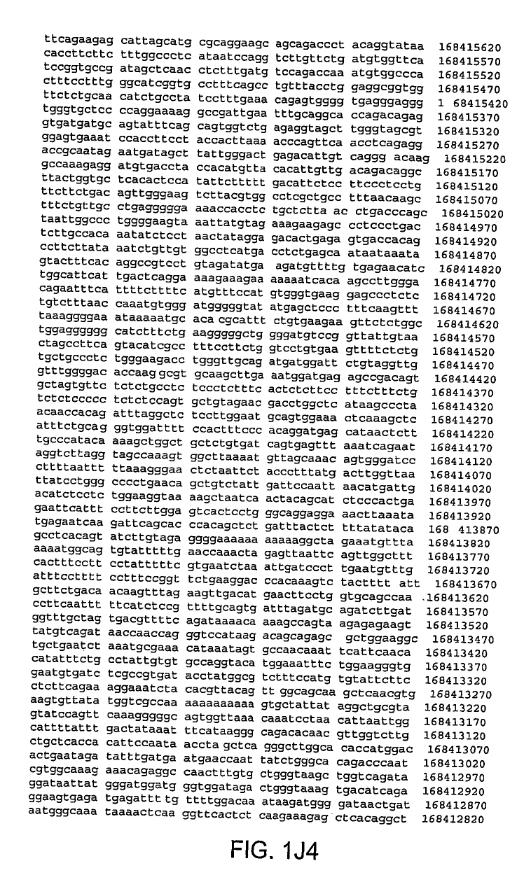

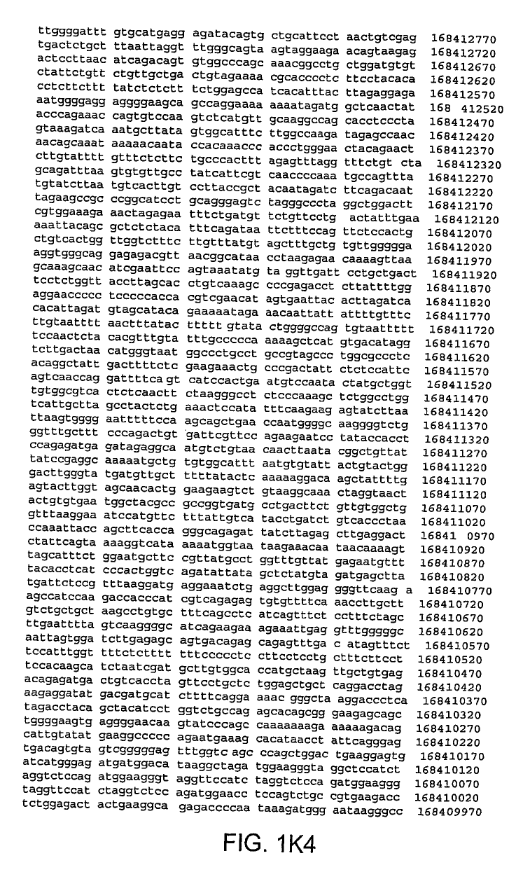

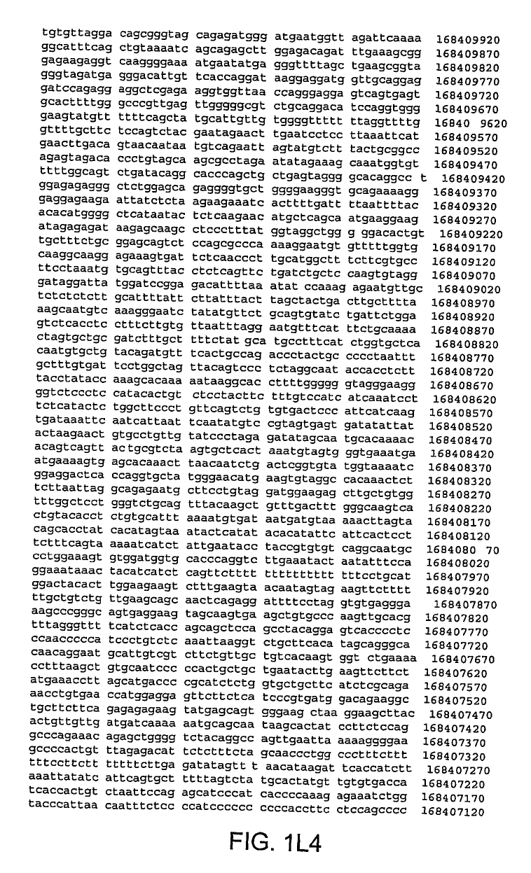

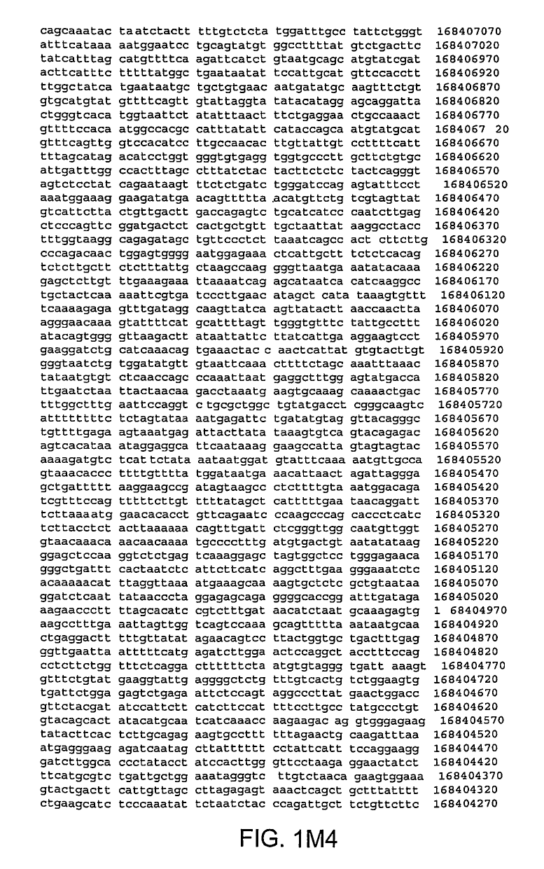

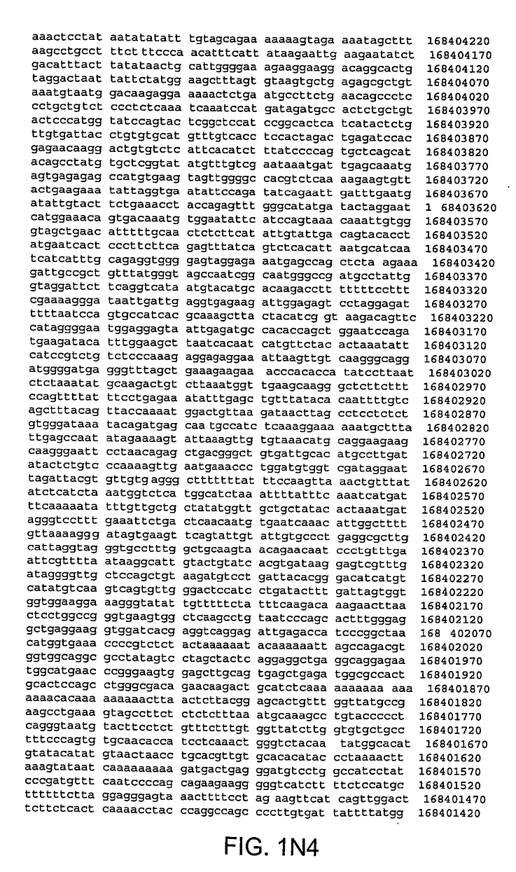

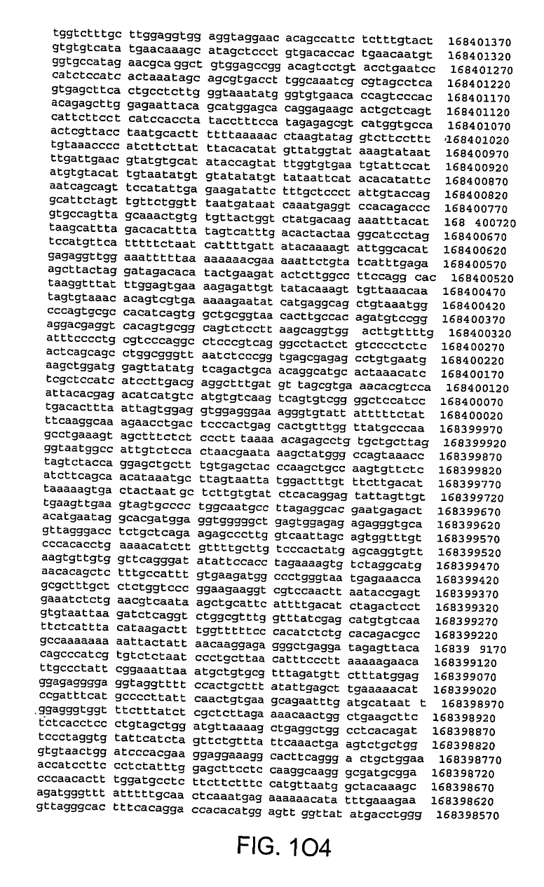

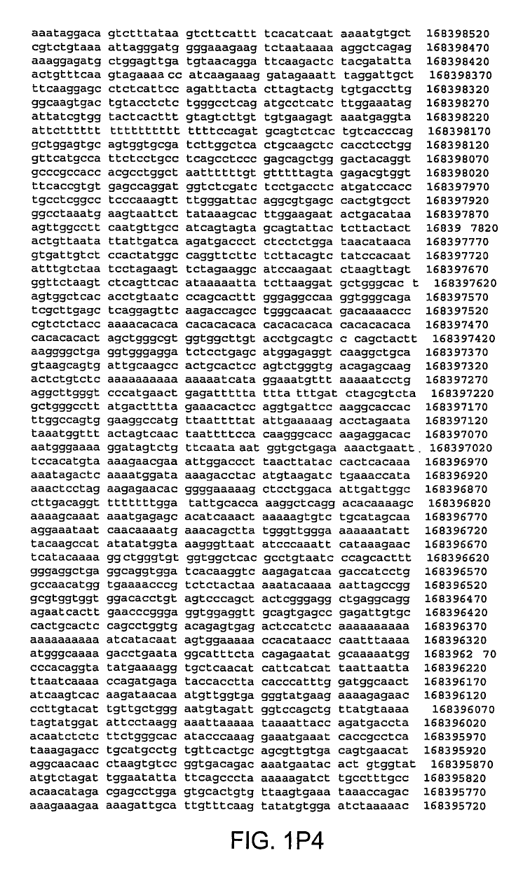

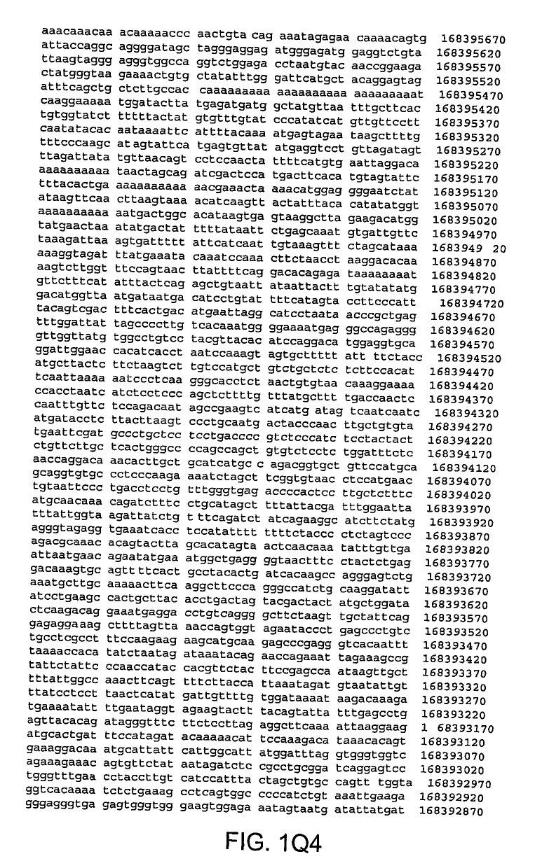

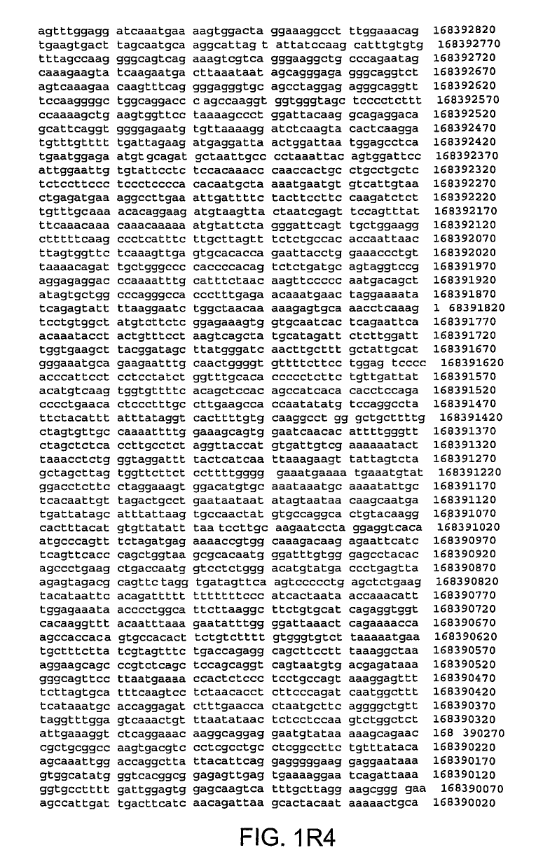

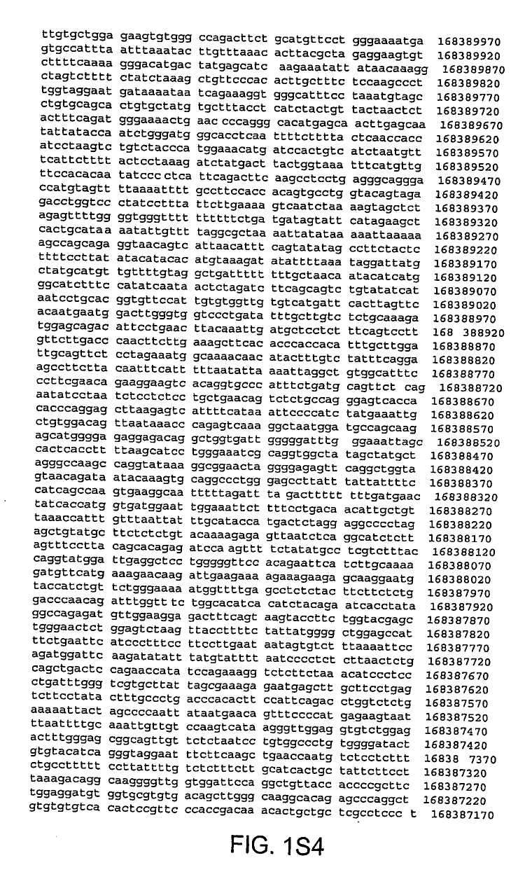

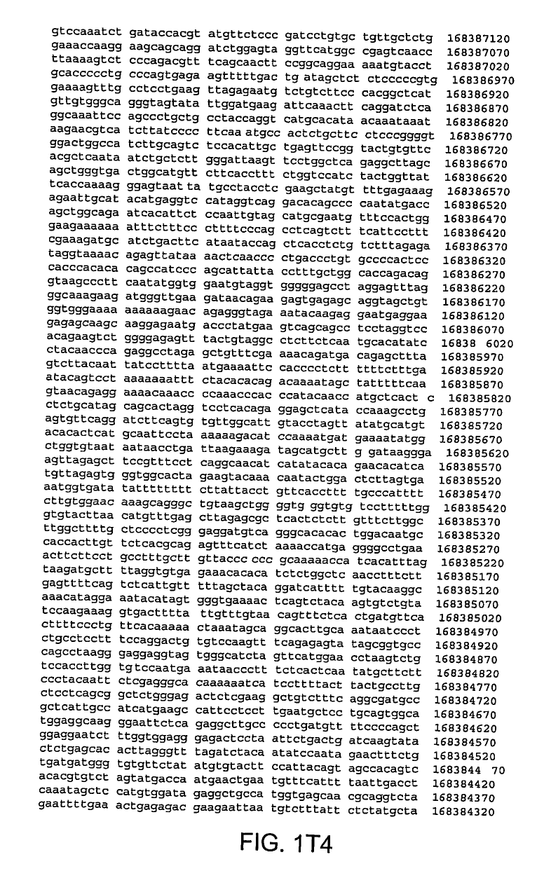

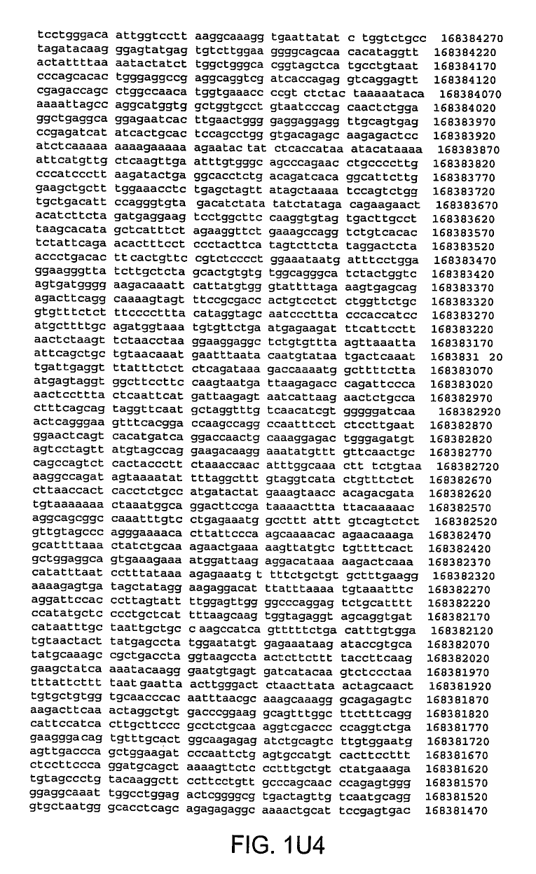

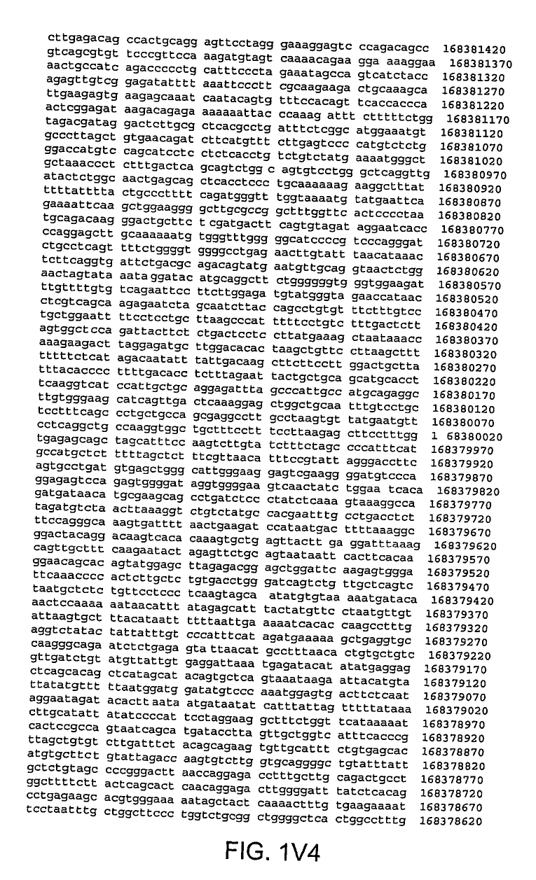

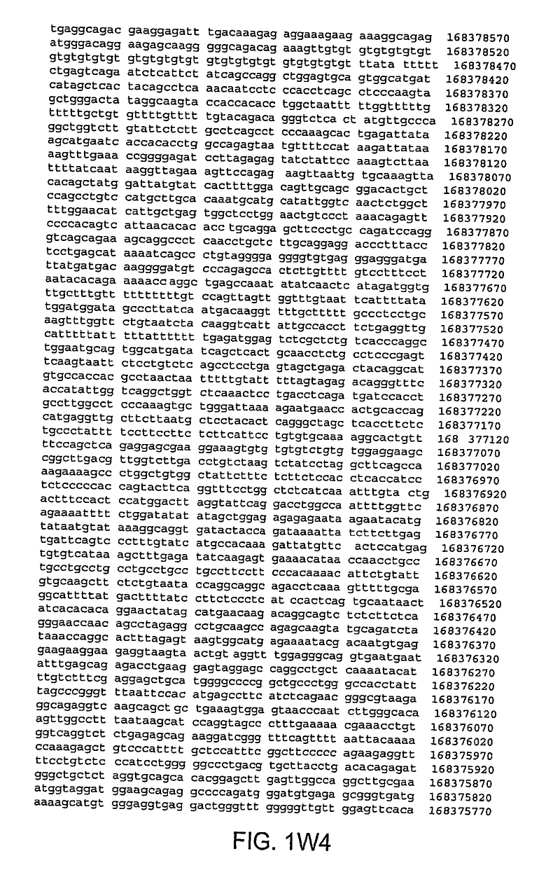

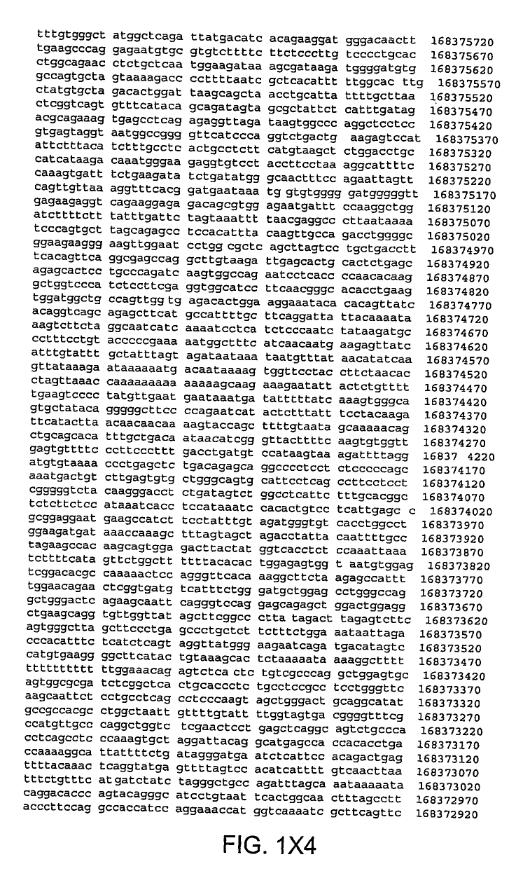

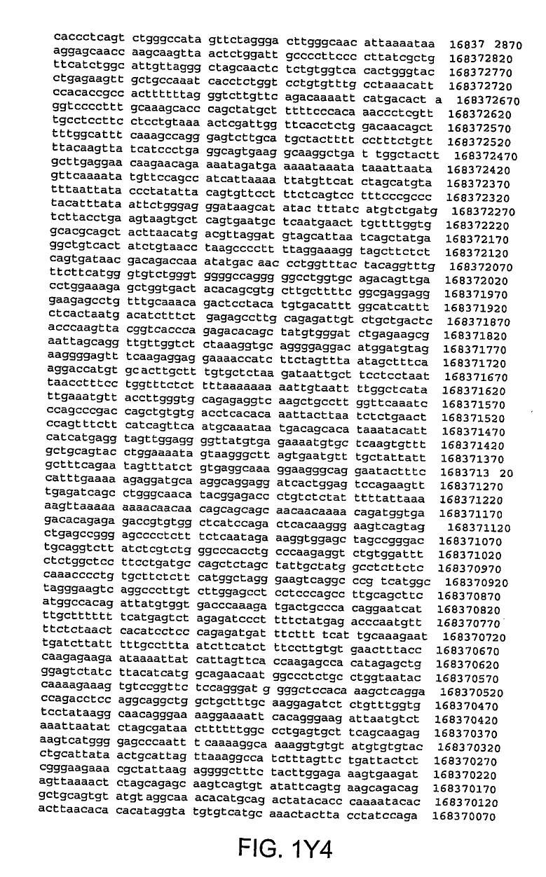

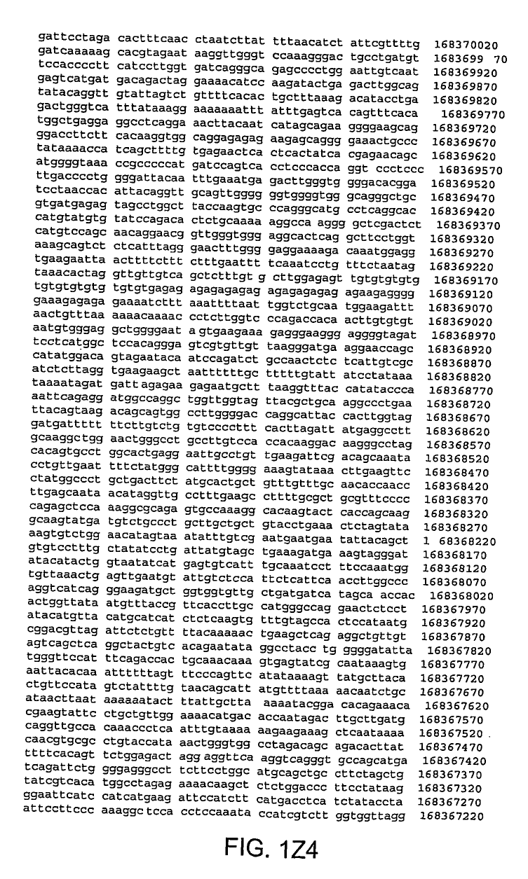

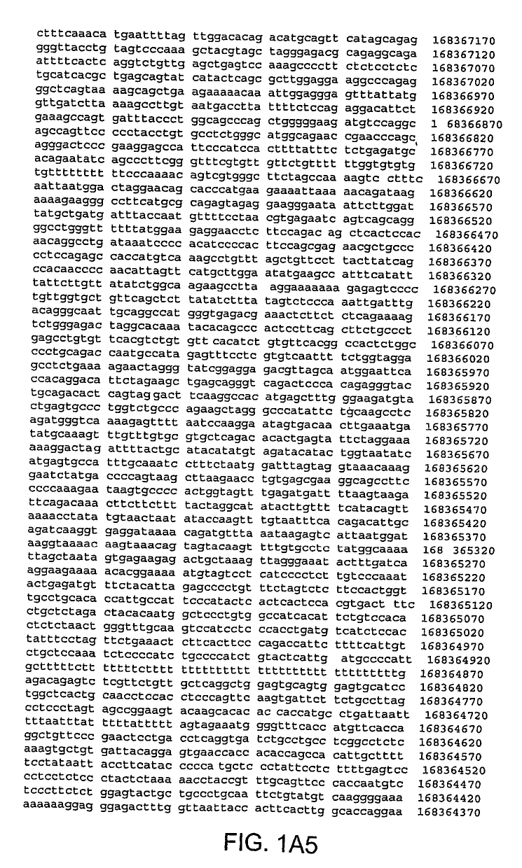

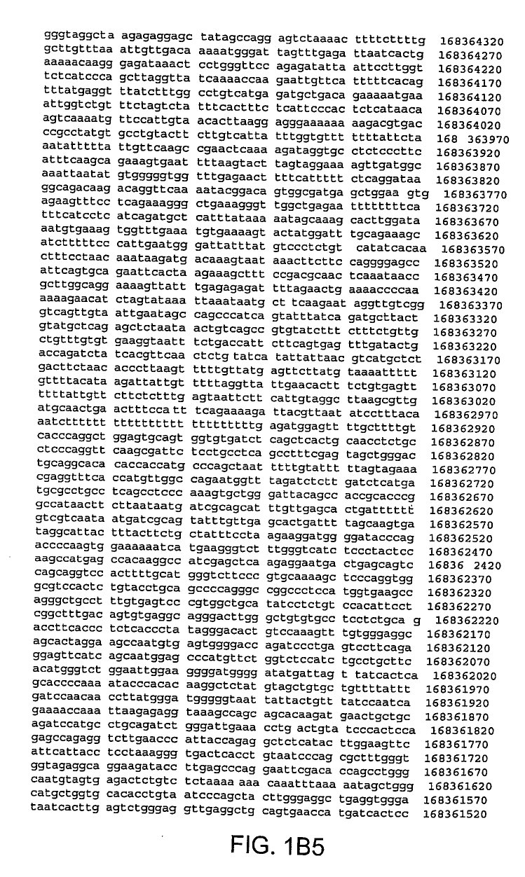

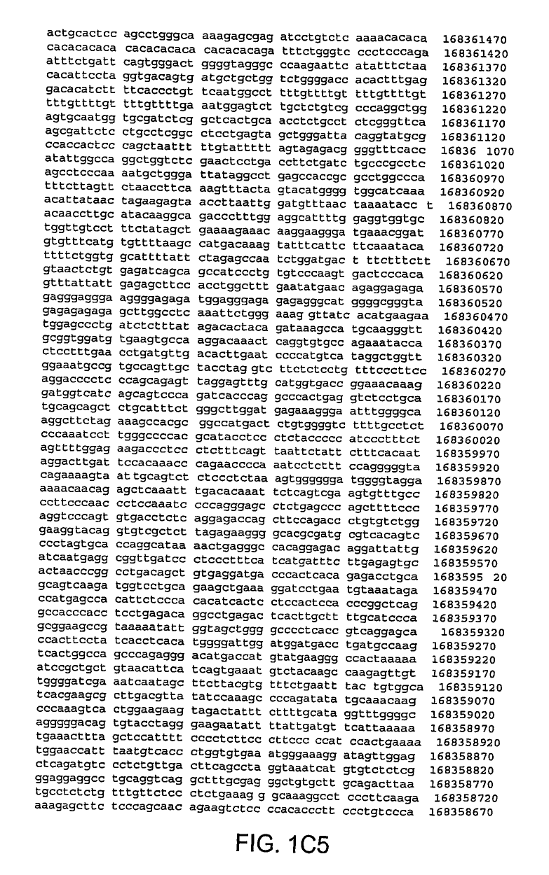

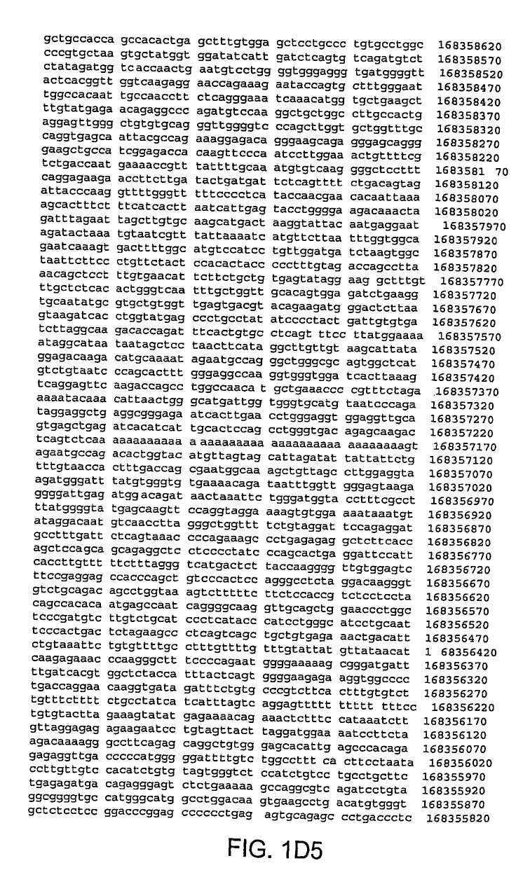

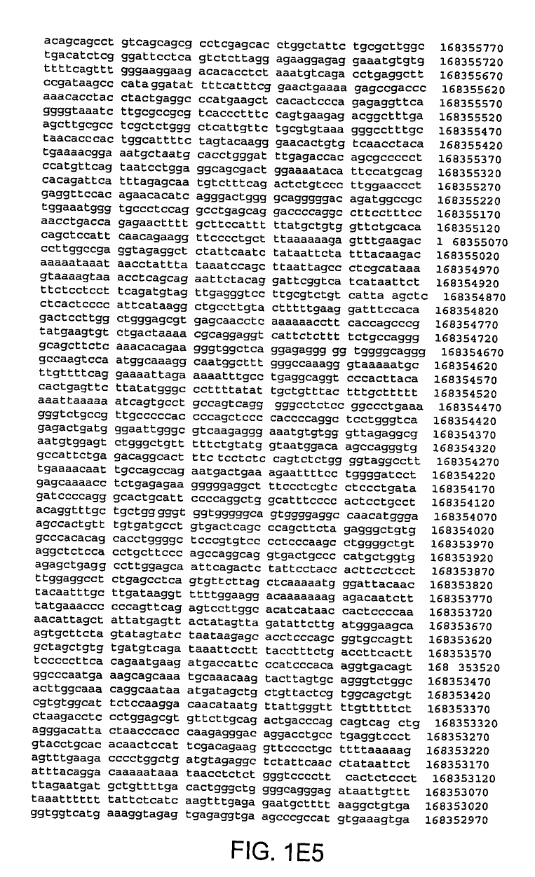

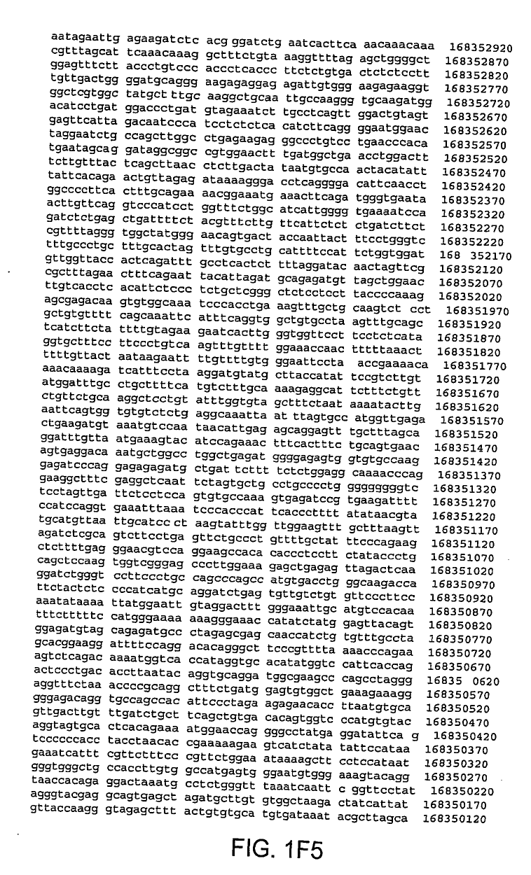

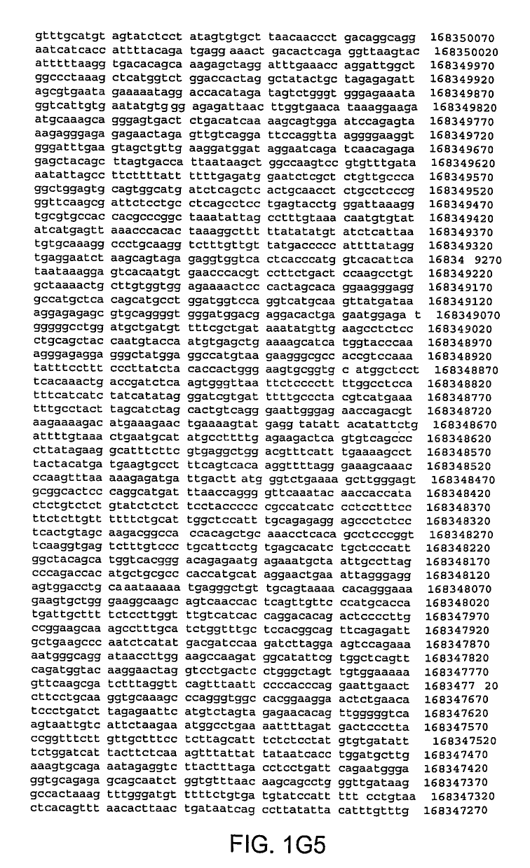

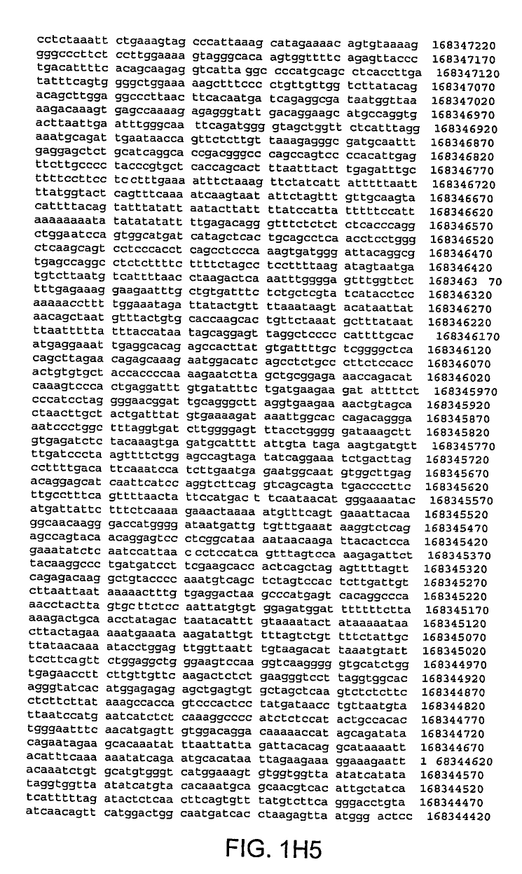

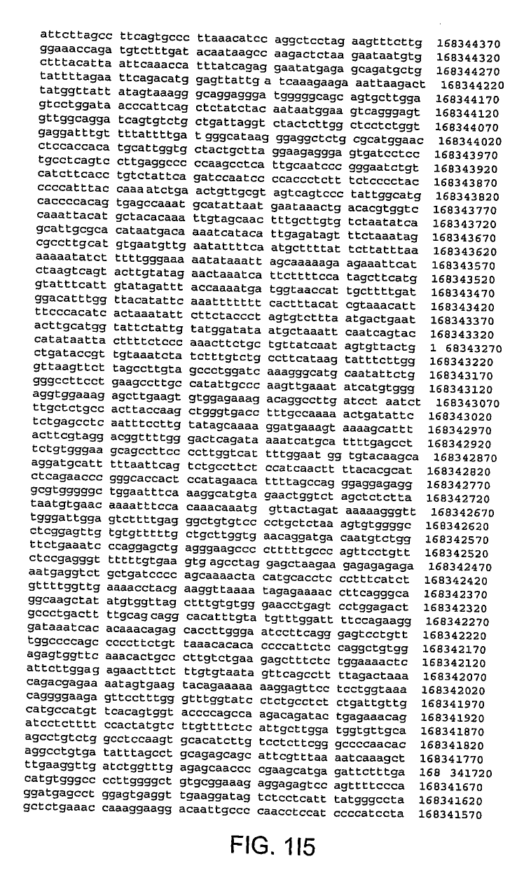

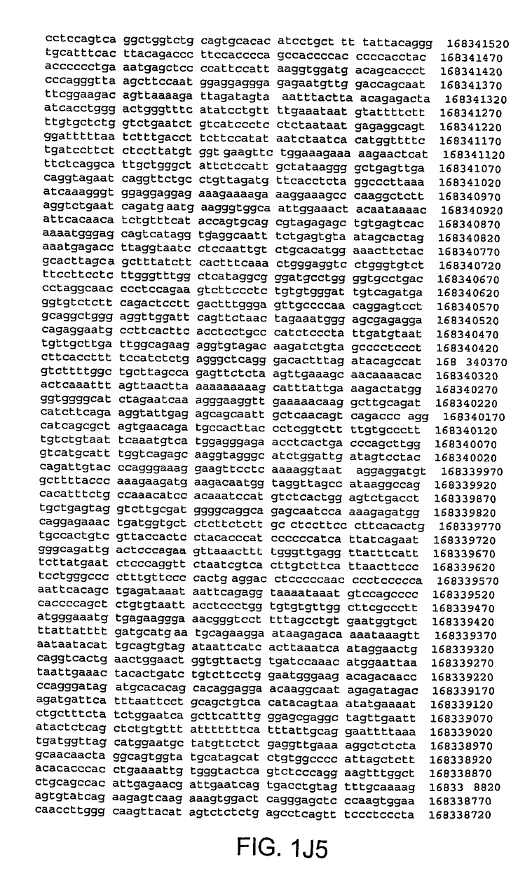

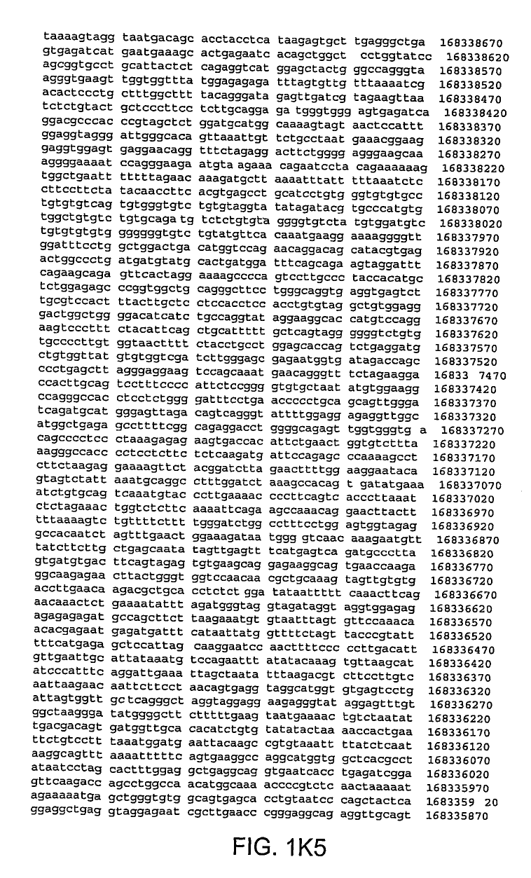

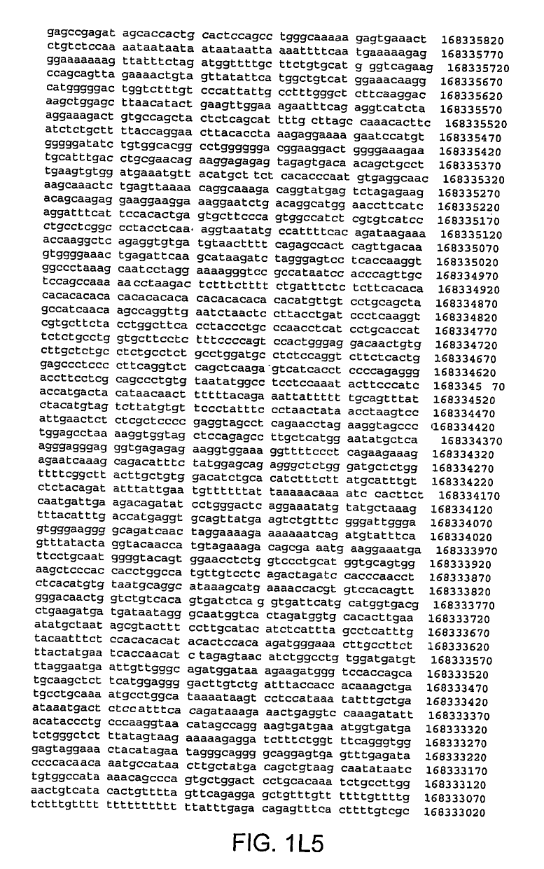

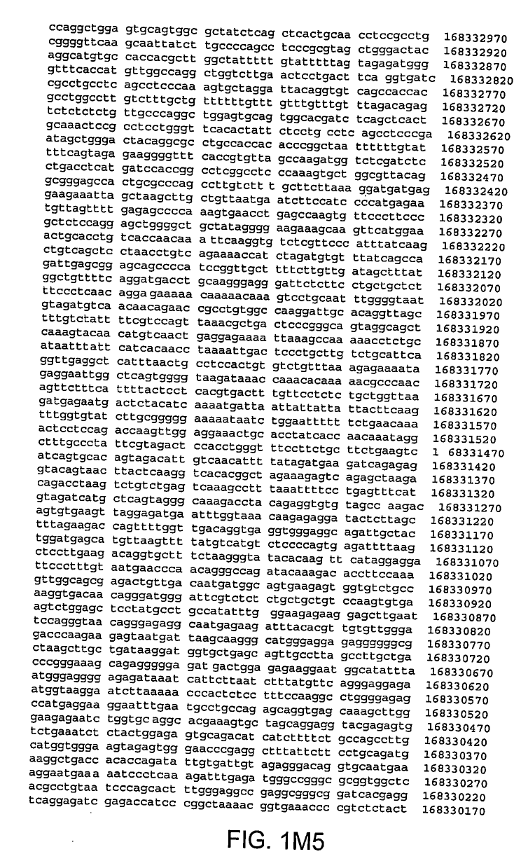

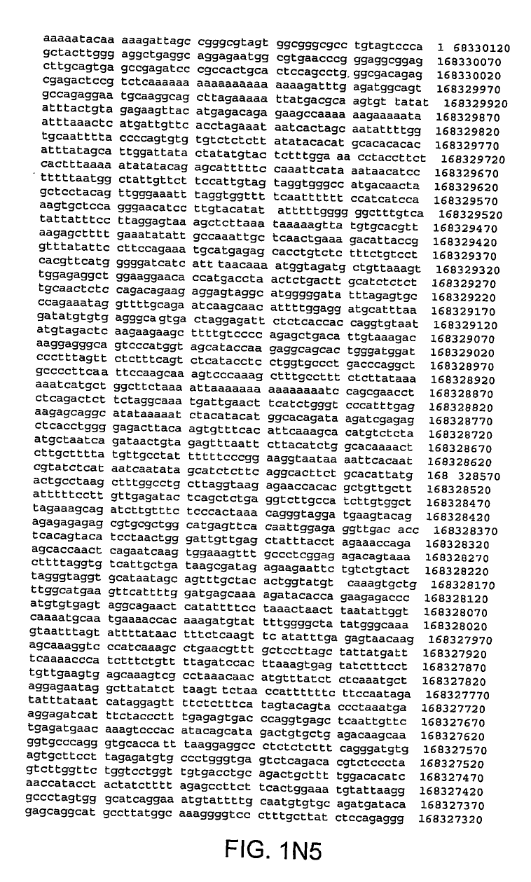

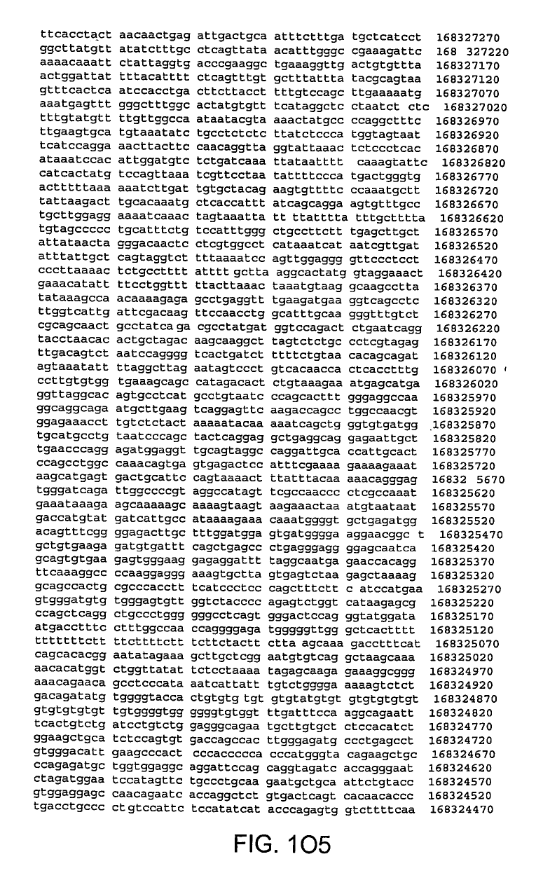

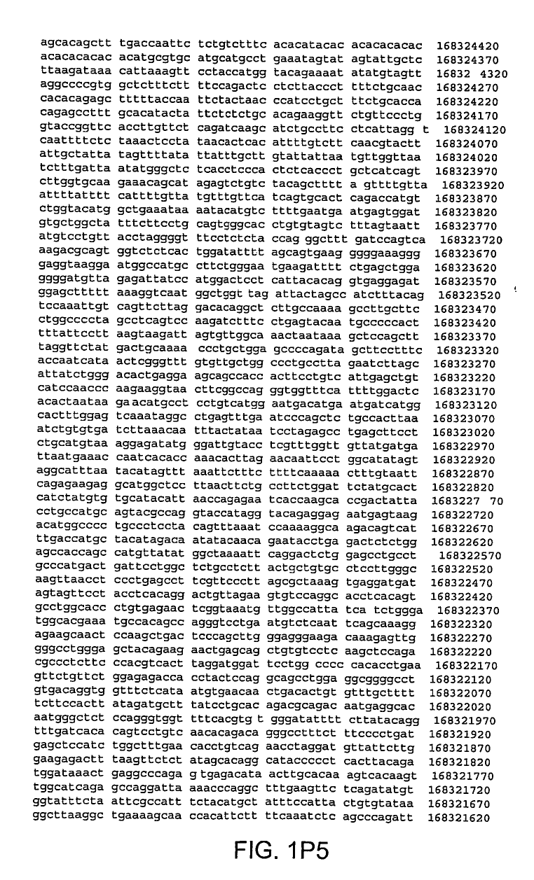

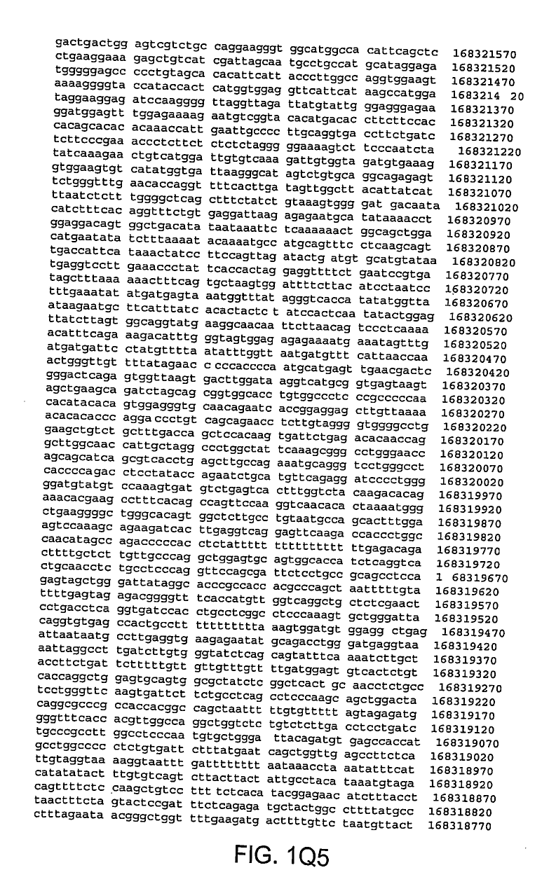

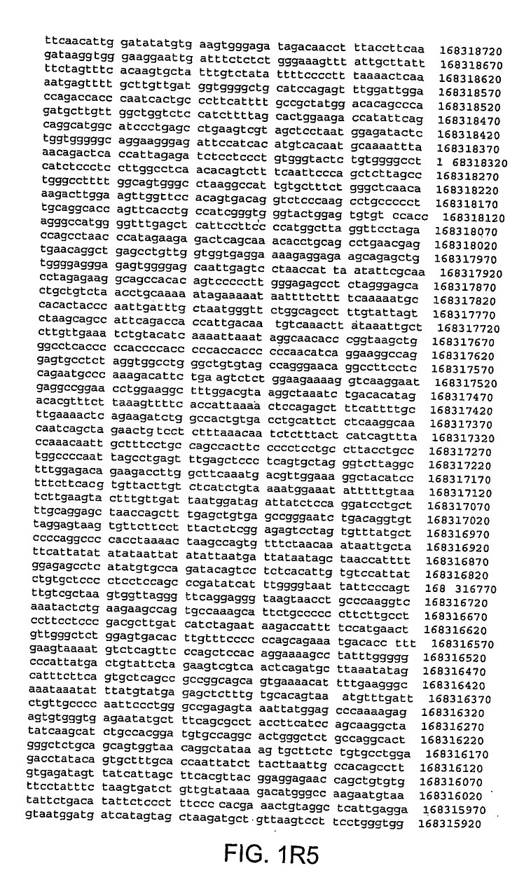

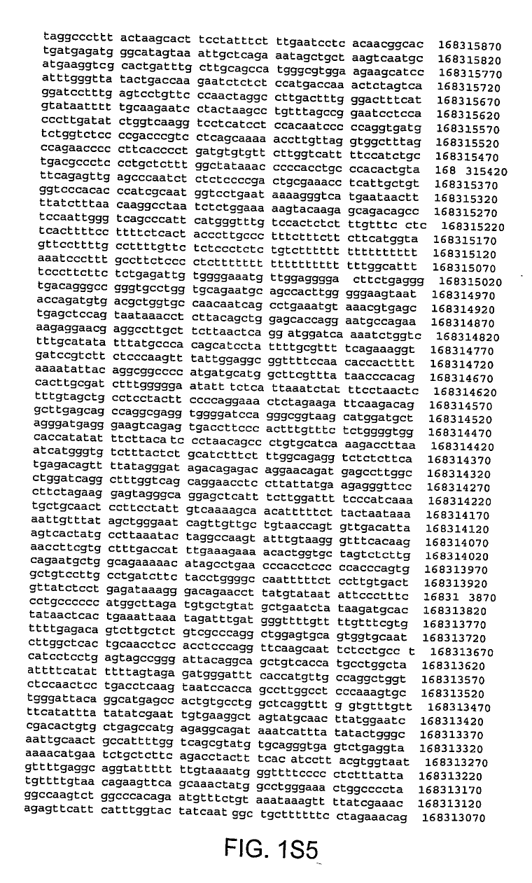

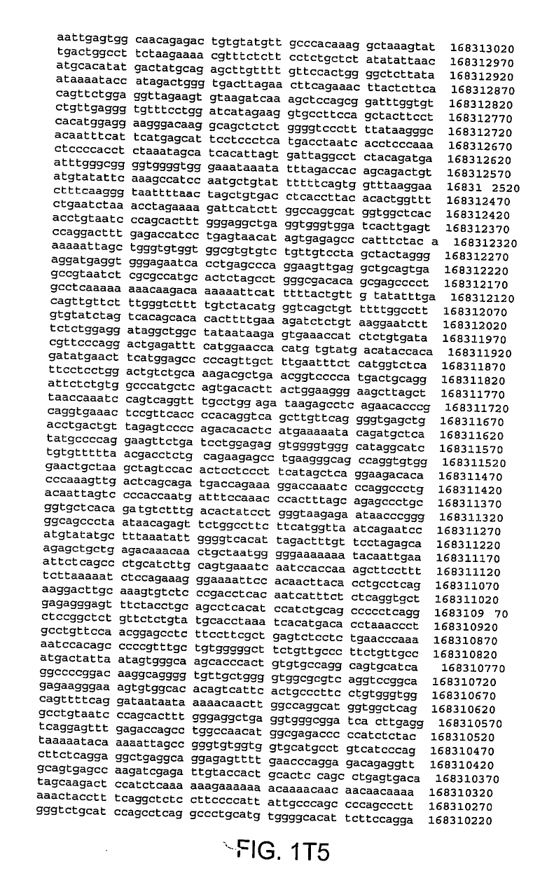

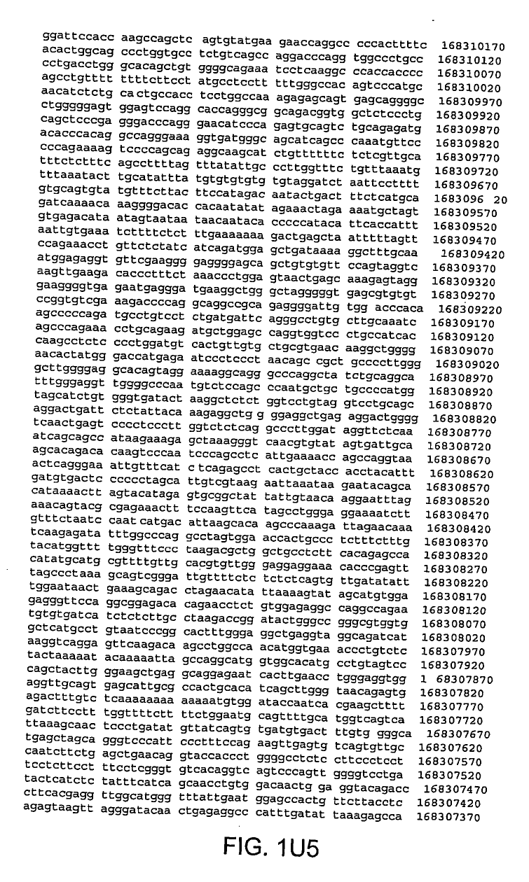

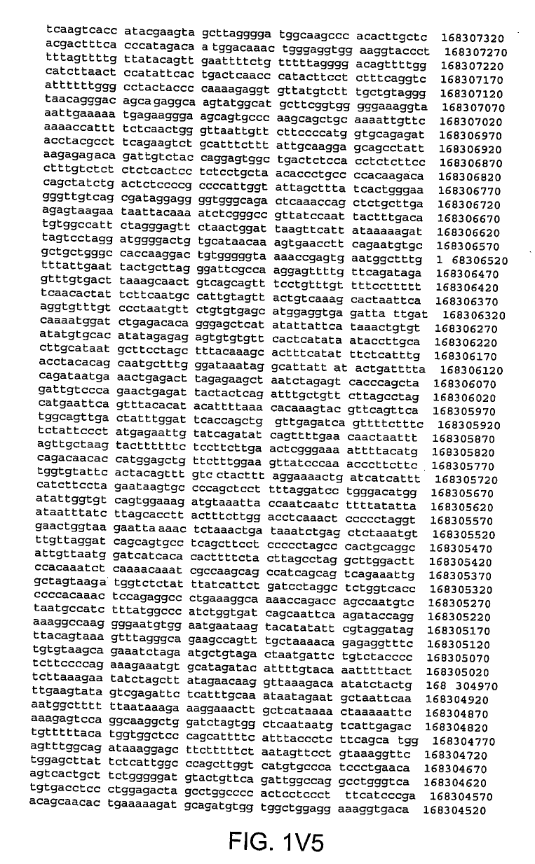

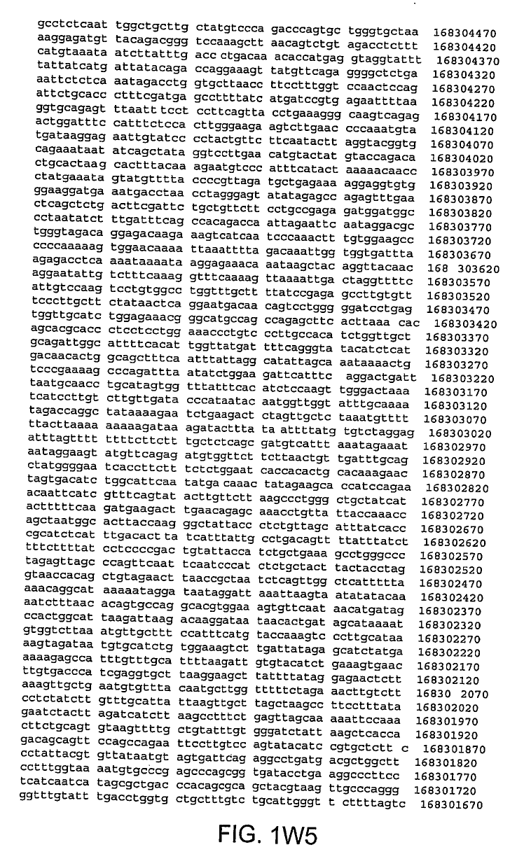

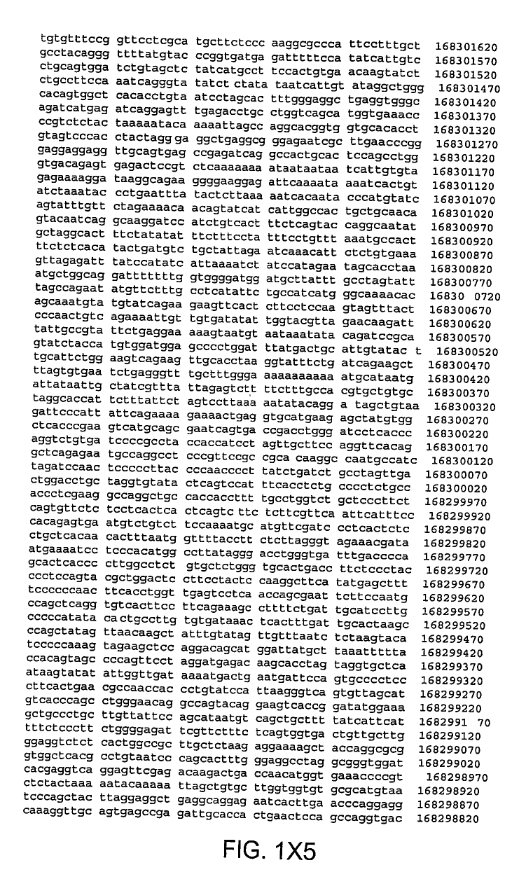

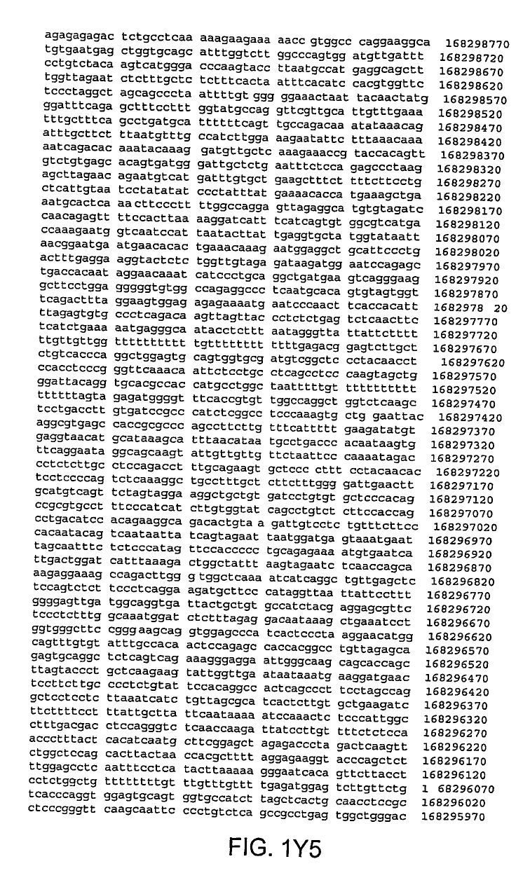

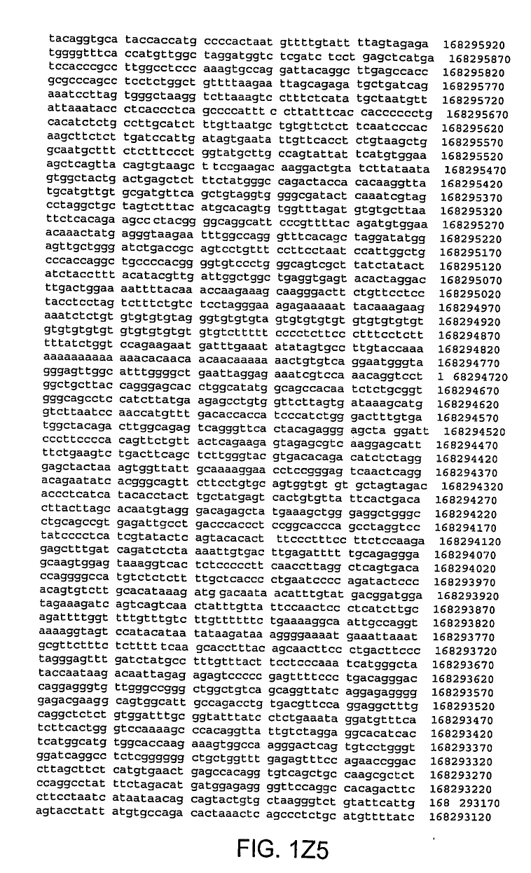

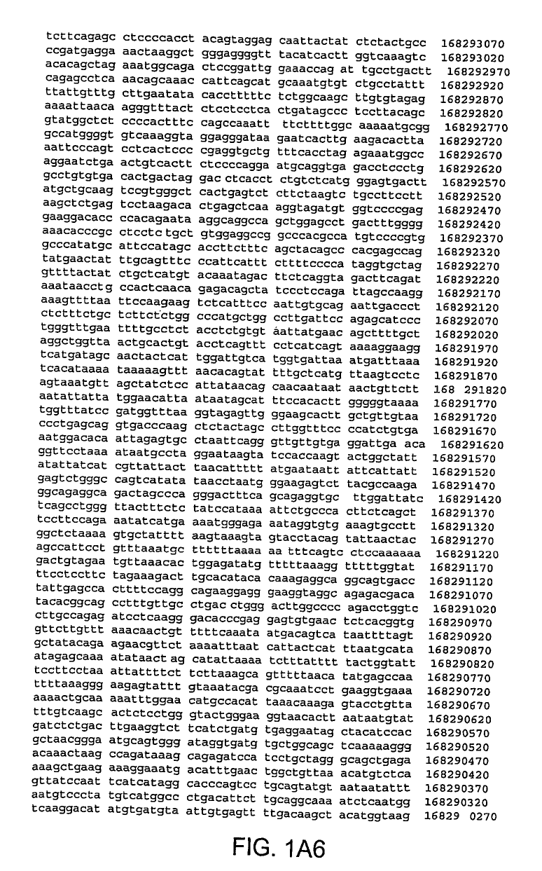

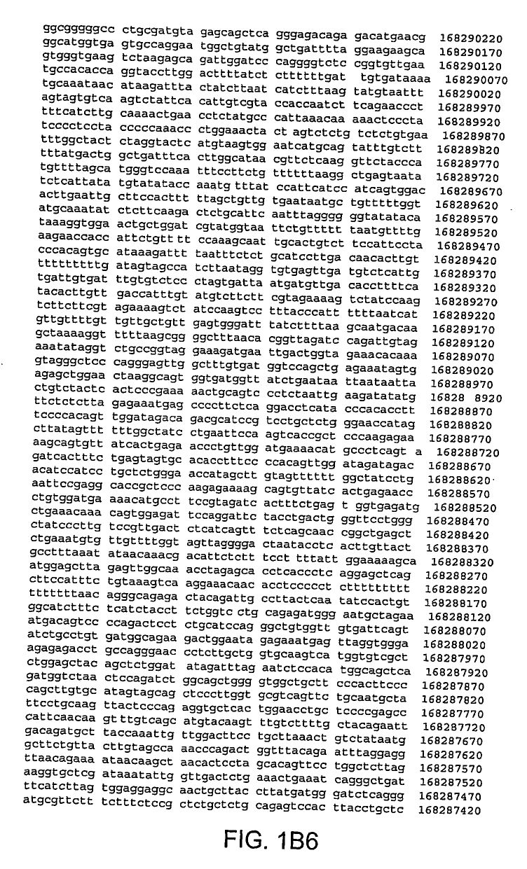

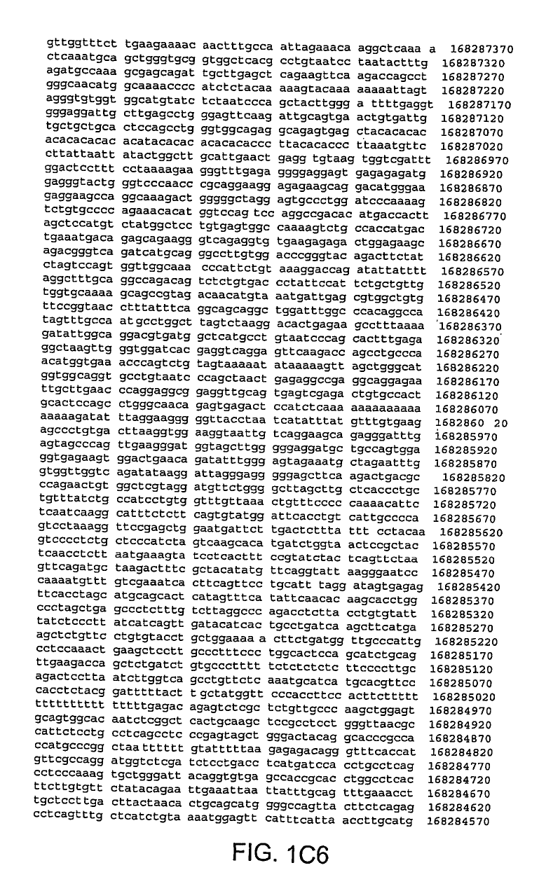

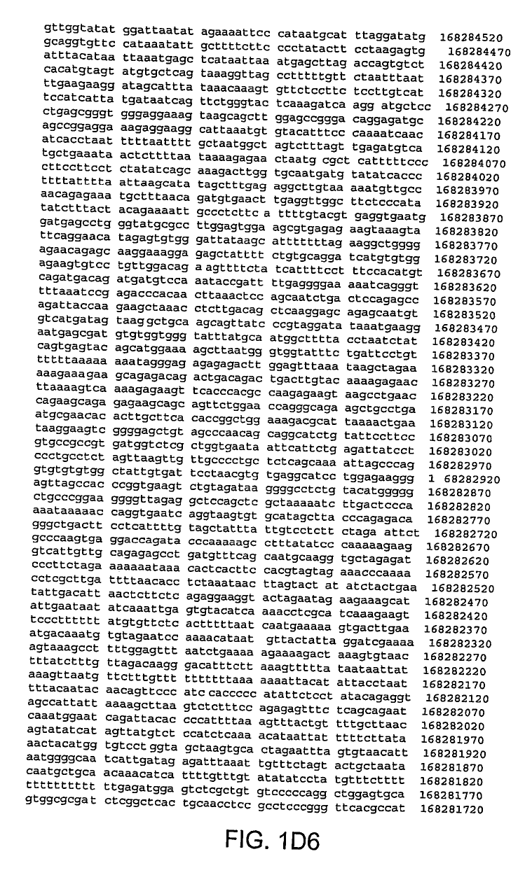

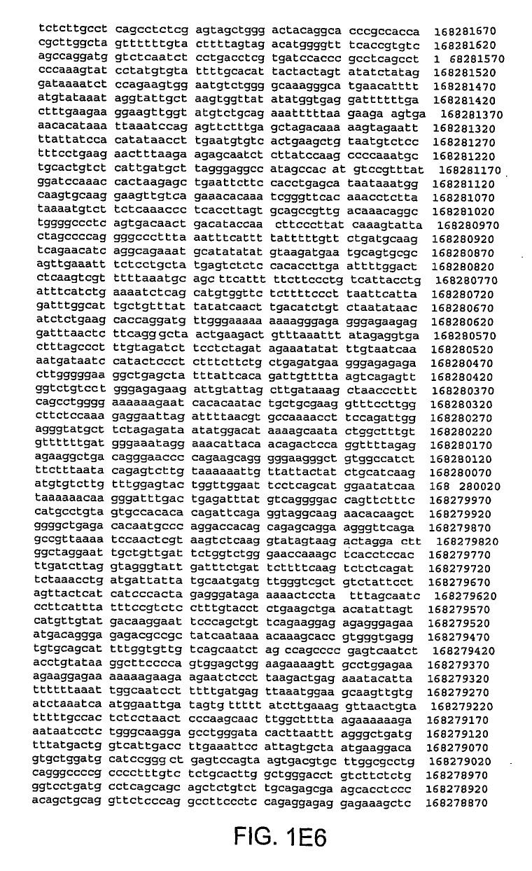

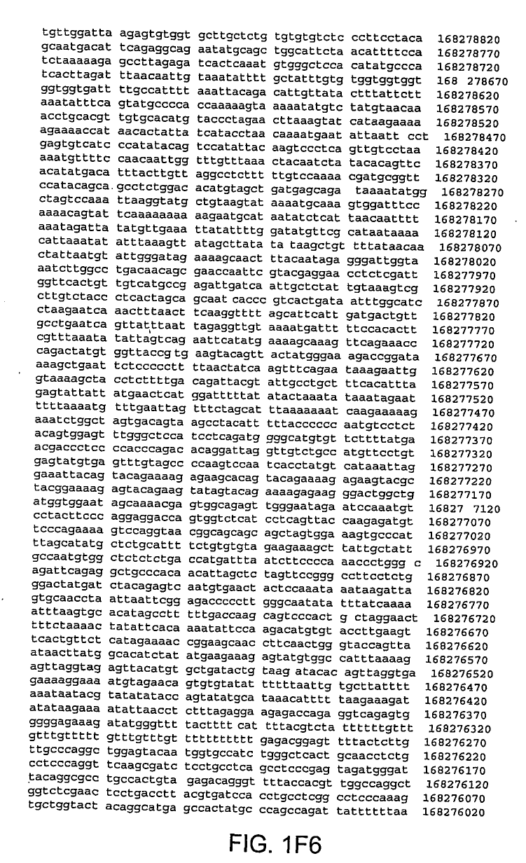

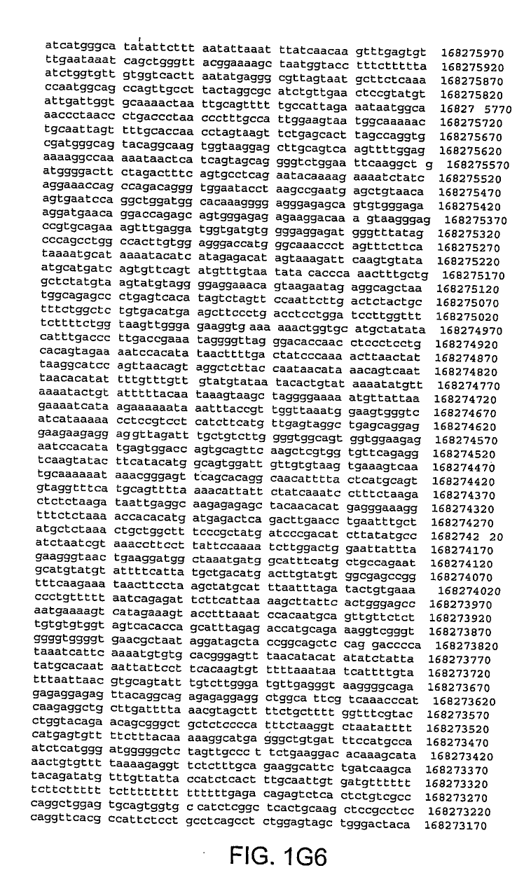

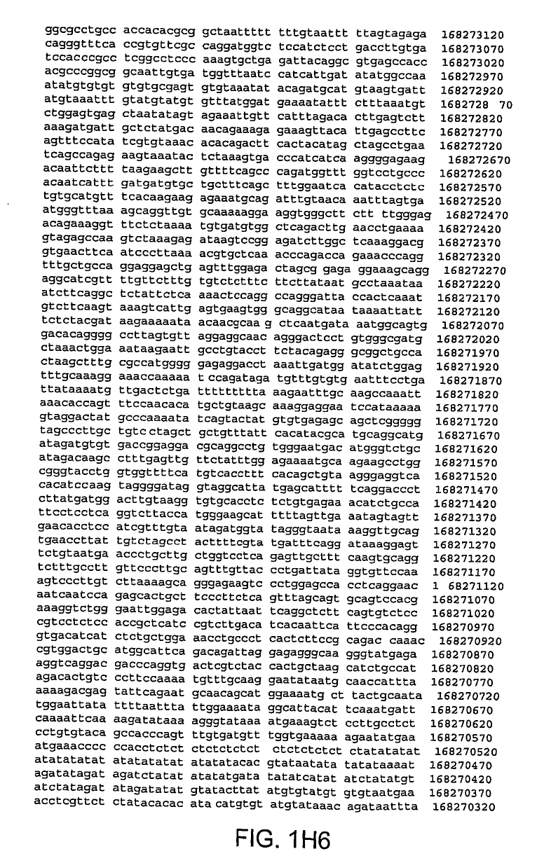

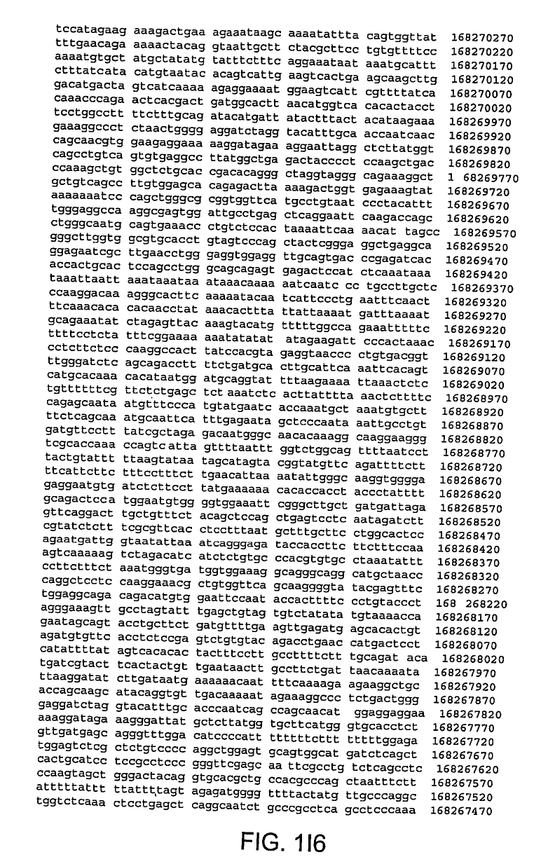

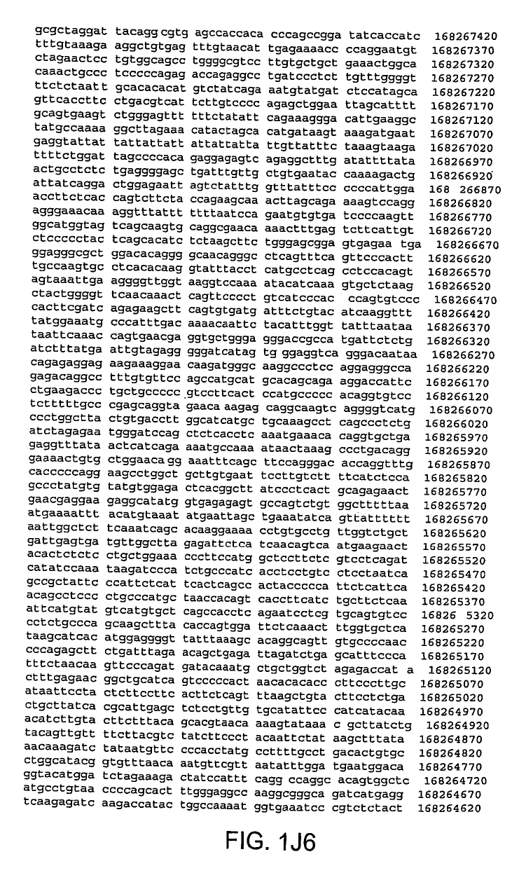

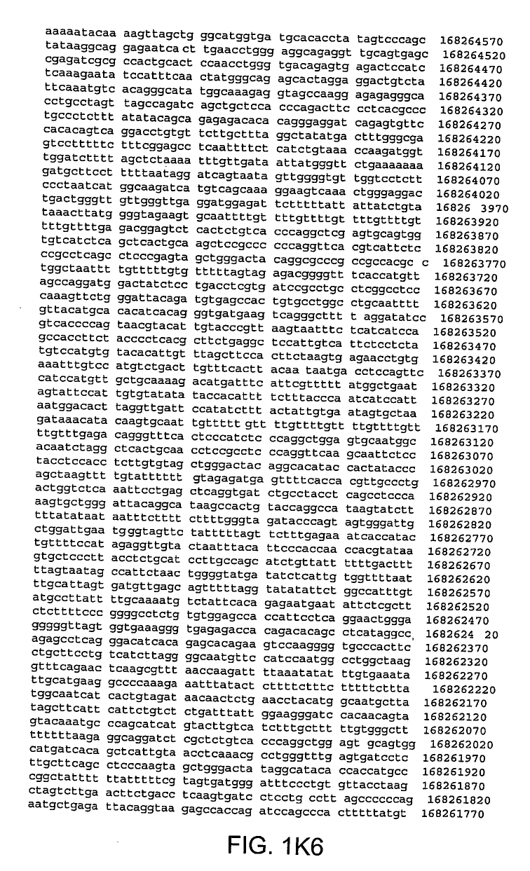

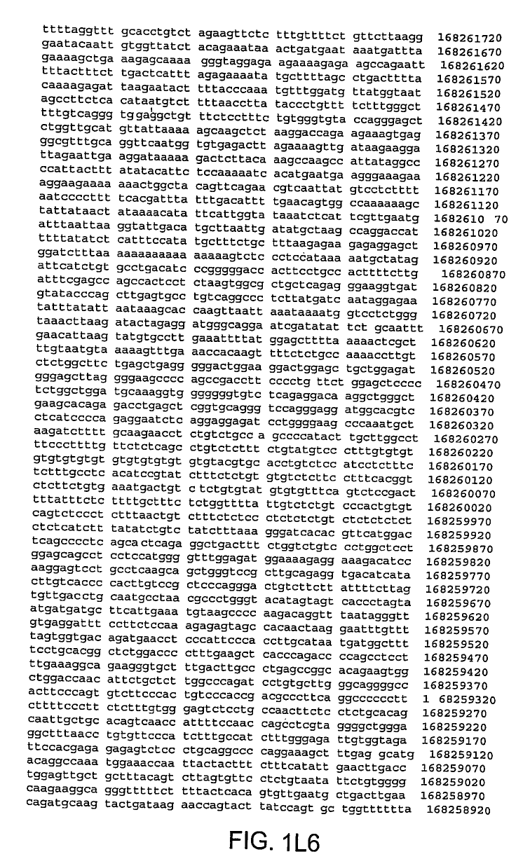

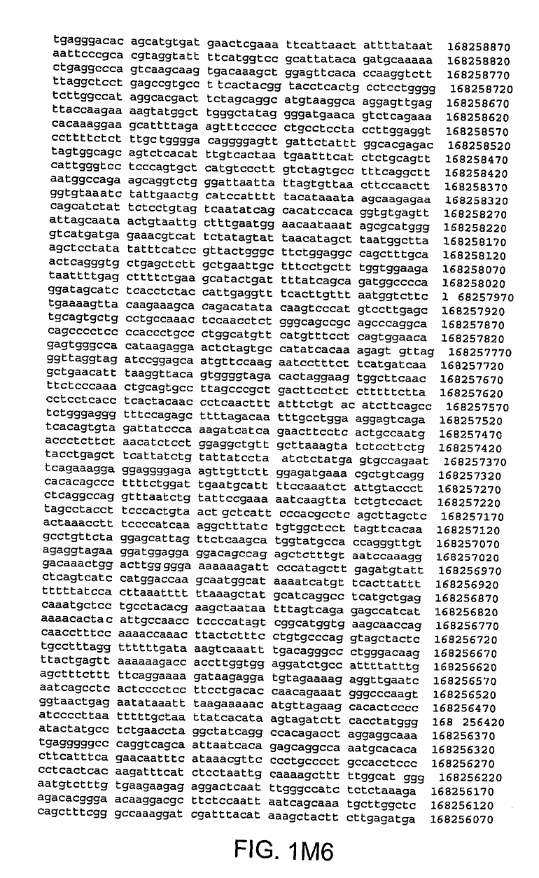

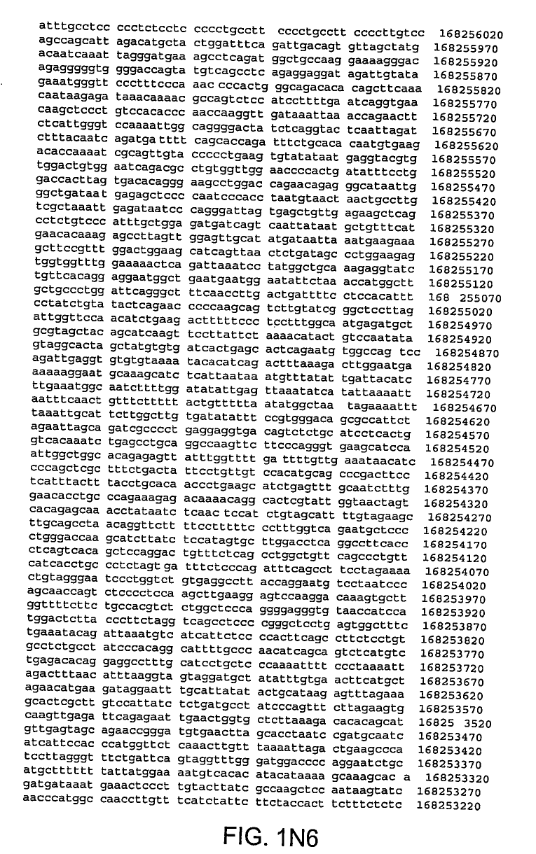

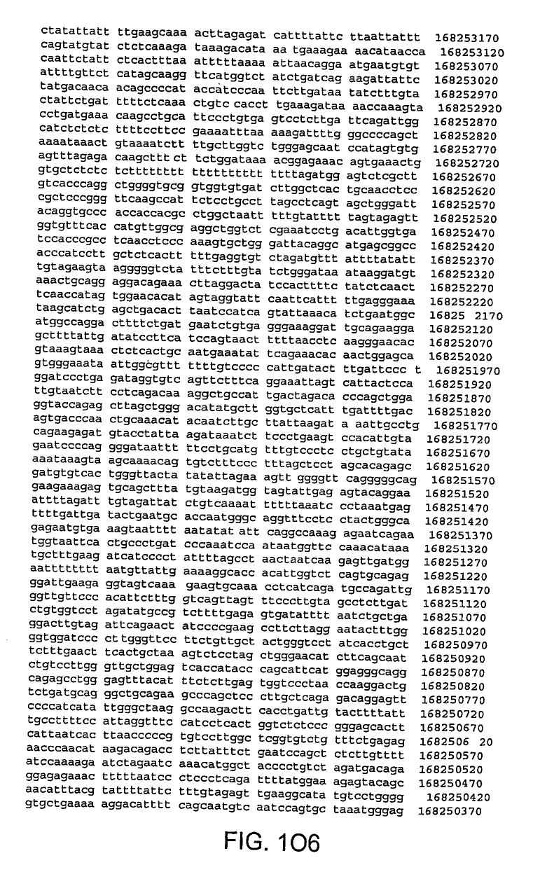

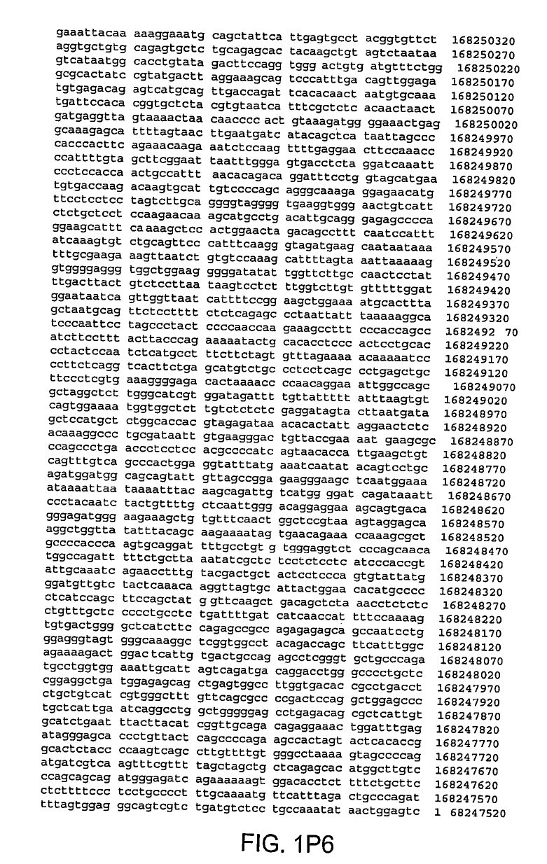

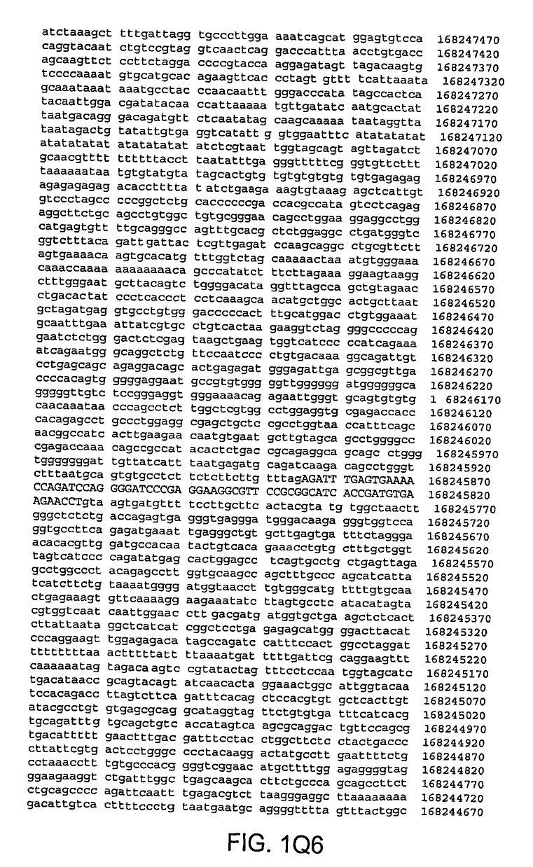

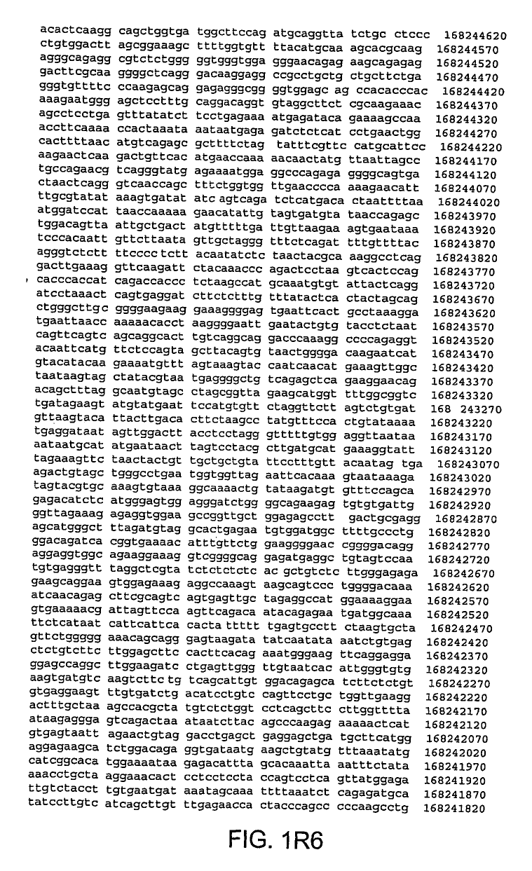

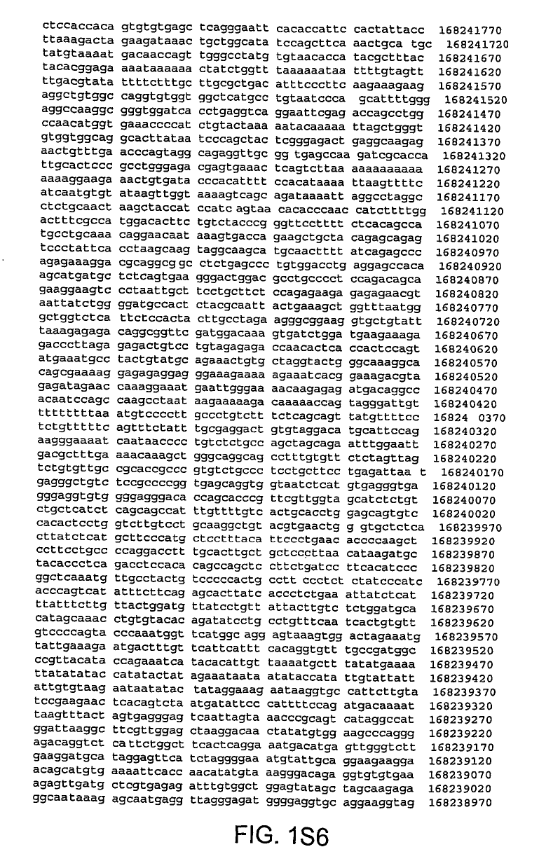

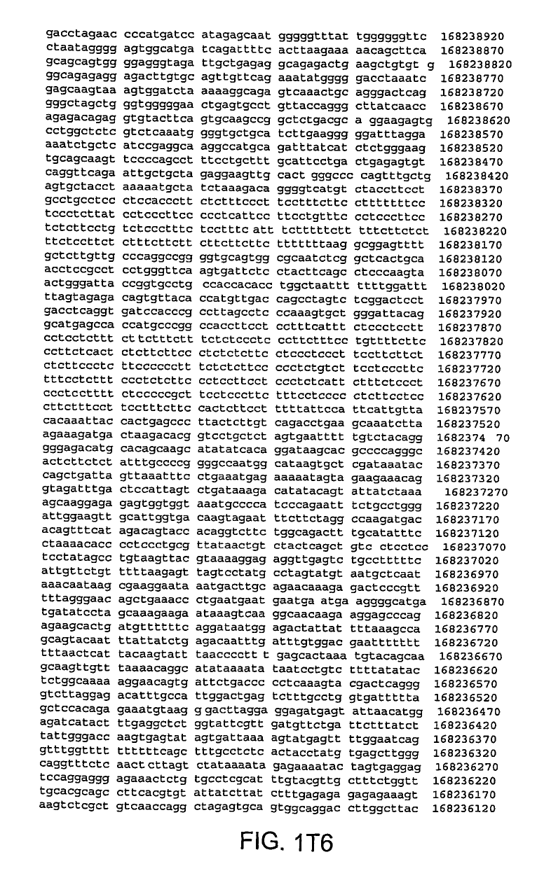

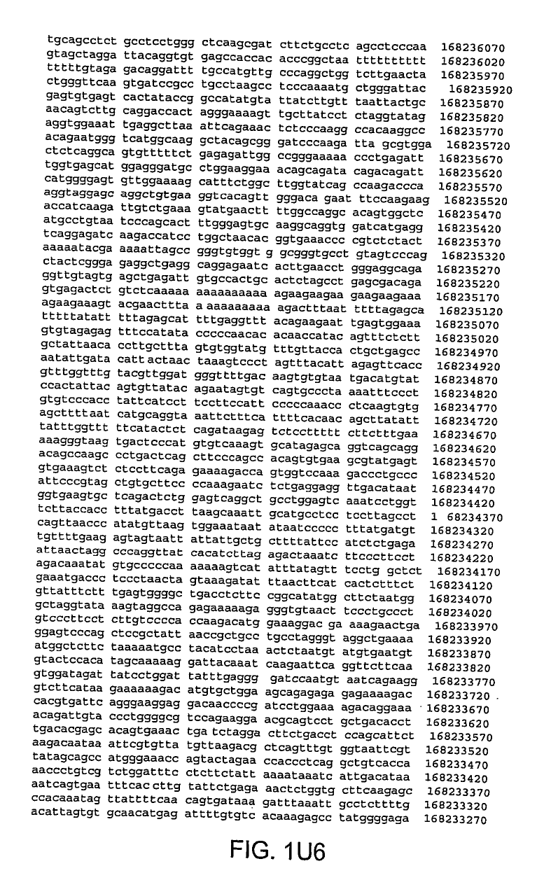

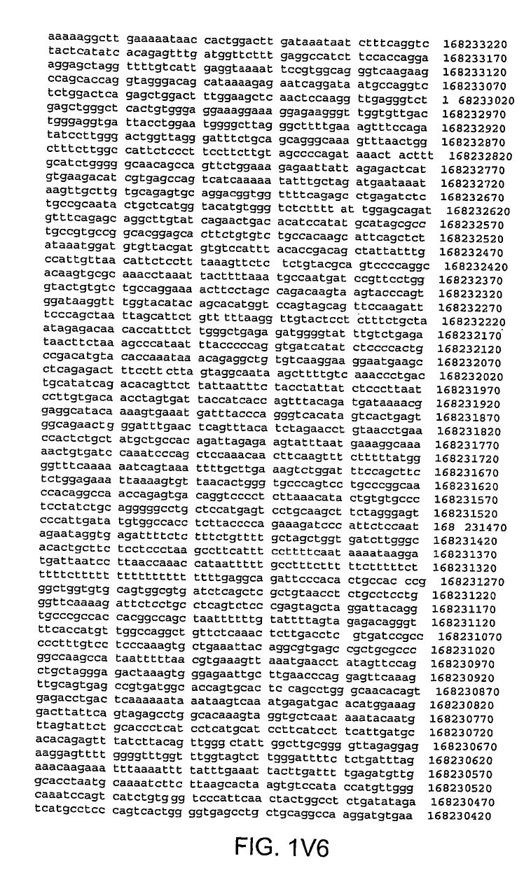

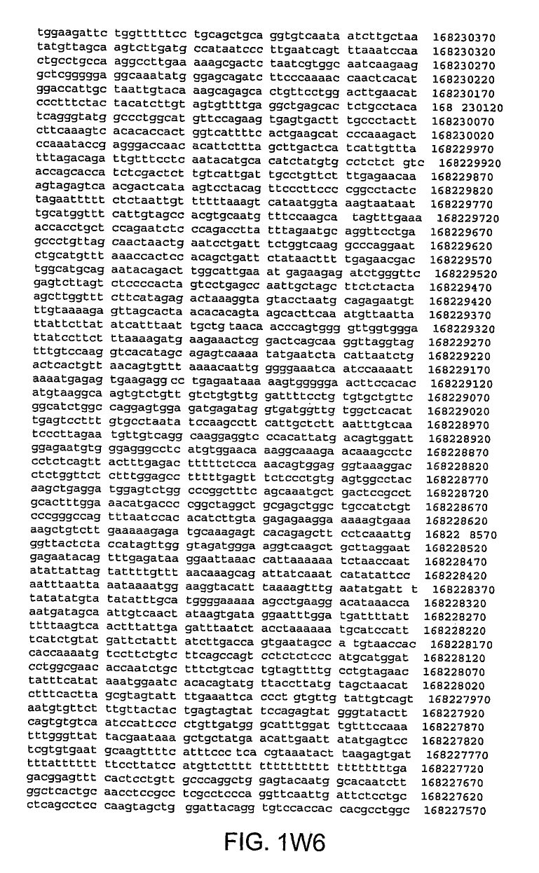

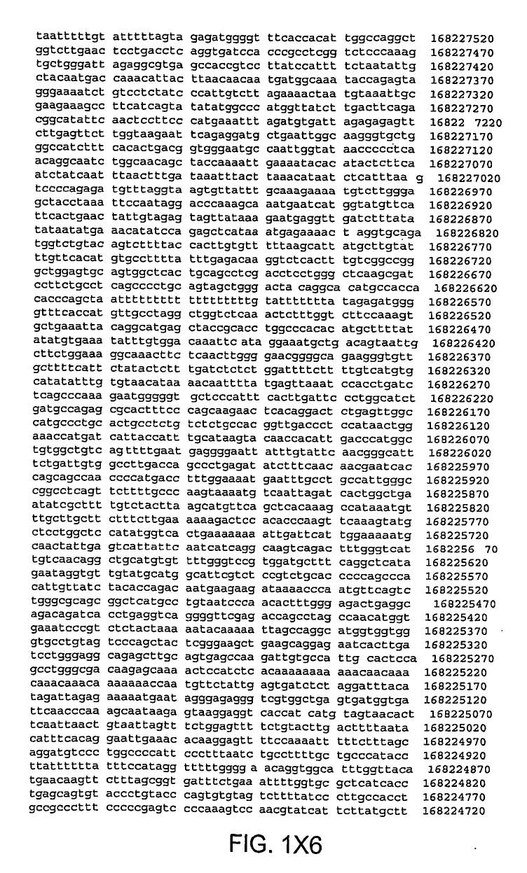

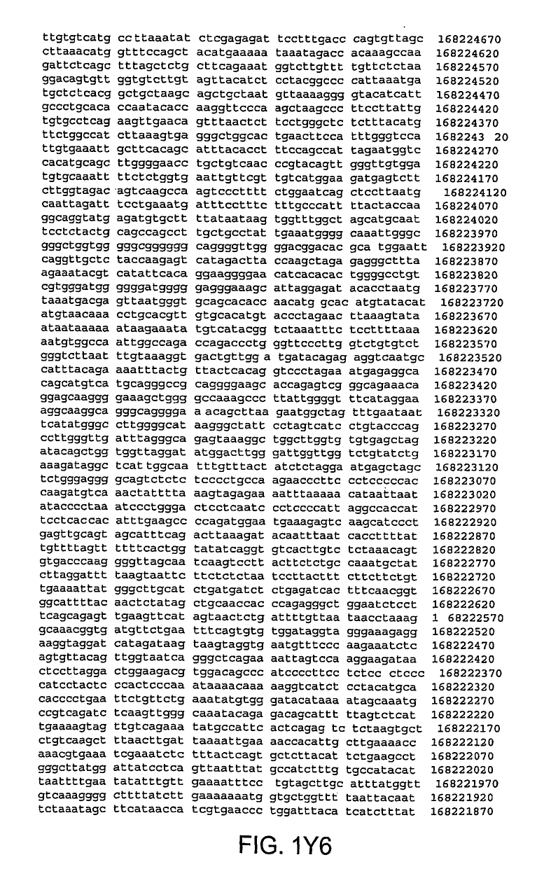

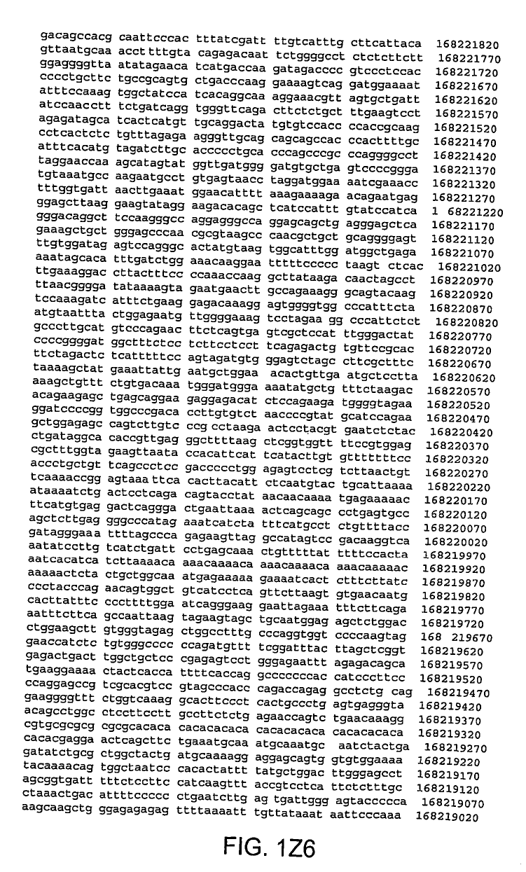

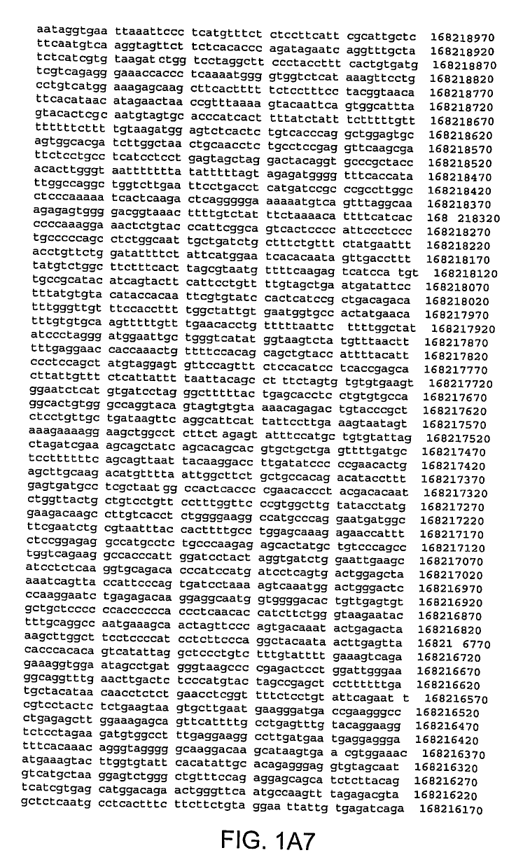

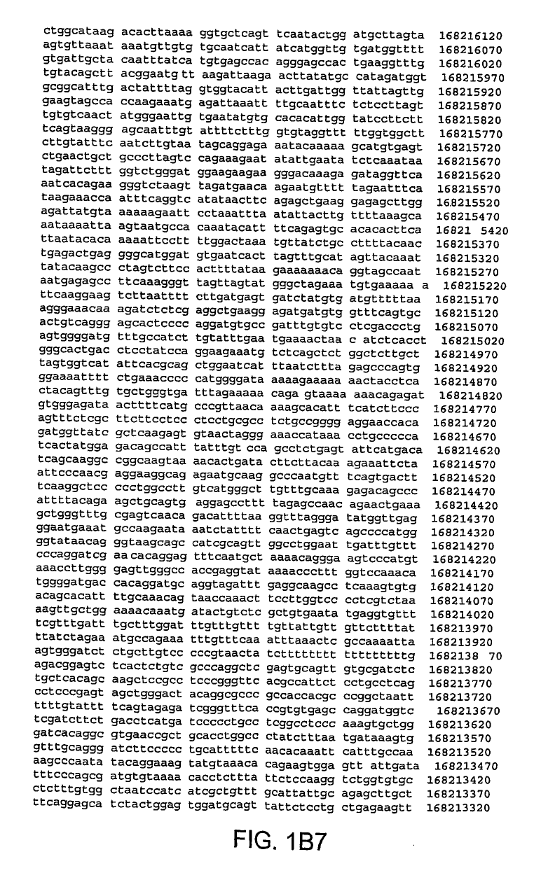

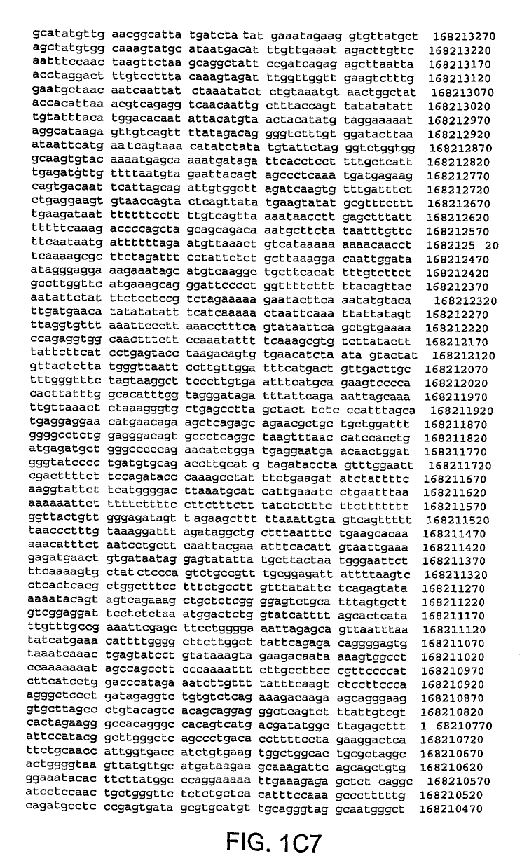

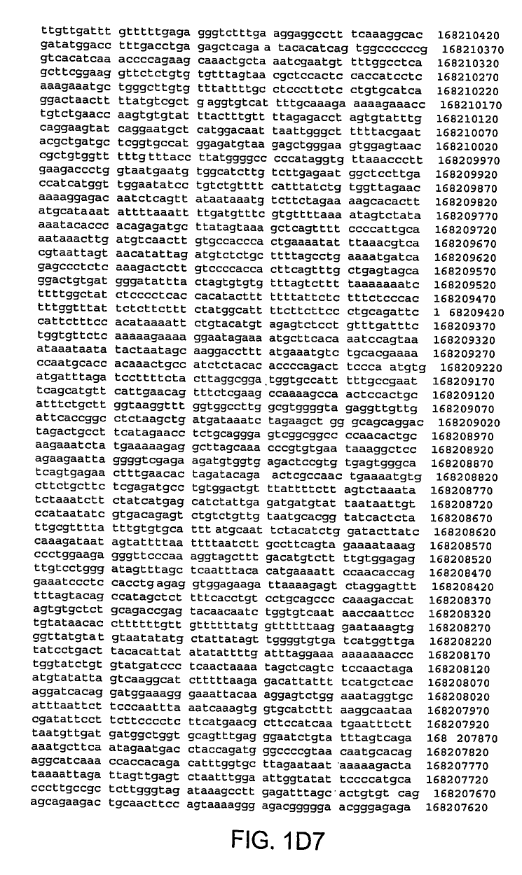

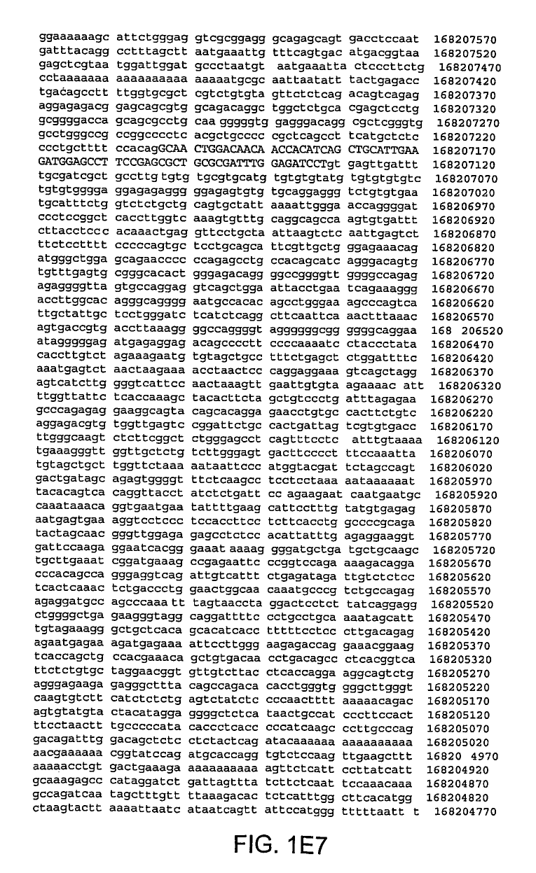

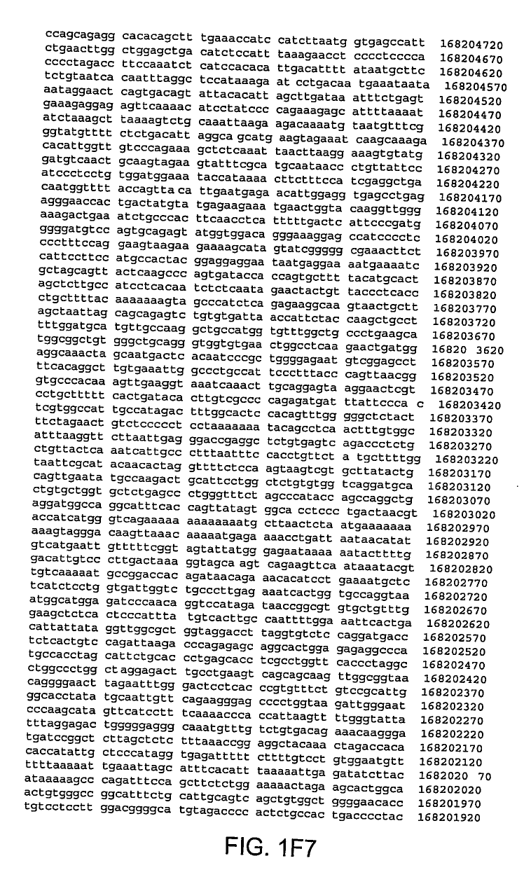

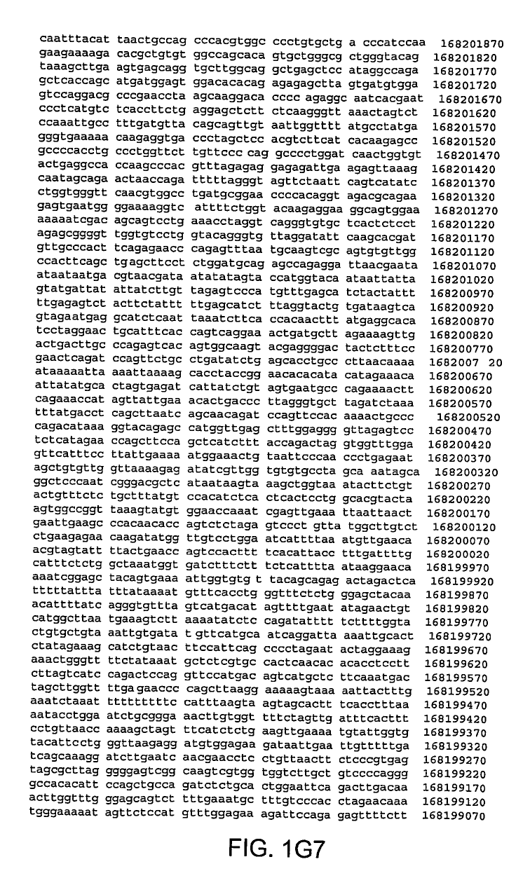

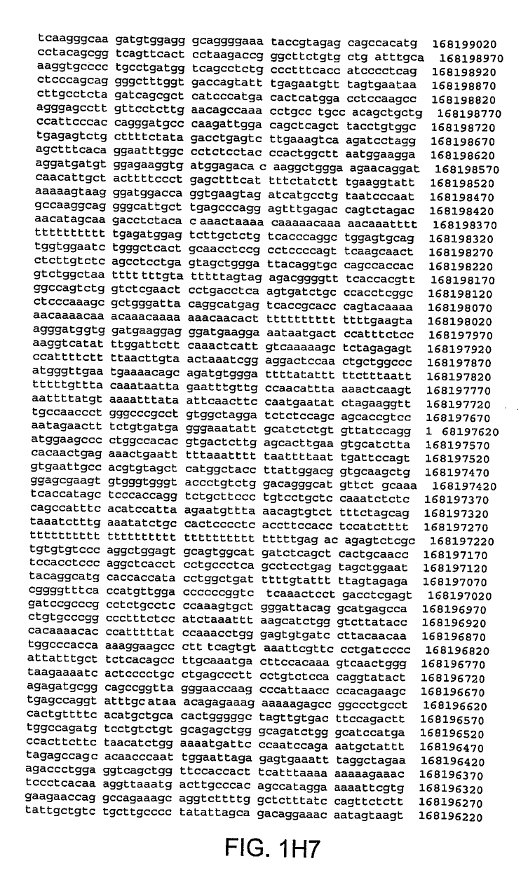

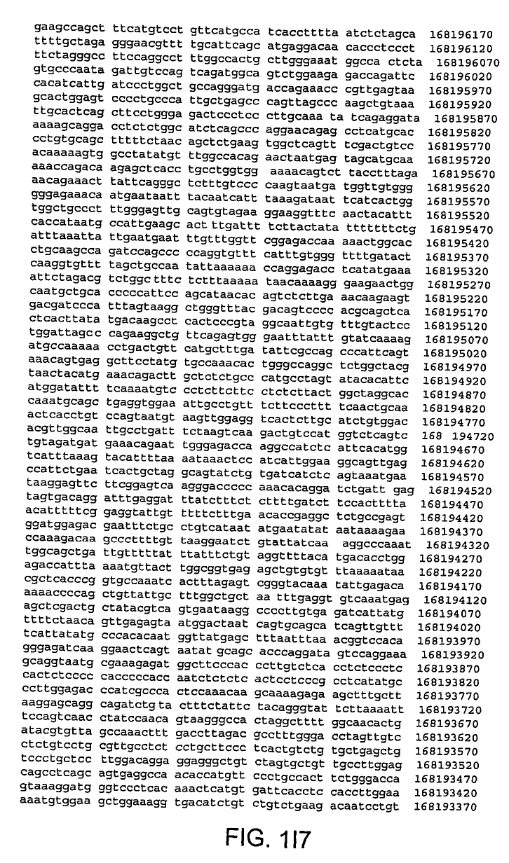

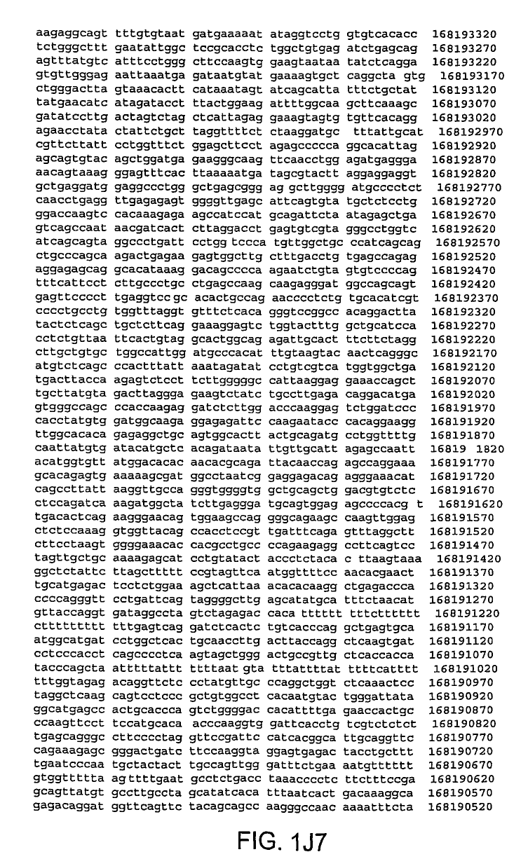

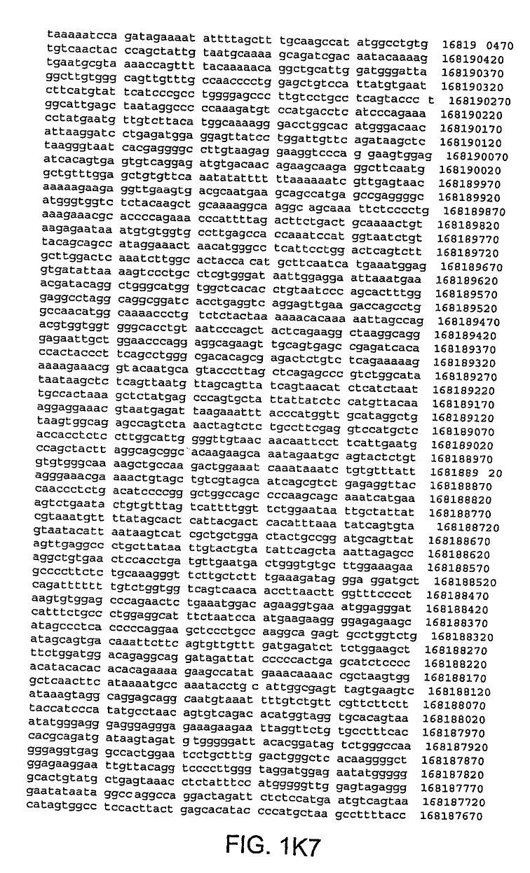

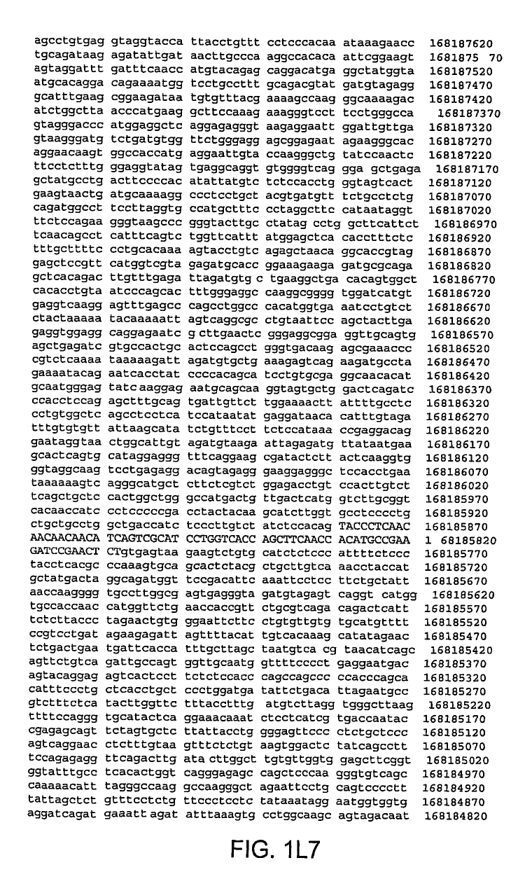

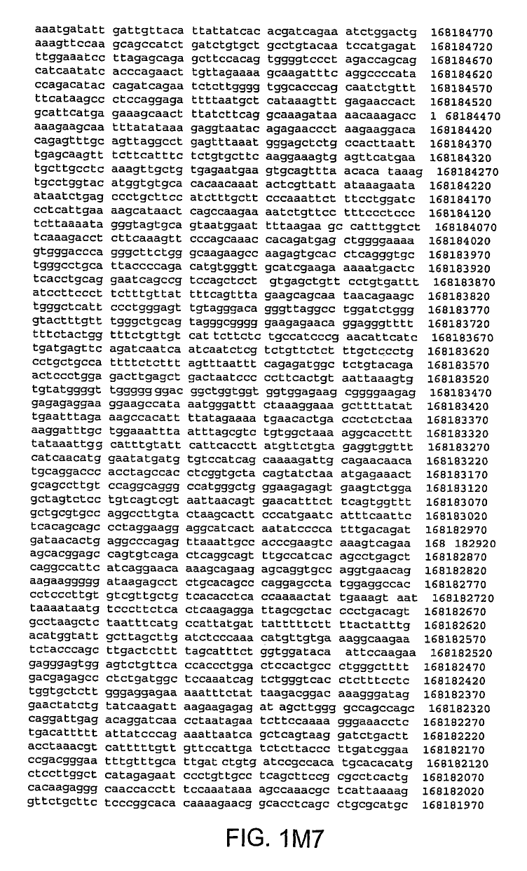

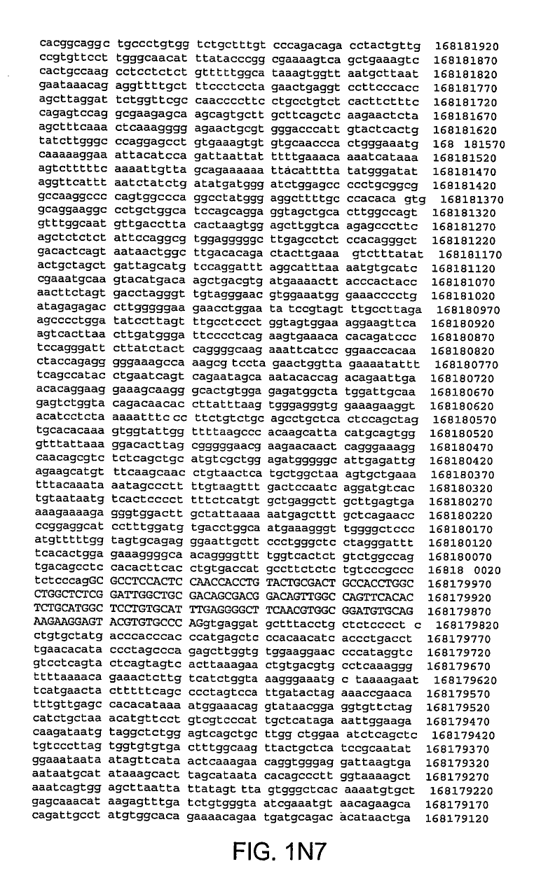

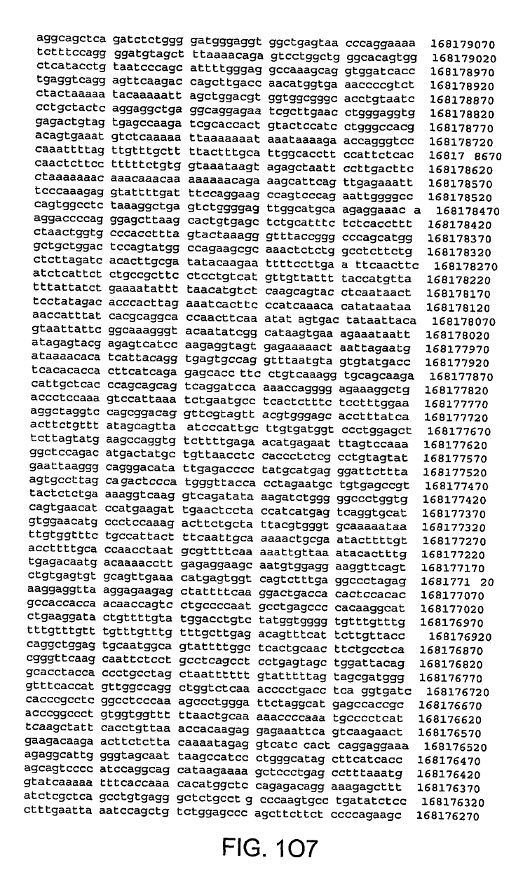

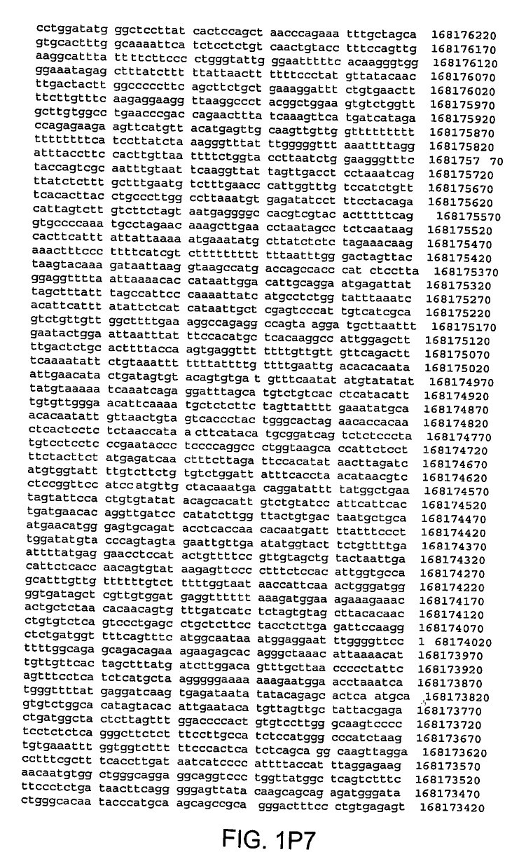

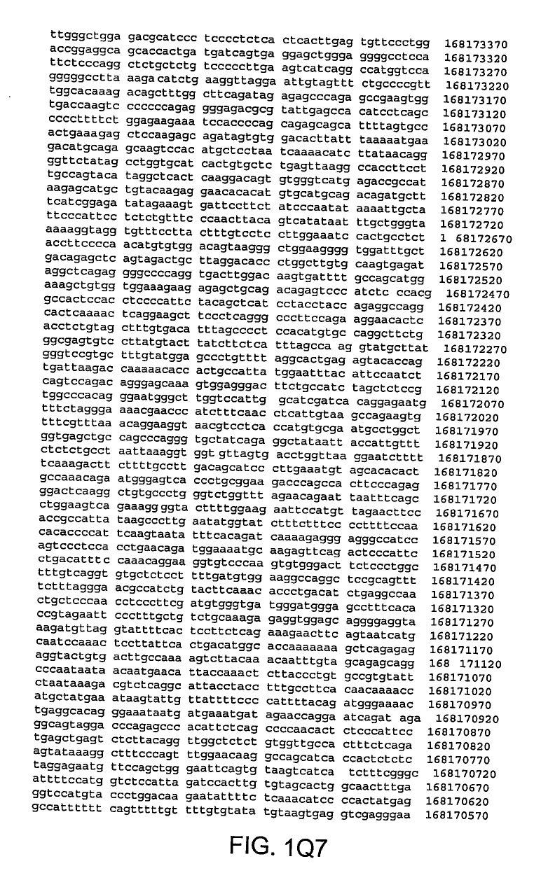

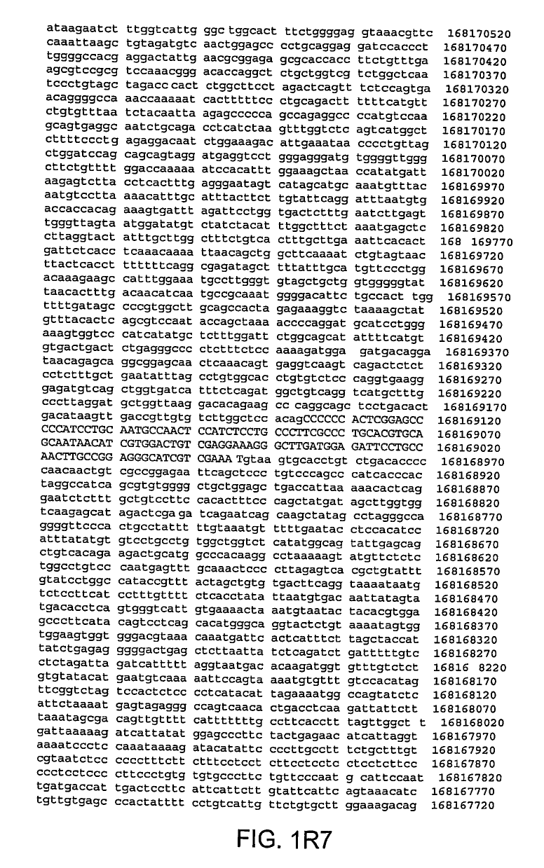

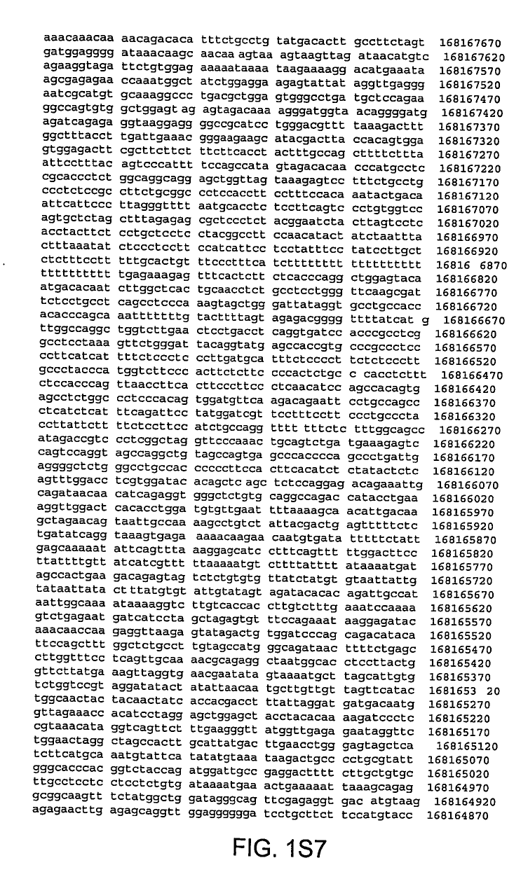

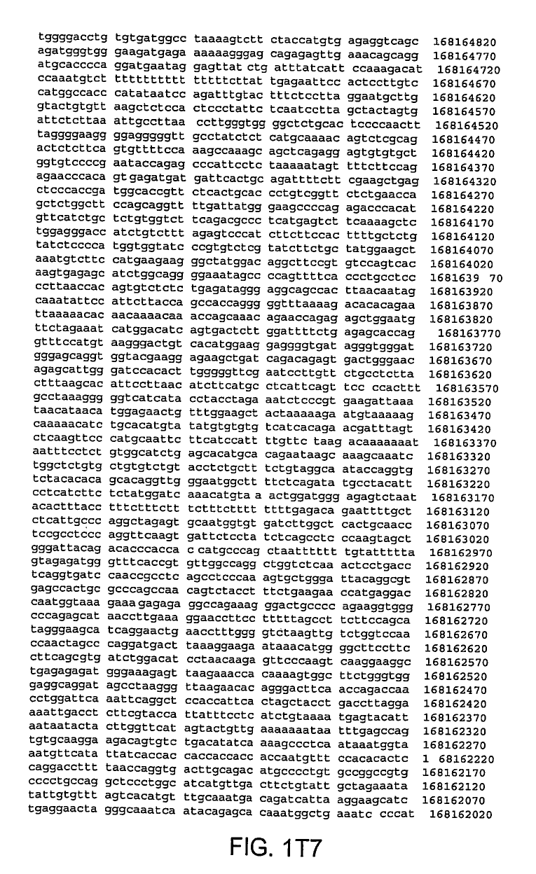

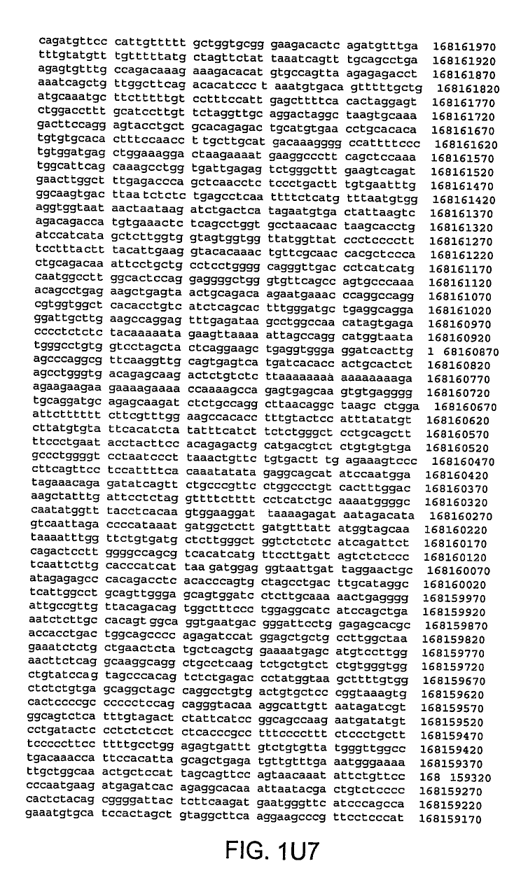

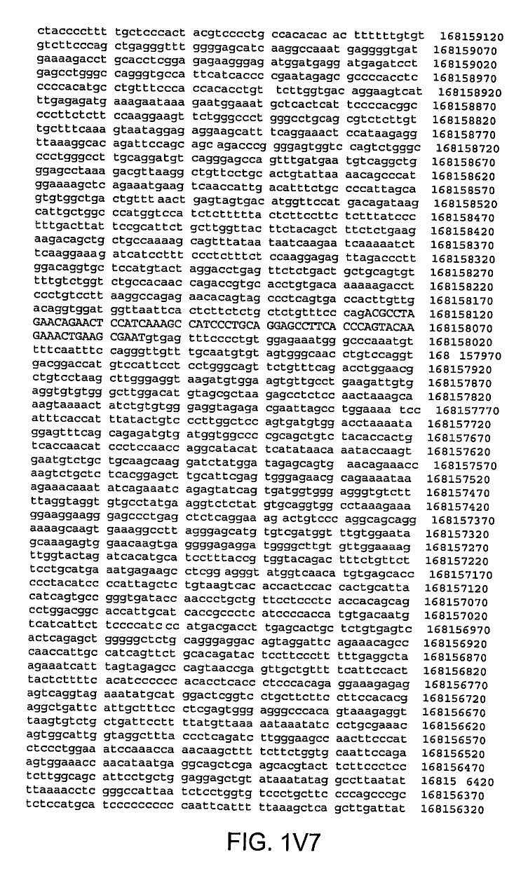

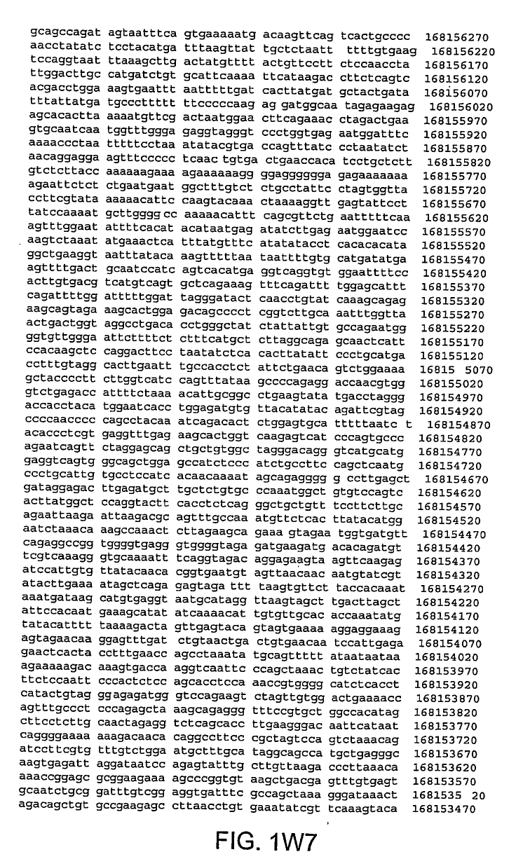

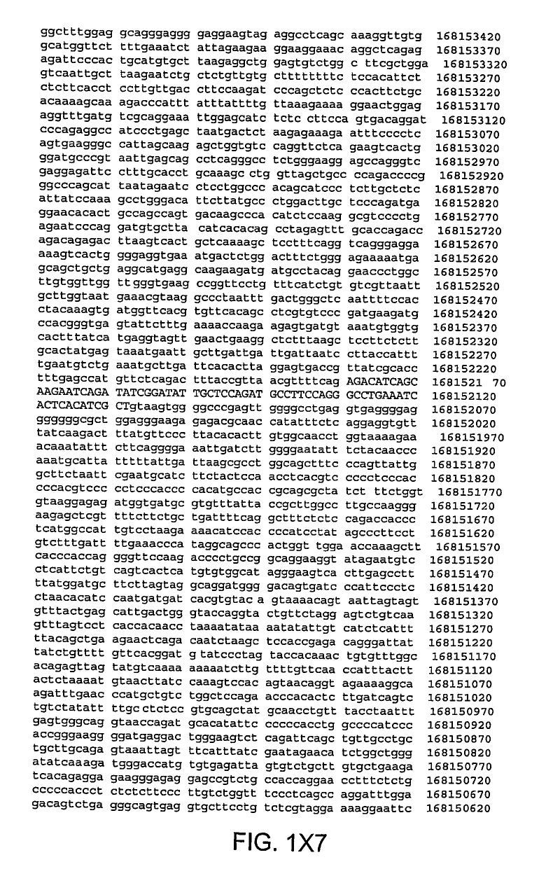

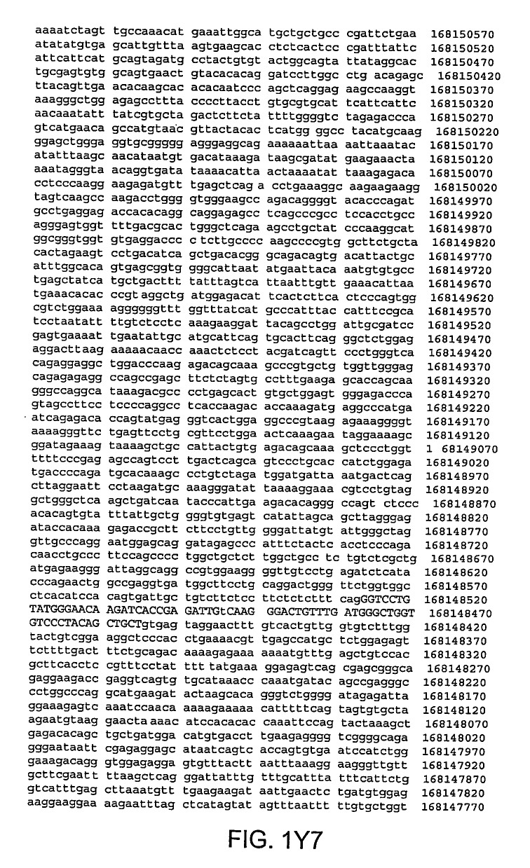

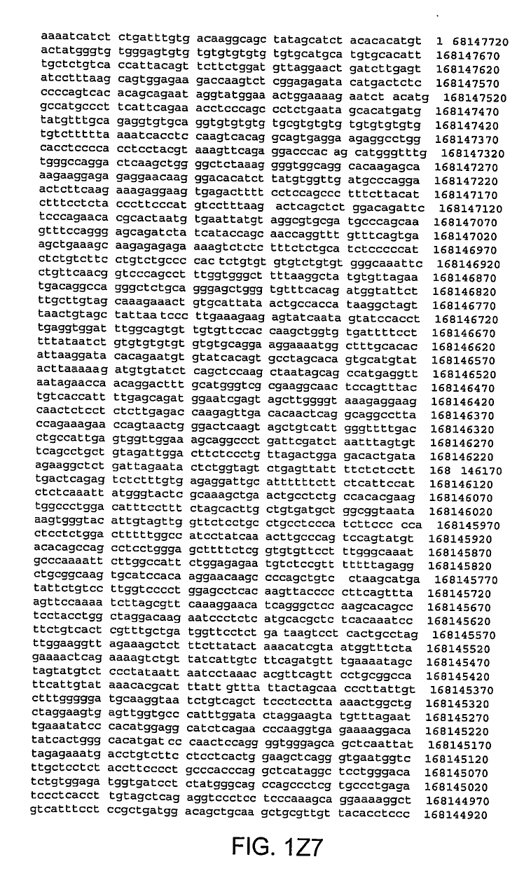

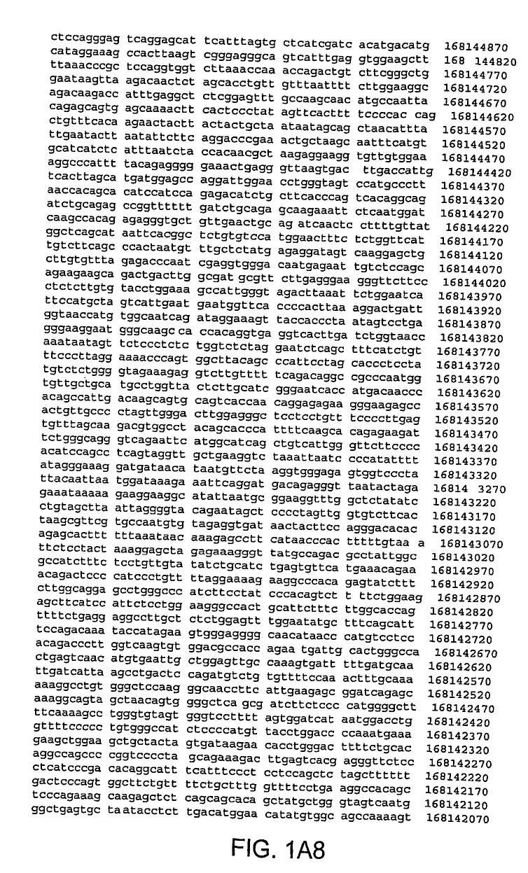

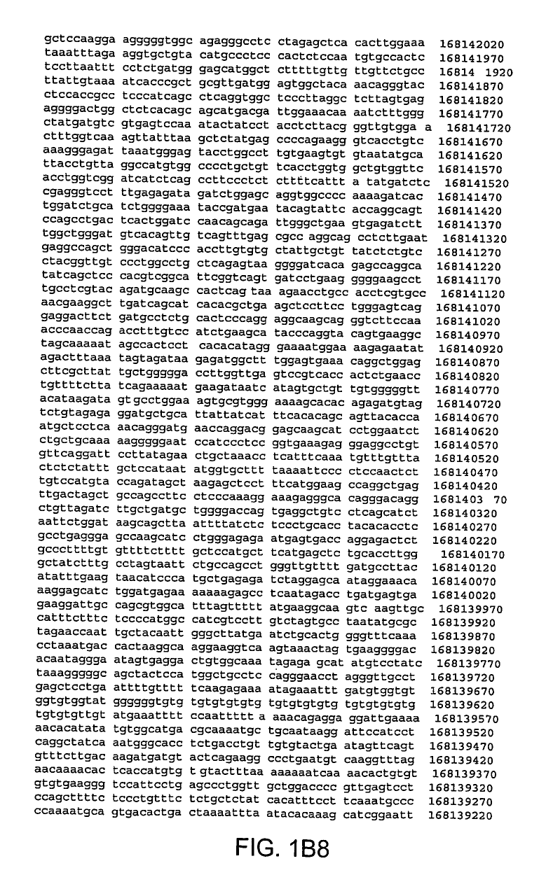

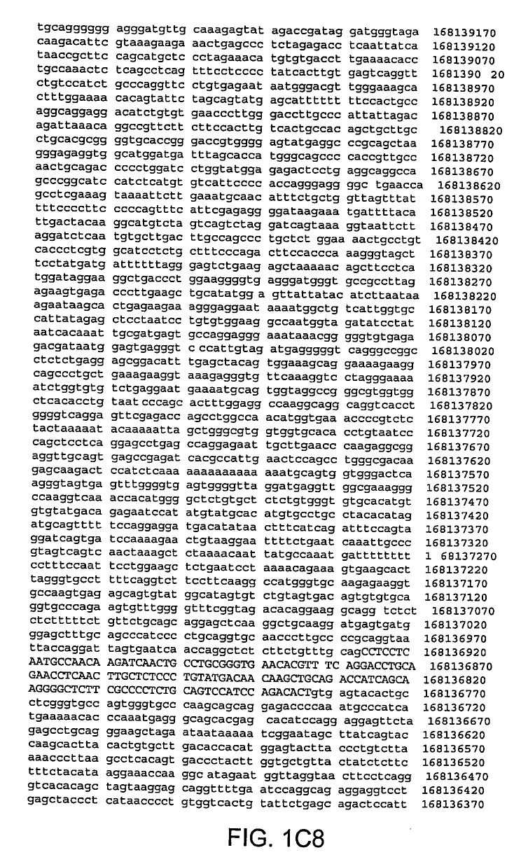

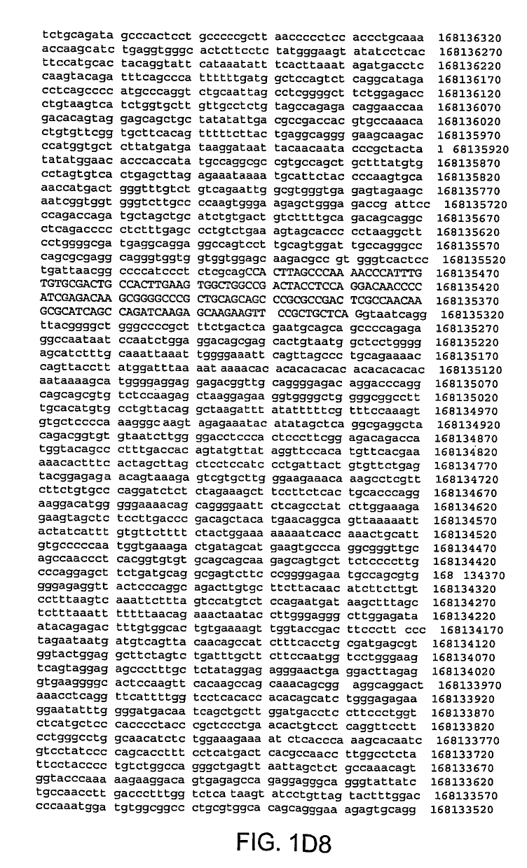

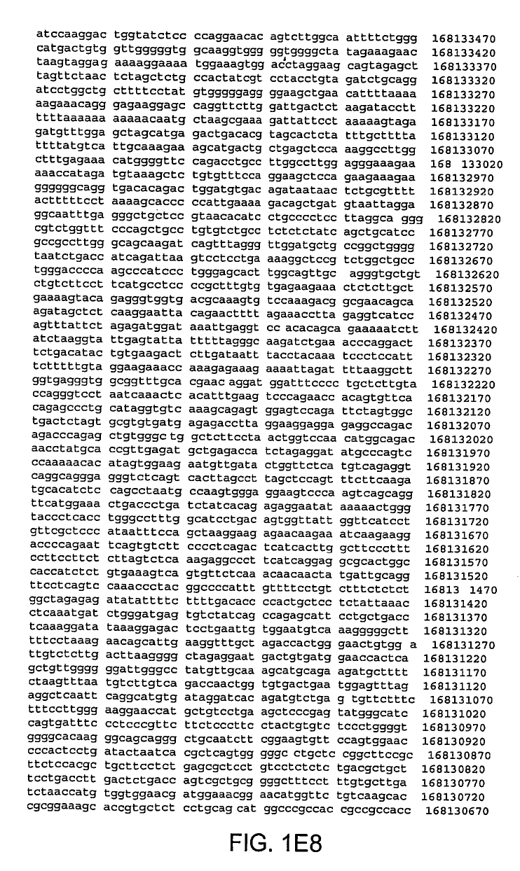

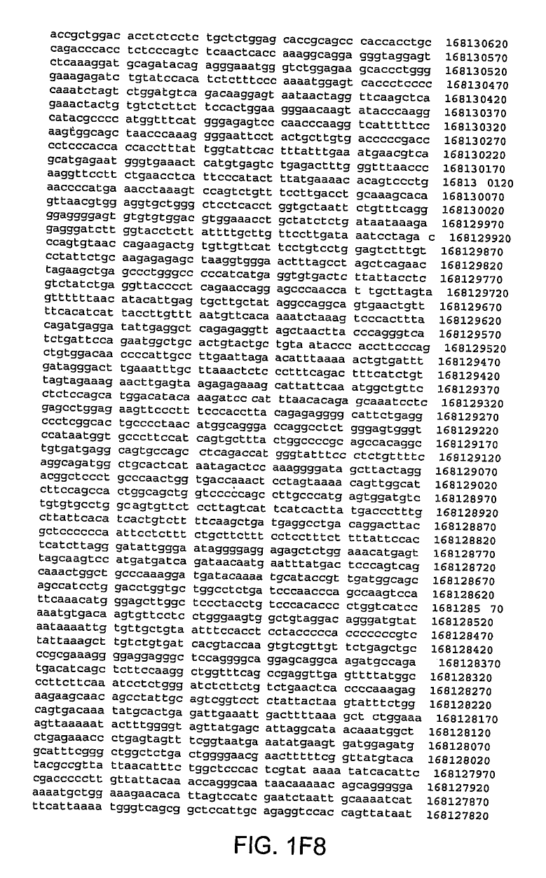

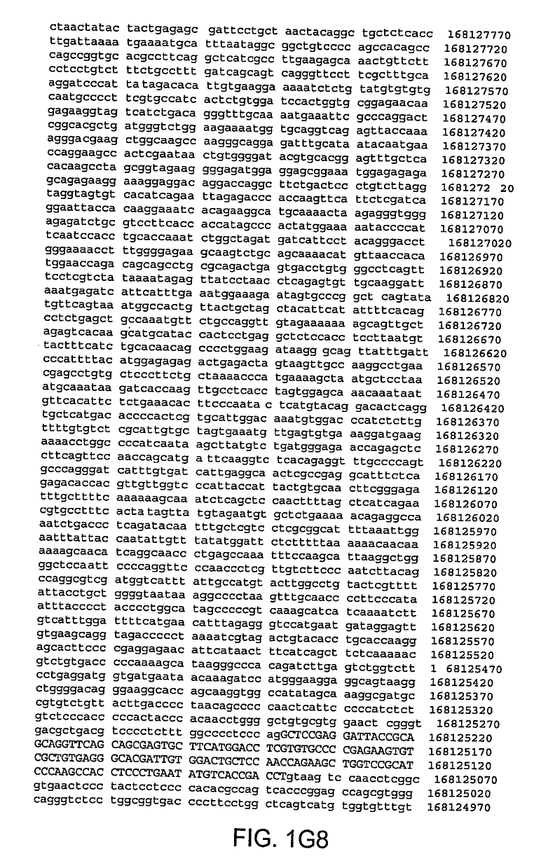

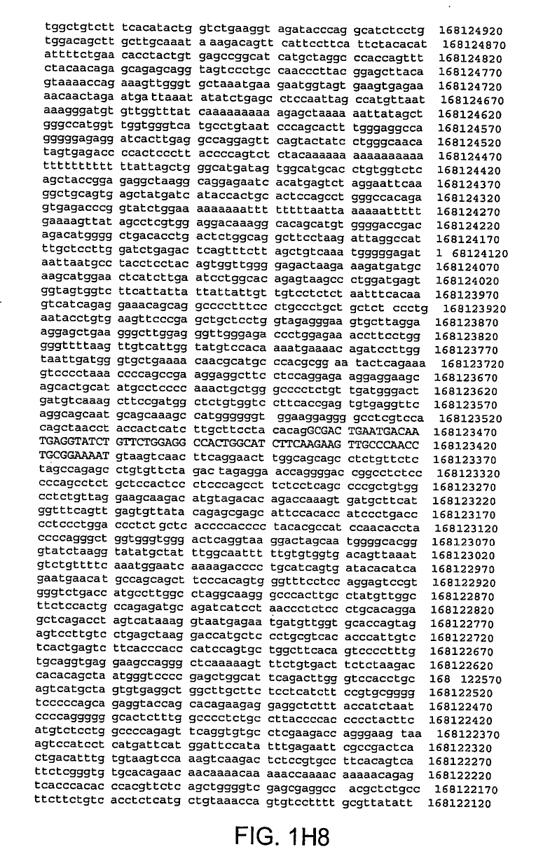

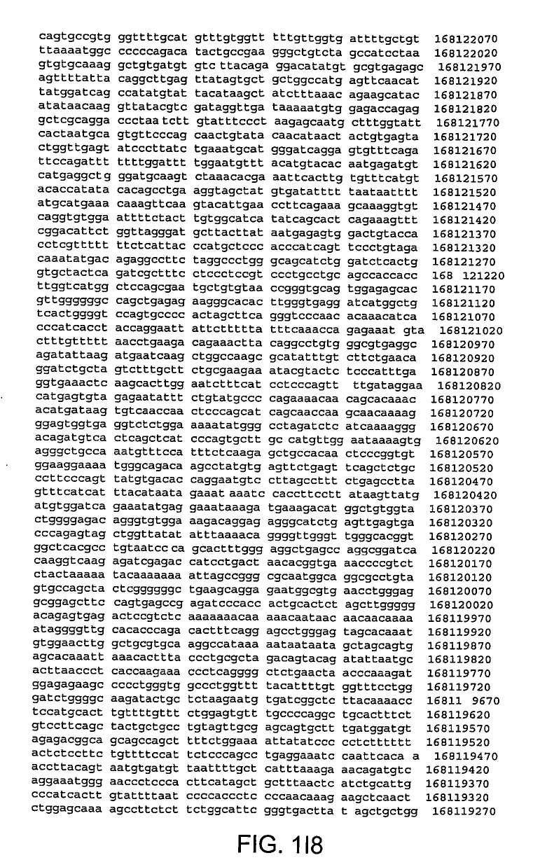

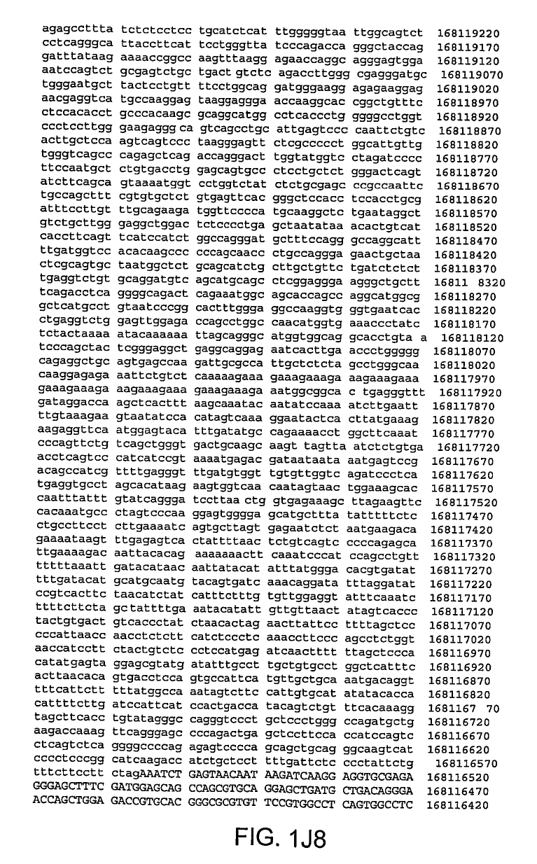

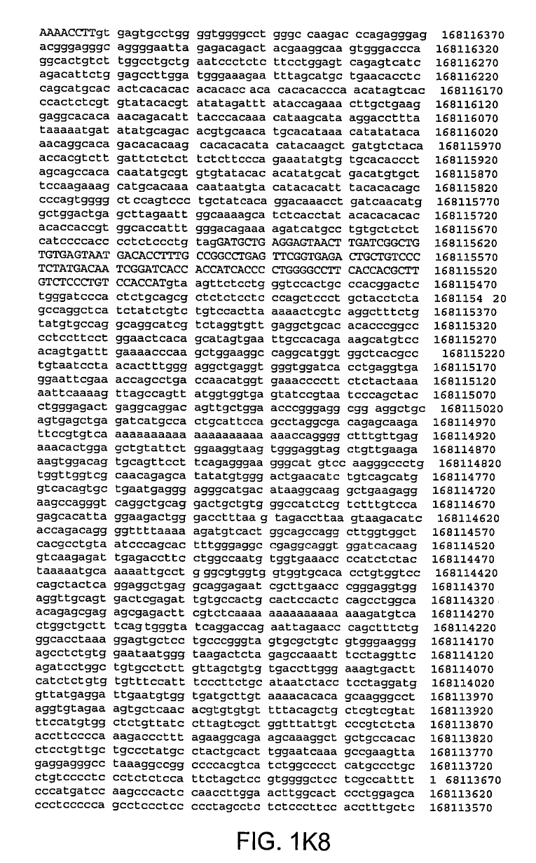

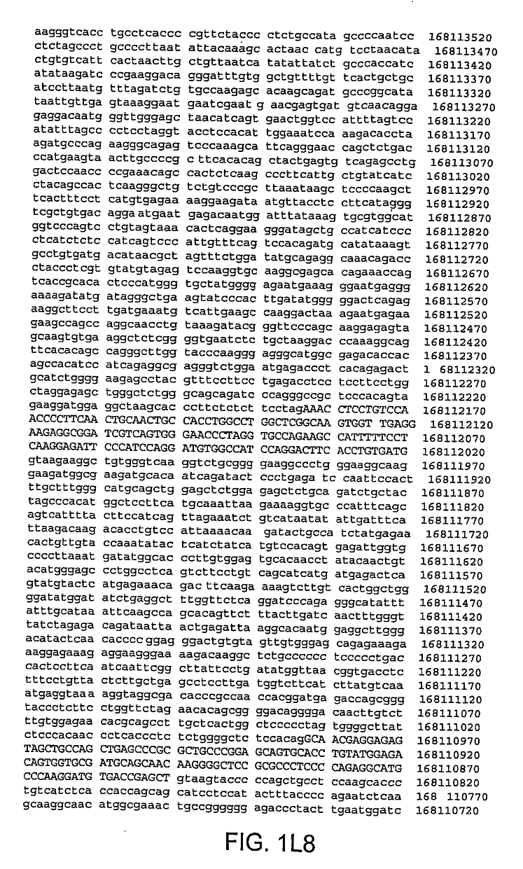

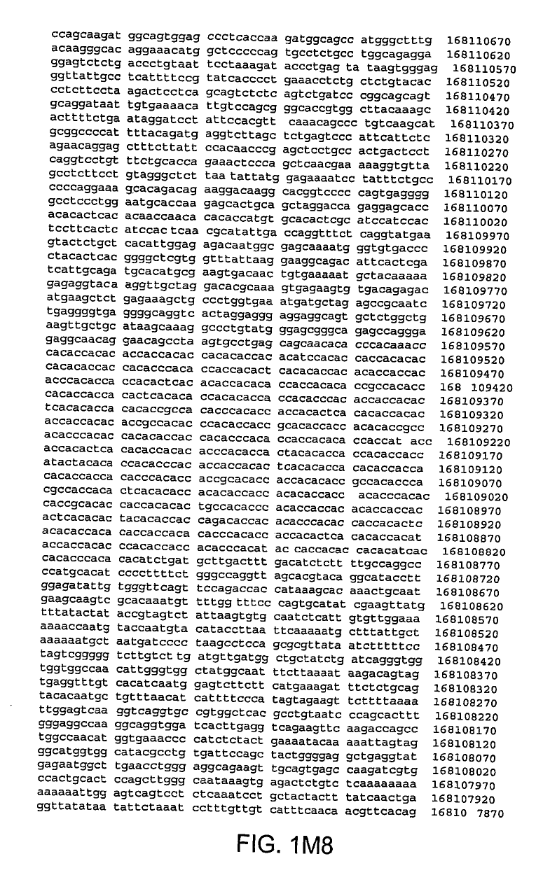

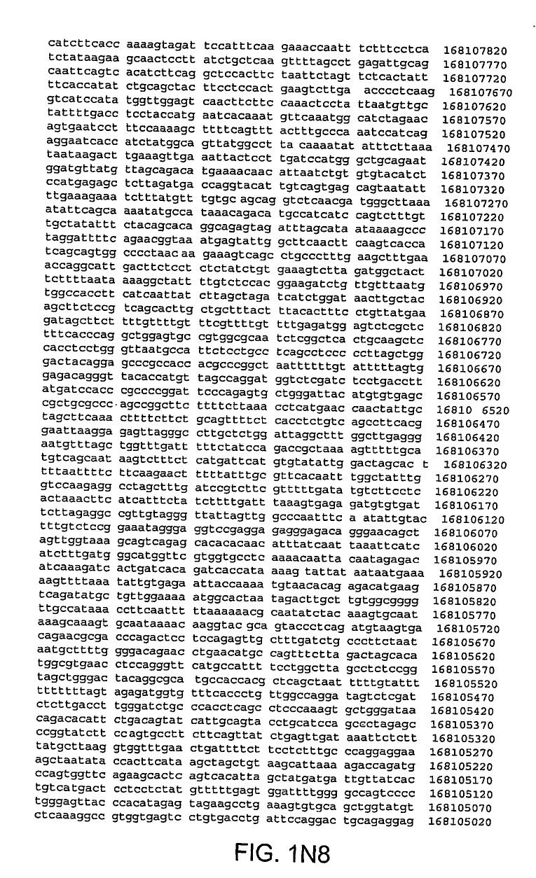

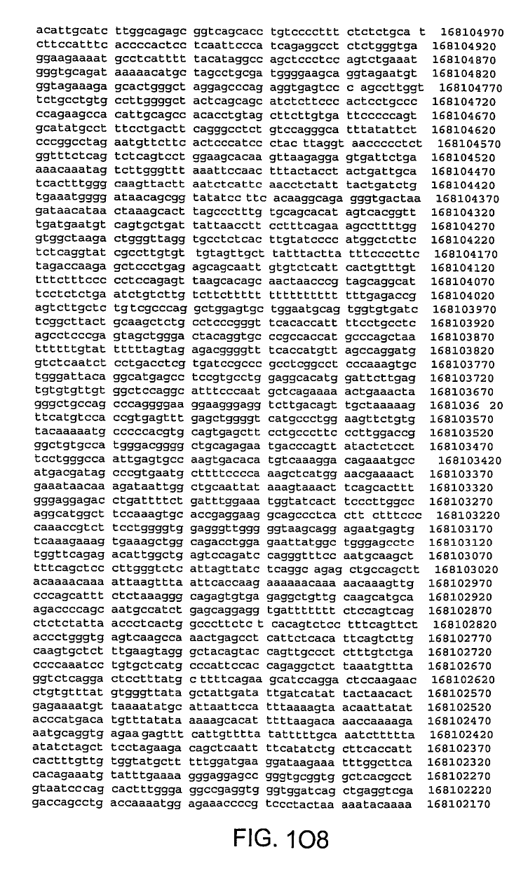

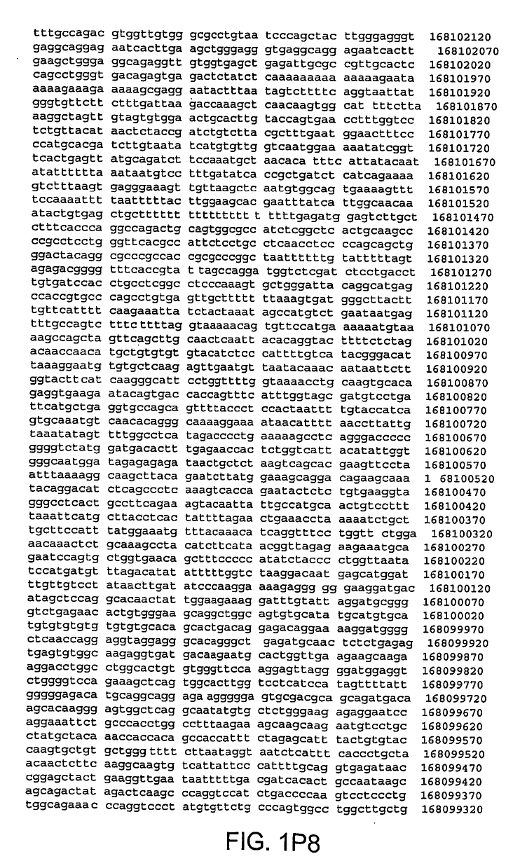

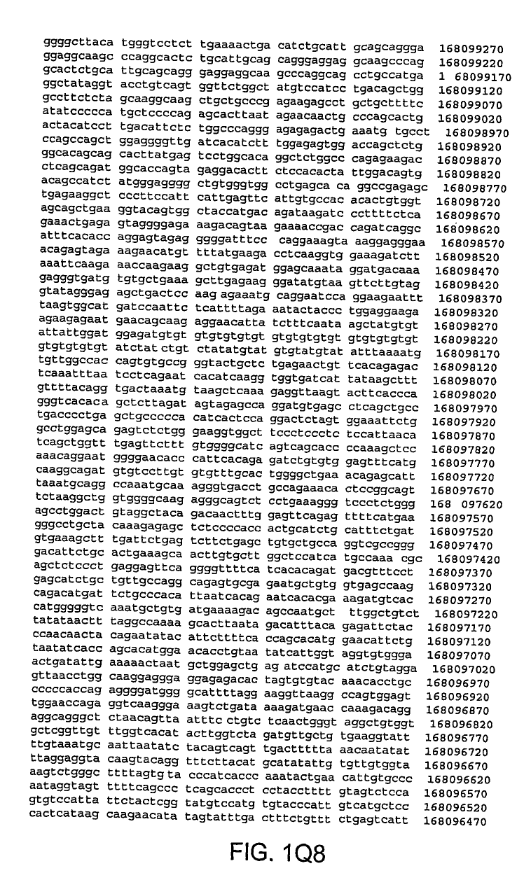

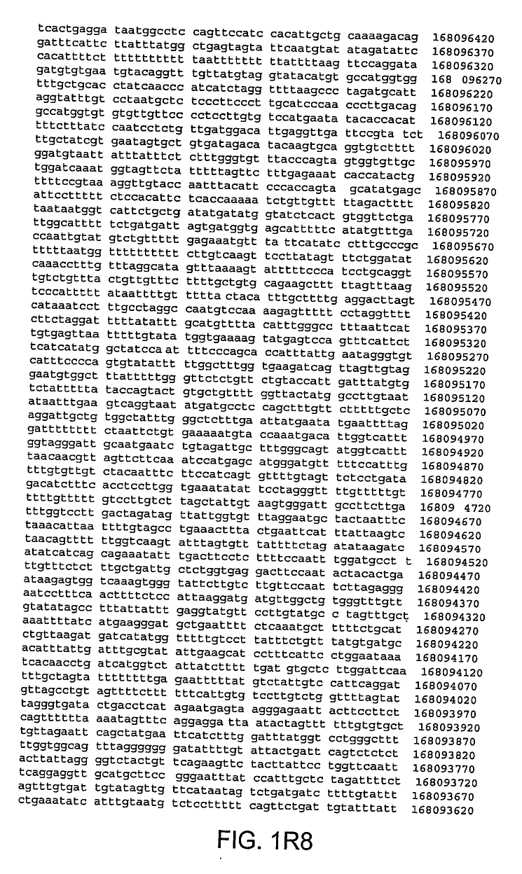

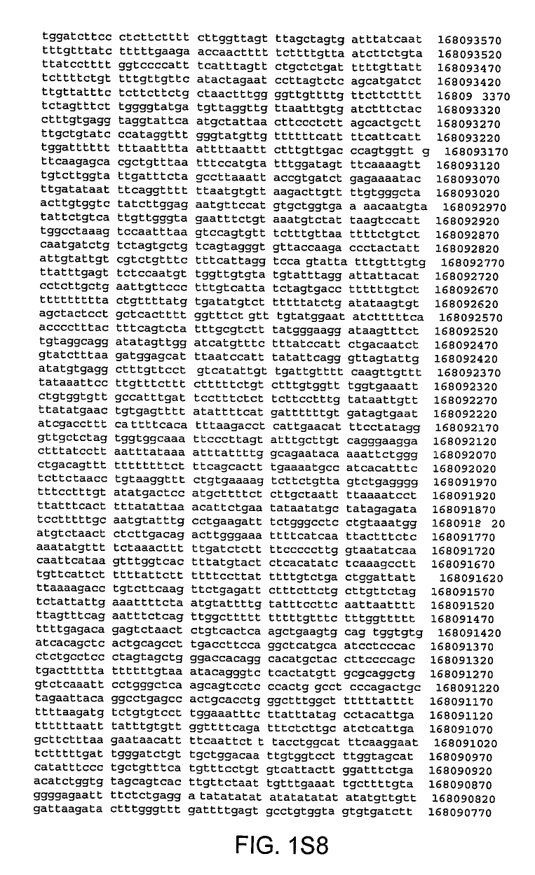

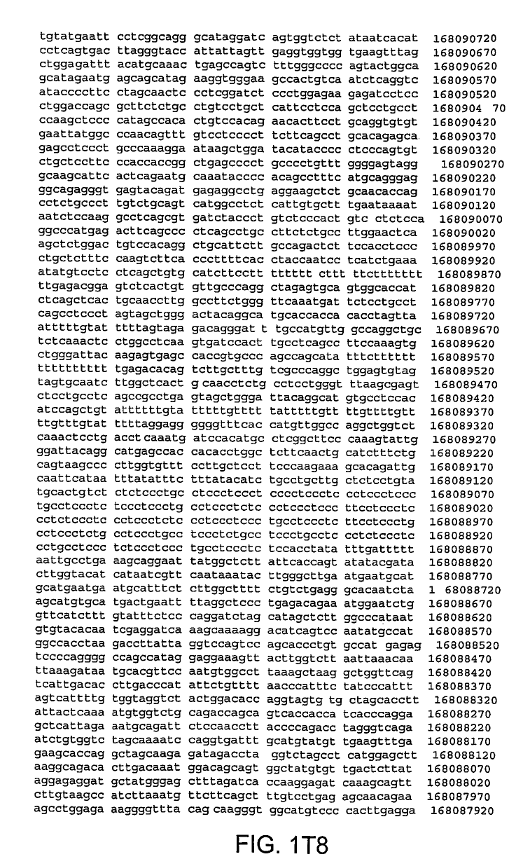

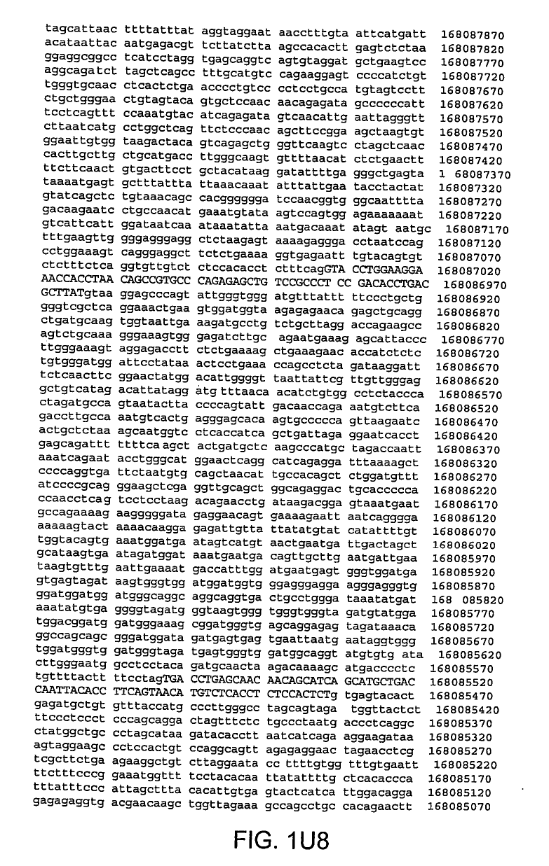

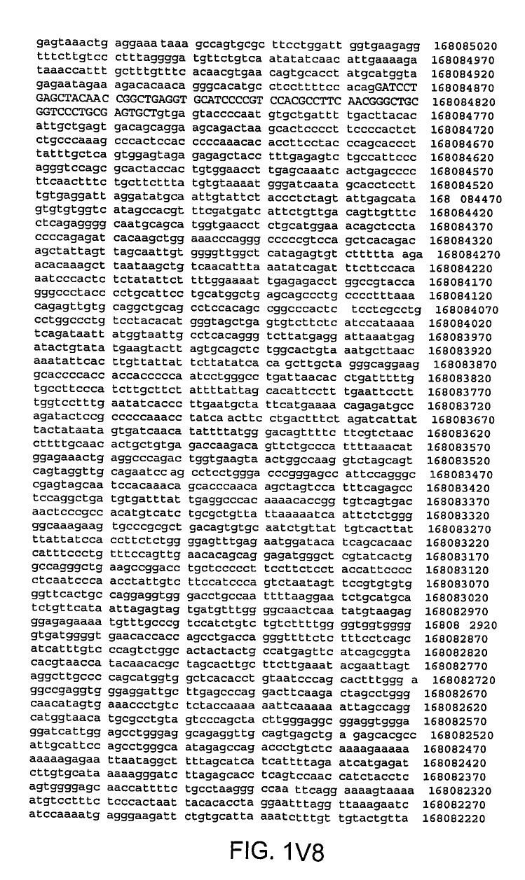

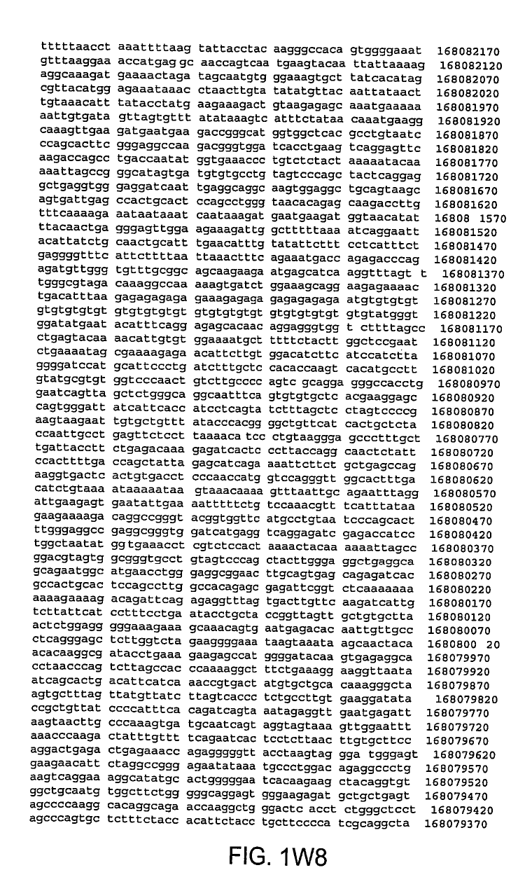

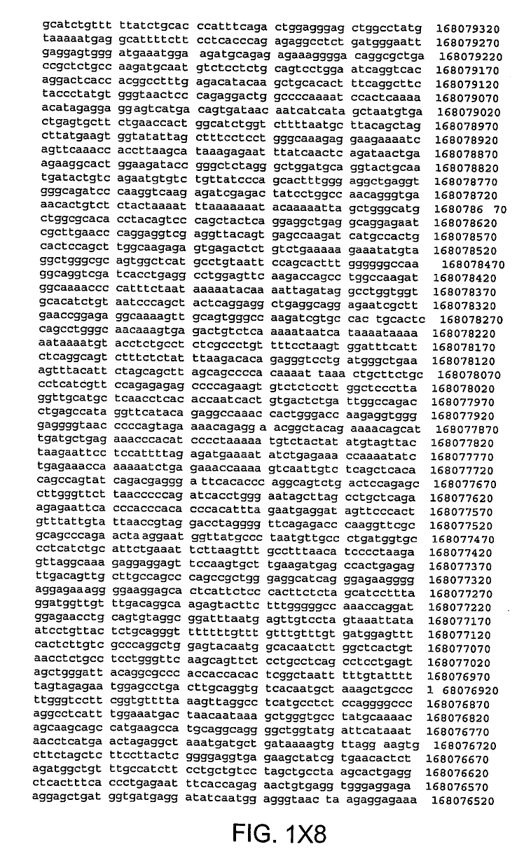

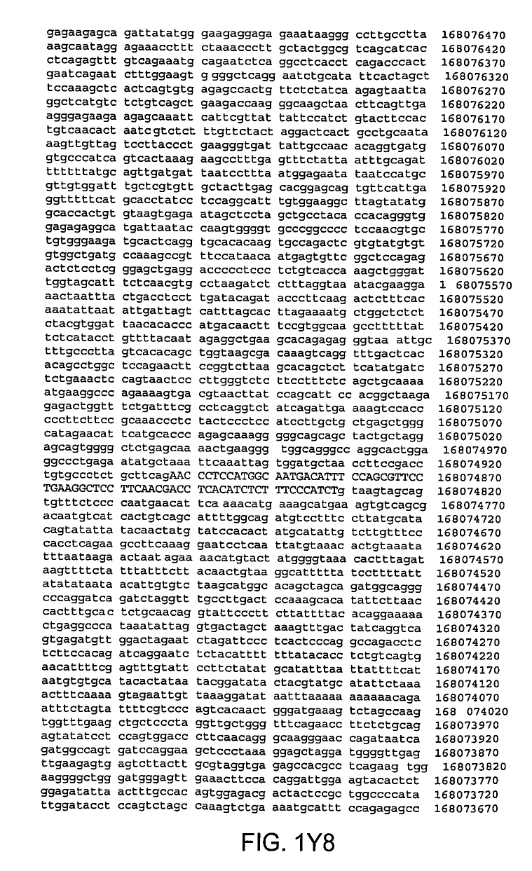

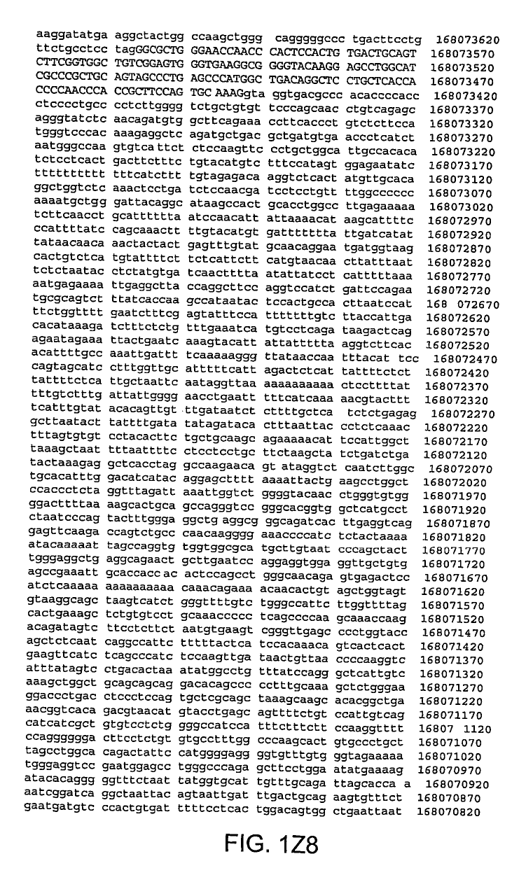

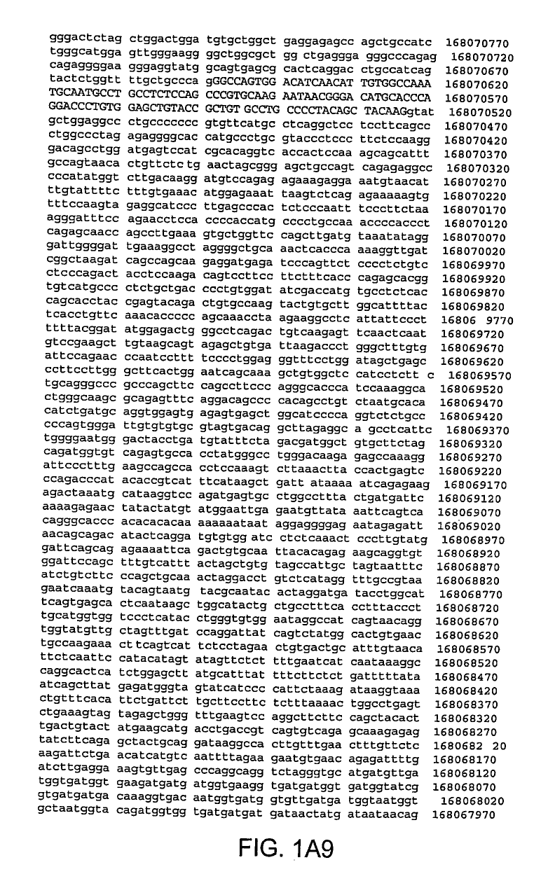

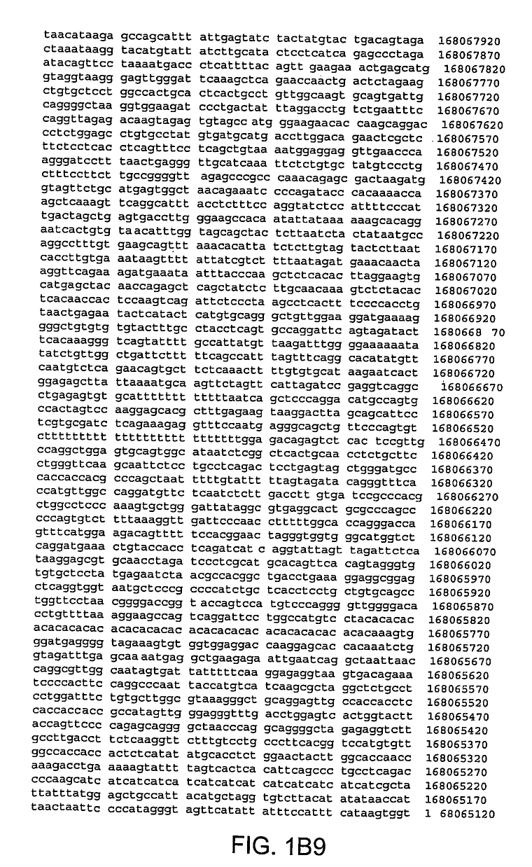

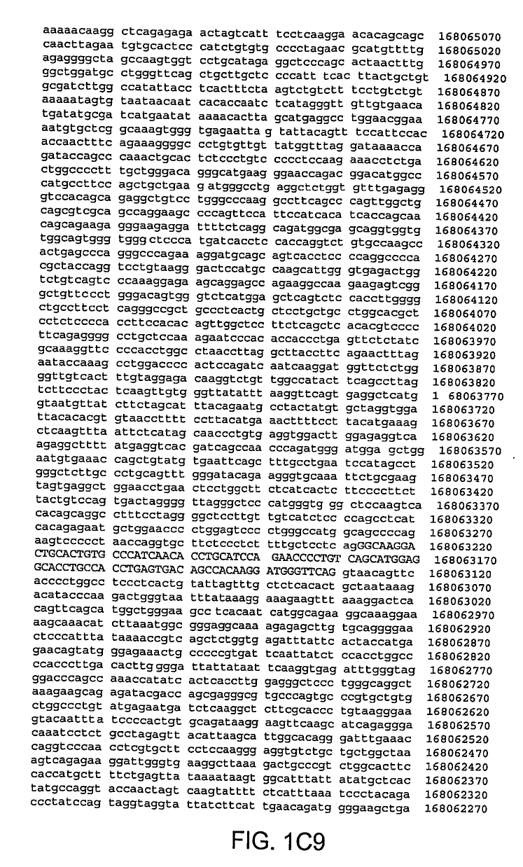

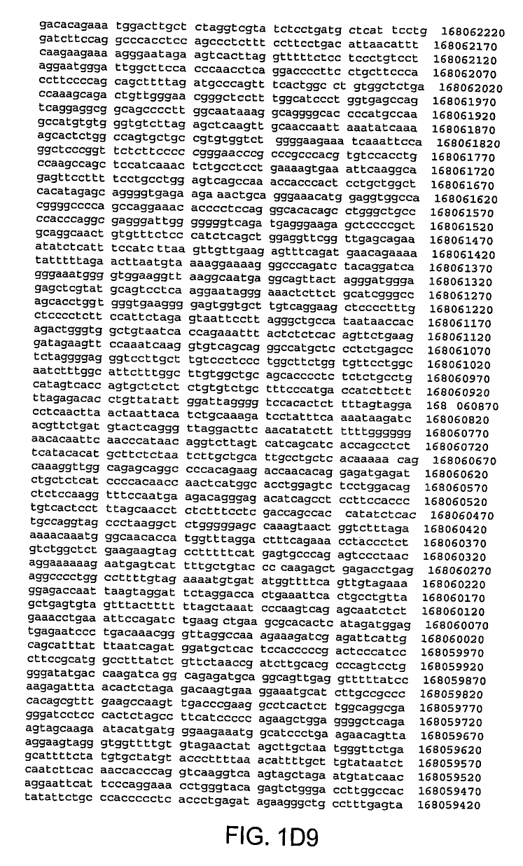

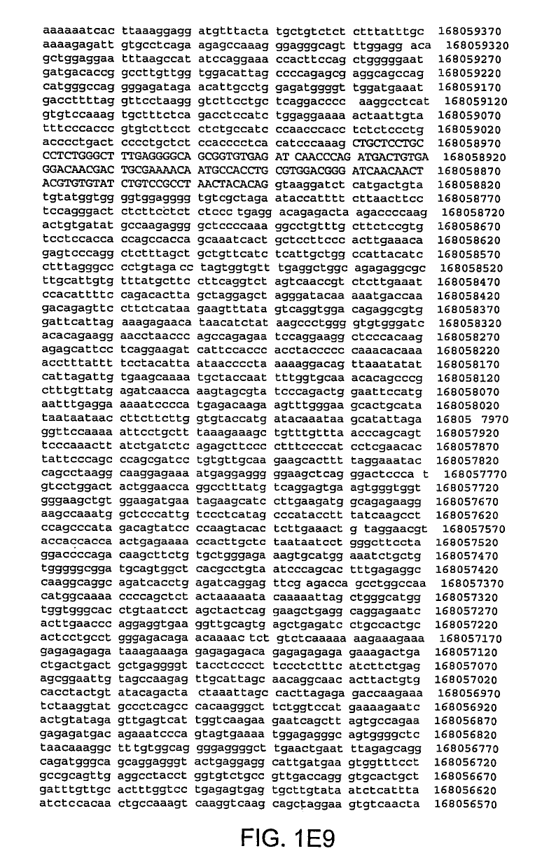

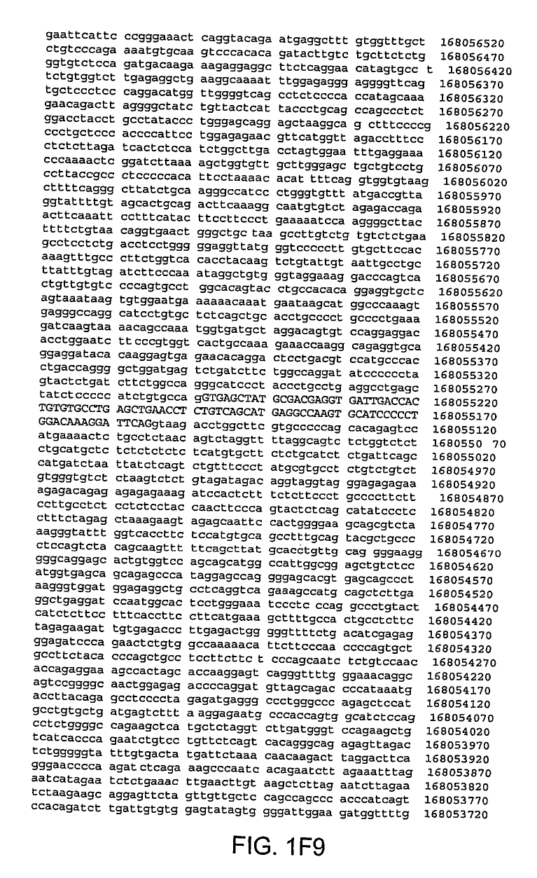

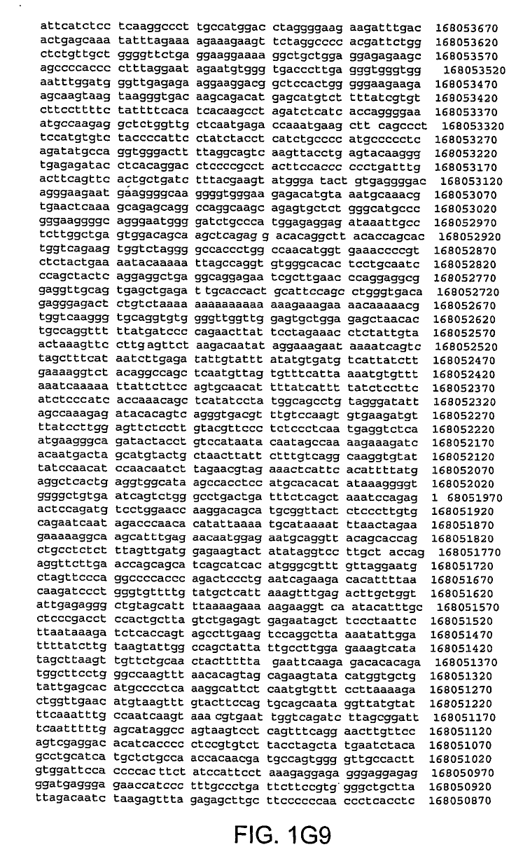

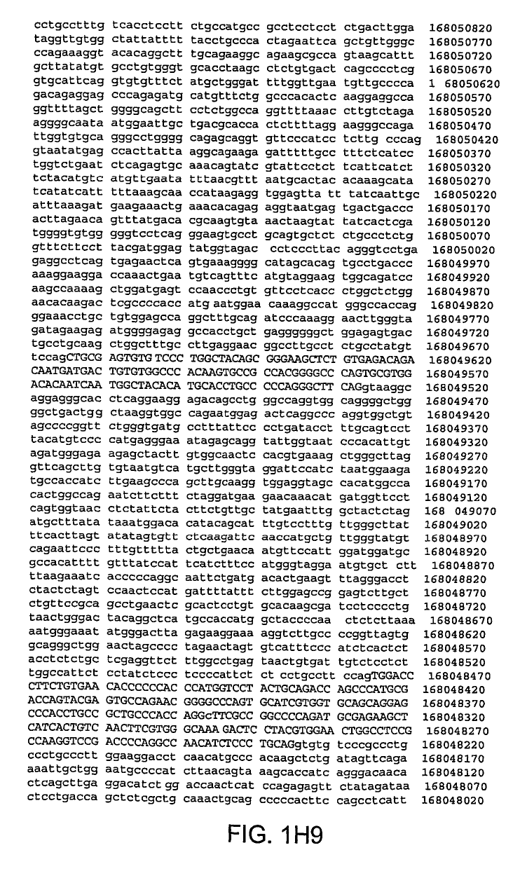

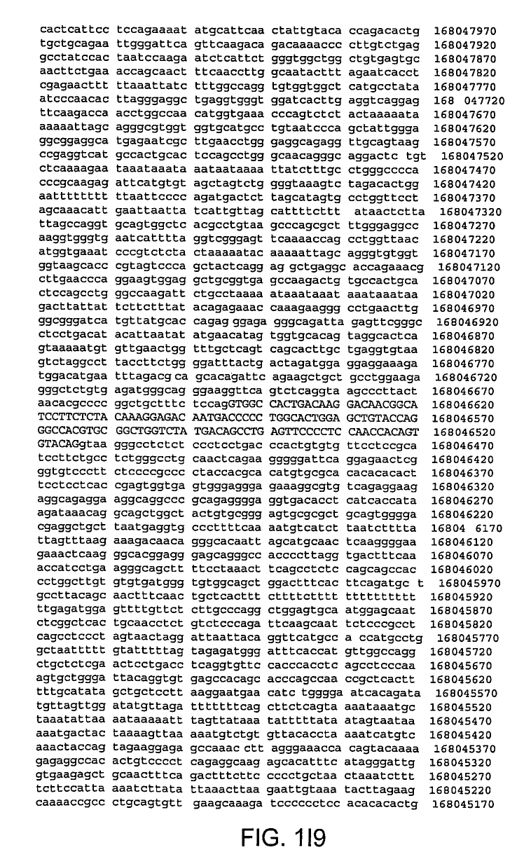

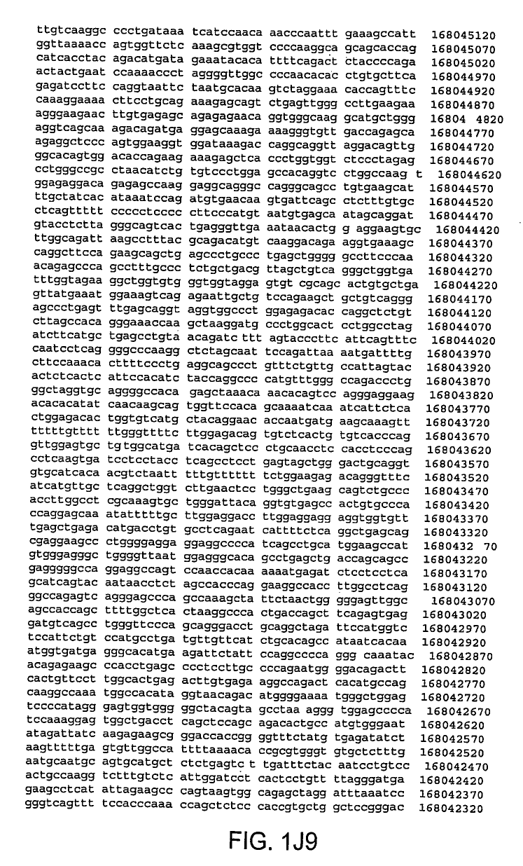

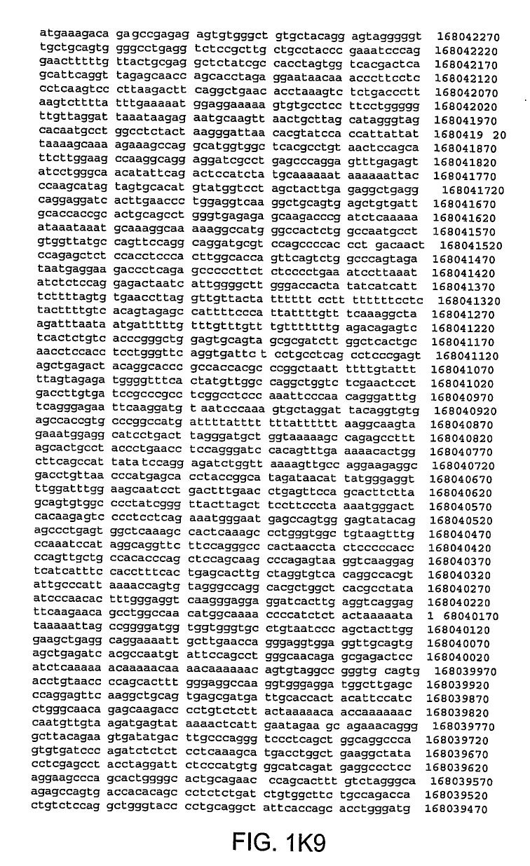

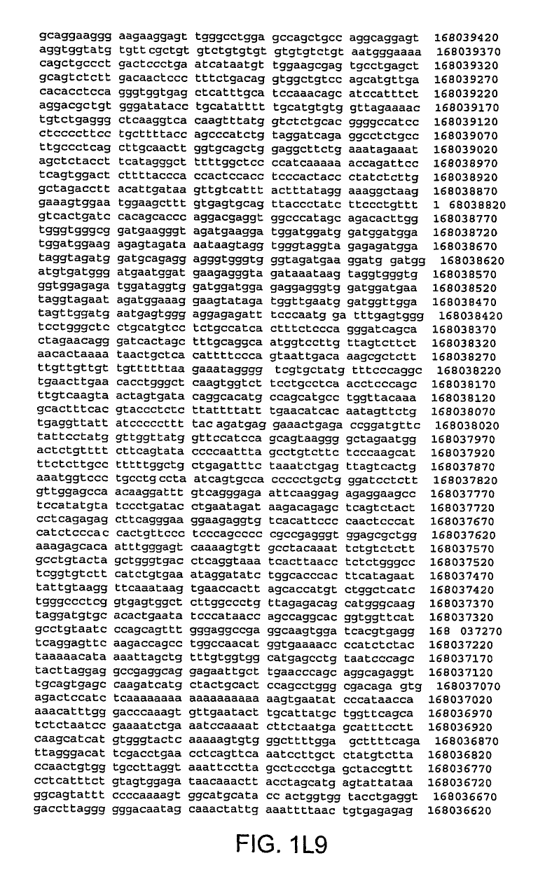

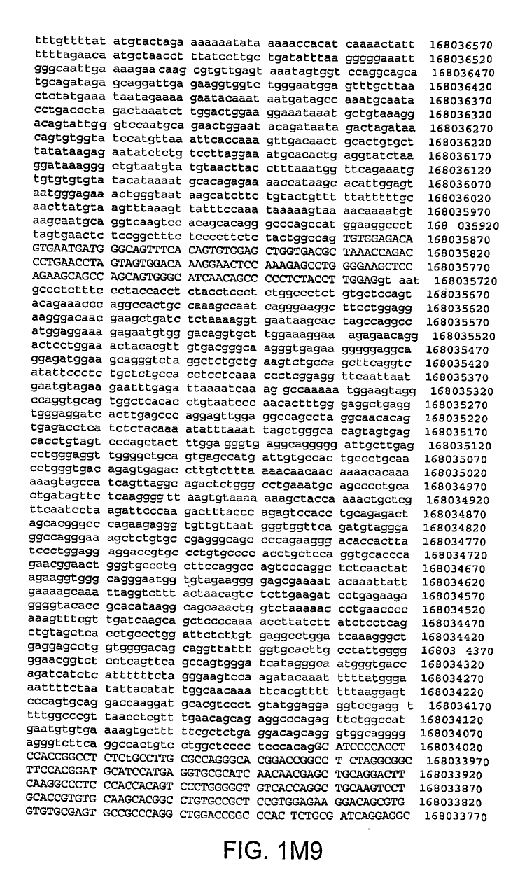

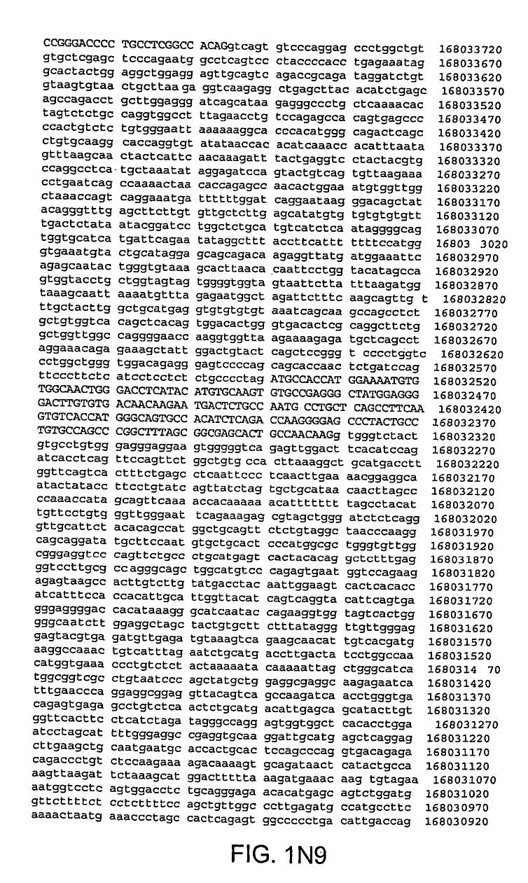

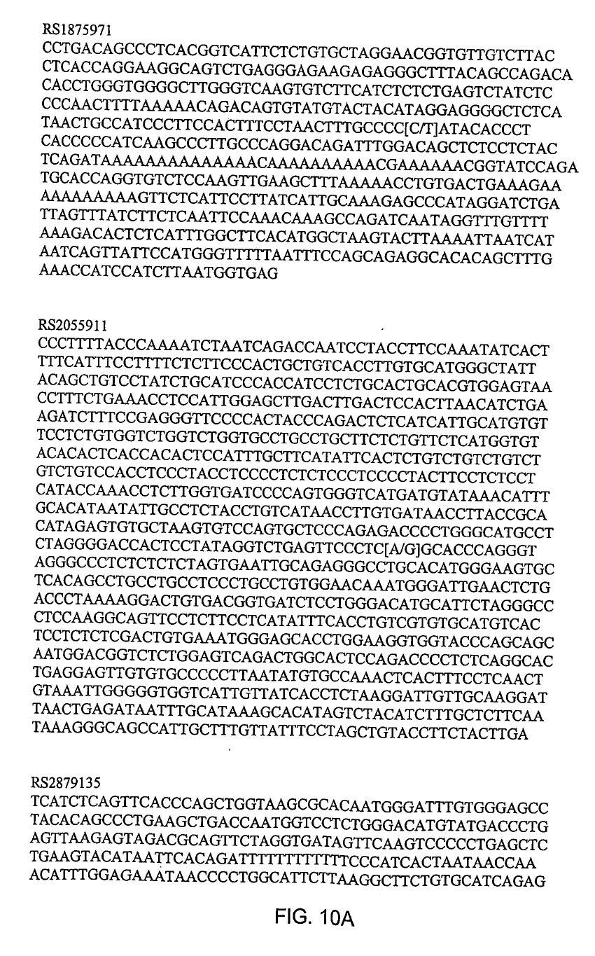

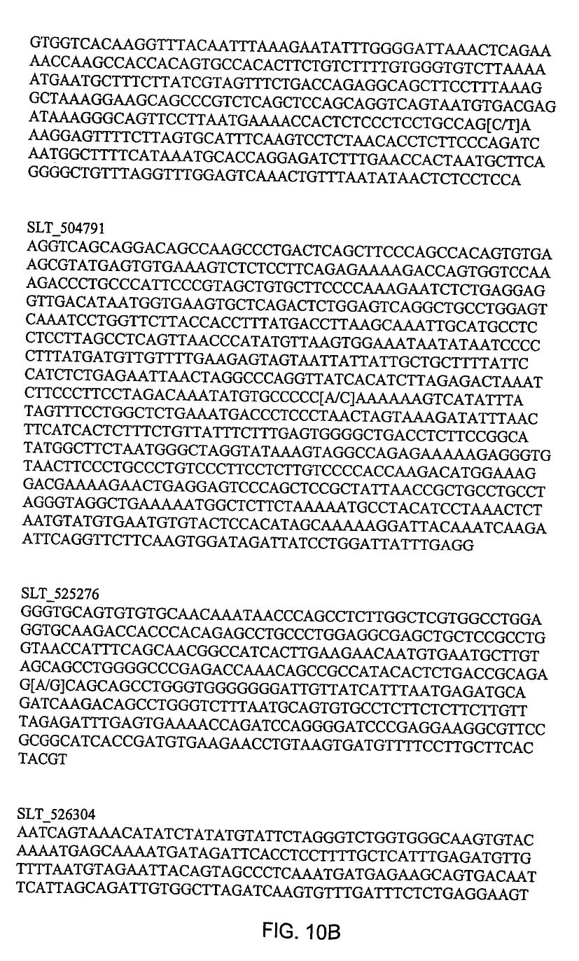

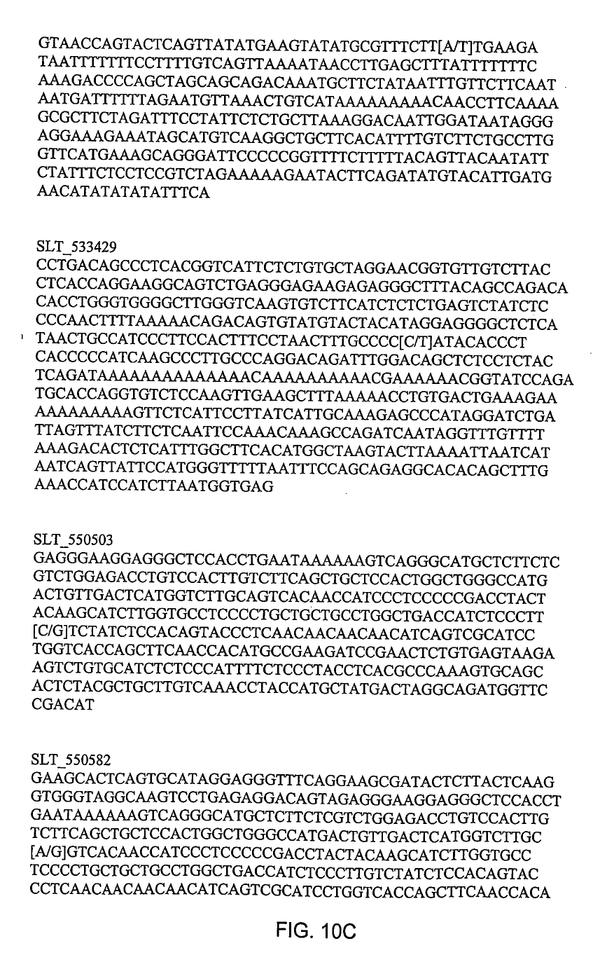

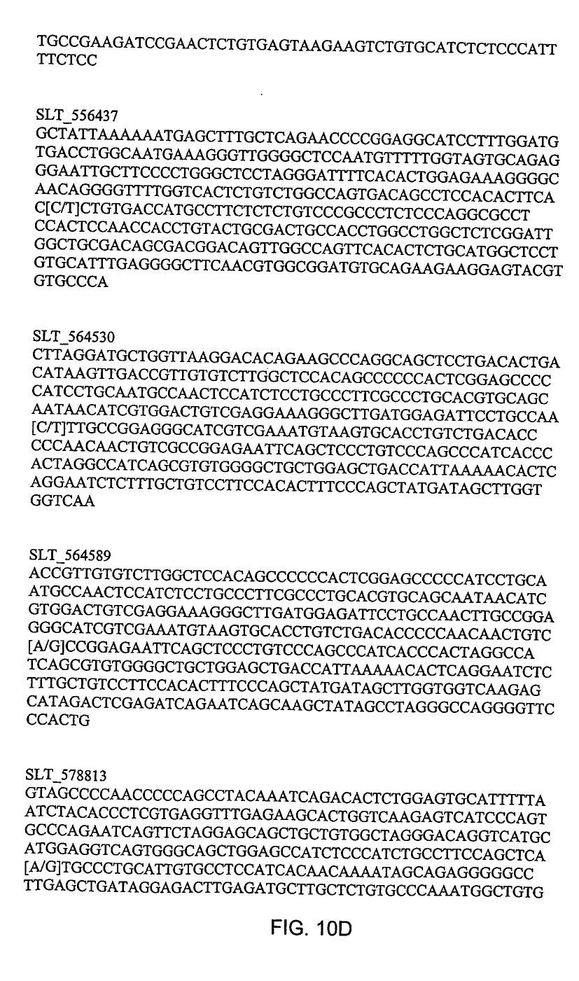

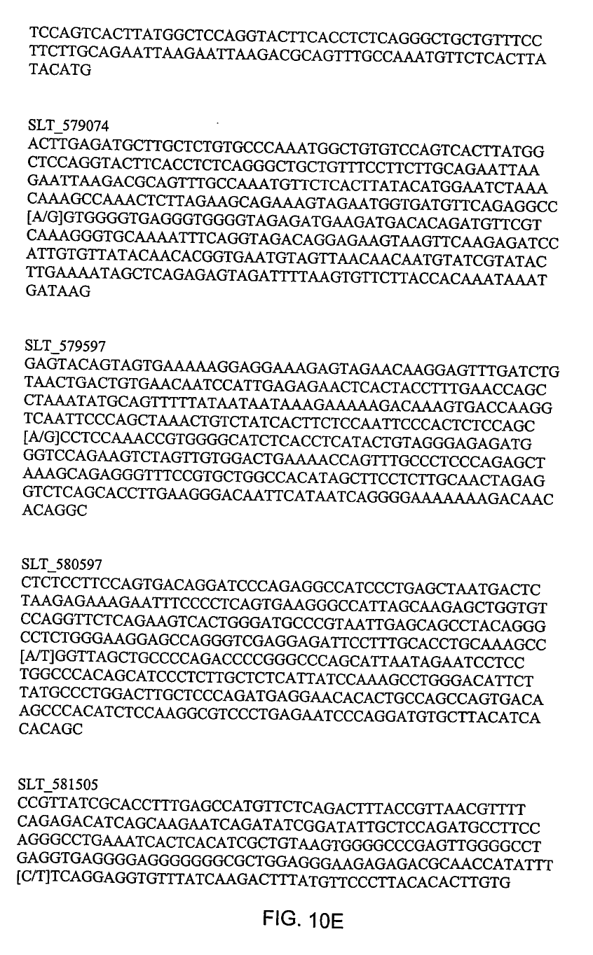

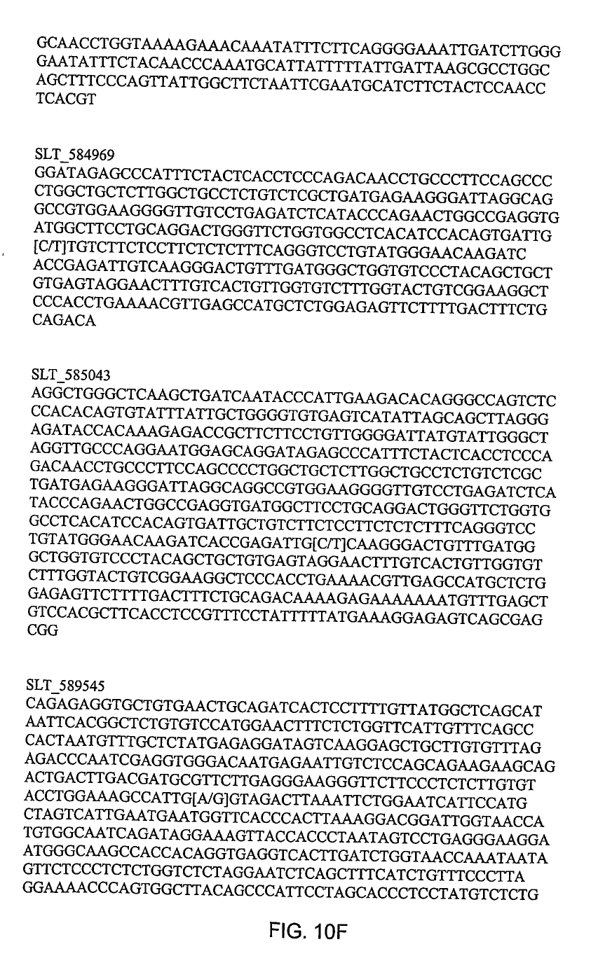

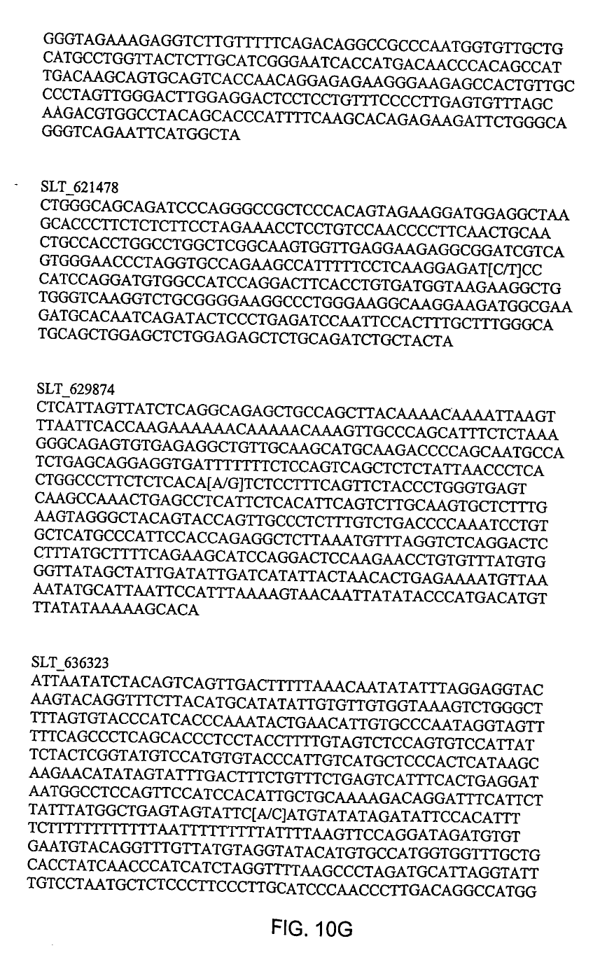

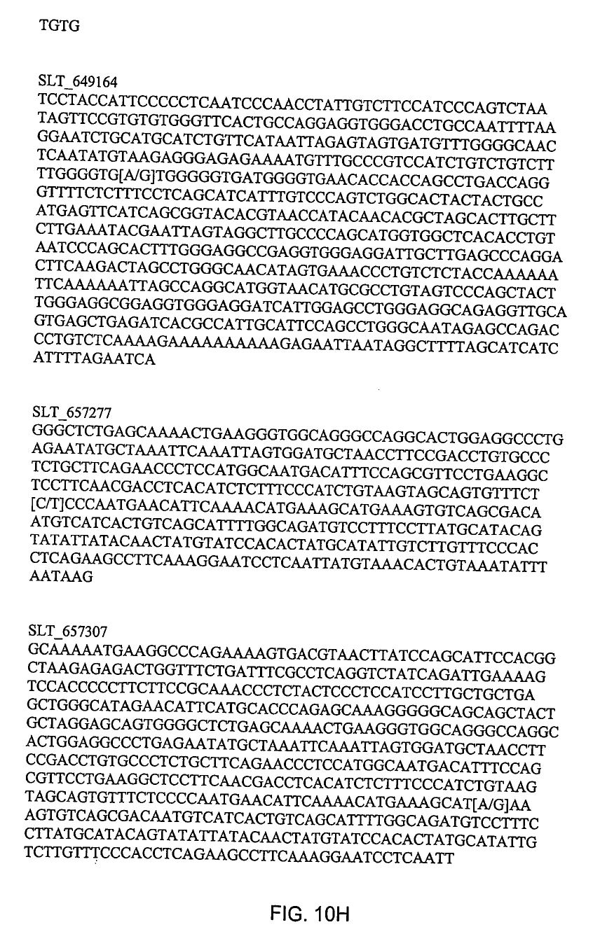

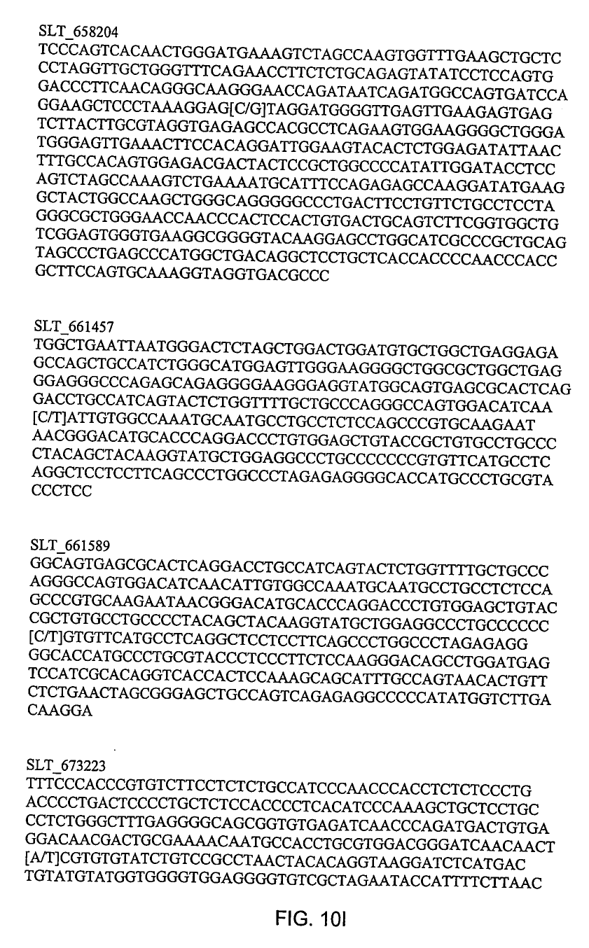

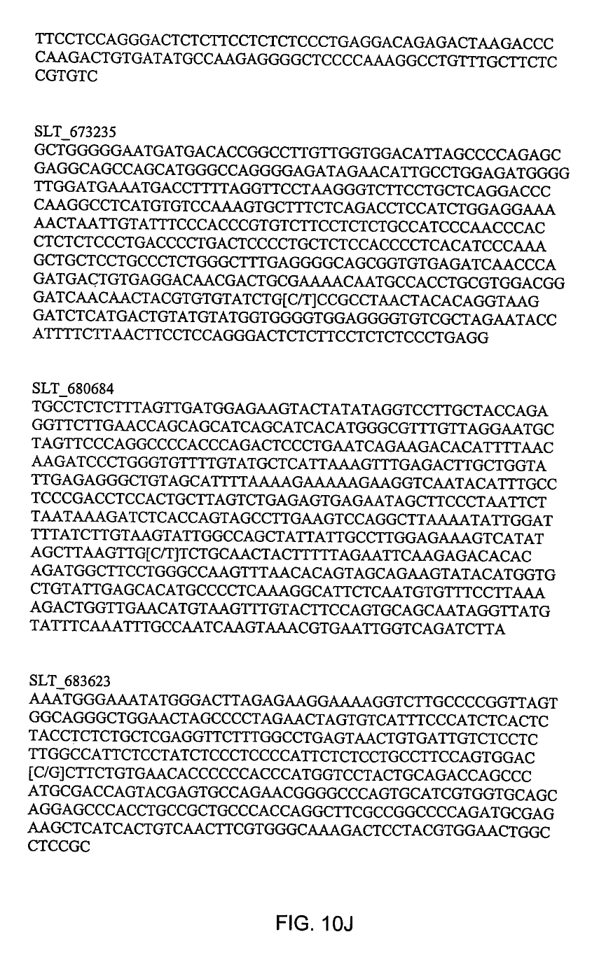

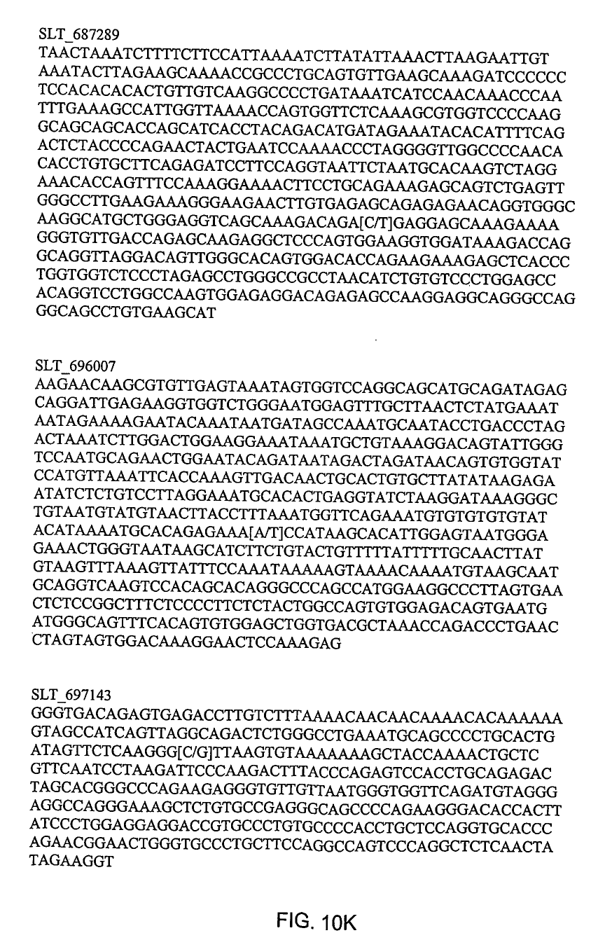

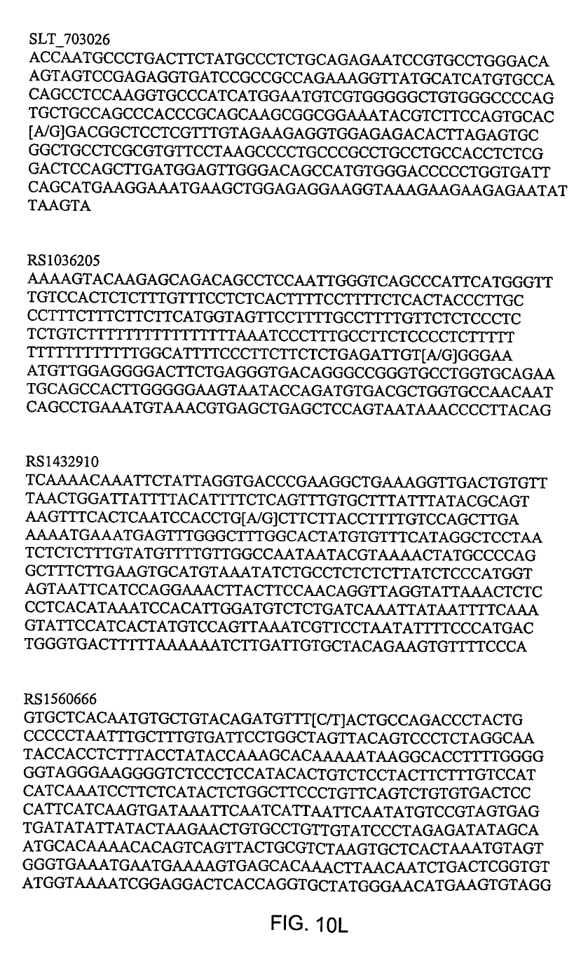

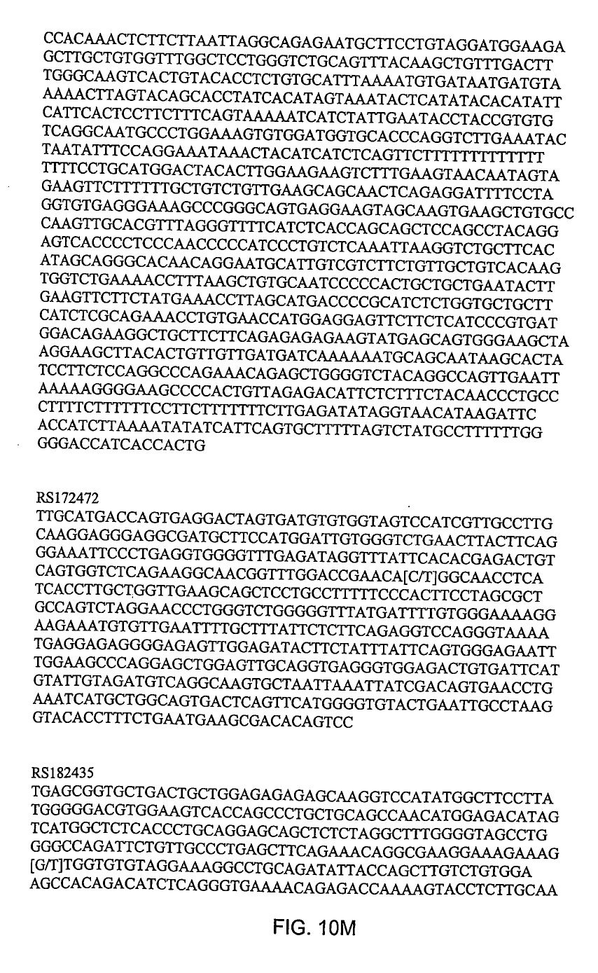

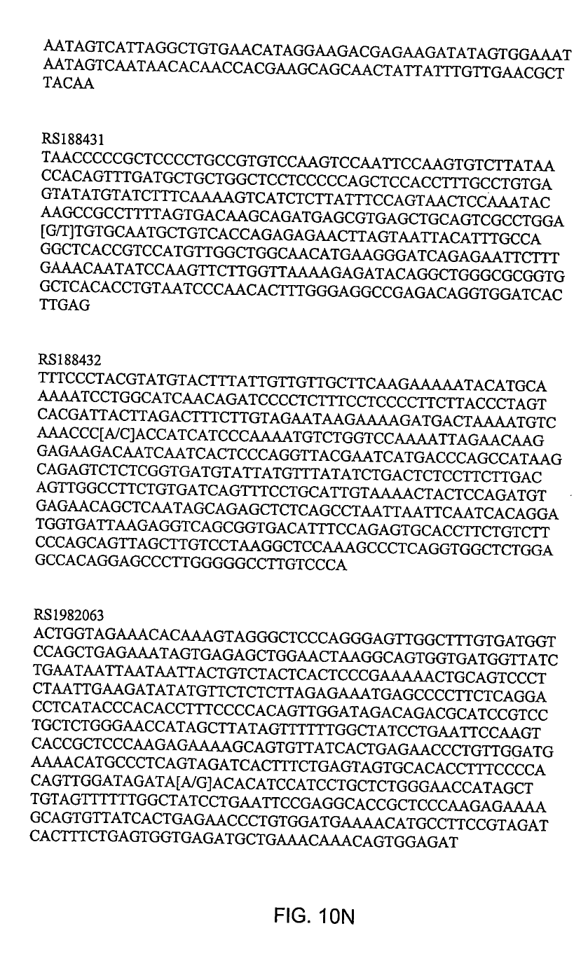

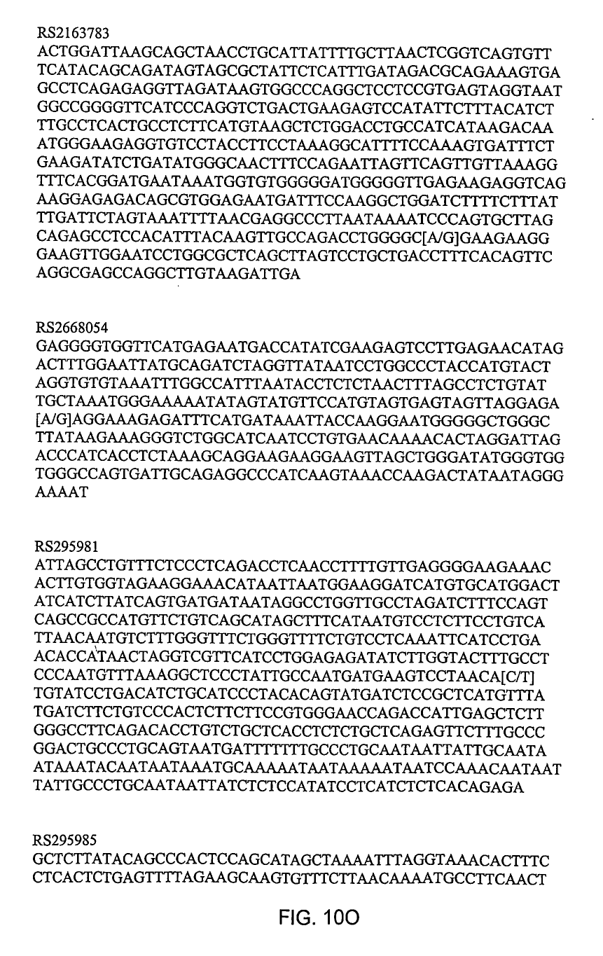

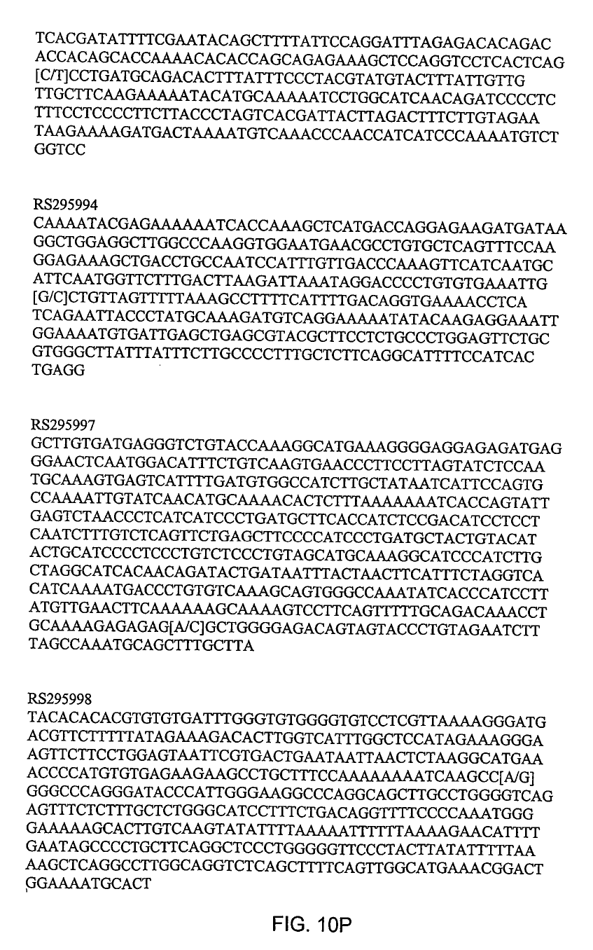

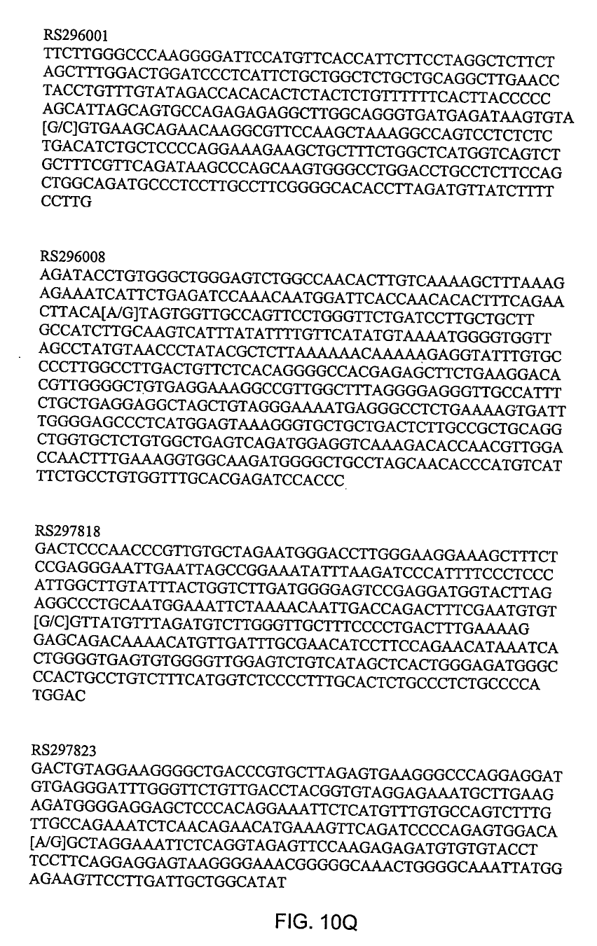

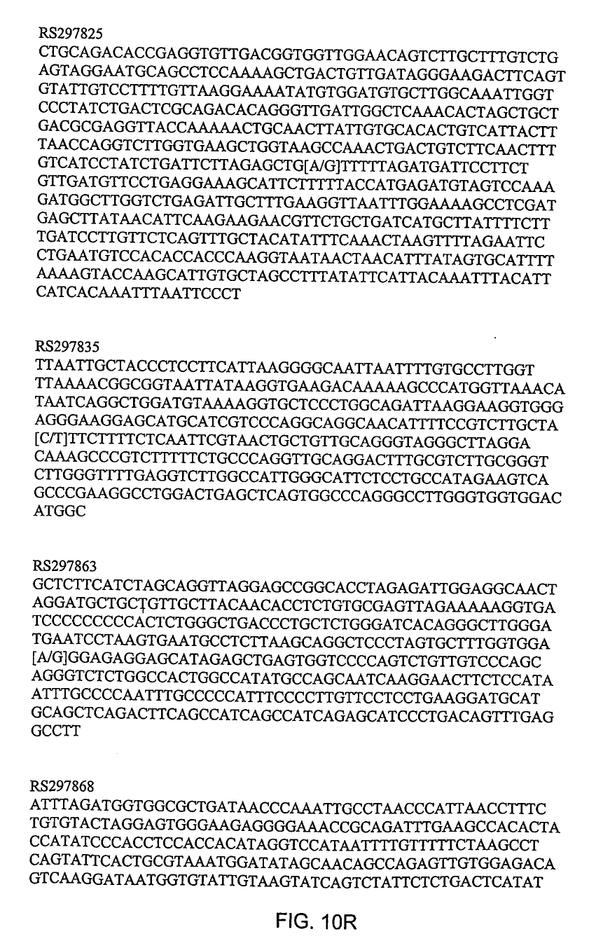

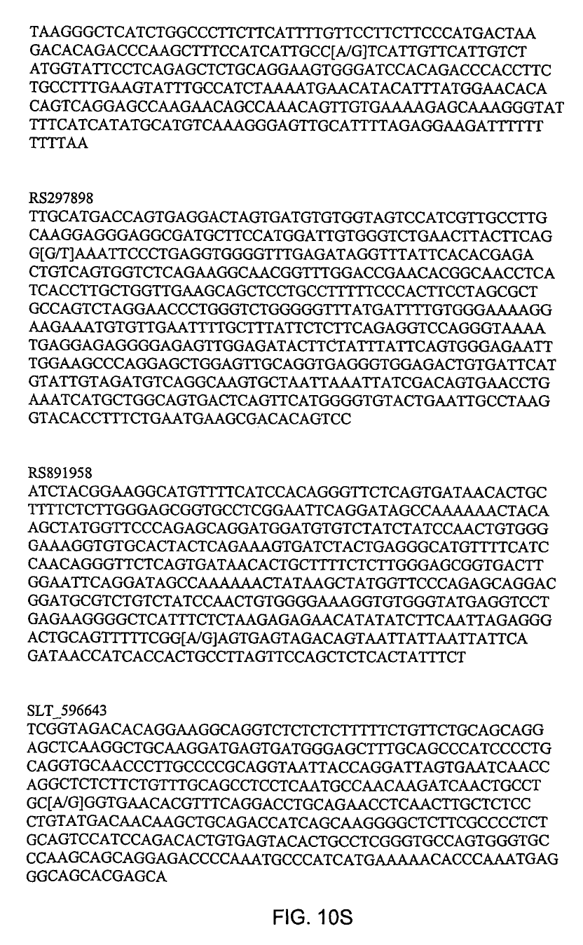

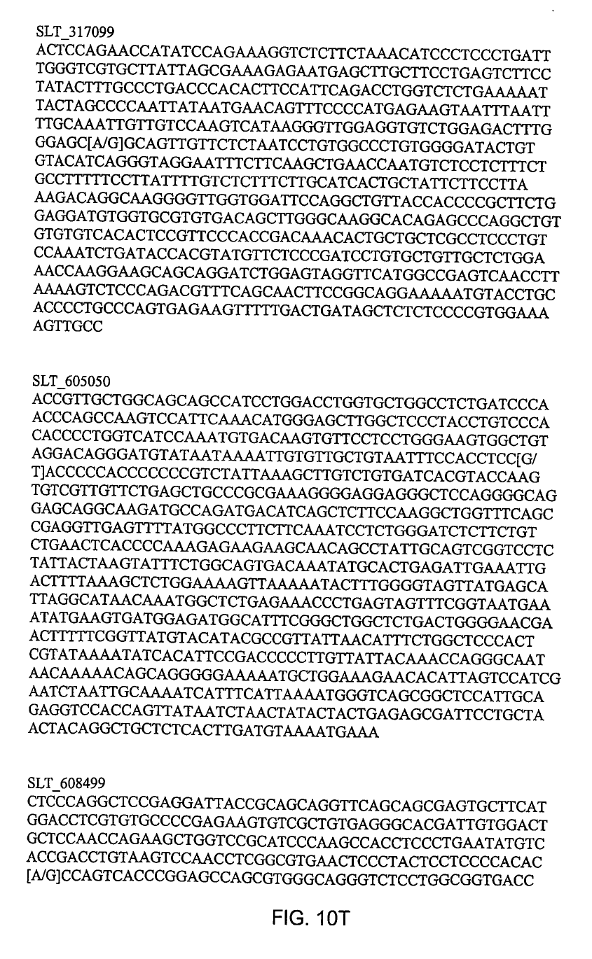

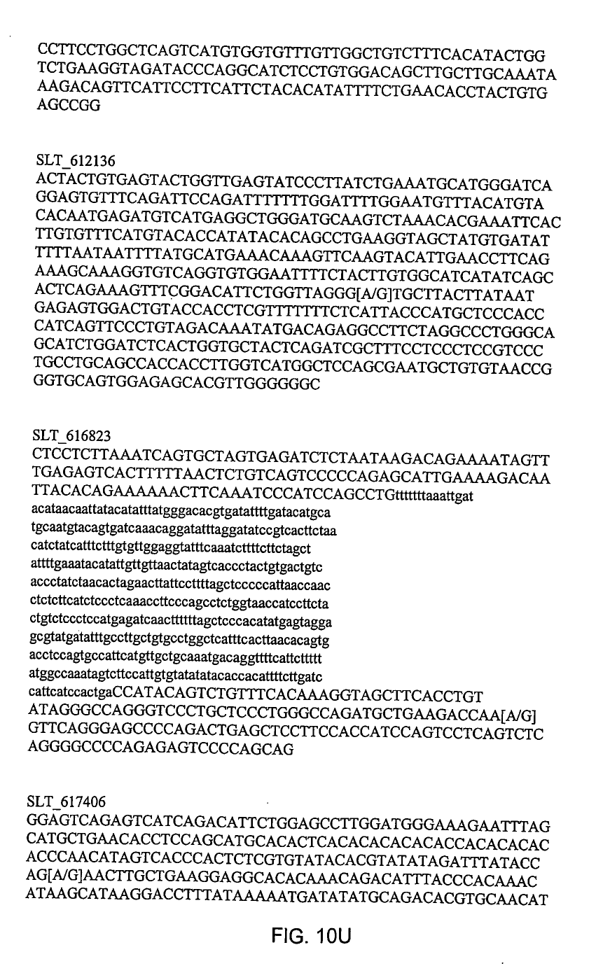

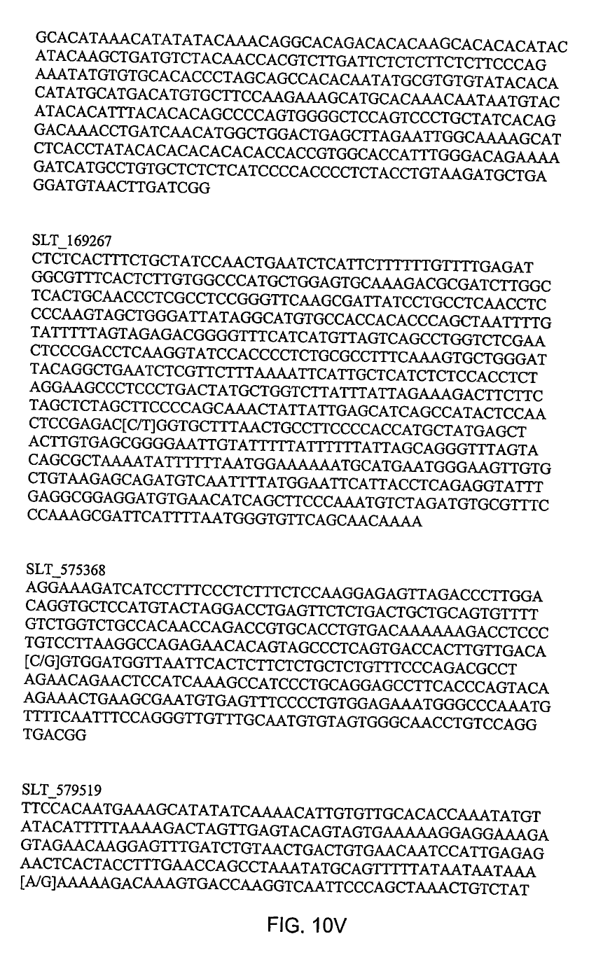

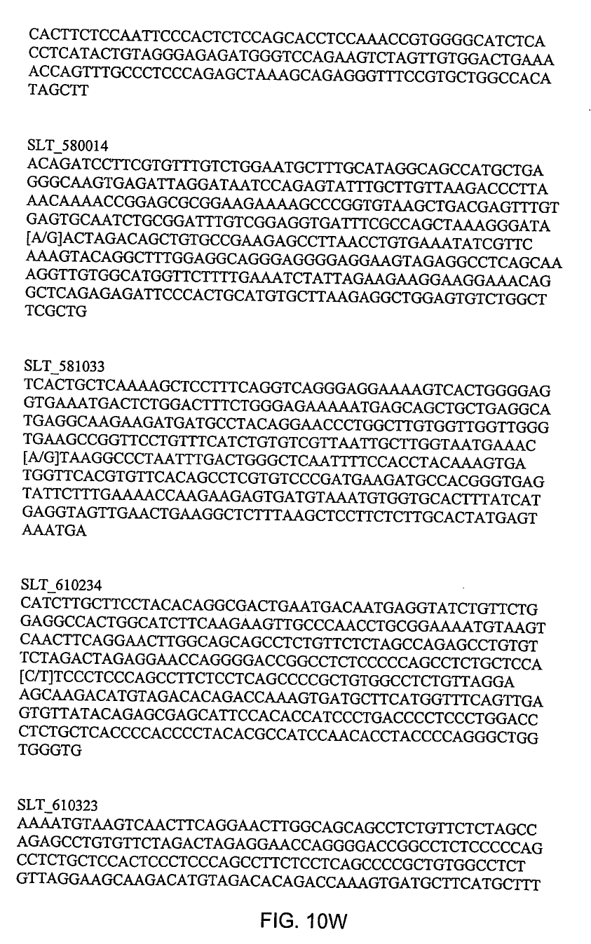

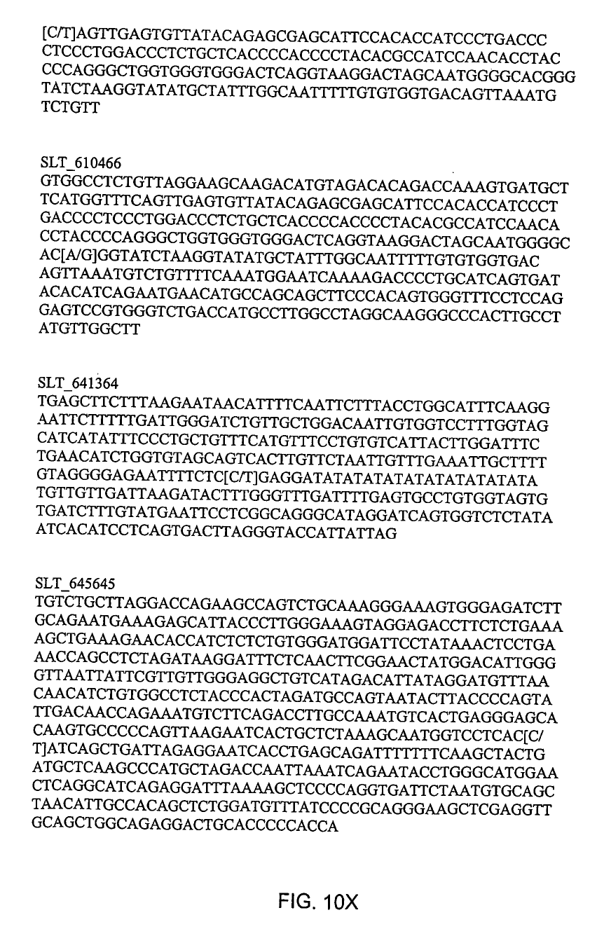

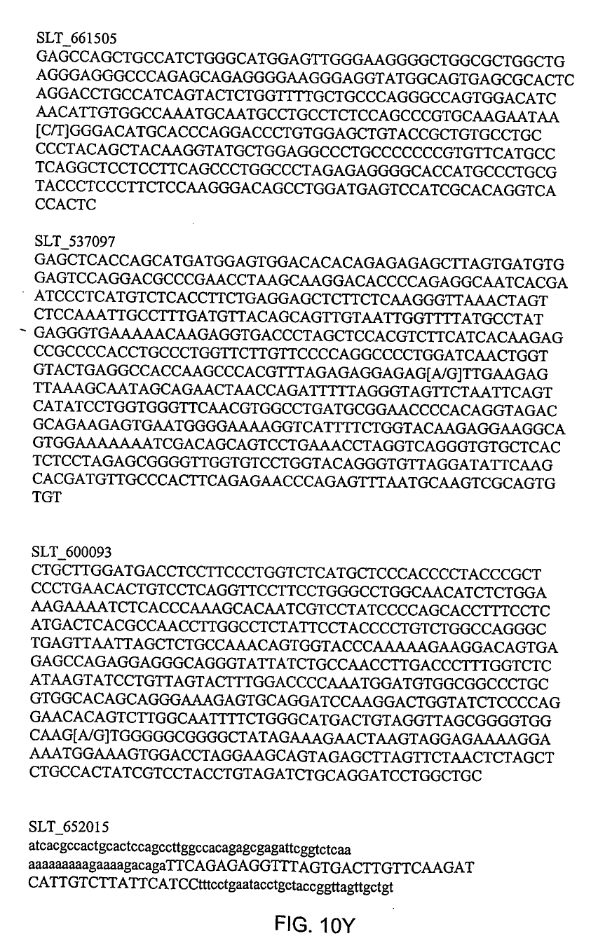

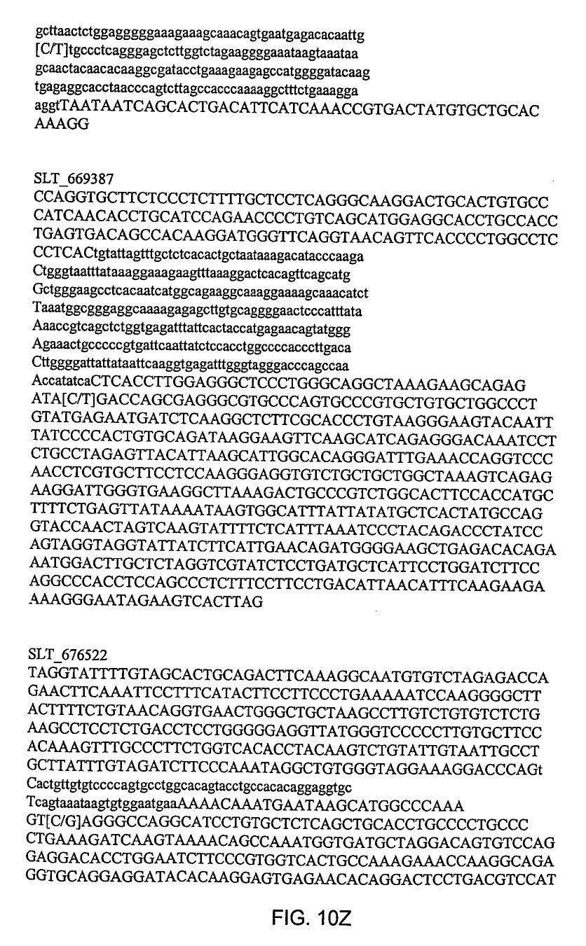

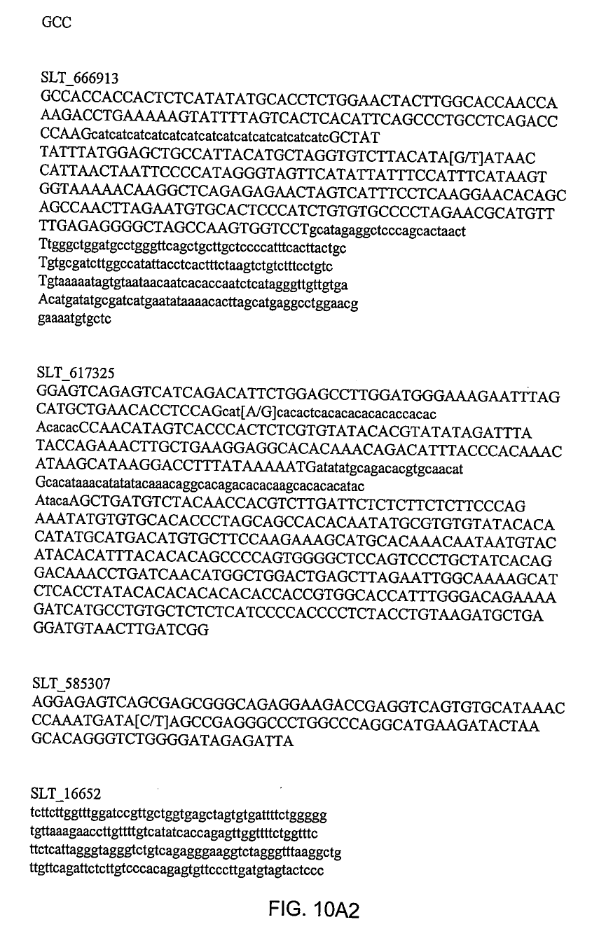

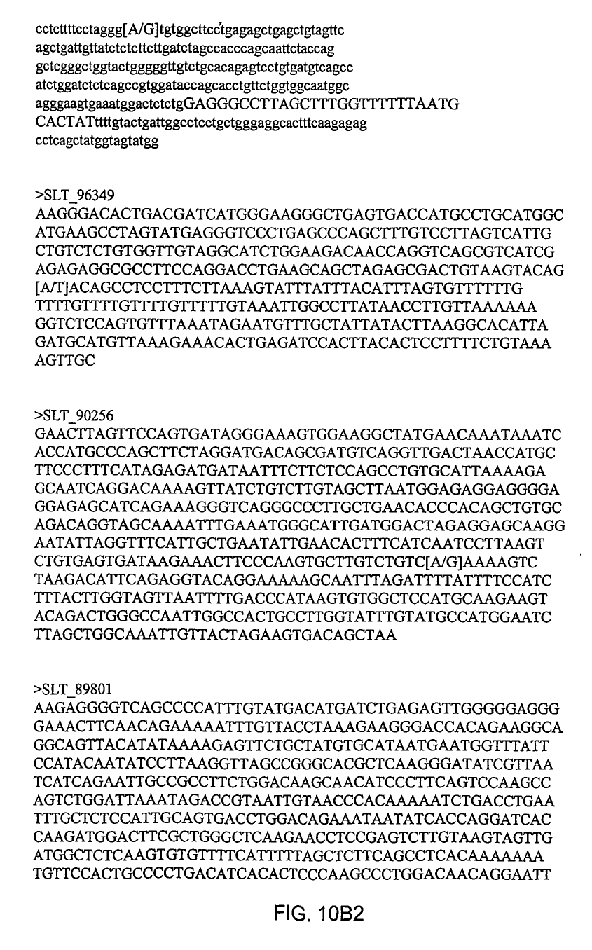

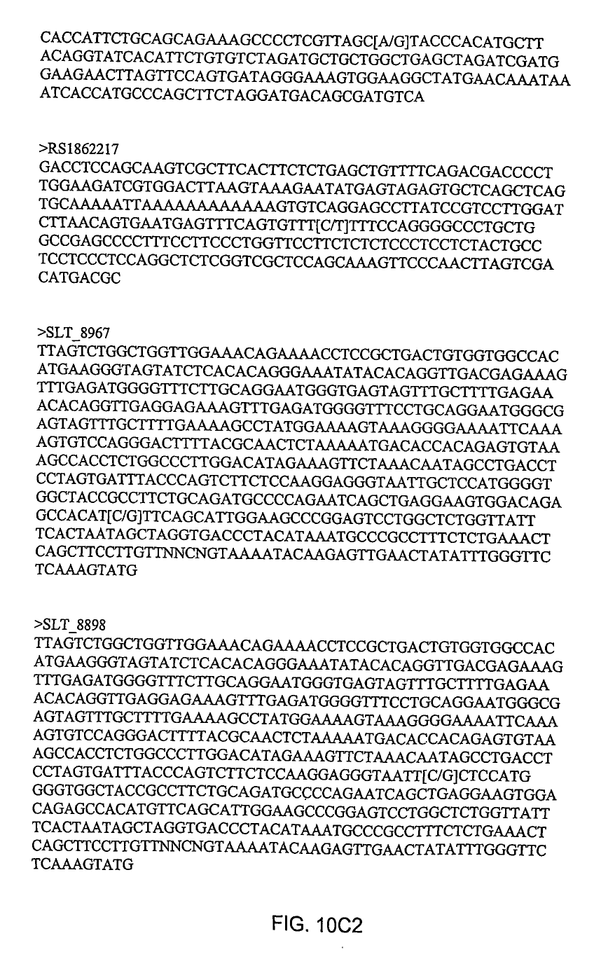

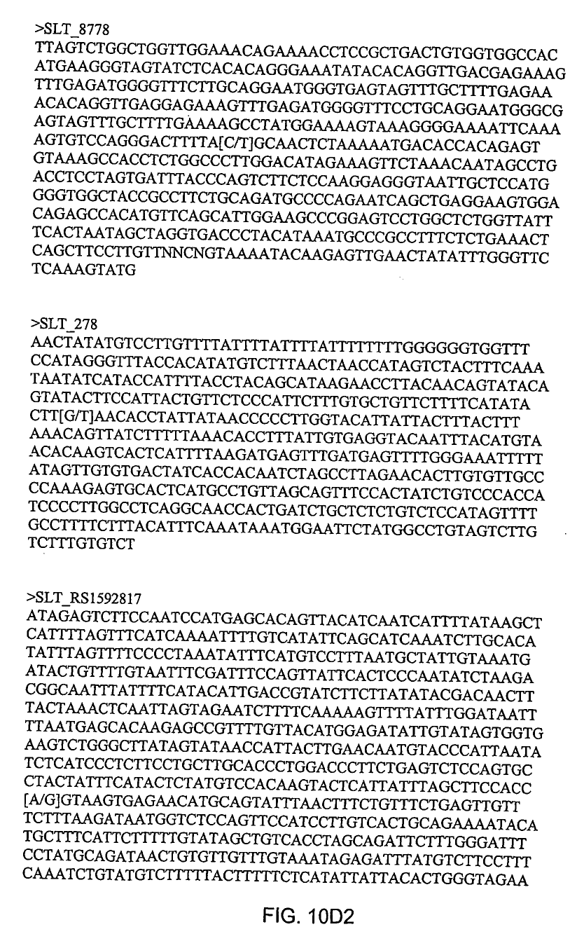

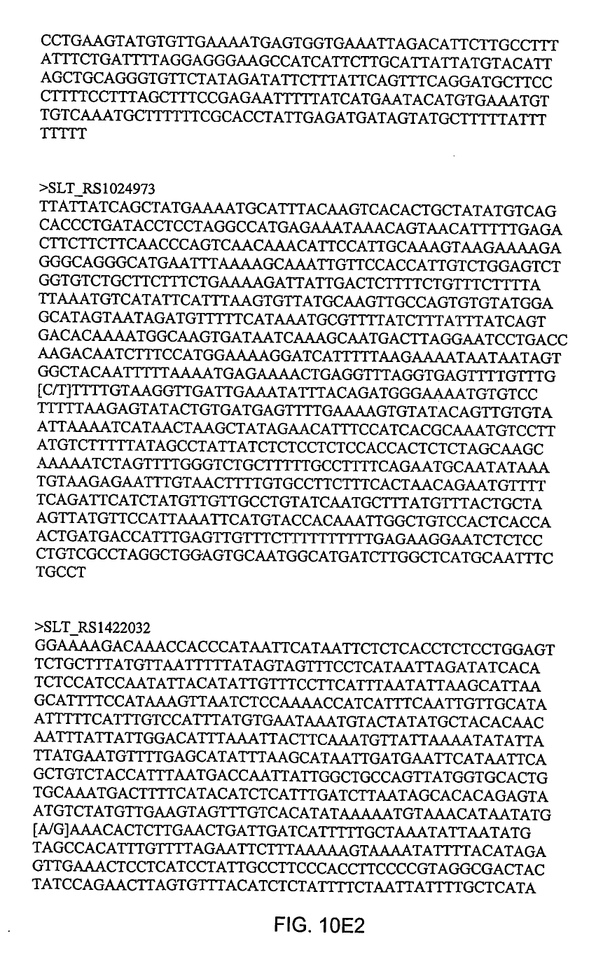

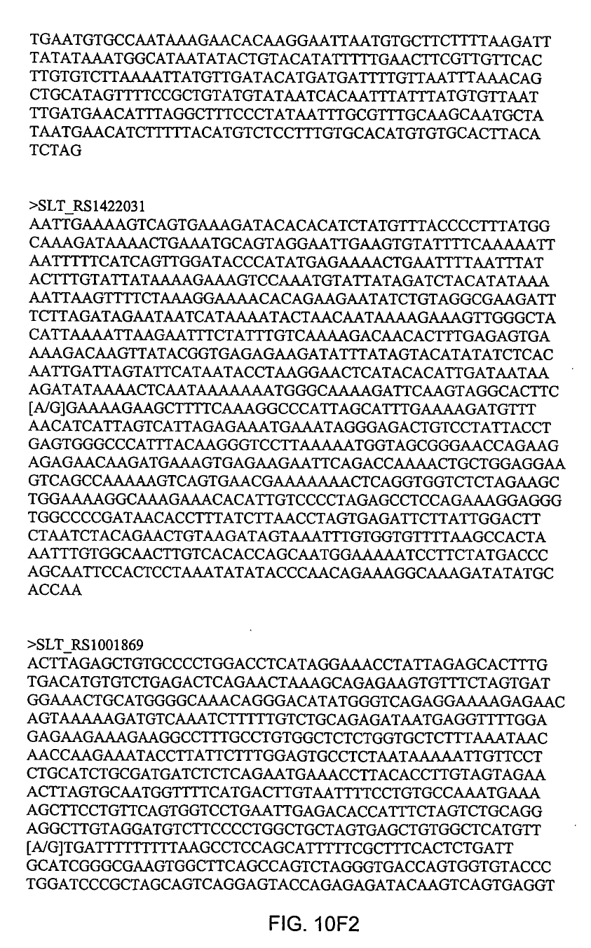

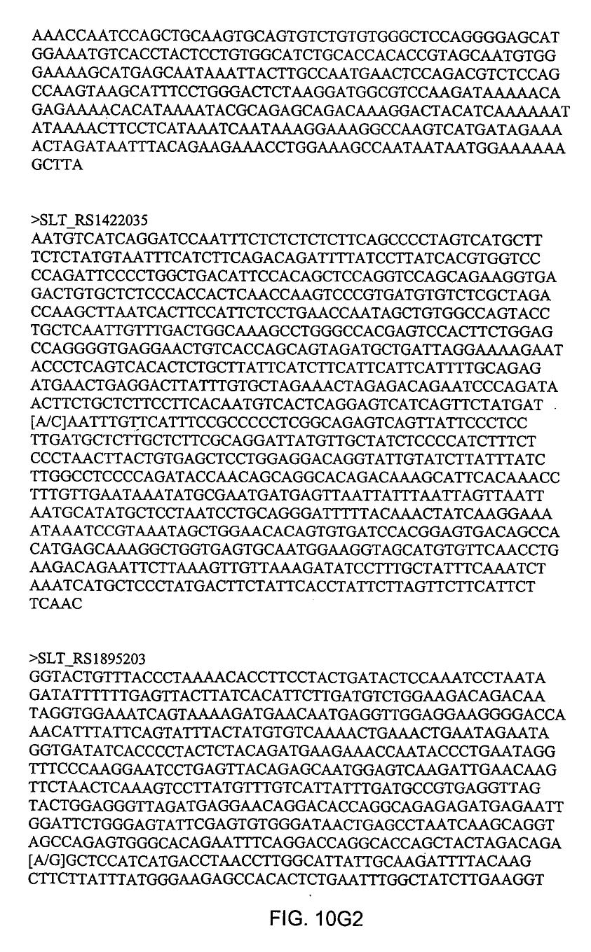

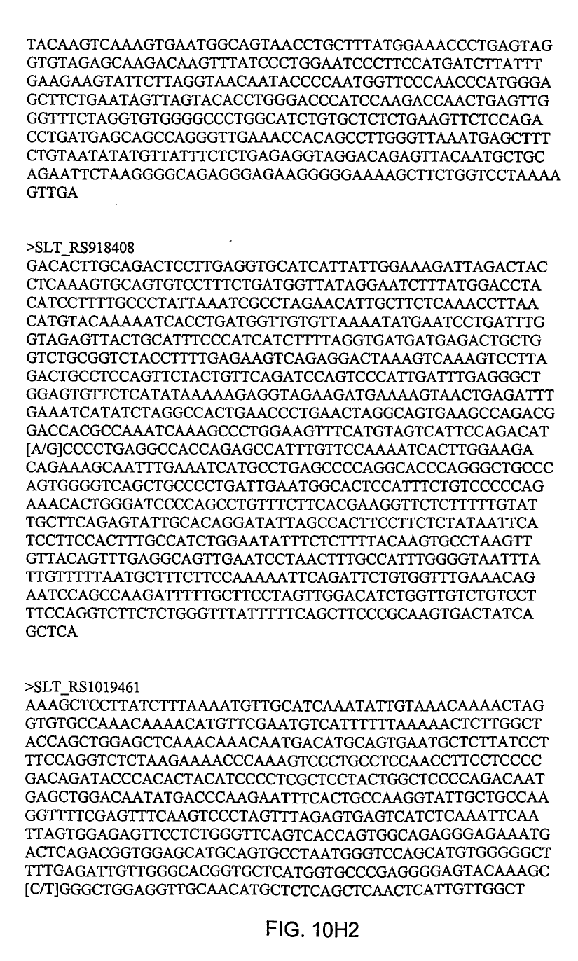

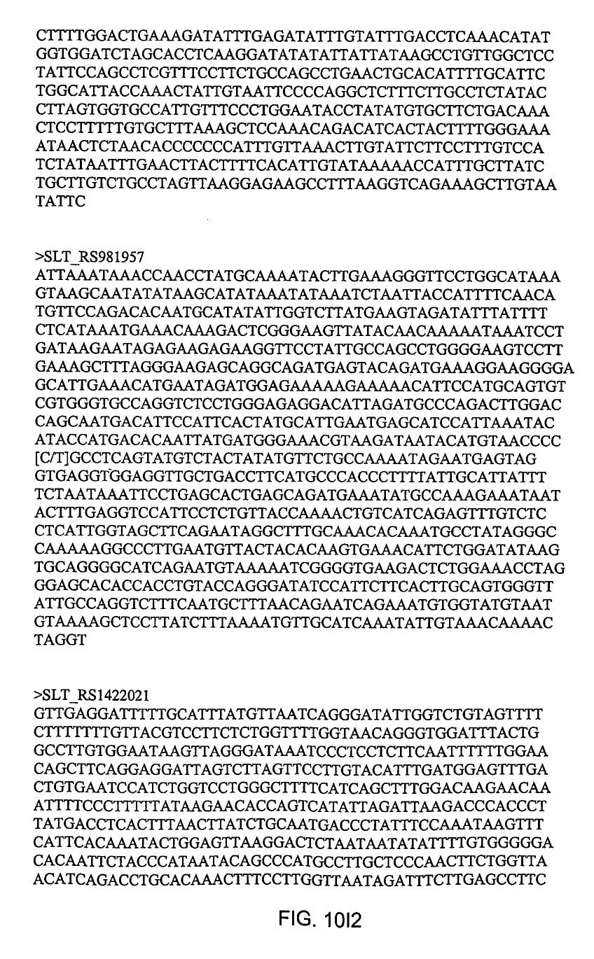

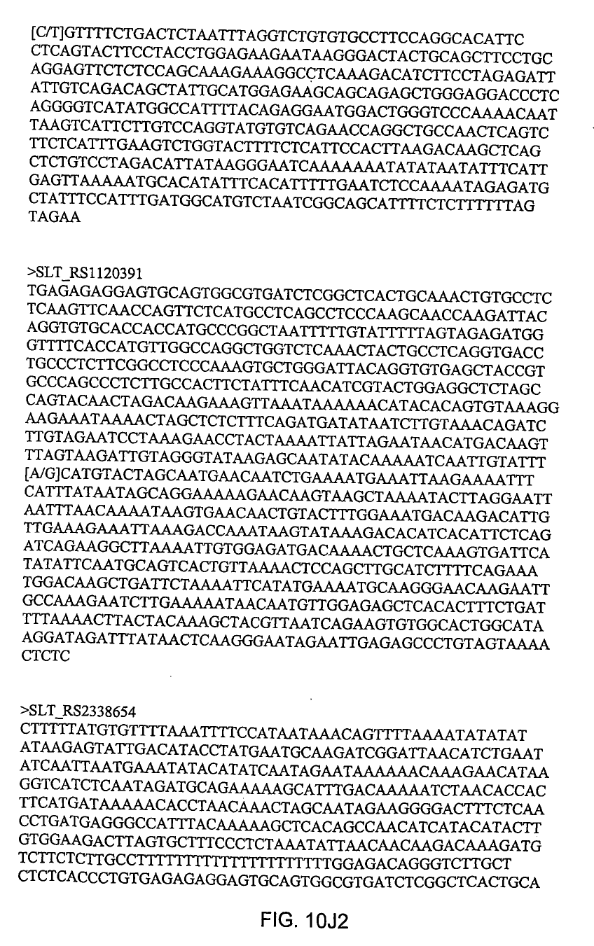

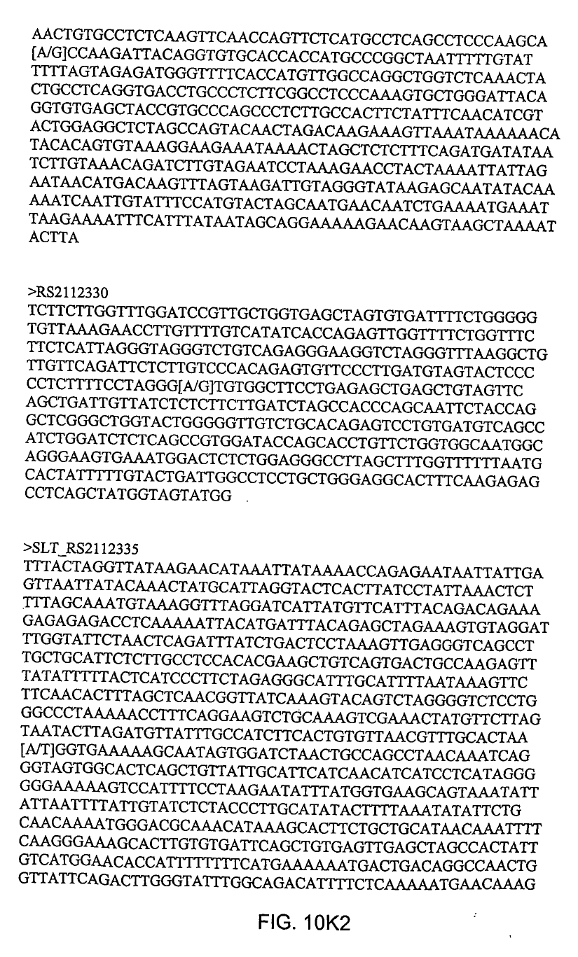

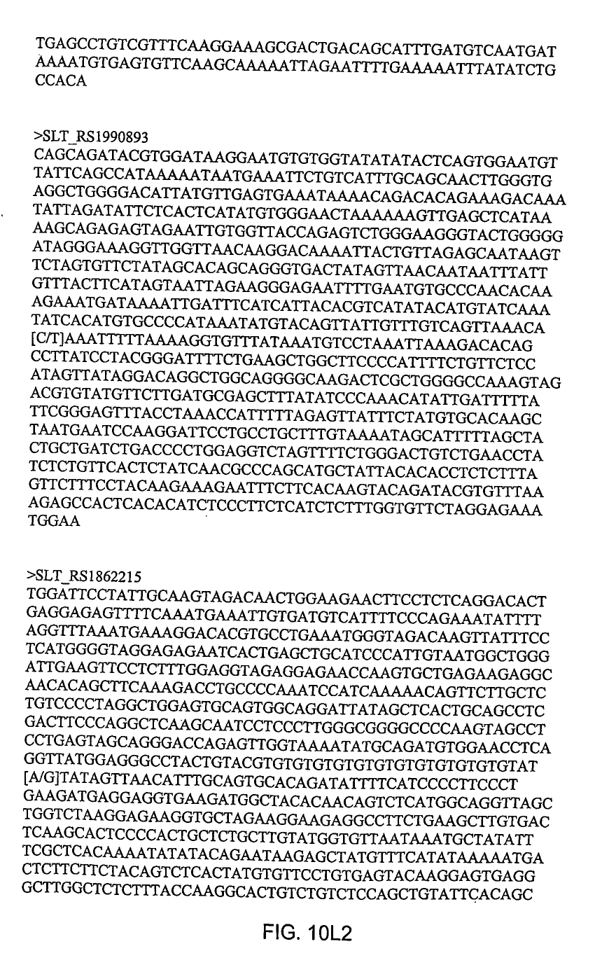

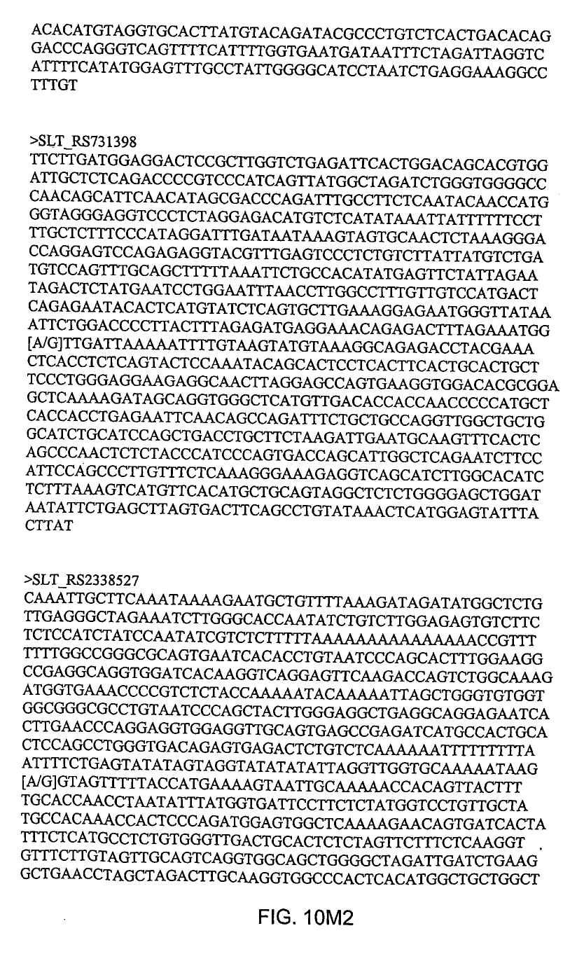

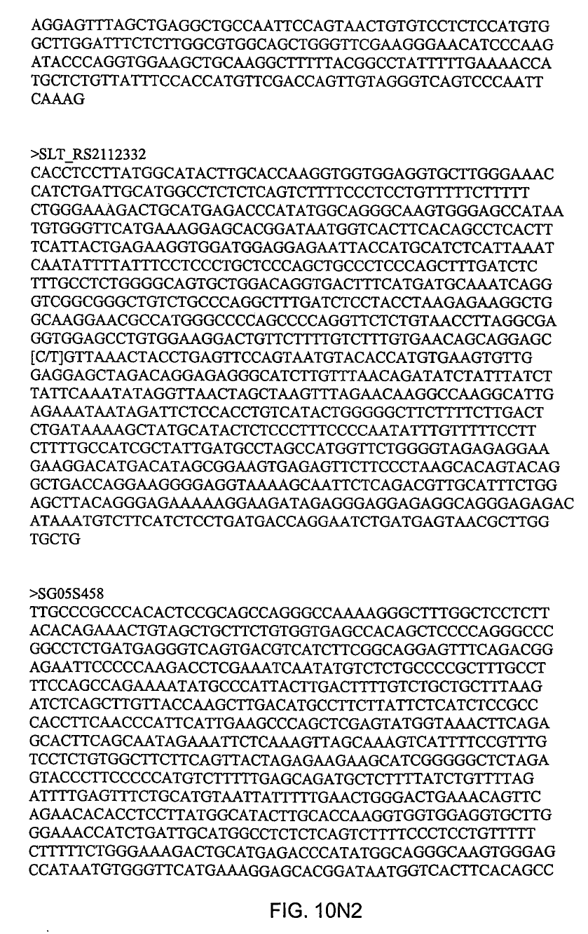

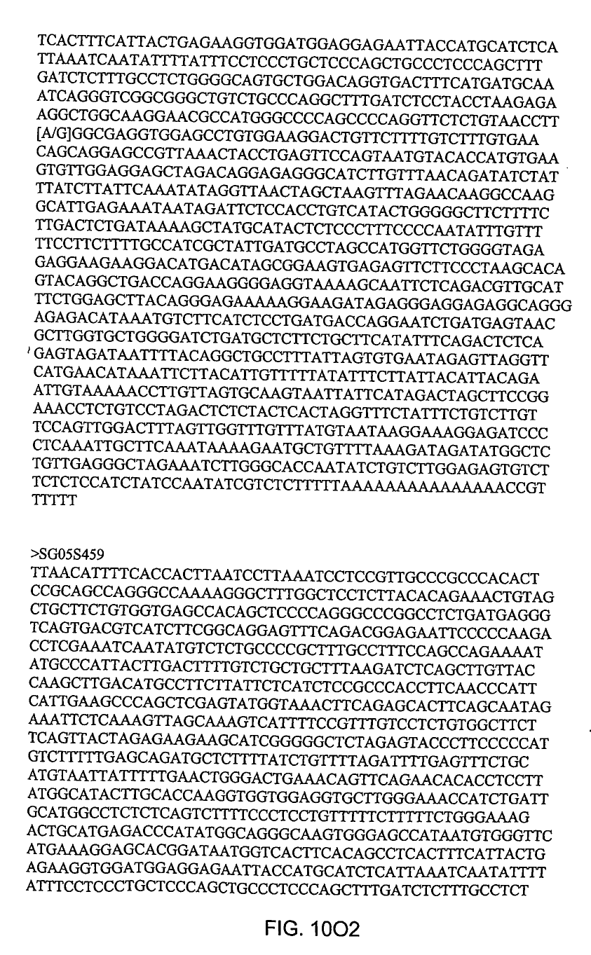

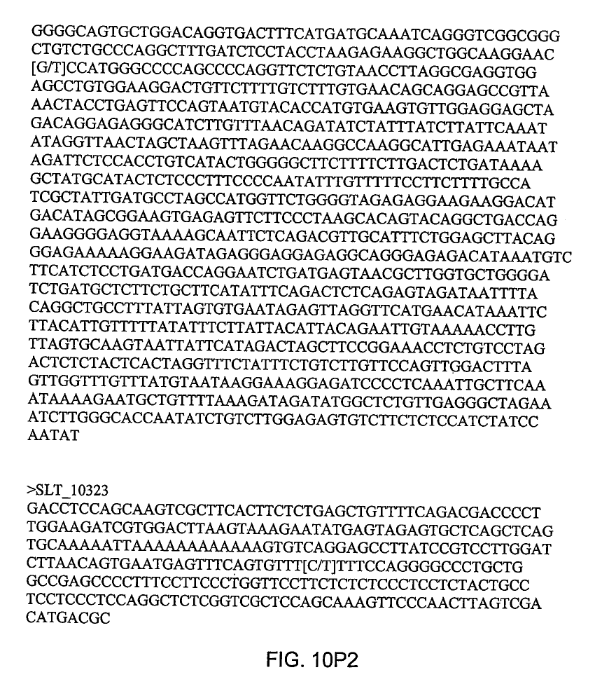

For example, the SLIT-3 nucleic acid can be the genomic sequence shown in FIG. 1, or a portion or fragment of the isolated nucleic acid molecule (e.g. cDNA or the gene) that encodes SLIT-3 polypeptide. In certain embodiments, the isolated nucleic acid molecule comprises a nucleic acid molecule selected from the group consisting of the sequences shown in FIG. 10, or the complement of such a nucleic acid molecule.

Additionally, nucleic acid molecules of the invention can be fused to a marker sequence, for example, a sequence that encodes a polypeptide to assist in isolation or purification of the polypeptide. Such sequences include, but are not limited to, those that encode a glutathione-S-transferase (GST) fusion protein and those that encode a hemagglutinin A (HA) polypeptide marker from influenza.

An “isolated” nucleic acid molecule, as used herein, is one that is separated from nucleic acids that normally flank the gene or nucleotide sequence (as in genomic sequences) and/or has been completely or partially purified from other transcribed sequences (e.g., as in an RNA library). For example, an isolated nucleic acid of the invention may be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system or reagent mix. In other circumstances, the material may be purified to essential homogeneity, for example as determined by PAGE or column chromatography such as HPLC. Preferably, an isolated nucleic acid molecule comprises at least about 50, 80 or 90% (on a molar basis) of all macromolecular species present. With regard to genomic DNA, the term “isolated” also can refer to nucleic acid molecules that are separated from the chromosome with which the genomic DNA is naturally associated. For example, the isolated nucleic acid molecule can contain less than about 5 kb but not limited to 4 kb, 3 kb, 2 kb, 1 kb, 0.5 kb or 0.1 kb of nucleotides which flank the nucleic acid molecule in the genomic DNA of the cell from which the nucleic acid molecule is derived.

The nucleic acid molecule can be fused to other coding or regulatory sequences and still be considered isolated. Thus, recombinant DNA contained in a vector is included in the definition of “isolated” as used herein. Also, isolated nucleic acid molecules include recombinant DNA molecules in heterologous host cells, as well as partially or substantially purified DNA molecules in solution. “Isolated” nucleic acid molecules also encompass in vivo and in vitro RNA transcripts of the DNA molecules of the present invention. An isolated nucleic acid molecule can include a nucleic acid molecule or nucleic acid sequence that is synthesized chemically or by recombinant means. Therefore, recombinant DNA contained in a vector is included in the definition of “isolated” as used herein. Also, isolated nucleic acid molecules include recombinant DNA molecules in heterologous organisms, as well as partially or substantially purified DNA molecules in solution. In vivo and in vitro RNA transcripts of the DNA molecules of the present invention are also encompassed by “isolated” nucleic acid sequences. Such isolated nucleic acid molecules are useful in the manufacture of the encoded polypeptide, as probes for isolating homologous sequences (e.g., from other mammalian species), for gene mapping (e.g., by in situ hybridization with chromosomes), or for detecting expression of the gene in tissue (e.g., human tissue), such as by Northern blot analysis.

The present invention also pertains to nucleic acid molecules which are not necessarily found in nature but which encode a SLIT-3 polypeptide, or another splicing variant of a SLIT polypeptide or polymorphic variant thereof. Thus, for example, the invention pertains to DNA molecules comprising a sequence that is different from the naturally occurring nucleotide sequence but which, due to the degeneracy of the genetic code, encode a SLIT polypeptide of the present invention. The invention also encompasses nucleic acid molecules encoding portions (fragments), or encoding variant polypeptides such as analogues or derivatives of a SLIT-3 polypeptide. Such variants can be naturally occurring, such as in the case of allelic variation or single nucleotide polymorphisms, or non-naturally-occurring, such as those induced by various mutagens and mutagenic processes. Intended variations include, but are not limited to, addition, deletion and substitution of one or more nucleotides that can result in conservative or non-conservative amino acid changes, including additions and deletions. Preferably the nucleotide (and/or resultant amino acid) changes are silent or conserved; that is, they do not alter the characteristics or activity of a SLIT-3 polypeptide. In one embodiment, the nucleic acid sequences are fragments that comprise one or more polymorphic microsatellite markers. In another embodiment, the nucleotide sequences are fragments that comprise one or more single nucleotide polymorphisms in a SLIT-3 gene.

Other alterations of the nucleic acid molecules of the invention can include, for example, labeling, methylation, internucleotide modifications such as uncharged linkages (e.g., methyl phosphonates, phosphotriesters, phosphoanidates, carbamates), charged linkages (e.g., phosphorothioates, phosphorodithioates), pendent moieties (e.g., polypeptides), intercalators (e.g., acridine, psoralen), chelators, alkylators, and modified linkages (e.g., alpha anomeric nucleic acids). Also included are synthetic molecules that mimic nucleic acid molecules in the ability to bind to a designated sequence via hydrogen bonding and other chemical interactions. Such molecules include, for example, those in which peptide linkages substitute for phosphate linkages in the backbone of the molecule.

The invention also pertains to nucleic acid molecules that hybridize under high stringency hybridization conditions, such as for selective hybridization, to a nucleotide sequence described herein (e.g. nucleic acid molecules which specifically hybridize to a nucleotide sequence encoding polypeptides described herein, and, optionally, have an activity of the polypeptide). In one embodiment, the invention includes variants described herein which hybridize under high stringency hybridization conditions (e.g., for selective hybridization) to a nucleotide sequence comprising a nucleotide sequence selected from the group consisting of the sequences shown in FIG. 10. In another embodiment, the invention includes variants described herein that hybridize under high stringency hybridization conditions (e.g. for selective hybridization) to a nucleotide sequence encoding an amino acid sequence or a polymorphic variant thereof. In a preferred embodiment, the variant that hybridizes under high stringency hybridizations has an activity of a SLIT polypeptide.

Such nucleic acid molecules can be detected and/or isolated by specific hybridization (e.g., under high stringency conditions). “Specific hybridization,” as used herein, refers to the ability of a first nucleic acid to hybridize to a second nucleic acid in a manner such that the first nucleic acid does not hybridize to any nucleic acid other than to the second nucleic acid (e.g., when the first nucleic acid has a higher similarity to the second nucleic acid than to any other nucleic acid in a sample wherein the hybridization is to be performed). “Stringency conditions” for hybridization is a term of art which refers to the incubation and wash conditions, e.g., conditions of temperature and buffer concentration, which permit hybridization of a particular nucleic acid to a second nucleic acid; the first nucleic acid may be perfectly (i.e., 100%) complementary to the second, or the first and second may share some degree of complementarity which is less than perfect (e.g., 70%, 75%, 85%, 95%). For example, certain high stringency conditions can be used which distinguish perfectly complementary nucleic acids from those of less complementarity. “High stringency conditions”, “moderate stringency conditions” and “low stringency conditions” for nucleic acid hybridizations are explained on pages 2.10.1-2.10.16 and pages 6.3.1-6.3.6 in Current Protocols in Molecular Biology (Ausubel, F. M. et al., “Current Protocols in Molecular Biology”, John Wiley & Sons, (2001)), the entire teachings of which are incorporated by reference herein). The exact conditions which determine the stringency of hybridization depend not only on ionic strength (e.g., 0.2×SSC, 0.1×SSC), temperature (e.g., room temperature, 42° C., 68° C.) and the concentration of destabilizing agents such as formamide or denaturing agents such as SDS, but also on factors such as the length of the nucleic acid sequence, base composition, percent mismatch between hybridizing sequences and the frequency of occurrence of subsets of that sequence within other non-identical sequences. Thus, equivalent conditions can be determined by varying one or more of these parameters while maintaining a similar degree of identity or similarity between the two nucleic acid molecules. Typically, conditions are used such that sequences at least about 60%, at least about 70%, at least about 80%, at least about 90% or at least about 95% or more identical to each other remain hybridized to one another. By varying hybridization conditions from a level of stringency at which no hybridization occurs to a level at which hybridization is first observed, conditions which will allow a given sequence to hybridize (e.g., selectively) with the most similar sequences in the sample can be determined.

Exemplary conditions are described in Krause, M. H. and S. A. Aaronson, Methods in Enzymology 200:546-556 (1991), and in, Ausubel, et al., “Current Protocols in Molecular Biology”, John Wiley & Sons, (2001), which describes the determination of washing conditions for moderate or low stringency conditions. Washing is the step in which conditions are usually set so as to determine a minimum level of complementarity of the hybrids. Generally, starting from the lowest temperature at which only homologous hybridization occurs, each ° C. by which the final wash temperature is reduced (holding SSC concentration constant) allows an increase by 1% in the maximum extent of mismatching among the sequences that hybridize. Generally, doubling the concentration of SSC results in an increase in Tₘ of −17° C. Using these guidelines, the washing temperature can be determined empirically for high, moderate or low stringency, depending on the level of mismatch sought.

For example, a low stringency wash can comprise washing in a solution containing 0.2×SSC/0.1% SDS for 10 minutes at room temperature; a moderate stringency wash can comprise washing in a pre-warmed solution (42° C.) solution containing 0.2×SSC/0.1% SDS for 15 minutes at 42° C.; and a high stringency wash can comprise washing in pre-warmed (68° C.) solution containing 0.1×SSC/0.1% SDS for 15 minutes at 68° C. Furthermore, washes can be performed repeatedly or sequentially to obtain a desired result as known in the art. Equivalent conditions can be determined by varying one or more of the parameters given as an example, as known in the art, while maintaining a similar degree of identity or similarity between the target nucleic acid molecule and the primer or probe used.

The percent homology or identity of two nucleotide or amino acid sequences can be determined by aligning the sequences for optimal comparison purposes (e.g., gaps can be introduced in the sequence of a first sequence for optimal alignment). The nucleotides or amino acids at corresponding positions are then compared, and the percent identity between the two sequences is a function of the number of identical positions shared by the sequences (i.e., % identity=# of identical positions/total # of positions×100). When a position in one sequence is occupied by the same nucleotide or amino acid residue as the corresponding position in the other sequence, then the molecules are homologous at that position. As used herein, nucleic acid or amino acid “homology” is equivalent to nucleic acid or amino acid “identity”. In certain embodiments, the length of a sequence aligned for comparison purposes is at least 30%, for example, at least 40%, in certain embodiments at least 60%, and in other embodiments at least 70%, 80%, 90% or 95% of the length of the reference sequence. The actual comparison of the two sequences can be accomplished by well-known methods, for example, using a mathematical algorithm. A preferred, non-lirniting example of such a mathematical algorithm is described in Karlin et al., Proc. Natl. Acad. Sc. USA 90:5873-5877 (1993). Such an algorithm is incorporated into the NBLAST and XBLAST programs (version 2.0) as described in Altschul et al., Nucleic Acids Res. 25:389-3402 (1997). When utilizing BLAST and Gapped BLAST programs, the default parameters of the respective programs (e.g., NBLAST) can be used. In one embodiment, parameters for sequence comparison can be set at score=100, wordlength=12, or can be varied (e.g., W=5 or W=20).

Another preferred, non-limiting example of a mathematical algorithm utilized for the comparison of sequences is the algorithm of Myers and Miller, CABIOS 4(1): 11-17 (1988). Such an algorithm is incorporated into the ALIGN program (version 2.0) which is part of the GCG sequence alignment software package (Accelrys, Cambridge, UK). When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti, Comput. Appl. Biosci. 10:3-5 (1994); and FASTA described in Pearson and Lipman, Proc. Natl. Acad. Sci. USA 85:2444-8 (1988).

In another embodiment, the percent identity between two amino acid sequences can be accomplished using the GAP program in the GCG software package using either a BLOSUM63 matrix or a PAM250 matrix, and a gap weight of 12, 10, 8, 6, or 4 and a length weight of 2, 3, or 4. In yet another embodiment, the percent identity between two nucleic acid sequences can be accomplished using the GAP program in the GCG software package using a gap weight of 50 and a length weight of 3.

The present invention also provides isolated nucleic acid molecules that contain a fragment or portion that hybridizes under highly stringent conditions to a nucleotide sequence comprising a nucleotide sequence selected from the group consisting of the sequences shown in FIG. 10, or the complement of such a sequence, and also provides isolated nucleic acid molecules that contain a fragment or portion that hybridizes under highly stringent conditions to a nucleotide sequence encoding an amino acid sequence or polymorphic variant thereof. The nucleic acid fragments of the invention are at least about 15, preferably at least about 18, 20, 23 or 25 nucleotides, and can be 30, 40, 50, 100, 200 or more nucleotides in length. Longer fragments, for example, 30 or more nucleotides in length, that encode antigenic polypeptides described herein are particularly useful, such as for the generation of antibodies as described below.

Probes and Primers

In a related aspect, the nucleic acid fragments of the invention are used as probes or primers in assays such as those described herein. “Probes” or “primers” are oligonucleotides that hybridize in a base-specific manner to a complementary strand of nucleic acid molecules. Such probes and primers include polypeptide nucleic acids, as described in Nielsen et al., Science 254:1497-1500 (1991).

A probe or primer comprises a region of nucleotide sequence that hybridizes to at least about 15, for example about 20-25, and in certain embodiments about 40, 50 or 75, consecutive nucleotides of a nucleic acid molecule comprising a contiguous nucleotide sequence selected from the group consisting of the sequences shown in FIG. 10, or polymorphic variant thereof. In other embodiments, a probe or primer comprises 100 or fewer nucleotides, in certain embodiments from 6 to 50 nucleotides, for example from 12 to 30 nucleotides. In other embodiments, the probe or primer is at least 70% identical to the contiguous nucleotide sequence or to the complement of the contiguous nucleotide sequence, for example at least 80% identical, in certain embodiments at least 90% identical, and in other embodiments at least 95% identical, or even capable of selectively hybridizing to the contiguous nucleotide sequence or to the complement of the contiguous nucleotide sequence. Often, the probe or primer further comprises a label, e.g., radioisotope, fluorescent compound, enzyme, or enzyme co-factor.

The nucleic acid molecules of the invention such as those described above can be identified and isolated using standard molecular biology techniques and the sequence information provided herein. For example, nucleic acid molecules can be amplified and isolated by the polymerase chain reaction using synthetic oligonucleotide primers designed based on one or more of the sequences selected from the group consisting of the sequences shown in FIG. 10, or the complement of such a sequence, or designed based on nucleotides based on sequences encoding one or more of the amino acid sequences provided herein. See generally PCR Technology: Principles and Applications for DNA Amplification (ed. H. A. Erlich, Freeman Press, NY, N.Y., 1992); PCR Protocols: A Guide to Methods and Applications (Eds. Innis et al., Academic Press, San Diego, Calif., 1990); Mattila et al., Nucl. Acids Res. 19: 4967 (1991); Eckert et al., PCR Methods and Applications 1:17 (1991); PCR (eds. McPherson et al., IRL Press, Oxford); and U.S. Pat. No. 4,683,202. The nucleic acid molecules can be amplified using cDNA, mRNA or genomic DNA as a template, cloned into an appropriate vector and characterized by DNA sequence analysis.

Other suitable amplification methods include the ligase chain reaction (LCR) (see Wu and Wallace, Genomics 4:560 (1989), Landegren et al., Science 241:1077 (1988), transcription amplification (Kwoh et al., Proc. Natl. Acad. Sci. USA 86:1173 (1989)), and self-sustained sequence replication (Guatelli et al., Proc. Nat. Acad. Sci. USA 87:1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

The amplified DNA can be labeled, for example, radiolabeled, and used as a probe for screening a cDNA library derived from human cells, mRNA in zap express, ZIPLOX or other suitable vector. Corresponding clones can be isolated, DNA can obtained following in vivo excision, and the cloned insert can be sequenced in either or both orientations by art recognized methods to identify the correct reading frame encoding a polypeptide of the appropriate molecular weight. For example, the direct analysis of the nucleotide sequence of nucleic acid molecules of the present invention can be accomplished using well-known methods that are commercially available. See, for example, Sambrook et al., Molecular Cloning, A Laboratory Manual (2nd Ed., CSHP, New York 1989); Zyslind et al., Recombinant DNA Laboratory Manual, (Acad. Press, 1988)). Additionally, fluorescence methods are also available for analyzing nucleic acids (Chen et al., Genome Res. 9, 492 (1999)) and polypeptides. Using these or similar methods, the polypeptide and the DNA encoding the polypeptide can be isolated, sequenced and further characterized.

Antisense nucleic acid molecules of the invention can be designed using the nucleotide sequences of one or more of the sequences shown in FIG. 10, and/or the complement of one or more of the sequences shown in FIG. 10, and/or a portion of one or more of the sequences shown in FIG. 10, or the complement of one or more of the sequences shown in FIG. 10, and constructed using chemical synthesis and enzymatic ligation reactions using procedures known in the art. For example, an antisense nucleic acid molecule (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Alternatively, the antisense nucleic acid molecule can be produced biologically using an expression vector into which a nucleic acid molecule has been subcloned in an antisense orientation (i.e., RNA transcribed from the inserted nucleic acid molecule will be of an antisense orientation to a target nucleic acid of interest).

The nucleic acid sequences can also be used to compare with endogenous DNA sequences in patients to identify one or more of the disorders described above, and as probes, such as to hybridize and discover related DNA sequences or to subtract out known sequences from a sample. The nucleic acid sequences can further be used to derive primers for genetic fingerprinting, to raise anti-polypeptide antibodies using DNA immunization techniques, and as an antigen to raise anti-DNA antibodies or elicit immune responses. Portions or fragments of the nucleotide sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Additionally, the nucleotide sequences of the invention can be used to identify and express recombinant polypeptides for analysis, characterization or therapeutic use, or as markers for tissues in which the corresponding polypeptide is expressed, either constitutively, during tissue differentiation, or in diseased states. The nucleic acid sequences can additionally be used as reagents in the screening and/or diagnostic assays described herein, and can also be included as components of kits (e.g., reagent kits) for use in the screening and/or diagnostic assays described herein.

Vectors and Host Cells

Another aspect of the invention pertains to nucleic acid constructs containing a nucleic acid molecule selected from the group consisting of the sequences shown in FIG. 10, and the complements thereof (or a portion thereof). The constructs comprise a vector (e.g., an expression vector) into which a sequence of the invention has been inserted in a sense or antisense orientation As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Expression vectors are capable of directing the expression of genes to which they are operably linked. In general, expression vectors of utility in recombinant DNA techniques are often in the form of plasmids. However, the invention is intended to include such other forms of expression vectors, such as viral vectors (e.g., replication defective retroviruses, adenoviruses and adeno-associated viruses) that serve equivalent functions.

In certain embodiments, recombinant expression vectors of the invention comprise a nucleic acid molecule of the invention in a form suitable for expression of the nucleic acid molecule in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” or “operatively linked” is intended to mean that the nucleotide sequence of interest is linked to the regulatory sequence(s) in a manner which allows for expression of he nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, “Gene Expression Technology”, Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed and the level of expression of polypeptide desired. The expression vectors of the invention can be introduced into host cells to thereby produce polypeptides, including fusion polypeptides, encoded by nucleic acid molecules as described herein.

The recombinant expression vectors of the invention can be designed for expression of a polypeptide of the invention in prokaryotic or eukaryotic cells, e.g., bacterial cells such as E. coli, insect cells (using baculovirus expression vectors), yeast cells or mammalian cells. Suitable host cells are discussed further in Goeddel, supra. Alternatively, the recombinant expression vector can be transcribed and translated in vitro, for example using T7 promoter regulatory sequences and T7 polymerase.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a nucleic acid molecule of the invention can be expressed in bacterial cells (e.g., E. coli), insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing a foreign nucleic acid molecule (e.g. DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g. for resistance to antibiotics) is generally introduced into the host cells along with the gene of interest. Preferred selectable markers include those that confer resistance to drugs, such as G418, hygromycin and methotrexate. Nucleic acid molecules encoding a selectable marker can be introduced into a host cell on the same vector as the nucleic acid molecule of the invention or can be introduced on a separate vector. Cells stably transfected with the introduced nucleic acid molecule can be identified by drug selection (e.g., cells that have incorporated the selectable marker gene will survive, while the other cells die).

A host cell of the invention, such as a prokaryotic or eukaryotic host cell in culture, can be used to produce (i.e., express) a polypeptide of the invention. Accordingly, the invention further provides methods for producing a polypeptide using the host cells of the invention. In one embodiment, the method comprises culturing the host cell of invention (into which a recombinant expression vector encoding a polypeptide of the invention has been introduced) in a suitable medium such that the polypeptide is produced. In another embodiment, the method further comprises isolating the polypeptide from the medium or the host cell.

The host cells of the invention can also be used to produce nonhuman transgenic animals. For example, in one embodiment, a host cell of the invention is a fertilized oocyte or an embryonic stem cell into which a nucleic acid molecule of the invention has been introduced (e.g., an exogenous SLIT gene, or an exogenous nucleic acid encoding a SLIT polypeptide). Such host cells can then be used to create non-human transgenic animals in which exogenous nucleotide sequences have been introduced into the genome or homologous recombinant animals in which endogenous nucleotide sequences have been altered. Such animals are useful for studying the function and/or activity of the nucleotide sequence and polypeptide encoded by the sequence and for identifying and/or evaluating modulators of their activity. As used herein, a “transgenic animal” is a non-human animal, preferably a mammal, more preferably a rodent such as a rat or mouse, in which one or more of the cells of the animal include a transgene. Other examples of transgenic animals include non-human primates, sheep, dogs, cows, goats, chickens and amphibians. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal prior to development of the animal.

Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191 and in Hogan, Manipulating the Mouse Embryo (Cold Spring Harbor Laboratory Press, Cold Spring Harbor, N.Y., 1986). Methods for constructing homologous recombination vectors and homologous recombinant animals are described further in Bradley, Current Opinion in BioTechnology 2:823-829 (1991) and in PCT Publication Nos. WO 90/11354, WO 91/01140, WO 92/0968, and WO 93/04169. Clones of the non-human transgenic animals described herein can also be produced according to the methods described in Wilmut et al., Nature 385:810-813 (1997) and PCT Publication Nos. WO 97/07668 and WO 97/07669.

### POLYPEPTIDES OF THE INVENTION

The present invention also pertains to isolated polypeptides encoded by SLIT-3 nucleic acids (“SLIT-3 polypeptides”) and fragments and variants thereof, as well as polypeptides encoded by nucleotide sequences described herein (e.g., other splicing variants). The term “polypeptide” refers to a polymer of amino acids, and not to a specific length; thus, peptides, oligopeptides and proteins are included within the definition of a polypeptide. As used herein, a polypeptide is said to be “isolated” or “purified” when it is substantially free of cellular material when it is isolated from recombinant and non-recombinant cells, or free of chemical precursors or other chemicals when it is chemically synthesized. A polypeptide, however, can be joined to another polypeptide with which it is not normally associated in a cell (e.g., in a “fusion protein”) and still be “isolated” or “purified.”

The polypeptides of the invention can be purified to homogeneity. It is understood, however, that preparations in which the polypeptide is not purified to homogeneity are useful. The critical feature is that the preparation allows for the desired function of the polypeptide, even in the presence of considerable amounts of other components. Thus, the invention encompasses various degrees of purity. In one embodiment, the language “substantially free of cellular material” includes preparations of the polypeptide having less than about 30% (by dry weight) other proteins (i.e., contaminating protein), less than about 20% other proteins, less than about 10% other proteins, or less than about 5% other proteins.

When a polypeptide is recombinantly produced, it can also be substantially free of culture medium, i.e., culture medium represents less than about 20%, less than about 10%, or less than about 5% of the volume of the polypeptide preparation. The language “substantially free of chemical precursors or other chemicals” includes preparations of the polypeptide in which it is separated from chemical precursors or other chemicals that are involved in its synthesis. In one embodiment, the language “substantially free of chemical precursors or other chemicals” includes preparations of the polypeptide having less than about 30% (by dry weight) chemical precursors or other chemicals, less than about 20% chemical precursors or other chemicals, less than about 10% chemical precursors or other chemicals, or less than about 5% chemical precursors or other chemicals.

In one embodiment, a polypeptide of the invention comprises an amino acid sequence encoded by a nucleic acid molecule comprising a nucleotide sequence selected from the group consisting of the sequences shown in FIG. 10, or the complement of such a nucleic acid, or portions thereof, e.g., the sequences shown in FIG. 10, or a portion or polymorphic variant thereof. However, the polypeptides of the invention also encompass fragment and sequence variants. Variants include a substantially homologous polypeptide encoded by the same genetic locus in an organism, i.e., an allelic variant, as well as other splicing variants. Variants also encompass polypeptides derived from other genetic loci in an organism, but having substantial homology to a polypeptide encoded by a nucleic acid molecule comprising a nucleotide sequence selected from the group consisting of the sequences shown in FIG. 10, or a complement of such a sequence, or portions thereof or polymorphic variants thereof. Variants also include polypeptides substantially homologous or identical to these polypeptides but derived from another organism, i.e., an ortholog. Variants also include polypeptides that are substantially homologous or identical to these polypeptides that are produced by chemical synthesis. Variants also include polypeptides that are substantially homologous or identical to these polypeptides that are produced by recombinant methods.

As used herein, two polypeptides (or a region of the polypeptides) are substantially homologous or identical when the amino acid sequences are at least about 45-55%, in certain embodiments at least about 70-75%, and in other embodiments, at least about 80-85%, an in other embodiments greater than about 90% or more homologous or identical. A substantially homologous amino acid sequence, according to the present invention, will be encoded by a nucleic acid molecule hybridizing to one or more of the sequences shown in FIG. 10, or portion thereof, under stringent conditions as more particularly described above, or will be encoded by a nucleic acid molecule hybridizing to a nucleic acid sequence encoding one of the sequences shown in FIG. 10, a portion thereof or polymorphic variant thereof, under stringent conditions as more particularly described thereof.

The invention also encompasses polypeptides having a lower degree of identity but having sufficient similarity so as to perform one or more of the same functions performed by a polypeptide encoded by a nucleic acid molecule of the invention.

Similarity is determined by conserved amino acid substitution where a given amino acid in a polypeptide is substituted by another amino acid of like characteristics. Conservative substitutions are likely to be phenotypically silent. Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe and Tyr. Guidance concerning which amino acid changes are likely to be phenotypically silent are found in Bowie et al., Science 247:1306-1310 (1990).

A variant polypeptide can differ in amino acid sequence by one or more substitutions, deletions, insertions, inversions, fusions, and truncations or a combination of any of these. Further, variant polypeptides can be fully functional or can lack function in one or more activities. Fully functional variants typically contain only conservative variation or variation in non-critical residues or in noncritical regions. Functional variants can also contain substitution of similar amino acids that result in no change or an insignificant change in function. Alternatively, such substitutions may positively or negatively affect function to some degree. Non-functional variants typically contain one or more non-conservative amino acid substitutions, deletions, insertions, inversions, or truncation or a substitution, insertion, inversion, or deletion in a critical residue or critical region.

Amino acids that are essential for function can be identified by methods known in the art, such as site-directed mutagenesis or alanine-scanning mutagenesis (Cunningham et al., Science 244:1082-1185 (1989)). The latter procedure introduces single alanine mutations at every residue in the molecule. The resulting mutant molecules are then tested for biological activity in vitro, or in vitro proliferative activity. Sites that are critical for polypeptide activity can also be determined by structural analysis such as crystallization, nuclear magnetic resonance or photoaffinity labeling (Smith et al., J. Mol. Biol. 224:899-904 (1992); de Vos et al., Science 255:306-312 (1992)).

The invention also includes polypeptide fragments of the polypeptides of the invention. Fragments can be derived from a polypeptide encoded by a nucleic acid molecule comprising one of the sequences shown in FIG. 10, or a complement of such a nucleic acid or other variants. However, the invention also encompasses fragments of the variants of the polypeptides described herein. As used herein, a fragment comprises at least 6 contiguous amino acids. Useful fragments include those that retain one or more of the biological activities of the polypeptide as well as fragments that can be used as an immunogen to generate polypeptide-specific antibodies.

Biologically active fragments (peptides which are, for example, 6, 9, 12, 15, 16, 20, 30, 35, 36, 37, 38, 39, 40, 50, 100 or more amino acids in length) can comprise a domain, segment, or motif that has been identified by analysis of the polypeptide sequence using well-known methods, e.g., signal peptides, extracellular domains, one or more transmembrane segments or loops, ligand binding regions, zinc finger domains, DNA binding domains, acylation sites, glycosylation sites, or phosphorylation sites.

Fragments can be discrete (not fused to other amino acids or polypeptides) or can be within a larger polypeptide. Further, several fragments can be comprised within a single larger polypeptide. In one embodiment a fragment designed for expression in a host can have heterologous pre- and pro-polypeptide regions fused to the amino terminus of the polypeptide fragment and an additional region fused to the carboxyl terminus of the fragment.

The invention thus provides chimeric or fusion polypeptides. These comprise a polypeptide of the invention operatively linked to a heterologous protein or polypeptide having an amino acid sequence not substantially homologous to the polypeptide.

“Operatively linked” indicates that the polypeptide and the heterologous protein are fused in-frame. The heterologous protein can be fused to the N-terminus or C-terminus of the polypeptide. In one embodiment the fusion polypeptide does not affect function of the polypeptide per se. For example, the fusion polypeptide can be a GST-fusion polypeptide in which the polypeptide sequences are fused to the C-terminus of the GST sequences. Other types of fusion polypeptides include, but are not limited to, enzymatic fusion polypeptides, for example beta-galactosidase fusions, yeast two-hybrid GAL fusions, poly-His fusions and Tg fusions. Such fusion polypeptides, particularly poly-His fusions, can facilitate the purification of recombinant polypeptide. In certain host cells (e.g., mammalian host cells), expression and/or secretion of a polypeptide can be increased using a heterologous signal sequence. Therefore, in another embodiment, the fusion polypeptide contains a heterologous signal sequence at its N-terminus.

EP-A-O 464 533 discloses fusion proteins comprising various portions of immunoglobulin constant regions. The Fc is useful in therapy and diagnosis and thus results, for example, in improved pharmacokinetic properties (EP-A 0232 262). In drug discovery, for example, human proteins have been fused with Fc portions for the purpose of high-throughput screening assays to identify antagonists. Bennett et al., Journal of Molecular Recognition, 8:52-58 (1995) and Johanson et al., The Journal of Biological Chemistry, 270,16:9459-9471 (1995). Thus, this invention also encompasses soluble fusion polypeptides containing a polypeptide of the invention and various portions of the constant regions of heavy or light chains of immunoglobulins of various subclasses (IgG, IgM, IgA, IgE).

A chimeric or fusion polypeptide can be produced by standard recombinant DNA techniques. For example, DNA fragments coding for the different polypeptide sequences are ligated together in-frame in accordance with conventional techniques. In another embodiment, the fusion gene can be synthesized by conventional techniques including automated DNA synthesizers. Alternatively, PCR amplification of nucleic acid fragments can be carried out using anchor primers which give rise to complementary overhangs between two consecutive nucleic acid fragments which can subsequently be annealed and re-amplified to generate a chimeric nucleic acid sequence (see Ausubel et al., Current Protocols in Molecular Biology, 1992).

Moreover, many expression vectors are commercially available that already encode a fusion moiety (e.g., a GST protein). A nucleic acid molecule encoding a polypeptide of the invention can be cloned into such an expression vector such that the fusion moiety is linked in-frame to the polypeptide.

The isolated polypeptide can be purified from cells that naturally express it, can be purified from cells that have been altered to express it (recombinant), or synthesized using known protein synthesis methods. In one embodiment, the polypeptide is produced by recombinant DNA techniques. For example, a nucleic acid molecule encoding the polypeptide is cloned into an expression vector, the expression vector introduced into a host cell and the polypeptide expressed in the host cell. The polypeptide can then be isolated from the cells by an appropriate purification scheme using standard protein purification techniques.

The polypeptides of the present invention can be used to raise antibodies or to elicit an immune response. The polypeptides can also be used as a reagent, e.g., a labeled reagent, in assays to quantitatively determine levels of the polypeptide or a molecule to which it binds (e.g., a ligand) in biological fluids. The polypeptides can also be used as markers for cells or tissues in which the corresponding polypeptide is preferentially expressed, either constitutively, during tissue differentiation, or in a diseased state. The polypeptides can be used to isolate a corresponding binding agent, e.g., ligand, such as, for example, in an interaction trap assay, and to screen for peptide or small molecule antagonists or agonists of the binding interaction.

### ANTIBODIES OF THE INVENTION

Polyclonal antibodies and/or monoclonal antibodies that specifically bind one form of the gene or nucleic acid product but not to the other form of the gene or nucleic acid product are also provided. Antibodies are also provided which bind a portion of either the variant or the reference gene product that contains the polymorphic site or sites. The term “antibody” as used herein refers to immunoglobulin molecules and immunologically active portions of immunoglobulin molecules, i.e., molecules that contain an antigen binding site that specifically bind an antigen. A molecule that specifically binds to a polypeptide of the invention is a molecule that binds to that polypeptide or a fragment thereof, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind to a polypeptide of the invention. The term “monoclonal antibody” or “monoclonal antibody composition”, as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared as described above by immunizing a suitable subject with a desired immunogen, e.g., polypeptide of the invention or a fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein, Nature 256:495-497 (1975), the human B cell hybridoma technique (Kozbor et al., Immunol. Today 4: 72 (1983)), the EBV-hybridoma technique (Cole et al., Monoclonal Antibodies and Cancer Therapy, Alan R. Liss, 1985, Inc., pp. 77-96) or trioma techniques. The technology for producing hybridomas is well known (see generally Current Protocols in Immunology (1994) Coligan et al. (eds.) John Wiley & Sons, Inc., New York, N.Y.). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a polypeptide of the invention (see, e.g., Current Protocols in Immunology, supra; Galfre et al., Nature 266:55052 (1977); R. H. Kenneth, in Monoclonal Antibodies: A New Dimension In Biological Analyses, Plenum Publishing Corp., New York, N.Y. (1980); and Lerner, Yale J. Biol. Med. 54:387-402 (1981)). Moreover, the ordinarily skilled worker will appreciate that there are many variations of such methods that also would be useful.

Alternative to preparing monoclonal antibody-secreting hybridomas, a monoclonal antibody to a polypeptide of the invention can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., Bio/Technology 9: 1370-1372 (1991); Hay et al., Hum. Antibod. Hybridomas 3:81-85 (1992); Huse et al., Science 246: 1275-1281 (1989); and Griffiths et al., EMBO J. 12:725-734 (1993).

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In general, antibodies of the invention (e.g., a monoclonal antibody) can be used to isolate a polypeptide of the invention by standard techniques, such as affinity chromatography or immunoprecipitation. A polypeptide-specific antibody can facilitate the purification of natural polypeptide from cells and of recombinantly produced polypeptide expressed in host cells. Moreover, an antibody specific for a polypeptide of the invention can be used to detect the polypeptide (e.g., in a cellular lysate, cell supernatant, or tissue sample) in order to evaluate the abundance and pattern of expression of the polypeptide. Antibodies can be used diagnostically to monitor protein levels in tissue as part of a clinical testing procedure, e.g., to, for example, determine the efficacy of a given treatment regimen. The antibody can be coupled to a detectable substance to facilitate its detection. Examples of detectable substances include various enzymes, prosthetic groups, fluorescent materials, luminescent materials, bioluminescent materials, and radioactive materials. Examples of suitable enzymes include horseradish peroxidase, alkaline phosphatase, -galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Diagnostic Assays

The nucleic acids, probes, primers, polypeptides and antibodies described herein can be used in methods of diagnosis of Type II diabetes or of a susceptibility to Type II diabetes, or of a condition associated with a SLIT-3 gene, as well as in kits (e.g., useful for diagnosis of Type II diabetes, of a susceptibility to Type II diabetes, or of a condition associated with a SLIT-3 gene). In one embodiment, the kit comprises primers that contain one or more of the SNP's identified in FIG. 11.

In one embodiment of the invention, diagnosis of a disease or condition associated with a SLIT-3 gene (e.g., diagnosis of Type II diabetes, or of a susceptibility to Type II diabetes) is made by detecting a polymorphism in a SLIT nucleic acid as described herein. The polymorphism can be a change in a SLIT-3 nucleic acid, such as the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of the gene; duplication of all or a part of the gene; transposition of all or a part of the gene; or rearrangement of all or a part of the gene. More than one such change may be present in a single gene. Such sequence changes cause a difference in the polypeptide encoded by a SLUT-3 nucleic acid. For example, if the difference is a frame shift change, the frame shift can result in a change in the encoded amino acids, and/or can result in the generation of a premature stop codon, causing generation of a truncated polypeptide. Alternatively, a polymorphism associated with a disease or condition or a susceptibility to a disease or condition associated with a SLIT-3 nucleic acid can be a synonymous alteration in one or more nucleotides (i.e., an alteration that does not result in a change in the polypeptide encoded by a SLIT-3 nucleic acid). Such a polymorphism may alter splicing sites, affect the stability or transport of mRNA, or otherwise affect the transcription or translation of the gene. A SLIT-3 nucleic acid that has any of the changes or alterations described above is referred to herein as an “altered nucleic acid.”

In a first method of diagnosing Type II diabetes or a susceptibility to Type II diabetes, or another disease or condition associated with a SLIT-3 gene, hybridization methods, such as Southern analysis, Northern analysis, or in situ hybridizations, can be used (see Current Protocols in Molecular Biology, Ausubel, F. et al., eds, John Wiley & Sons, including all supplements through 1999). For example, a biological sample (a “test sample”) from a test subject (the “test individual”) of genomic DNA, RNA, or cDNA, is obtained from an individual, such as an individual suspected of having, being susceptible to or predisposed for, or carrying a defect for, the disease or condition, or the susceptibility to the disease or condition, associated with a SLIT-3 gene (e.g., Type II diabetes). The individual can be an adult, child, or fetus. The test sample can be from any source which contains genomic DNA, such as a blood sample, sample of amniotic fluid, sample of cerebrospinal fluid, or tissue sample from skin, muscle, buccal or conjunctival mucosa, placenta, gastrointestinal tract or other organs. A test sample of DNA from fetal cells or tissue can be obtained by appropriate methods, such as by amniocentesis or chorionic villus sampling. The DNA, RNA, or cDNA sample is then examined to determine whether a polymorphism in a SLIT-3 nucleic acid is present, and/or to determine which splicing variant(s) encoded by the SLIT-3 is present. The presence of the polymorphism or splicing variant(s) can be indicated by hybridization of the gene in the genomic DNA, RNA, or cDNA to a nucleic acid probe. A “nucleic acid probe”, as used herein, can be a DNA probe or an RNA probe; the nucleic acid probe can contain, for example, at least one polymorphism in a SLIT-3 nucleic acid (e.g., as set forth in FIG. 11) and/or contain a nucleic acid encoding a particular splicing variant of a SLIT-3 nucleic acid. The probe can be any of the nucleic acid molecules described above (e.g., the gene or nucleic acid, a fragment, a vector comprising the gene or nucleic acid, a probe or primer, etc.).

To diagnose Type II diabetes, or a susceptibility to Type II diabetes, or another condition associated with a SLIT-3 gene, a hybridization sample is formed by contacting the test sample containing a SLIT-3 nucleic acid with at least one nucleic acid probe. A preferred probe for detecting mRNA or genomic DNA is a labeled nucleic acid probe capable of hybridizing to mRNA or genomic DNA sequences described herein. The nucleic acid probe can be, for example, a full-length nucleic acid molecule, or a portion thereof, such as an oligonucleotide of at least 15, 30, 50, 100, 250 or 500 nucleotides in length and sufficient to specifically hybridize under stringent conditions to appropriate mRNA or genomic DNA. For example, the nucleic acid probe can be all or a portion of one of the sequences shown in FIG. 10, or the complement thereof, or a portion thereof. Other suitable probes for use in the diagnostic assays of the invention are described above (see e.g., probes and primers discussed under the heading, “Nucleic Acids of the Invention”).

The hybridization sample is maintained under conditions that are sufficient to allow specific hybridization of the nucleic acid probe to a SLIT-3 nucleic acid “Specific hybridization”, as used herein, indicates exact hybridization (e.g., with no mismatches). Specific hybridization can be performed under high stringency conditions or moderate stringency conditions, for example, as described above. In a particularly preferred embodiment, the hybridization conditions for specific hybridization are high stringency.

Specific hybridization, if present, is then detected using standard methods. If specific hybridization occurs between the nucleic acid probe and SLIT-3 nucleic acid in the test sample, then the SLIT-3 has the polymorphism, or is the splicing variant, that is present in the nucleic acid probe. More than one nucleic acid probe can also be used concurrently in this method. Specific hybridization of any one of the nucleic acid probes is indicative of a polymorphism in the SLIT-3 nucleic acid, or of the presence of a particular splicing variant encoding the SLIT-3 nucleic acid and is therefore diagnostic for a susceptibility to a disease or condition associated with a SLIT-3 nucleic acid (e.g., Type II diabetes).

In Northern analysis (see Current Protocols in Molecular Biology, Ausubel, F. et al., eds., John Wiley & Sons, supra) the hybridization methods described above are used to identify the presence of a polymorphism or a particular splicing variant, associated with a susceptibility to a disease or condition associated with a SLIT-3 gene (e.g., Type II diabetes). For Northern analysis, a test sample of RNA is obtained from the individual by appropriate means. Specific hybridization of a nucleic acid probe, as described above, to RNA from the individual is indicative of a polymorphism in a SLIT-3 nucleic acid, or of the presence of a particular splicing variant encoded by a SLIT-3 nucleic acid and is therefore diagnostic for Type II diabetes or a susceptibility to Type II diabetes or a condition associated with a SLIT-3 nucleic acid (e.g., Type II diabetes).

For representative examples of use of nucleic acid probes, see, for example, U.S. Pat. Nos. 5,288,611 and 4,851,330.

Alternatively, a peptide nucleic acid (PNA) probe can be used instead of a nucleic acid probe in the hybridization methods described above. PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen, P. E. et al., Bioconjugate Chemistry 5, American Chemical Society, p. 1 (1994). The PNA probe can be designed to specifically hybridize to a gene having a polymorphism associated with a susceptibility to a disease or condition associated with a SLIT-3 nucleic acid (e.g., Type II diabetes). Hybridization of the PNA probe to a SLIT-3 gene is diagnostic for Type II diabetes or a susceptibility to Type II diabetes or a condition associated with a SLIT-3 nucleic acid.

In another method of the invention, alteration analysis by restriction digestion can be used to detect an altered gene, or genes containing a polymorphism(s), if the alteration (mutation) or polymorphism in the gene results in the creation or elimination of a restriction site. A test sample containing genomic DNA is obtained from the individual. Polymerase chain reaction (PCR) can be used to amplify a SLIT-3 nucleic acid (and, if necessary, the flanking sequences) in the test sample of genomic DNA from the test individual. RFLP analysis is conducted as described (see Current Protocols in Molecular Biology, supra). The digestion pattern of the relevant DNA fragment indicates the presence or absence of the alteration or polymorphism in the SLIT-3 nucleic acid, and therefore indicates the presence or absence of Type II diabetes or the susceptibility to a disease or condition associated with a SLIT-3 nucleic acid.

Sequence analysis can also be used to detect specific polymorphisms in a SLIT-3 nucleic acid. A test sample of DNA or RNA is obtained from the test individual. PCR or other appropriate methods can be used to amplify the gene or nucleic acid, and/or its flanking sequences, if desired. The sequence of a SLIT-3 nucleic acid, or a fragment of the nucleic acid, or cDNA, or fragment of the cDNA, or mRNA, or fragment of the mRNA, is determined, using standard methods. The sequence of the nucleic acid, nucleic acid fragment, cDNA, cDNA fragment, mRNA, or mRNA fragment is compared with the known nucleic acid sequence of the gene, cDNA (e.g., one or more of the sequences shown in FIG. 10, or a complement thereof or mRNA, as appropriate. The presence of a polymorphism in the SLIT-3 indicates that the individual has Type II diabetes or a susceptibility to Type II diabetes.

Allele-specific oligonucleotides can also be used to detect the presence of a polymorphism in a SLIT-3 nucleic acid, through the use of dot-blot hybridization of amplified oligonucleotides with allele-specific oligonucleotide (ASO) probes (see, for example, Saiki, R. et al., Nature 324:163-166 (1986)). An “allele-specific oligonucleotide” (also referred to herein as an “allele-specific oligonucleotide probe”) is an oligonucleotide of approximately 10-50 base pairs, preferably approximately 15-30 base pairs, that specifically hybridizes to a SLIT-3 nucleic acid, and that contains a polymorphism associated with a susceptibility to a disease or condition associated with a SLIT-3 nucleic acid. An allele-specific oligonucleotide probe that is specific for particular polymorphisms in a SLIT-3 nucleic acid can be prepared, using standard methods (see Current Protocols in Molecular Biology, supra). To identify polymorphisms in the gene that are associated with a disease or condition associated with a SLIT-3 nucleic acid or a susceptibility to a disease or condition associated with a SLIT-3 nucleic acid a test sample of DNA is obtained from the individual. PCR can be used to amplify all or a fragment of a SLIT-3 nucleic acid and its flanking sequences. The DNA containing the amplified SLIT-3 nucleic acid (or fragment of the gene or nucleic acid) is dot-blotted, using standard methods (see Current Protocols in Molecular Biology, supra), and the blot is contacted with the oligonucleotide probe. The presence of specific hybridization of the probe to the amplified SLIT-3 nucleic acid is then detected. Hybridization of an allele-specific oligonucleotide probe to DNA from the individual is indicative of a polymorphism in the SLIT-3 nucleic acid, and is therefore indicative of a disease or condition to associated with a SLIT-3 nucleic acid or susceptibility to a disease or condition associated with a SLIT-3 nucleic acid (e.g., Type II diabetes).

The invention further provides allele-specific oligonucleotides that hybridize to the reference or variant allele of a gene or nucleic acid comprising a single nucleotide polymorphism or to the complement thereof. These oligonucleotides can be probes or primers.

An allele-specific primer hybridizes to a site on target DNA overlapping a polymorphism and only primes amplification of an allelic form to which the primer exhibits perfect complementarity. See Gibbs, Nucleic Acid Res. 17, 2427-2448 (1989). This primer is used in conjunction with a second primer, which hybridizes at a distal site. Amplification proceeds from the two primers, resulting in a detectable product, which indicates the particular allelic form is present. A control is usually performed with a second pair of primers, one of which shows a single base mismatch at the polymorphic site and the other of which exhibits perfect complementarity to a distal site. The single-base mismatch prevents amplification and no detectable product is formed. The method works best when the mismatch is included in the 3′-most position of the oligonucleotide aligned with the polymorphism because this position is most destabilizing to elongation from the primer (see, e.g., WO 93/22456).

With the addition of such analogs as locked nucleic acids (LNAs), the size of primers and probes can be reduced to as few as 8 bases. LNAs are a novel class of bicyclic DNA analogs in which the 2′ and 4′ positions in the furanose ring are joined via an O-methylene (oxy-LNA), S-methylene (thio-LNA), or amino methylene (amino-LNA) moiety. Common to all of these LNA variants is an affinity toward complementary nucleic acids, which is by far the highest reported for a DNA analog. For example, particular all oxy-LNA nonamers have been shown to have melting temperatures of 64° C. and 74° C. when in complex with complementary DNA or RNA, respectively, as oposed to 28° C. for both DNA and RNA for the corresponding DNA nonamer. Substantial increases in Tₘ are also obtained when LNA monomers are used in combination with standard DNA or RNA monomers. For primers and probes, depending on where the LNA monomers are included (e.g., the 3′ end, the 5′end, or in the middle), the Tₘ could be increased considerably.

In another embodiment, arrays of oligonucleotide probes that are complementary to target nucleic acid sequence segments from an individual, can be used to identify polymorphisms in a SLIT-3 nucleic acid. For example, in one embodiment, an oligonucleotide array can be used. Oligonucleotide arrays typically comprise a plurality of different oligonucleotide probes that are coupled to a surface of a substrate in different known locations. These oligonucleotide arrays, also described as “Genechips™,” have been generally described in the art, for example, U.S. Pat. No. 5,143,854 and PCT patent publication Nos. WO 90/15070 and 92/10092. These arrays can generally be produced using mechanical synthesis methods or light directed synthesis methods that incorporate a combination of photolithographic methods and solid phase oligonucleotide synthesis methods. See Fodor et al., Science 251:767-777 (1991), Pirrung et al., U.S. Pat. No. 5,143,854 (see also PCT Application No. WO 90/15070) and Fodor et al., PCT Publication No. WO 92/10092 and U.S. Pat. No. 5,424,186, the entire teachings of each of which are incorporated by reference herein. Techniques for the synthesis of these arrays using mechanical synthesis methods are described in, e.g., U.S. Pat. No. 5,384,261; the entire teachings of which are incorporated by reference herein. In another example, linear arrays can be utilized.

Once an oligonucleotide array is prepared, a nucleic acid of interest is hybridized with the array and scanned for polymorphisms. Hybridization and scanning are generally carried out by methods described herein and also in, e.g., published PCT Application Nos. WO 92/10092 and WO 95111995, and U.S. Pat. No. 5,424,186, the entire teachings of which are incorporated by reference herein. In brief, a target nucleic acid sequence that includes one or more previously identified polymorphic markers is amplified by well-known amplification techniques, e.g. PCR. Typically, this involves the use of primer sequences that are complementary to the two strands of the target sequence both upstream and downstream from the polymorphism. Asymmetric PCR techniques may also be used. Amplified target, generally incorporating a label, is then hybridized with the array under appropriate conditions. Upon completion of hybridization and washing of the array, the array is scanned to determine the position on the array to which the target sequence hybridizes. The hybridization data obtained from the scan is typically in the form of fluorescence intensities as a function of location on the array.

Although primarily described in terms of a single detection block, e.g., for detection of a single polymorphism, arrays can include multiple detection blocks, and thus be capable of analyzing multiple, specific polymorphisms. In alternative arrangements, it will generally be understood that detection blocks may be grouped within a single array or in multiple, separate arrays so that varying, optimal conditions may be used during the hybridization of the target to the array. For example, it may often be desirable to provide for the detection of those polymorphisms that fall within G-C rich stretches of a genomic sequence, separately from those falling in A-T rich segments. This allows for the separate optimization of hybridization conditions for each situation.

Additional uses of oligonucleotide arrays for polymorphism detection can be found, for example, in U.S. Pat. Nos. 5,858,659 and 5,837,832, the entire teachings of which are incorporated by reference herein. Other methods of nucleic acid analysis can be used to detect polymorphisms in a Type II diabetes gene or variants encoding by a Type II diabetes gene. Representative methods include direct manual sequencing (Church and Gilbert, Proc. Natl. Acad. Sci. USA 81:1991-1995 (1988); Sanger, F. et al., Proc. Natl. Acad. Sci. USA 74:5463-5467 (1977); Beavis et al. U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield, V. C. et al., Proc. Natl. Acad. Sci. USA 86:232-236 (1989)), mobility shift analysis (Orita, M. et al., Proc. Natl. Acad Sci. USA 86:2766-2770 (1989)), restriction enzyme analysis (Flavell et al., Cell 15:25 (1978); Geever, et al., Proc. Natl. Acad. Sci. USA 78:5081 (1981)); heteroduplex analysis; chemical mismatch cleavage (CMC) (Cotton et al., Proc. Natl. Acad. Sci. USA 85:4397-4401 (1985)); RNase protection assays (Myers, R. M. et al., Science 230:1242 (1985)); use of polypeptides which recognize nucleotide mismatches, such as E. coli mutS protein; allele-specific PCR, for example.

In one embodiment of the invention, diagnosis of a disease or condition associated with a SLIT-3 nucleic acid (e.g., Type II diabetes) or a susceptibility to a disease or condition associated with a SLIT-3 nucleic acid (e.g., Type II diabetes) can also be made by expression analysis by quantitative PCR (kinetic thermal cycling). This technique, utilizing TaqMan®, can be used to allow the identification of polymorphisms and whether a patient is homozygous or heterozygous. The technique can assess the presence of an alteration in the expression or composition of the polypeptide encoded by a SLIT-3 nucleic acid or splicing variants encoded by a SLIT-3 nucleic acid. Further, the expression of the variants can be quantified as physically or functionally different.

In another embodiment of the invention, diagnosis of Type II diabetes or a susceptibility to Type II diabetes 9or a condition associated with a SLIT-3 gene) can be made by examining expression and/or composition of a SLIT-3 polypeptide, by a variety of methods, including enzyme linked immunosorbent assays (ELISAs), Western blots, immunoprecipitations and immunofluorescence. A test sample from an individual is assessed for the presence of an alteration in the expression and/or an alteration in composition of the polypeptide encoded by a SLIT-3 nucleic acid, or for the presence of a particular variant encoded by a SLIT-3 nucleic acid. An alteration in expression of a polypeptide encoded by a SLIT-3 nucleic acid can be, for example, an alteration in the quantitative polypeptide expression (i.e., the amount of polypeptide produced); an alteration in the composition of a polypeptide encoded by a SLIT-3 nucleic acid is an alteration in the qualitative polypeptide expression (e.g., expression of an altered SLIT-3 polypeptide or of a different splicing variant). In a preferred embodiment diagnosis of the disease or condition associated with SLIT-3 nucleic acid or a susceptibility to a disease or condition associated with a SLIT-3 nucleic acid is made by detecting a particular splicing variant encoded by that SLIT-3 nucleic acid, or a particular pattern of splicing variants.

Both such alterations (quantitative and qualitative) can also be present. The term “alteration” in the polypeptide expression or composition, as used herein, refers to an alteration in expression or composition in a test sample, as compared with the expression or composition of polypeptide by a SLIT-3 nucleic acid in a control sample. A control sample is a sample that corresponds to the test sample (e.g., is from the same type of cells), and is from an individual who is not affected by a susceptibility to a disease or condition associated with a SLIT-3 nucleic acid. An alteration in the expression or composition of the polypeptide in the test sample, as compared with the control sample, is indicative of a susceptibility to a disease or condition associated with a SLIT-3 nucleic acid. Similarly, the presence of one or more different splicing variants in the test sample, or the presence of significantly different amounts of different splicing variants in the test sample, as compared with the control sample, is indicative of a disease or condition associated with a SLIT-3 nucleic acid or a susceptibility to a disease or condition associated with a SLIT-3 nucleic acid. Various means of examining expression or composition of the polypeptide encoded by a SLIT-3 nucleic acid can be used, including: spectroscopy, colorimetry, lectrophoresis, isoelectric focusing, and immunoassays (e.g., David et al., U.S. Pat. No. 4,376,110) such as immunoblotting (see also Current Protocols in Molecular Biology, particularly Chapter 10). For example, in one embodiment, an antibody capable of binding to the polypeptide (e.g., as described above), preferably an antibody with a detectable label, can be used. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

Western blotting analysis, using an antibody as described above that specifically binds to a polypeptide encoded by an altered SLIT-3 nucleic acid (e.g., a SLIT-3 nucleic acid having one or more alterations as shown in FIG. 11), or an antibody that specifically binds to a polypeptide encoded by a non-altered nucleic acid, or an antibody that specifically binds to a particular splicing variant encoded by a nucleic acid, can be used to identify the presence in a test sample of a particular splicing variant or of a polypeptide encoded by a polymorphic or altered SLIT-3 nucleic acid, or the absence in a test sample of a particular splicing variant or of a polypeptide encoded by a non-polymorphic or non-altered nucleic acid. The presence of a polypeptide encoded by a polymorphic or altered nucleic acid, or the absence of a polypeptide encoded by a non-polymorphic or non-altered nucleic acid, is diagnostic for a disease or condition associated with a SLIT-3 nucleic acid or a susceptibility to a disease or condition associated with a SLIT-3 nucleic acid (e.g. Type II diabetes), as is the presence (or absence) of particular splicing variants encoded by the SLIT-3 nucleic acid.

In one embodiment of this method, the level or amount of polypeptide encoded by a SLIT-3 nucleic acid in a test sample is compared with the level or amount of the polypeptide encoded by the SLIT-3 in a control sample. A level or amount of the polypeptide in the test sample that is higher or lower than the level or amount of the polypeptide in the control sample, such that the difference is statistically significant, is indicative of an alteration in the expression of the polypeptide encoded by the SLIT-3 nucleic acid, and is diagnostic for a disease or condition associated with a SLIT-3 nucleic acid or a susceptibility to a disease or condition associated with that SLIT-3 nucleic acid (e.g. Type II diabetes). Alternatively, the composition of the polypeptide encoded by a SLIT-3 nucleic acid in a test sample is compared with the composition of the polypeptide encoded by the SLIT-3 nucleic acid in a control sample (e.g., the presence of different splicing variants). A difference in the composition of the polypeptide in the test sample, as compared with the composition of the polypeptide in the control sample, is diagnostic for a disease or condition associated with a SLIT-3 nucleic acid or a susceptibility to a disease or condition associated with that SLIT-3 nucleic acid (e.g., Type II diabetes). In another embodiment, both the level or amount and the composition of the polypeptide can be assessed in the test sample and in the control sample. A difference in the amount or level of the polypeptide in the test sample, compared to the control sample; a difference in composition in the test sample, compared to the control sample; or both a difference in the amount or level, and a difference in the composition, is indicative of a disease or condition associated with a SLIT-3 nucleic acid or a susceptibility to a disease or condition associated with that SLIT-3 nucleic acid.

The invention further pertains to a method for the diagnosis or identification of a susceptibility to Type II diabetes in an individual, by identifying an at-risk haplotype. A “haplotype,” as described herein, refers to a combination of genetic markers (“alleles”), such as those set forth in FIG. 11. In a certain embodiment, the haplotype can comprise one or more alleles, two or more alleles, three or more alleles, four or more alleles, or five or more alleles. The genetic markers are particular “alleles” at “polymorphic sites” associated with SLIT3. A nucleotide position at which more than one sequence is possible in a population (either a natural population or a synthetic population, e.g., a library of synthetic molecules) is referred to herein as a “polymorphic site”. Where a polymorphic site is a single nucleotide in length, the site is referred to as a single nucleotide polymorphism (“SNP”). For example, if at a particular chromosomal location, one member of a population has an adenine and another member of the population has a thymine at the same position, then this position is a polymorphic site, and, more specifically, the polymorphic site is a SNP. Polymorphic sites can allow for differences in sequences based on substitutions, insertions or deletions. Each version of the sequence with respect to the polymorphic site is referred to herein as an “allele” of the polymorphic site. Thus, in the previous example, the SNP allows for both an adenine allele and a thymine allele.

Typically, a reference sequence is referred to for a particular sequence. Alleles that differ from the reference are referred to as “variant” alleles. For example, the reference SLIT3 sequence is described herein by SEQ ID NO: 1 (FIG. 1). The term, “variant SLIT3”, as used herein, refers to a sequence that differs from SEQ ID NO: 1 but is otherwise substantially similar. The genetic markers that make up the haplotypes described herein are SLIT3 variants. Additional variants can include changes that affect a polypeptide, e.g. the SLIT3 polypeptide. These sequence differences, when compared to a reference nucleotide sequence, can include the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of a reading frame; duplication of all or a part of a sequence; transposition; or a rearrangement of a nucleotide sequence, as described in detail above. Such sequence changes alter the polypeptide encoded by a SLIT3 nucleic acid. For example, if the change in the nucleic acid sequence causes a frame shift, the frame shift can result in a change in the encoded amino acids, and/or can result in the generation of a premature stop codon, causing generation of a truncated polypeptide. Alternatively, a polymorphism associated with Type II diabetes or a susceptibility to Type II diabetes can be a synonymous change in one or more nucleotides (i.e., a change that does not result in a change in the amino acid sequence). Such a polymorphism can, for example, alter splice sites, affect the stability or transport of mRNA, or otherwise affect the transcription or translation of the polypeptide. The polypeptide encoded by the reference nucleotide sequence is the “reference” polypeptide with a particular reference amino acid sequence, and polypeptides encoded by variant alleles are referred to as “variant” polypeptides with variant amino acid sequences.

Haplotypes are a combination of genetic markers, e.g., particular alleles at polymorphic sites. Haplotypes described herein, e.g., those shown in Tables 2 and 5, are found more frequently in individuals with Type II diabetes than in individuals without Type II diabetes. Therefore, these haplotypes have predictive value for detecting Type II diabetes or a susceptibility to Type II diabetes in an individual. The haplotypes described herein are a combination of various genetic markers, e.g., SNPs and microsatellites. Therefore, detecting haplotypes can be accomplished by methods known in the art for detecting sequences at polymorphic sites, such as the methods described above.

In certain methods described herein, an individual who is at risk for Type II diabetes is an individual in whom an at-risk haplotype is identified. In one embodiment, the at-risk haplotype is one that confers a significant risk of Type II diabetes. In one embodiment, significance associated with a haplotype is measured by an odds ratio. In a further embodiment, the significance is measured by a percentage. In one embodiment, a significant risk is measured as an odds ratio of at least about 1.2, including by not limited to: 1.2, 1.3, 1.4, 1.5, 1.6, 1.7, 1.8, and 1.9. In a further embodiment, an odds ratio of at least 1.2 is significant. In a further embodiment, an odds ratio of at least about 1.5 is significant. In a further embodiment, a significant increase in risk is at least about 1.7 is significant In a further embodiment, a significant increase in risk is at least about 20%, including but not limited to about 25%, 30%, 35%, 40%, 45%, 50%, 55%, 60%, 65%, 70%, 75%, 80%, 85%, 90%, 95%, and 98%. In a further embodiment, a significant increase in risk is at least about 50%. It is understood however, that identifying whether a risk is medically significant may also depend on a variety of factors, including the specific disease, the haplotype, and often, environmental factors.

The invention also pertains to methods of diagnosing Type II diabetes or a susceptibility to Type II diabetes in an individual, comprising screening for an at-risk haplotype in the SLIT-3 nucleic acid that is more frequently present in an individual susceptible to Type II diabetes (affected), compared to the frequency of its presence in a healthy individual (control), wherein the presence of the haplotype is indicative of Type II diabetes or susceptibility to Type II diabetes. Standard techniques for genotyping for the presence of SNPs and/or microsatellite markers that are associated with Type II diabetes can be used, such as fluorescent based techniques (Chen, et al., Genome Res. 9, 492 (1999), PCR, LCR, Nested PCR and other techniques for nucleic acid amplification. In a preferred embodiment, the method comprises assessing in an individual the presence or frequency of SNPs and/or microsatellites in the SLIT-3 nucleic acid that are associated with Type II diabetes, wherein an excess or higher frequency of the SNPs and/or microsatellites compared to a healthy control individual is indicative that the individual has Type II diabetes or is susceptible to Type II diabetes. See 4FIG. 11, for SNPs and markers that comprise haplotypes that can be used as screening tools. See also FIG. 11, which sets forth SNPs and markers for use in design of diagnostic tests for determining Type II diabetes or a susceptibility to Type II diabetes. For example, an at-risk haplotype can include microsatellite markers and/or SNPs such as those set forth in FIG. 11. The presence of the haplotype is diagnostic of Type II diabetes or of a susceptibility to Type II diabetes. Haplotype analysis involves defining a candidate susceptibility locus using LOD scores. The defined regions are then ultra-fine mapped with microsatellite markers with an average spacing between markers of less than 100 kb. All usable microsatellite markers that found in public databases and mapped within that region can be used. In addition, microsatellite markers identified within the deCODE genetics sequence assembly of the human genome can be used.

The frequencies of haplotypes in the patient and the control groups using an expectation-maximization algorithm can be estimated (Dempster A. et al., 1977. J. R. Stat. Soc. B, 39:1-389). An implementation of this algorithm that can handle missing genotypes and uncertainty with the phase can be used. Under the null hypothesis, the patients and the controls are assumed to have identical frequencies. Using a likelihood approach, an alternative hypothesis where a candidate at-risk-haplotype, which can include the markers described herein, is allowed to have a higher frequency in patients than controls, while the ratios of the frequencies of other haplotypes are assumed to be the same in both groups is tested. Likelihoods are maximized separately under both hypotheses and a corresponding 1-df likelihood ratio statistics is used to evaluate the statistic significance.

To look for at-risk-haplotypes in the 1-lod drop, for example, association of all possible combinations of genotyped markers is studied, provided those markers span a practical region. The combined patient and control groups can be randomly divided into two sets, equal in size to the original group of patients and controls. The haplotype analysis is then repeated and the most significant p-value registered is determined. This randomization scheme can be repeated, for example, over 100 times to construct an empirical distribution of p-values

The at-risk haplotypes identified in Table 2 (haplotypes identified as A1, A2, A3, A4, A5, A6, B1, B2, B3, D4 and B5) or Table 5 (haplotypes identified as C1, C2, C3, C4, and C5) are associated with Type II diabetes or a susceptibility to Type II diabetes. In certain embodiments, a haplotype associated with Type II diabetes or a susceptibility to Type II diabetes comprises markers DG5S879, DG5S881, D5S2075, DG5S883, DG5S38 at the 5q35 locus; comprises markers DG5S1058 and DG5S37 at the 5q35 locus; comprises markers DG5S1058, DG5S37, DG5SI01 at the 5q35 locus; comprises markers DG5S881, DG5S1058, D5S2075, DG5S883, DG5S38 at the 5q35 locus; comprises markers DG5S879, DG5S1058, DG5S37 at the 5q35 locus; comprises markers DG5S881, D5S2075, DG5S883, DG5S38 at the 5q35 locus; comprises markers DG5S953, DG5S955, DG5S13, DG5S959 at the 5q35 locus; comprises markers DG5S888 and DG5S953 at the 5q35 locus; comprises markers DG5S953, DG5S955, DG5S124 at the 5q35 locus; comprises markers DG5S888, DG5S44, DG5S953at the 5q35 locus; comprises markers DG5S953, DG5S955, DG5S13, DG5S123, DG5S959 at the 5q35 locus; comprises markers DG5S881, SLT_{—}90256, SLT_{—}89801, SLT_{—}8967, SLT_{—}278 at the 5q35 locus; comprises markers DG5S881, SLT_{—}89801, DG5S1645, SLT_{—}8967, SLT_{—}278 at the 5q35 locus; comprises markers DG5S881, SLT_{—}89801, DG5S1645, SLT_{—}8967, SLT_{—}8778 at the 5q35 locus; comprises markers DG5S881, SLT_{—}90256, SLT_{—}89801, SLT_{—}8967, SLT_{—}8778 at the 5q35 locus; or comprises markers DG5S881, rs297898, SLT_{—}89801, DG5S1645, SLT_{—}8967 at the 5q35 locus.

The presence of the haplotype is diagnostic of Type II diabetes or of a susceptibility to Type II diabetes.

In particular embodiments, the presence of the haplotype 0, 4, −4, 0, 4 at DG5S879, DG5S881, D5S2075, DG5S883, DG5S38; of the haplotype 4, −6 at DG5S1058 and DG5S37; of the haplotype 4, −6, 0 at DG5S1058, DG5S37, DG5S101; of the haplotype 4, 4, −4, 0, 4 at DG5S881, DG5S1058, D5S2075, DG5S883, DG5S38; of the haplotype 0, 4, −6 at DG5S879, DG5S1058, DG5S37; of the haplotype 4, −4, 0, 4 at DG5S881, D5S2075, DG5S883, DG5S38; of the haplotype 0, 0, 0, 5 at DG5S953, DG5S955, DG5S13, DG5S959, of the haplotype 27, 0 at DG5S888 and DG5S953; of the haplotype 0, 0, 4 at DG5S953, DG5S955, DG5S124; of the haplotype 27, 0, 0 at DG5S888, DG5S44, DG5S953; of the haplotype 0, 0, 0, 0, 5 at DG5S953, DG5S955, DG5S13, DG5S123, DG5S959; of the haplotype 4, G, G, C, G at DG5S881, SLT_{—}90256, SLT_{—}89801, SLT_{—}8967, SLT_{—}278; of the haplotype 4, G, 0, C, G at DG5S881, SLT_{—}89801, DG5S1645, SLT_{—}8967, SLT_{—}278; of the haplotype 4, G, 0, C, T at DG5S881, SLT_{—}89801, DG5S1645, SLT_{—}8967, SLT_{—}8778; of the haplotype 4, G, G, C, T at DG5S881, SLT_{—}90256, SLT_{—}89801, SLT_{—}8967, SLT_{—}8778; of the haplotype at 4, T, G, 0, C DG5S881, rs297898, SLT_{—}89801, DG5S1645, SLT_{—}8967; is diagnostic of Type U diabetes or of susceptibility to Type II diabetes.

In another embodiment, the at-risk haplotype is characterized by a significant marker and SNP haplotype defined by the following microsatellite markers and SNPs: one or more of the markers set forth in the haplotypes in Table 2 and/or Table 5, and/or one ore more of the markers set forth in Table 4. These markers and SNPs represent an at-risk haplotype which can be used to design diagnostic tests for determining Type II diabetes or a susceptibility to Type II diabetes, as described above.

In another embodiment, the at-risk haplotype is the presence of polymorphism(s) represented in FIG. 11. The SNPs are characterized by the position indicated in FIG. 11 and the alleles indicated.

Kits (e.g., reagent kits) useful in the methods of diagnosis comprise components useful in any of the methods described herein, including for example, hybridization probes or primers as described herein (e.g., labeled probes or primers), reagents for detection of labeled molecules, restriction enzymes (e.g., for RFLP analysis), allele-specific oligonucleotides, antibodies which bind to altered or to non-altered (native) SLIT-3 polypeptide, means for amplification of nucleic acids comprising a SLIT-3, or means for analyzing the nucleic acid sequence of a SLIT-3 nucleic acid or for analyzing the amino acid sequence of a SLIT-3 polypeptide as described herein, etc. In one embodiment, the kit for diagnosing a Type II diabetes or a susceptibility to Type II diabetes can comprise primers for nucleic acid amplification of a region in the SLIT-3 nucleic acid comprising an at-risk haplotype that is more frequently present in an individual having Type II diabetes or is susceptible to Type II diabetes. The primers can be designed using portions of the nucleic acids flanking SNPs that are indicative of Type II diabetes. In a certain embodiment, the primers are designed to amplify regions of the SLIT gene associated with an at-risk haplotype for Type II diabetes, shown in FIG. 11, or more particularly the haplotype comprising the following markers and SNPs: one or more of the markers set forth in the haplotypes in Table 2 and/or Table 5, and/or one ore more of the markers set forth in Table 4, in the locus of 5q35.

Screening Assay and Agents Identified Thereby

The invention provides methods (also referred to herein as “screening assays”) for identifying the presence of a nucleotide that hybridizes to a nucleic acid of the invention, as well as for identifying the presence of a polypeptide encoded by a nucleic acid of the invention. In one embodiment, the presence (or absence) of a nucleic acid molecule of interest (e.g., a nucleic acid that has significant homology with a nucleic acid of the invention) in a sample can be assessed by contacting the sample with a nucleic acid comprising a nucleic acid of the invention (e.g., a nucleic acid having the sequence of one of the sequences shown in FIG. 10, or the complement thereof, or a nucleic acid encoding an amino acid having the sequence of one of the sequences shown in FIG. 10, or a fragment or variant of such nucleic acids), under stringent conditions as described above, and then assessing the sample for the presence (or absence) of hybridization. In one embodiment, high stringency conditions are conditions appropriate for selective hybridization. In another embodiment, a sample containing the nucleic acid molecule of interest is contacted with a nucleic acid containing a contiguous nucleotide sequence (e.g., a primer or a probe as described above) that is at least partially complementary to a part of the nucleic acid molecule of interest (e.g., a SLIT-3 nucleic acid), and the contacted sample is assessed for the presence or absence of hybridization. In another embodiment, the nucleic acid containing a contiguous nucleotide sequence is completely complementary to a part of the nucleic acid molecule of interest.

In any of these embodiments, all or a portion of the nucleic acid of interest can be subjected to amplification prior to performing the hybridization.

In another embodiment, the presence (or absence) of a polypeptide of interest, such as a polypeptide of the invention or a fragment or variant thereof, in a sample can be assessed by contacting the sample with an antibody that specifically hybridizes to the polypeptide of interest (e.g., an antibody such as those described above), and then assessing the sample for the presence (or absence) of binding of the antibody to the polypeptide of interest.

In another embodiment, the invention provides methods for identifying agents (e.g., fusion proteins, polypeptides, peptidomimetics, prodrugs, receptors, binding agents, antibodies, small molecules or other drugs, or ribozymes which alter (e.g., increase or decrease) the activity of the polypeptides described herein, or which otherwise interact with the polypeptides herein. For example, such agents can be agents which bind to polypeptides described herein (e.g., SLIT-3 binding agents); which have a stimulatory or inhibitory effect on, for example, activity of polypeptides of the invention; or which change (e.g., enhance or inhibit) the ability of the polypeptides of the invention to interact with SLIT-3 binding agents (e.g., receptors or other binding agents); or which alter posttranslational processing of a SLIT-3 polypeptide (e.g., agents that alter proteolytic processing to direct the polypeptide from where it is normally synthesized to another location in the cell, such as the cell surface; agents that alter proteolytic processing such that more polypeptide is released from the cell, etc.

In one embodiment, the invention provides assays for screening candidate or test agents that bind to or modulate the activity of polypeptides described herein (or biologically active portion(s) thereof), as well as agents identifiable by the assays. Test agents can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to polypeptide libraries, while the other four approaches are applicable to polypeptide, non-peptide oligomer or small molecule libraries of compounds (Lam, K. S., Anticancer Drug Des. 12:145 (1997)).

In one embodiment, to identify agents which alter the activity of a SLIT-3 polypeptide, a cell, cell lysate, or solution containing or expressing a SLIT-3 polypeptide, or another splicing variant encoded by a SLIT-3 nucleic acid (such as a nucleic acid comprising one or more polymorphism(s) as shown in FIG. 11), or a fragment or derivative thereof (as described above), can be contacted with an agent to be tested; alternatively, the polypeptide can be contacted directly with the agent to be tested. The level (amount) of SLIT-3 activity is assessed (e.g., the level (amount) of SLIT-3 activity is measured, either directly or indirectly), and is compared with the level of activity in a control (i.e., the level of activity of the SLIT-3 polypeptide or active fragment or derivative thereof in the absence of the agent to be tested). If the level of the activity in the presence of the agent differs, by an amount that is statistically significant, from the level of the activity in the absence of the agent, then the agent is an agent that alters the activity of a SLIT-3 polypeptide. An increase in the level of SLIT-3 activity relative to a control indicates that the agent is an agent that enhances (is an agonist of) SLIT-3 activity. Similarly, a decrease in the level of SLIT-3 activity relative to a control indicates that the agent is an agent that inhibits (is an antagonist of) SLIT-3 activity. In another embodiment, the level of activity of a SLIT-3 polypeptide or derivative or fragment thereof in the presence of the agent to be tested, is compared with a control level that has previously been established. A level of the activity in the presence of the agent that differs from the control level by an amount that is statistically significant indicates that the agent alters SLIT-3 activity.

The present invention also relates to an assay for identifying agents which alter the expression of a SLIT-3 nucleic acid (e.g., antisense nucleic acids, fusion proteins, polypeptides, peptidomimetics, prodrugs, receptors, binding agents, antibodies, small molecules or other drugs, or ribozymes) which alter (e.g., increase or decrease) expression (e.g., transcription or translation) of the gene or which otherwise interact with the nucleic acids described herein, as well as agents identifiable by the assays. For example, a solution containing a nucleic acid encoding a SLIT-3 polypeptide (e.g., a SLIT-3 nucleic acid) can be contacted with an agent to be tested. The solution can comprise, for example, cells containing the nucleic acid or cell lysate containing the nucleic acid; alternatively, the solution can be another solution that comprises elements necessary for transcription/translation of the nucleic acid. Cells not suspended in solution can also be employed, if desired. The level and/or pattern of SLIT-3 expression (e.g., the level and/or pattern of mRNA or of protein expressed, such as the level and/or pattern of different splicing variants) is assessed, and is compared with the level and/or pattern of expression in a control (i.e., the level and/or pattern of the SLIT-3 expression in the absence of the agent to be tested). If the level and/or pattern in the presence of the agent differ, by an amount or in a manner that is statistically significant, from the level and/or pattern in the absence of the agent, then the agent is an agent that alters the expression of a Type II diabetes gene. Enhancement of SLIT-3 expression indicates that the agent is an agonist of SLIT-3 activity. Similarly, inhibition of SLIT-3 expression indicates that the agent is an antagonist of SLIT-3 activity. In another embodiment, the level and/or pattern of SLIT-3 polypeptide(s) (e.g., different splicing variants) in the presence of the agent to be tested, is compared with a control level and/or pattern that have previously been established A level and/or pattern in the presence of the agent that differs from the control level and/or pattern by an amount or in a manner that is statistically significant indicates that the agent alters SLIT-3 expression.

In another embodiment of the invention, agents which alter the expression of a SLIT-3 nucleic acid or which otherwise interact with the nucleic acids described herein, can be identified using a cell, cell lysate, or solution containing a nucleic acid encoding the promoter region of the SLIT-3 nucleic acid operably linked to a reporter gene. After contact with an agent to be tested, the level of expression of the reporter gene (e.g., the level of mRNA or of protein expressed) is assessed, and is compared with the level of expression in a control (i.e., the level of the expression of the reporter gene in the absence of the agent to be tested). If the level in the presence of the agent differs, by an amount or in a manner that is statistically significant, from the level in the absence of the agent, then the agent is an agent that alters the expression of the SLIT-3, as indicated by its ability to alter expression of a gene that is operably linked to the SLIT-3 nucleic acid promoter. Enhancement of the expression of the reporter indicates that the agent is an agonist of SLIT-3. Similarly, inhibition of the expression of the reporter indicates that the agent is an antagonist of SLIT-3. In another embodiment, the level of expression of the reporter in the presence of the agent to be tested is compared with a control level that has previously been established. A level in the presence of the agent that differs from the control level by an amount or in a manner that is statistically significant indicates that the agent alters expression.

Agents which alter the amounts of different splicing variants encoded by a SLIT-3 nucleic acid (e.g., an agent which enhances activity of a first splicing variant, and which inhibits activity of a second splicing variant), as well as agents which are agonists of activity of a first splicing variant arid antagonists of activity of a second splicing variant, can easily be identified using these methods described above.

In other embodiments of the invention, assays can be used to assess the impact of a test agent on the activity of a polypeptide in relation to a SLIT-3 binding agent. For example, a cell that expresses a compound that interacts with a SLIT-3 polypeptide (herein referred to as a “SLIT-3 binding agent”, which can be a polypeptide or other molecule that interacts with a SLIT-3 polypeptide, such as a receptor) is contacted with a SLIT-3 in the presence of a test agent, and the ability of the test agent to alter the interaction between the SLIT-3 and the SLIT-3 binding agent is determined. Alternatively, a cell lysate or a solution containing the SLIT-3 binding agent, can be used. An agent which binds to the SLIT-3 or the SLIT-3 binding agent can alter the interaction by interfering with, or enhancing the ability of the SLIT-3 to bind to, associate with, or otherwise interact with the SLIT-3 binding agent. Determining the ability of the test agent to bind to a SLIT-3 polypeptide or a SLIT-3 binding agent can be accomplished, for example, by coupling the test agent with a radioisotope or enzymatic label such that binding of the test agent to the polypeptide can be determined by detecting the labeled with ¹²⁵I, ³⁵S, ¹⁴C or ³H, either directly or indirectly, and the radioisotope detected by direct counting of radioemmission or by scintillation counting. Alternatively, test agents can be enzymatically labeled with, for example, horseradish peroxidase, alkaline phosphatase, or luciferase, and the enz [symbol] conversion of an appropriate substrate to product. It is also within the scope of this invention to determine the ability of a test agent to interact with the polypeptide without the labeling of any of the interactants. For example, a microphysiometer can be used to detect the interaction of a test agent with a SLIT-3 nucleic acid or a SLIT-3 binding agent without the labeling of the test agent, SLIT-3 nucleic acid, or the SLIT-3 binding agent. McConnell, H. M. et al., Science 257:1906-1912 (1992). As used herein, a “microphysiometer” (e.g., Cytosensor™) is an analytical instrument that measures the rate at which a cell acidifies its environment using a light-addressable potentiometric sensor (LAPS). Changes in this acidification rate can be used as an indicator of the interaction between ligand and polypeptide.

Thus, these receptors can be used to screen for compounds that are agonists or antagonists, for use in treating a disease or condition associated with a SLIT-3 gene, or for treating a susceptibility to a disease or condition associated with a SLIT-3 gene (e.g., Type II diabetes). Drugs can be designed to regulate SLIT-3 activation that in turn can be used to regulate signaling pathways and transcription events of genes downstream, or to alter interaction of proteins or polypeptides with SLIT-3.

In another embodiment of the invention, assays can be used to identify polypeptides that interact with one or more SLIT-3 polypeptides, as described herein. For example, a yeast two-hybrid system such as that described by Fields and Song (Fields, S. and Song, O., Nature 340:245-246 (1989)) can be used to identify polypeptides that interact with one or more SLIT-3 polypeptides. In such a yeast two-hybrid system, vectors are constructed based on the flexibility of a transcription factor that has two functional domains (a DNA binding domain and a transcription activation domain). If the two domains are separated but fused to two different proteins that interact with one another, transcriptional activation can be achieved, and transcription of specific markers (e.g., nutritional markers such as His and Ade, or color markers such as lacZ) can be used to identify the presence of interaction and transcriptional activation. For example, in the methods of the invention, a first vector is used which includes a nucleic acid encoding a DNA binding domain and also a SLIT-3 polypeptide, splicing variant, or fragment or derivative thereof, and a second vector is used which includes a nucleic acid encoding a transcription activation domain and also a nucleic acid encoding a polypeptide which potentially may interact with the SLIT-3 polypeptide, splicing variant, or fragment or derivative thereof (e.g., a SLIT-3 polypeptide binding agent or receptor). Incubation of yeast containing the first vector and the second vector under appropriate conditions (e.g., mating conditions such as used in the Matchmaker™ system from Clontech (Palo Alto, Calif., USA)) allows identification of colonies that express the markers of interest. These colonies can be examined to identify the polypeptide(s) that interact with the SLIT-3 polypeptide or fragment or derivative thereof. Such polypeptides may be useful as agents that alter the activity of expression of a SLIT-3 polypeptide, as described above.

In more than one embodiment of the above assay methods of the present invention, it may be desirable to immobilize either the SLIT-3 nucleic acid, the SLIT-3 binding agent, or other components of the assay on a solid support, in order to facilitate separation of complexed from uncomplexed forms of one or both of the polypeptides, as well as to accommodate automation of the assay. Binding of a test agent to the polypeptide, or interaction of the polypeptide with a binding agent in the presence and absence of a test agent, can be accomplished in any vessel suitable for containing the reactants. Examples of such vessels include microtitre plates, test tubes, and micro-centrifuge tubes. In one embodiment, a fusion protein (e.g. a glutathione-S-transferase fusion protein) can be provided which adds a domain that allows a SLIT-3 nucleic acid or a SLIT-3 binding agent to be bound to a matrix or other solid support.

In another embodiment, modulators of expression of nucleic acid molecules of the invention are identified in a method wherein a cell, cell lysate, or solution containing a nucleic acid encoding a SLIT-3 is contacted with a test agent and the expression of appropriate mRNA or polypeptide (e.g. splicing variant(s)) in the cell, cell lysate, or solution, is determined. The level of expression of appropriate mRNA or polypeptide(s) in the presence of the test agent is compared to the level of expression of mRNA or polypeptide(s) in the absence of the test agent. The test agent can then be identified as a modulator of expression based on this comparison. For example, when expression of mRNA or polypeptide is greater (statistically significantly greater) in the presence of the test agent than in its absence, the test agent is identified as a stimulator or enhancer of the mRNA or polypeptide expression Alternatively, when expression of the mRNA or polypeptide is less (statistically significantly less) in the presence of the test agent than in its absence, the test agent is identified as an inhibitor of the mRNA or polypeptide expression. The level of mRNA or polypeptide expression in the cells can be determined by methods described herein for detecting mRNA or polypeptide.

This invention further pertains to novel agents identified by the above-described screening assays. Accordingly, it is within the scope of this invention to further use an agent identified as described herein in an appropriate animal model. For example, an agent identified as described herein (e.g., a test agent that is a modulating agent, an antisense nucleic acid molecule, a specific antibody, or a polypeptide-binding agent) can be used in an animal model to determine the efficacy, toxicity, or side effects of treatment with such an agent Alternatively, an agent identified as described herein can be used in an animal model to determine the mechanism of action of such an agent.

Furthermore, this invention pertains to uses of novel agents identified by the above-described screening assays for treatments as described herein, as well as for the manufacture of medicaments for use in treatment, such as in the treatments described herein. In addition, an agent identified as described herein can be used to alter activity of a polypeptide encoded by a SLIT-3 nucleic acid, or to alter expression of a SLIT-3 nucleic acid, by contacting the polypeptide or the nucleic acid (or contacting a cell comprising the polypeptide or the nucleic acid) with the agent identified as described herein.

Pharmaceutical Compositions

The present invention also pertains to pharmaceutical compositions comprising nucleic acids described herein, particularly nucleotides encoding the polypeptides described herein; comprising polypeptides described herein and/or comprising other splicing variants encoded by a SLIT-3 nucleic acid; and/or an agent that alters (e.g., enhances or inhibits) SLIT-3 nucleic acid expression or SLIT-3 polypeptide activity as described herein. For instance, a polypeptide, protein (e.g. a SLIT-3 nucleic acid receptor), an agent that alters SLIT-3 nucleic acid expression, or a SLIT-3 binding agent or binding partner, fragment, fusion protein or pro-drug thereof, or a nucleotide or nucleic acid construct (vector) comprising a nucleotide of the present invention, or an agent that alters SLIT-3 polypeptide activity, can be formulated with a physiologically acceptable carrier or excipient to prepare a pharmaceutical composition. The carrier and composition can be sterile. The formulation should suit the mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymetaylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active agents.

The composition, if desired, can also contain minor amounts of wetting or emulsifying agents, or pH buffering agents. The composition can be a liquid solution, suspension, emulsion, tablet pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc.

Methods of introduction of these compositions include, but are not limited to, intradermal, intramuscular, intraperitoneal, intraocular, intravenous, subcutaneous, topical, oral and intranasal. Other suitable methods of introduction can also include gene therapy (as described below), rechargeable or biodegradable devices, particle acceleration devises (“gene guns”) and slow release polymeric devices. The pharmaceutical compositions of this invention can also be administered as part of a combinatorial therapy with other agents.

The composition can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., which are, if desired, sterilized or mixed with auxiliary agents, e.g., preservatives, stabilizers, wetting agents, buffers or salts for influencing osmotic pressure, etc. The agent may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

Agents described herein can be formulated as neutral or salt forms.

Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The agents are administered in a therapeutically effective amount. The amount of agents which will be therapeutically effective in the treatment of a particular disorder or condition will depend on the nature of the disorder or condition, and can be determined by standard clinical techniques. In addition, in vitro or in vivo assays may optionally be employed to help identify optimal dosage ranges. The precise dose to be employed in the formulation will also depend on the route of administration, and the seriousness of the symptoms of disease, and should be decided according to the judgment of a practitioner and each patient's circumstances. Effective doses may be extrapolated from dose-response curves derived from in vitro or animal model test systems.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use of sale for human administration. The pack or kit can be labeled with information regarding mode of administration, sequence of drug administration (e.g., separately, sequentially or concurrently), or the like. The pack or kit may also include means for reminding the patient to take the therapy. The pack or kit can be a single unit dosage of the combination therapy or it can be a plurality of unit dosages. In particular, the agents can be separated, mixed together in any combination, present in a single vial or tablet. Agents assembled in a blister pack or other dispensing means is preferred. For the purpose of this invention, unit dosage is intended to mean a dosage that is dependent on the individual pharmacodynamics of each agent and administered in FDA approved dosages in standard time courses.

Method of Therapy

The present invention also pertains to methods of treatment (prophylactic and/or therapeutic) for certain diseases and conditions associated with SLIT-3 or with members of the Roundabout or Robo family. This family includes polypeptides (e.g., receptors for robo 1, robo 2 and rig. 1) and other molecules that are associated with the interaction of SLIT-3 and members of the Robo family. The invention additionally pertains to use of polypeptides and other molecules that are associated with the interaction of SLIT-3 and members of the Robo family, for the manufacture of a medicament, such as for the treatment for certain diseases and conditions associated with SLIT-3 or with members of the Roundabout or Robo family, as described herein.

In particular, the invention relates to methods of treatment for Type II diabetes or a susceptibility to Type II diabetes, using a Type II diabetes therapeutic agent. A “Type II diabetes therapeutic agent” is an agent that alters (e.g., enhances or inhibits) SLIT-3 polypeptide activity and/or SLIT-3 nucleic acid expression, as described herein (e.g., a Type II diabetes nucleic acid agonist or antagonist). In certain embodiments, the Type II diabetes [symbol] activity and/or nucleic acid expression of SLIT-3 or members of the Robo receptor family, or alters the interaction between SLIT-3 and members of the Robo family.

Type II diabetes therapeutic agents can alter SLIT-3 polypeptide activity or nucleic acid expression by a variety of means, such as, for example, by providing additional SLIT-3 polypeptide or Robo family polypeptide or by upregulating the transcription or translation of the SLIT-3 nucleic acid or a nucleic acid encoding a polypeptide that is a member of the Robo family; by altering posttranslational processing of the SLIT-3 polypeptide or Robo family polypeptide; by altering transcription of SLIT-3 or Robo family splicing variants; or by interfering with SLIT-3 polypeptide activity (e.g., by binding to a SLIT-3 polypeptide), or by binding to another polypeptide that interacts with a member of the Robo family, by altering (e.g., downregulating) the expression, transcription or translation of a SLIT-3 nucleic acid, by altering the interaction of a SLIT-3 nucleic acid with a member of the Robo family (e.g., interaction between SLIT-3 and one or more of the members of the Robo family, for example, the robo 1 receptor, the robo 2 receptor and the rig-1 receptor); or by altering (e.g., agonizing or antagonizing) activity of a member of the Robo family.

Representative Type II diabetes therapeutic agents include the following:
nucleic acids or fragments or derivatives thereof described herein, particularly nucleotides encoding the polypeptides described herein and vectors comprising such nucleic acids (e.g., a gene, cDNA, and/or mRNA, such as a nucleic acid encoding a SLIT-3 polypeptide or active fragment or derivative thereof, or an oligonucleotide; or a complement thereof, or fragments or derivatives thereof, and/or other splicing variants encoded by a Type II diabetes nucleic acid, or fragments or derivatives thereof);
nucleic acids encoding a member of the Robo family, or fragments or derivatives thereof, including nucleic acids encoding robo 1, robo 2 or rig-1 or Robo family polypeptides, and vectors comprising such nucleic acids (e.g., a gene, nucleic acid, cDNA, and /or mRNA, or a nucleic acid encoding an active fragment or derivative thereof, or an oligonucleotide;
polypeptides described herein and/or splicing variants encoded by the SLIT-3 nucleic acid or fragments or derivatives thereof;
polypeptides encoded by genes for the members of the Robo family (e.g., robo 1), or fragments or derivatives thereof;
other polypeptides (e.g., SLIT-3 receptors, Robo family receptors, such as robo 1 receptor, robo 2 receptor and rig-1); SLIT-3 binding agents; binding agents of the Robo family, or affect (e.g., increase or decrease) activity of a member of the Robo family,
antibodies, such as an antibody to an altered SLIT-3 polypepted, or an antibody to a non-altered SLIT-3 polypeptide, or an antibody to a particular splicing variant encoded by a SLIT-3 nucleic acid as described above; or antibodies to members of the Robo family, such as an antibody to an altered robo 1 polypeptide, or an antibody to a non-altered robo 1 polypeptide, or an antibody to a particular splicing variant of robo 1;
peptidomimetics; fusion proteins or prodrugs thereof; ribozymes; other small molecules; and
other agents that alter (e.g., enhance or inhibit) expression of a SLIT-3 nucleic acid or a member of the Robo family or polypeptide activity, or that regulate transcription of SLIT-3 splicing variants or Robo family polypeptide variants (e.g., agents that affect which splicing variants are expressed, or that affect the amount of each splicing variant that is expressed).

More than one Type II diabetes therapeutic agent can be used concurrently, if desired.

A Type II diabetes nucleic acid therapeutic agent that is a nucleic acid is used in the treatment of Type II diabetes or in the treatment for a susceptibility to Type II diabetes. The term, “treatment” as used herein, refers not only to ameliorating symptoms associated with the disease or condition, but also preventing or delaying the onset of the disease or condition, and also lessening the severity or frequency of symptoms of the disease or condition. The therapy is designed to alter (e.g., inhibit or enhance), replace or supplement activity of a SLIT-3 polypeptide or a Robo family polypeptide in an individual. For example, a Type II diabetes therapeutic agent can be administered in order to upregulate or increase the expression or availability of the SLIT-3 nucleic acid or of specific splicing variants of SLIT-3 nucleic acid, or, conversely, to downregulate or decrease the expression or availability of the SLIT-3 nucleic acid or specific splicing variants of the SLIT-3 nucleic acid. Upregulation or increasing expression or availability of a native SLIT-3 gene or nucleic acid or of a particular splicing variant could interfere with or compensate for the expression or activity of a defective gene or another splicing variant; downregulation or decreasing expression or availability of a native SLIT-3 gene or of a particular splicing variant could minimize the expression or activity of a defective gene or the particular splicing variant and thereby minimize the impact of the defective gene or the particular splicing variant. Similarly, for example a Type II diabetes therapeutic agent can be administered in order to upregulate or increase the expression or availability of the nucleic acid encoding a member of the Robo family or of specific splicing variants of a Robo family member, or, conversely, to downregulate or decrease the expression or availability of the nucleic acid encoding a Robo family member or specific splicing variant of the nucleic acid encoding a Robo family member.

The Type II diabetes therapeutic agent(s) are administered in a therapeutically effective amount (i.e., an amount that is sufficient to treat the disease, such as by ameliorating symptoms associated with the disease, preventing or delaying the onset of the disease, and/or also lessening the severity or frequency of symptoms of the disease). The amount which will be therapeutically effective in the treatment of a particular individual's disorder or condition will depend on the symptoms and severity of the disease, and can be determined by standard clinical techniques. In addition, in vitro or in vivo assays may optionally be employed to help identify optimal dosage ranges. The precise dose to be employed in the formulation will also depend on the route of administration, and the seriousness of the disease or disorder, and should be decided according to the judgment of a practitioner and each patient's circumstances. Effective doses may be extrapolated from dose-response curves derived from in vitro or animal model test systems.

In one embodiment, a nucleic acid of the invention (e.g., a nucleic acid encoding a SLIT-3 polypeptide, such as one of the sequences shown in FIG. 10, or a complement thereof; or another nucleic acid that encodes a SLIT-3 polypeptide or a splicing variant, derivative or fragment thereof, can be used, either alone or in a pharmaceutical composition as described above. For example, a SLIT-3 gene or nucleic acid or a cDNA encoding a SLIT-3 polypeptide, either by itself or included within a vector, can be introduced into cells (either in vitro or in vivo) such that the cells produce native SLIT-3 polypeptide. If necessary, cells that have been transformed with the gene or cDNA or a vector comprising the gene, nucleic acid or cDNA can be introduced (or re-introduced) into an individual affected with the disease. Thus, cells which, in nature, lack native SLIT-3 expression and activity, or have altered SLIT-3 expression and activity, or have expression of a disease-associated SLIT-3 splicing variant, can be engineered to express the SLIT-3 polypeptide or an active fragment of the SLIT-3 polypeptide (or a different variant of the SLIT-3 polypeptide). In certain embodiments, nucleic acids encoding a SLIT-3 polypeptide, or an active fragment or derivative thereof, can be introduced into an expression vector, such as a viral vector, and the vector can be introduced into appropriate cells in an animal. Other gene transfer systems, including viral and nonviral transfer systems, can be used. Alternatively, nonviral gene transfer methods, such as calcium phosphate coprecipitation, mechanical techniques (e.g. microinjection); membrane fusion-mediated transfer via liposomes; or direct DNA uptake, can also be used.

In another embodiment, a nucleic acid encoding a Robo family polypeptide, or a splicing variant, derivative or fragment thereof, can be used, either alone or in a pharmaceutical composition as described above. For example, the nucleic acid, either by itself or included within a vector, can be introduced into cells (either in vitro or in vivo) such that the cells produce native Robo family polypeptide. If necessary, cells that have been transformed with the gene or cDNA or a vector comprising the gene, nucleic acid or cDNA can be introduced (or re-introduced) into an individual affected with the disease. Thus, cells which, in nature, lack native Robo family polypeptide expression and activity, or have altered Robo family polypeptide expression and activity, or have expression of a disease-associated Robo family polypeptide splicing variant, can be engineered to express the Robo family polypeptide or an active fragment of the Robo family polypeptide (or a different variant of the Robo family polypeptide). In certain embodiments, nucleic acids encoding a Robo family polypeptide, or an active fragment or derivative thereof, can be introduced into an expression vector, such as a viral vector, and the vector can be introduced into appropriate cells in an animal. Other gene transfer systems, including viral and nonviral transfer systems, can be used.

Alternatively, in another embodiment of the invention, a nucleic acid of the invention; a nucleic acid complementary to a nucleic acid of the invention; or a portion of such a nucleic acid (e.g. an oligonucleotide as described below); or a nucleic acid encoding a member of the Robo family, can be used in “antisense” therapy, in which a nucleic acid (e.g., an oligonucleotide) which specifically hybridizes to the mRNA and/or genomic DNA of a Type II diabetes gene is administered or generated in situ. The antisense nucleic acid that specifically hybridizes to the mRNA and/or DNA inhibits expression of the SLIT-3 polypeptide, e.g., by inhibiting translation and/or transcription. Binding of the antisense nucleic acid can be by conventional base pair complementarity, or, for example, in the case of binding to DNA duplexes, through specific interaction in the major groove of the double helix.

An antisense construct of the present invention can be delivered, for example, as an expression plasmid as described above. When the plasmid is transcribed in the cell, it produces RNA that is complementary to a portion of the mRNA and/or DNA which encodes the SLIT-3 polypeptide or Robo family polypeptide. Alternatively, the antisense construct can be an oligonucleotide probe that is generated ex vivo and introduced into cells; it then inhibits expression by hybridizing with the mRNA and/or genomic DNA of the polypeptide. In one embodiment, the oligonucleotide probes are modified oligonucleotides, which are resistant to endogenous nucleases, e.g., exonucleases and/or endonucleases, thereby rendering them stable in vivo. Exemplary nucleic acid molecules for use as antisense oligonucleotides are phosphoramidate, phosphorothioate and methylphosphonate analogs of DNA (see also U.S. Pat. Nos. 5,176,996; 5,264,564; and 5,256,775). Additionally, general approaches to constructing oligomers useful in antisense therapy are also described, for example, by Van der Krol et al. (Biotechniques 6:958-976 (1988)); and Stein et al. (Cancer Res. 48:2659-2668 (1988)). With respect to antisense DNA, oligodeoxyribonucleotides derived from the translation initiation site are preferred.

To perform antisense therapy, oligonucleotides (mRNA, cDNA or DNA) are designed that are complementary to mRNA encoding the SLIT-3. The antisense oligonucleotides bind to SLIT-3 mRNA transcripts and prevent translation. Absolute complementarity, although preferred, is not required. A sequence “complementary” to a portion of an RNA, as referred to herein, indicates that a sequence has sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid, as described in detail above. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures.

The oligonucleotides used in antisense therapy can be DNA, RNA, or chimeric mixtures or derivatives or modified versions thereof, single-stranded or double-stranded. The oligonucleotides can be modified at the base moiety, sugar moiety, or phosphate backbone, for example, to improve stability of the molecule, hybridization, etc. The oligonucleotides can include other appended groups such as peptides (e.g. for targeting host cell receptors in vivo), or agents facilitating transport across the cell membrane (see, e.g., Letsinger et al., Proc. Natl. Acad. Sci. USA 86:6553-6556 (1989); Lemaitre et al., Proc. Natl. Acad Sci. USA 84:648-652 (1987); PCT International Publication No. WO 88/09810) or the blood-brain barrier (see, e.g., PCT International Publication No. WO 89/10134), or hybridization-triggered cleavage agents (see, e.g., Krol et al., BioTechniques 6:958-976 (1988)) or intercalating agents. (See, e.g., Zon, Pharm. Res. 5:539-549 (1988)). To this end, the oligonucleotide may be conjugated to another molecule (e.g., a peptide, hybridization triggered cross-linking agent, transport agent, hybridization-triggered cleavage agent).

The antisense molecules are delivered to cells that express SLIT-3 in vivo. A number of methods can be used for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically. Alternatively, in a preferred embodiment, a recombinant DNA construct is utilized in which the antisense oligonucleotide is placed under the control of a strong promoter (e.g. pol III or pol II). The use of such a construct to transfect target cells in the patient results in the transcription of sufficient amounts of single stranded RNAs that will form complementary base pairs with the endogenous SLIT-3 transcripts and thereby prevent translation of the SLIT-3 mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art and described above. For example, a plasmid, cosmid, YAC or viral vector can be used to prepare the recombinant DNA construct that can be introduced directly into the tissue site. Alternatively, viral vectors can be used which selectively infect the desired tissue, in which case administration may be accomplished by another route (e.g., systemically).

Endogenous SLIT-3 or Robo family polypeptide expression can also be reduced by inactivating or “knocking out” the gene, nucleic acid or its promoter using targeted homologous recombination (e.g., see Smithies et al., Nature 317:230-234 (1985); Thomas & Capecchi, Cell 51:503-512 (1987); Thompson et al., Cell 5:313-321 (1989)). For example, an altered, non-functional gene or nucleic acid (or a completely unrelated DNA sequence) flanked by DNA homologous to the endogenous gene or nucleic acid (either the coding regions or regulatory regions of the nucleic acid) can be used, with or without a selectable marker and/or a negative selectable marker, to transfect cells that express the gene or nucleic acid in vivo. Insertion of the DNA construct, via targeted homologous recombination, results in inactivation of the gene or nucleic acid. The recombinant DNA constructs can be directly administered or targeted to the required site in vivo using appropriate vectors, as described above.

Alternatively, expression of non-altered genes or nucleic acids can be increased using a similar method: targeted homologous recombination can be used to insert a DNA construct comprising a non-altered functional gene or nucleic acid, e.g., a nucleic acid having one of the sequences shown in FIG. 10, or the complement thereof, or a portion thereof, in place of an altered SLIT-3 in the cell, as described above. In another embodiment, targeted homologous recombination can be used to insert a DNA construct comprising a nucleic acid that encodes a Type II diabetes polypeptide variant that differs from that present in the cell. Alternatively, endogenous SLIT-3 or Robo family nucleic acid expression can be reduced by targeting deoxyribonucleotide sequences complementary to the regulatory region of a SLIT-3 or Robo family nucleic acid (i.e., the SLIT-3 promoter and/or enhancers) to form triple helical structures that prevent transcription of the SLIT-3 or Robo Family nucleic acid in target cells in the body. (See generally, Helene, C., Anticancer Drug Des., 6(6):569-84 (1991); Helene, C. et al., Ann. N. Y. Acad. Sci. 660:27-36 (1992); and Maher, L. J., Bioassays 14(12):807-15 (1992)). Likewise, the antisense constructs described herein, by antagonizing the normal biological activity of one of the SLIT-3 or Robo family proteins, can be used in the manipulation of tissue, e.g., tissue differentiation, both in vivo and for ex vivo tissue cultures. Furthermore, the anti-sense techniques (e.g., microinjection of antisense molecules, or transfection with plasmids whose transcripts are anti-sense with regard to a Type II diabetes gene mRNA or gene sequence) can be used to investigate the role of one or SLIT-3 or Robo family members or the interaction of SLIT-3 and Robo family members in developmental events, as well as the normal cellular function of the SLIT-3s or Robo family members the interaction of SLIT-3 and Robo family members in adult tissue. Such techniques can be utilized in cell culture, but can also be used in the creation of transgenic animals.

In yet another embodiment of the invention, other Type II diabetes therapeutic agents as described herein can also be used in the treatment or prevention of a susceptibility to a disease or condition associated with a Type II diabetes gene. The therapeutic agents can be delivered in a composition, as described above, or by themselves. They can be administered systemically, or can be targeted to a particular tissue. The therapeutic agents can be produced by a variety of means, including chemical synthesis; recombinant production; it vivo production (e.g., a transgenic animal, such as U.S. Pat. No. 4,873,316 to Meade et al.), for example, and can be isolated using standard means such as those described herein.

A combination of any of the above methods of treatment (e.g., administration of non-altered polypeptide in conjunction with antisense therapy targeting altered mRNA or SLIT-3 or a member of the Robo family; administration of a first splicing variant encoded by a SLIT-3 or a member of the Robo family in conjunction with antisense therapy targeting a second splicing encoded by a SLIT-3 or a member of the Robo family) can also be used.

Monitoring Progress of Treatment

The current invention also pertains to methods of monitoring the effectiveness of treatment on the regulation of expression (e.g., relative or absolute expression) of SLIT3 or an isoform of SLIT3 at the RNA or protein level or its enzymatic activity. SLIT3 message or protein or enzymatic activity can be measured in a sample of peripheral blood or cells derived therefrom. An assessment of the levels of expression or activity can be made before and during treatment with SLIT3 therapeutic agents.

For example, in one embodiment of the invention, an individual who is a member of the target population can be assessed for response to treatment with a SLIT3 inhibitor, by examining, for example, absolute and/or relative levels of SLIT3 protein or mRNA, or isoforms thereof, in peripheral blood in general or specific cell subfractions or combination of cell subfractions. In addition, variation such as haplotypes or mutations within or near (within 100 to 200 kb) of the SLIT3 gene may be used to identify individuals who are at higher risk for Type II diabetes to increase the power and efficiency of clinical trials for pharmaceutical agents to prevent or treat Type II diabetes. The haplotypes and other variations may be used to exclude or fractionate patients in a clinical trial who are likely to have non-SLIT3 involvement in their Type II diabetes risk in order to enrich patients who have other genes or pathways involved and boost the power and sensitivity of the clinical trial. Such variation may be used as a pharmacogenomic test to guide selection of pharmaceutical agents for individuals.

Described herein is the first known linkage study of Type II diabetes showing a connection to chromosome 5q35. Based on the linkage studies conducted, a direct relationship between Type II diabetes and the locus on chromosome 5q35, in particular the SLIT3 gene, has been discovered.

The present invention is now illustrated by the following Exemplification, which is not intended to be limiting in any way.

Exemplification

A study was done in collaboration with the Icelandic Heart Association, who provided an encrypted list of 1350 diabetic patients. In 1967-1991 the Heart Association started a study of cardiovascular disease and its complications. Measurements of blood sugar were included in a thorough check-up of the participants, which led to many individuals being diagnosed with diabetes. The list of participants is an unbiased sample of about a third of the Icelandic population. Individuals diagnosed in the years following 1991 were either diagnosed at the Icelandic Heart Association or at one of two major hospitals in Reykjavík Iceland.

All participants in the Type II diabetes study visited the Icelandic Heart Association where each answered a questionnaire, had blood drawn, a blood sugar assessment, and had measurements taken. Height (m) and weight (kg) were measured to calculate the body mass index. In serum, the fasting blood glucose and triglyceride levels were measured as well. Diagnoses of Type II diabetes were based on the diagnostic criteria set by the World Health Organization (1999). All patients with fasting glucose above 7 mM were diagnosed as having Type II diabetes and individuals with fasting blood sugar between 6.1-6.9 mM were diagnosed with impaired fasting glucose. If the participants had no prior history of diabetes, they were requested to come in for another test to have their diagnosis confirmed. All individuals on diabetic medication were classified as Type II. The questionnaire included questions regarding age at diagnosis and type of medication. All patients were requested to bring two relatives whose DNA was used to confirm the gen-otypes of the patients.

Since the patients had participated in a study that was conducted between 1967-1991, a considerable time had passed in some instances since they had visited the Heart Association. Therefore, all the patients were required to have another fasting blood glucose test to check on their blood sugar level at the time of participation in the study. Thus, all patients were labeled unconfirmed, meaning that results of blood glucose levels were pending, for this particular study. A label of confirmed diabetics was given to the patient when the measurements were received. Linkage analyses were done with confirmed patients and unconfirmed patients were included only if they were close relatives of a confirmed index patient. The initial list of patients included 1350 Type II diabetics, but during this study, new patients were diagnosed who were relatives of the index patients. All participants with no previous history of diabetes but with elevated fasting glucose were diagnosed according to the WHO criteria as described above. At the present date, 1406 Type II diabetics and 266 patients with impaired fasting glucose have participated in the study, together with 3972 of their close relatives. This study was approved by the Data Protection Commission of Iceland and the National Bioethics Committee of Iceland. All patients and their relatives who participated in the Study gave informed consents.

Outline of the Study

This particular genetic study aimed at identifying a genetic variant or a gene that contributes to type II diabetes by using a positional cloning approach. Three steps were performed:
(i) Genome-wide linkage study, where excess allele sharing among related type II diabetics was used to identify a chromosomal segment, typically 2-8 Megabases long, that may harbor a disease susceptibility gene/genes.
(ii) Locus-wide association study, where a high-density of microsatellite markers was typed in a large patient and control cohort. By comparing the frequencies of individual alleles or haplotypes between the two cohorts, the location of the putative disease gene/genes was narrowed down to a few hundred kilobases.
(iii) Candidate gene assessment, where additional microsatellites and/or SNPs were typed in all genes that are identified within the smaller candidate region and further association analysis was used to identify which of the genes shows strong association to the disease.
   Linkage Analysis
   Pedigree Construction

For the linkage analysis, blood samples were obtained from 964 Type II diabetics and 203 individuals with impaired fasting glucose. The patients were clustered into families such that each patient is related to (within and including six meiotic events) at least one other patient. In this manner, 772 patients fell into families—705 Type II diabetics and 67 with impaired fasting glucose. The confirmed Type II patients were treated as probands and clustered into families that each proband is related to, within and including six meiotic events. The other patients, unconfirmed Type II and IFG patients, were added to the families if they were related to a proband within and including three meiotic events. The rational behind this was to include as many patients as possible in the study. Impaired fasting glucose is an immediate diagnosis, and it was assumed that the more closely related these patients are to the confirmed diabetics, the likelier they are to have or to develop, the disease.

The families were checked for relationship errors by comparing the identity-by state (IBS) distribution for the set of 906 markers, for each pair of related and genotyped individuals, to a reference distribution corresponding to the particular degree of relatedness. The reference distributions were constructed from a large subset of the Icelandic population. Individuals were excluded from the study if their relationship with the rest of the family was inconsistent with the relationship specified in the geneology database.

The remaining material that was available for the study was the following: 763 now confirmed Type II patients in 227 families together with 764 genotyped relatives. Of the patients, 667 were confirmed Type II patients, 35 unconfirmed Type II patients, 52 confirmed patients with impaired fasting glucose (IFG) and 9 unconfirmed patients with IFG.

Stratification of the Patient Material

The patients were classified into two sub-phenotypes based on their BMI: non-obese Type II diabetics are patients who have BMI less than 30, and obese Type II diabetics are patients who have BMI at or above 30. The reason for fractionating the diabetics into non-obese and obese groups is that other factors may be influencing the pathogenesis of disease in these two groups. Obesity alone could be contributing to the diabetic phenotype. Therefore, this factor was separated. Obesity is most likely due to a combination of environmental and genetic factors. This fractionation into non-obese and obese diabetics practically separates the material into two halves; 60% of the patients are in the non-obese category (20% with BMI below 25 (lean) and 40% with BMI between 25-30 (overweight)), and 40% of the patients are in the obese category (BMI above 30).

An affected-only linkage analysis for each of those sub-phenotypes was performed, using the same set of families as above, but classifying patients not belonging to the particular sub-group as having an unknown disease status. Restricted to a particular sub-phenotype, some families no longer contained a pair of related patients classified as affecteds and hence did not contribute in the linkage analysis. Such families were excluded from the analysis of the particular sub-phenotype. The number of patients and families used in the linkage analysis is summarized in Table 1 below.

Genome Wide Scan

A genome wide scan was performed on 772 patients and their relatives. Nine patients were excluded due to inheritance errors so the linkage analysis was performed with 763 patients and 764 relatives. The procedure was as described in Gretarsdóttir, et al., Am J Hum Genet., 70(3):593-603 (2002). In short, the DNA was genotyped with a framework marker set of 906 microsatellite markers with an average resolution of 4 cM. Alleles were called automatically with the TrueAllele program (Cybergenetics, Co., Pittsburgh, Pa.), and the program DecodeGT (deCODE genetics, ehf., Iceland), was used to fractionate according to quality and edit the called genotypes (Palsson, B., et al., Genome Res., 9(10):1002-1012 (1999)). The population allele frequencies for the markers were constructed from a cohort of more than 30,000 Icelanders that have participated in genome-wide studies of various disease projects at deCODE genetics. Additional markers were genotyped within the locus on chromosome 5q, where we observed the strongest linkage signal, to increase the information on identity by descent (IBD) sharing within the families. For those markers, at least 180 Icelandic controls were genotyped to derive the population allele frequencies.

The additional microsatellite markers that were genotyped within the locus were either publicly available or designed at deCODE genetics—those markers are indicated with a DG designation. Repeats within the DNA sequence were identified and allowed the selection or or of design primers that were evenly spaced across the locus. The identification of the repeats and location with respect to other markers utilized the physical mapping team at deCODE genetics.

For the markers used in the genomewide scan, the genetic positions were taken from the recently published high-resolution genetic map (HRGM), constructed at deCODE genetics (Kong A., et al., Nat Genet., 31: 241-247 (2002)). The genetic position of the additional markers were either taken from the HRGM, when available, or by applying the same genetic mapping methods as were used in constructing the HRGM map to the family material genotyped for this particular linkage study.

Statistical Methods for Linkage Analysis

The linkage analysis was done using the software Allegro (Gudbjartsson et al., Nat. Genet. 25:12-3, (2000)), that determines the statistical significance of excess sharing among related patients by applying non-parametric affected-only allele-sharing methods (without any particular disease inheritance model being specified). Allegro, a linkage program developed at deCODE genetics, calculates LOD scores based on multipoint calculations. The baseline linkage analysis used the Sₚₐᵢᵣₛ scoring function (Whittemore, A. S. and Halpern, J., Biometrics 50:118-27 (1994); Kruglyak L, et al., Am J Hum Genet 58:1347-63, (1996)), the exponential allele-sharing model (Kong, A. and Cox, N. J., Am. J. Hum. Genet., 61:1179 (1997)), and a family weighting scheme which was halfway on a log scale between weighting each affected pair equally and weighting each family equally. In the analysis, all genotyped individuals who were not affected were treated as “unknown”. Because of concern with small sample behavior, corresponding P-values were usually computed in two different ways for comparison. The first P-value was computed based on large sample theory; Z_{Ir}=√(2 logₑ (10) LOD) and was approximately distributed as a standard normal distribution under the null hypothesis of no linkage. A second P-value was computed by comparing the observed LOD score to its complete data sampling distribution under the null hypothesis. When a data set consisted of more than a handful of families, these two P-values tended to be very similar.

All suggestive loci with LOD scores greater than 2 were followed up with some extra markers to increase the information on the IBD-sharing within the families and to decrease the chance that a LOD score represents a false-positive linkage. The information measure used was defined by Nicolae (D. L. Nicolae, Thesis, University of Chicago (1999)) and is a part of the Allegro program output. This measure is closely related to a classical measure of information as previously described by Dempster et al. (Dempster, A. P., et al., J. R. Statist. Soc. B. 39:1 (1977))—the information equals zero if the marker genotypes are completely uninformative and equals one if the genotypes determine the exact amount of allele sharing by descent among the affected relatives. Using the framework marker set with average marker spacing of 4 cM typically resulted in information content of about 0.7 in the families used in the linkage analysis. Increasing the marker density to one marker every centimorgan usually increased the information content above 0.85.

Results

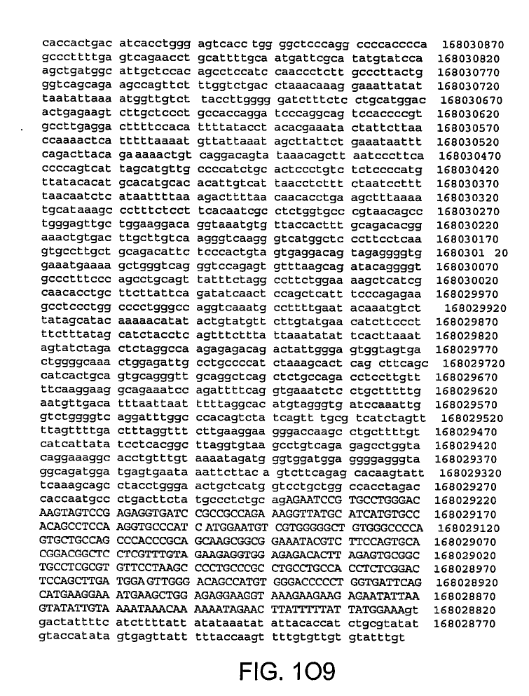

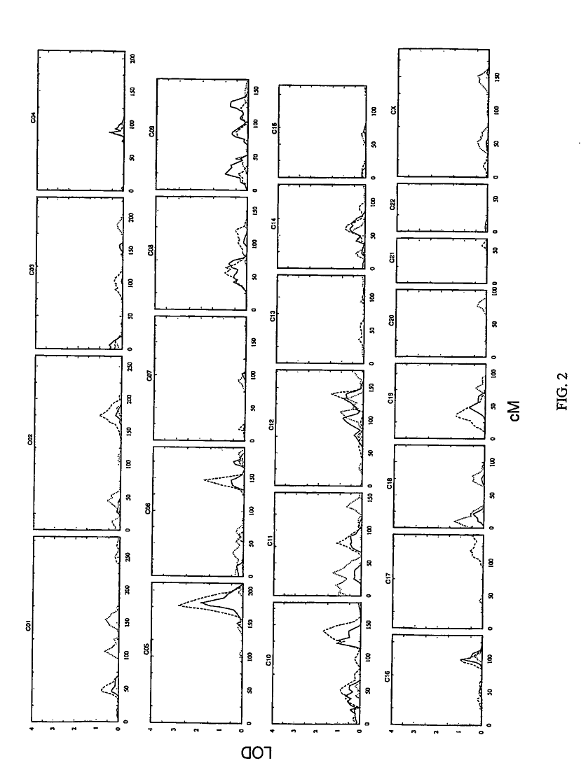

The results of the genome-wide linkage analysis with the framework marker set are shown in FIG. 2, which depicts the allele-sharing LOD-score versus the genetic distance from the p-terminus in centimorgan (cM) for each of the 23 chromosomes. The analysis was performed with the three phenotypes: all Type II diabetics (solid lines), non-obese diabetics (dashed lines) and obese diabetics (dotted lines). A LOD-score of 1.84 was observed on chromosome 5q34-q35.2 with the framework marker set when all Type II diabetics were used in the analysis. When the linkage analysis was restricted to non-obese diabetics, this LOD-score increases to 2.81. The obese diabetics did not show linkage in this region.

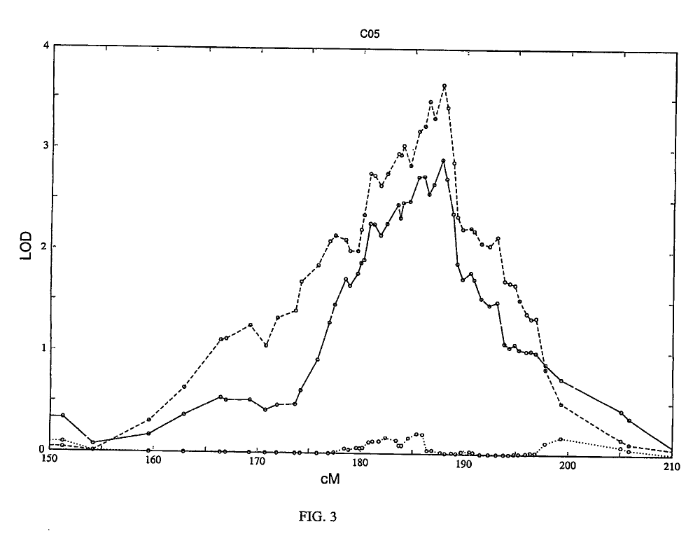

Additional markers were genotyped in this area to increase the information content and to confirm the linkage. The information on the IBD-sharing at this locus was about 78% with the framework marker set. In order to increase the information content, another 38 microsatellite markers were genotyped within a 40 cM region that includes the observed signal. Repeating the linkage analysis including the additional markers increased the LOD-score to 3.64 (P-value=3.18×10⁻⁵) for the non-obese diabetics. For all patients, the peak LOD-score increased to 2.9 (P-value=1.22×10⁻⁴). This is shown in FIG. 3.

The peak of the LOD-score is centered on marker D5S625 and the region determined by a drop of one in the LOD is from marker DG5S5 to marker D5S429, centromeric and telomeric respectively. The one-LOD-drop is about 9 cM and estimated to be about 3.5 Mb. This 1-LOD-drop roughly corresponds to the 80-90% confidence interval for the location of a putative disease associated gene.

Locus-Wide Association Study

Genotyping to Narrow Down the Region of Linkage

In order to narrow down the region of interest, the linkage analysis was followed by a comprehensive association study of the 1-LOD-drop. This was performed because the linkage analysis has limited resolution in that it compares sharing among closely related individuals that share, on average, large chromosomal segments. For the association analysis, identified a large number of additional microsatellite markers were identified as located in the 1-LOD-drop, and those markers were typed in both the patient cohort and in a large number of unrelated controls randomly selected from the Icelandic population.

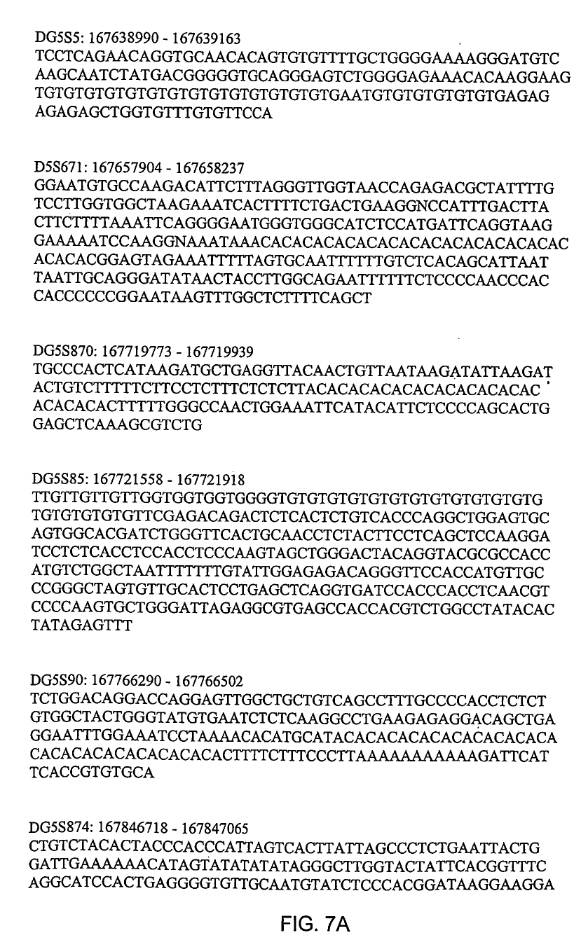

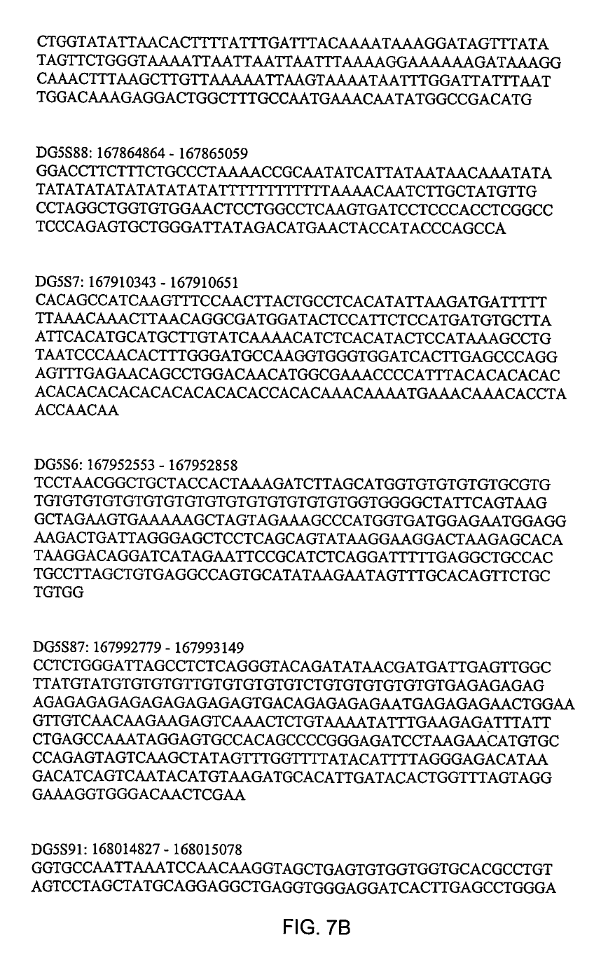

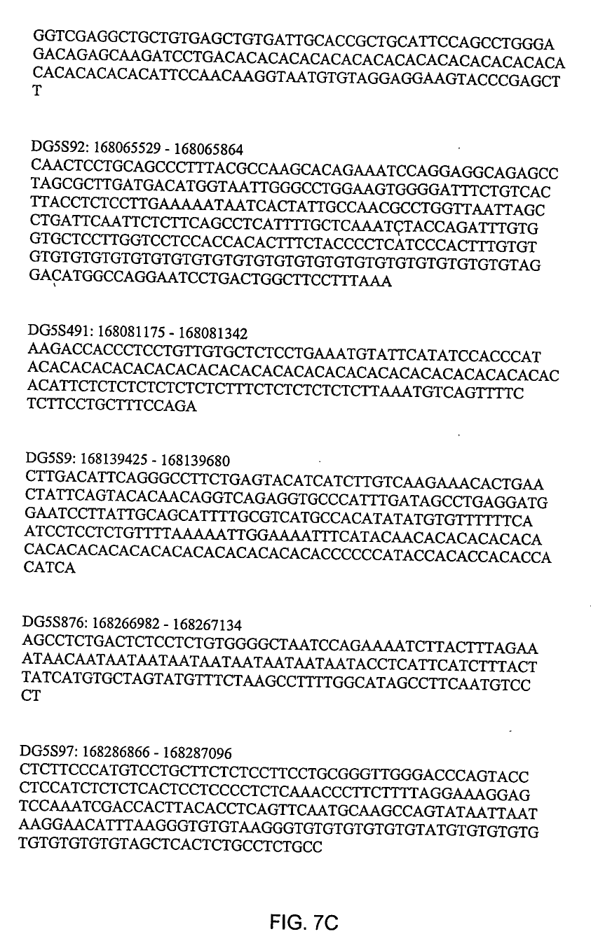

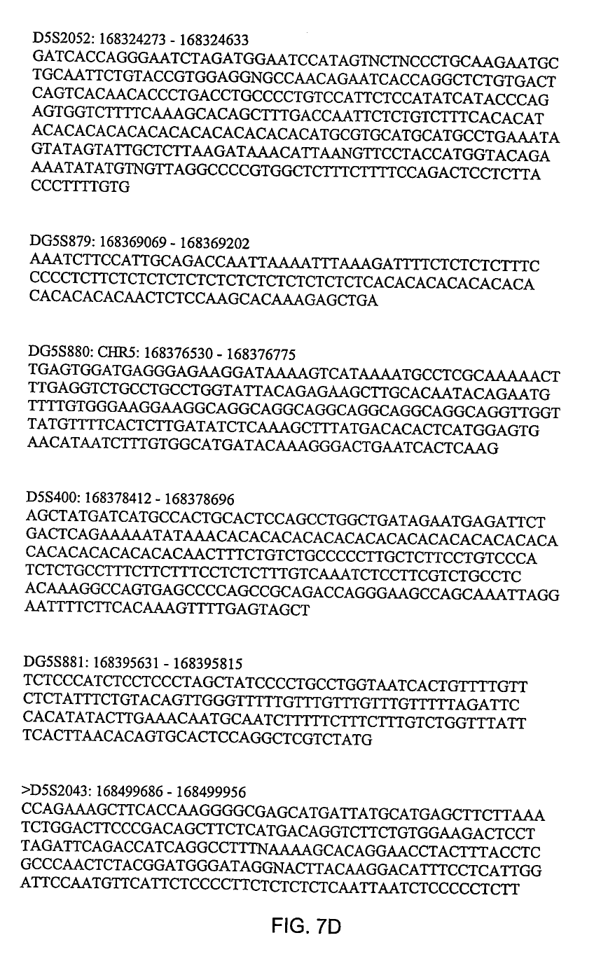

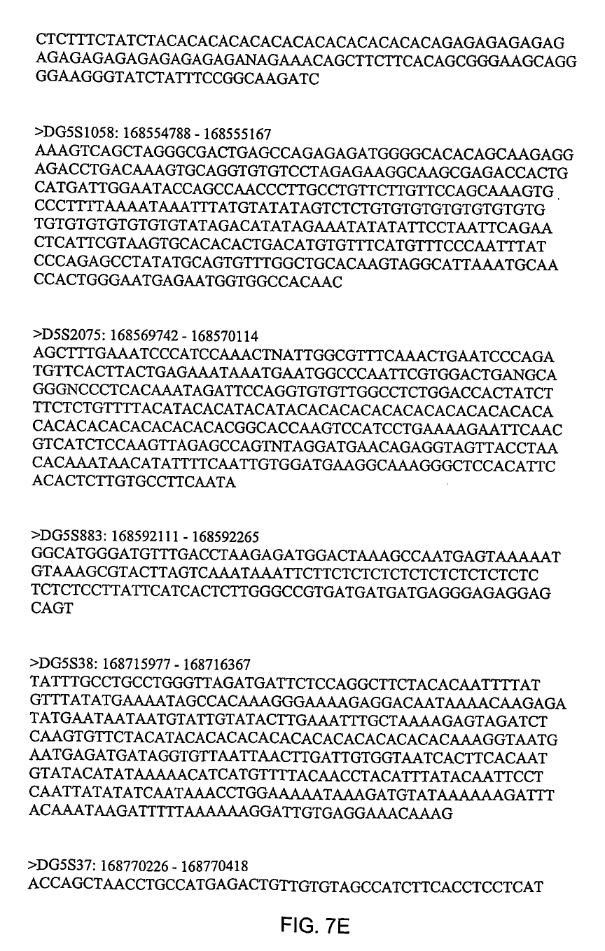

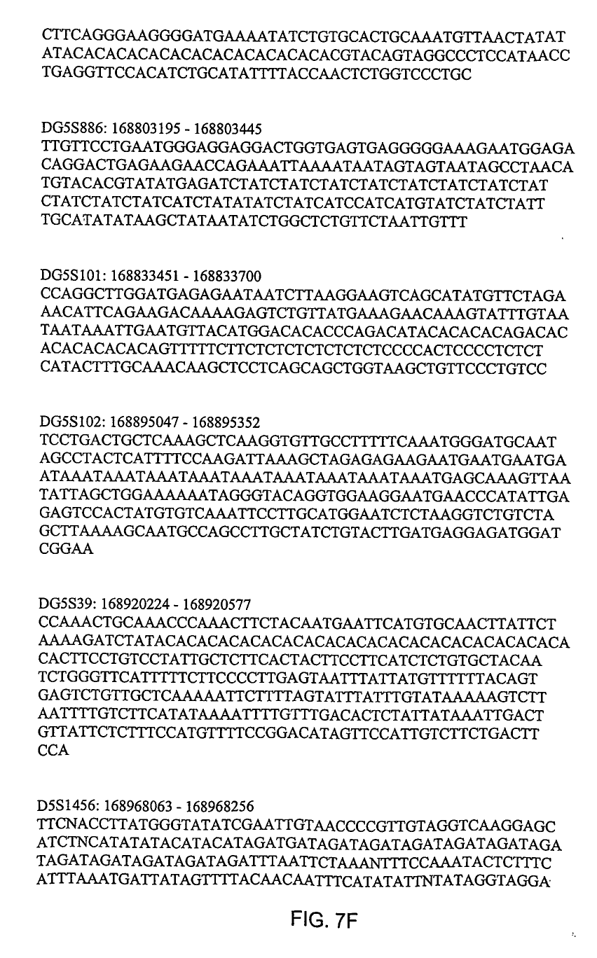

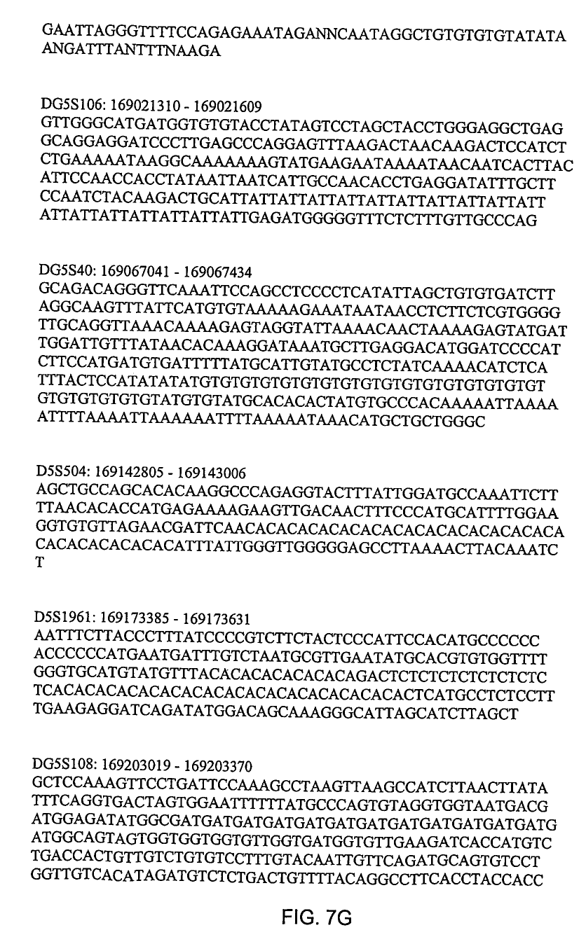

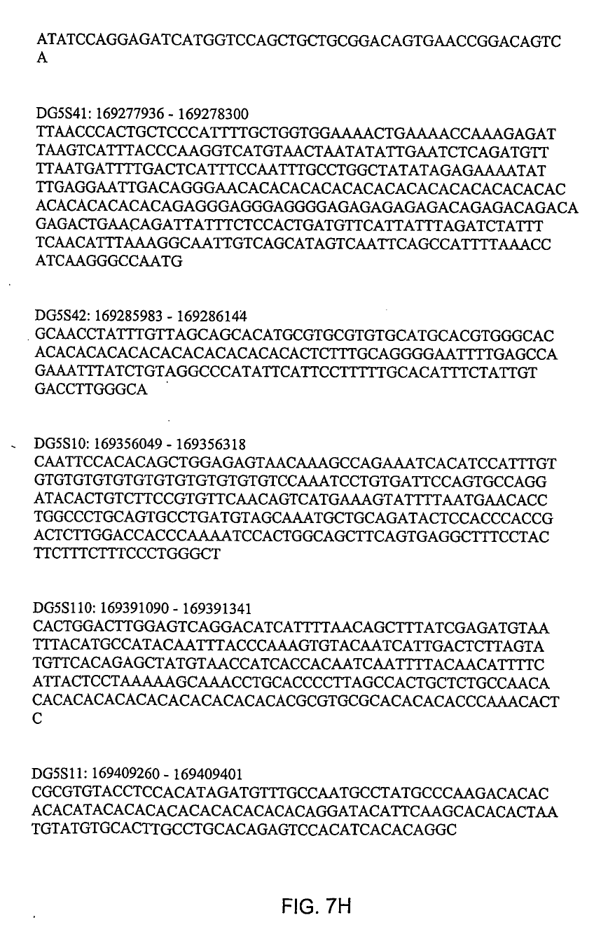

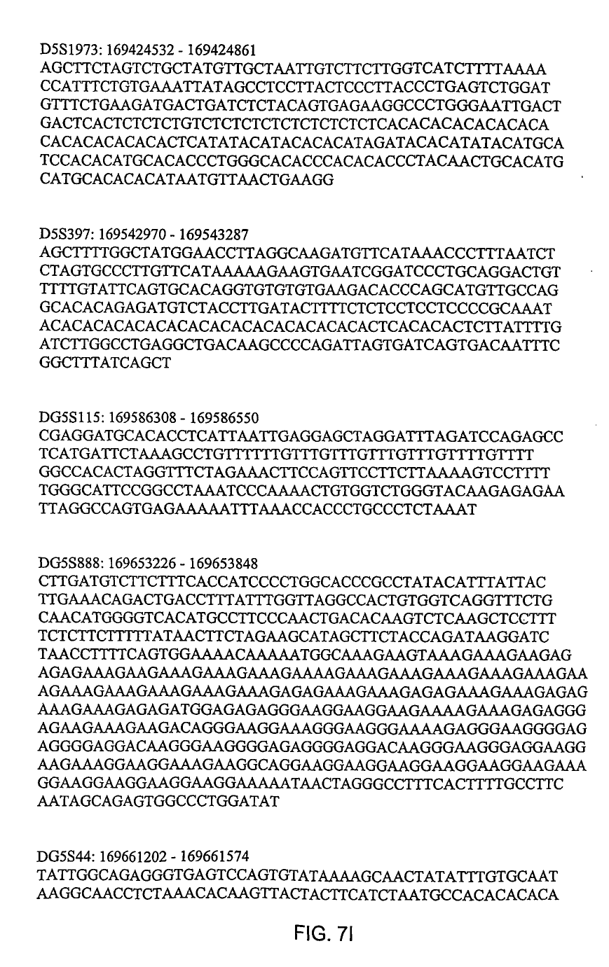

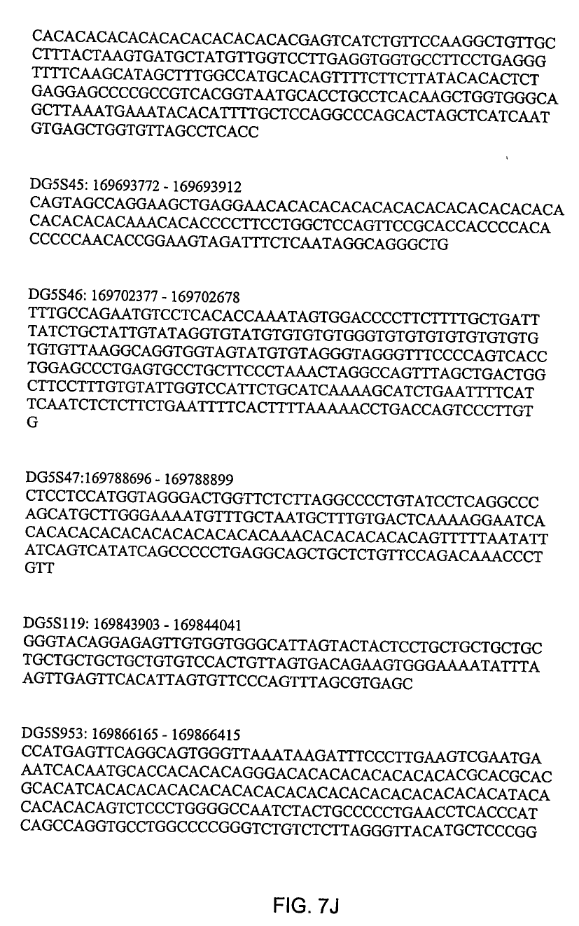

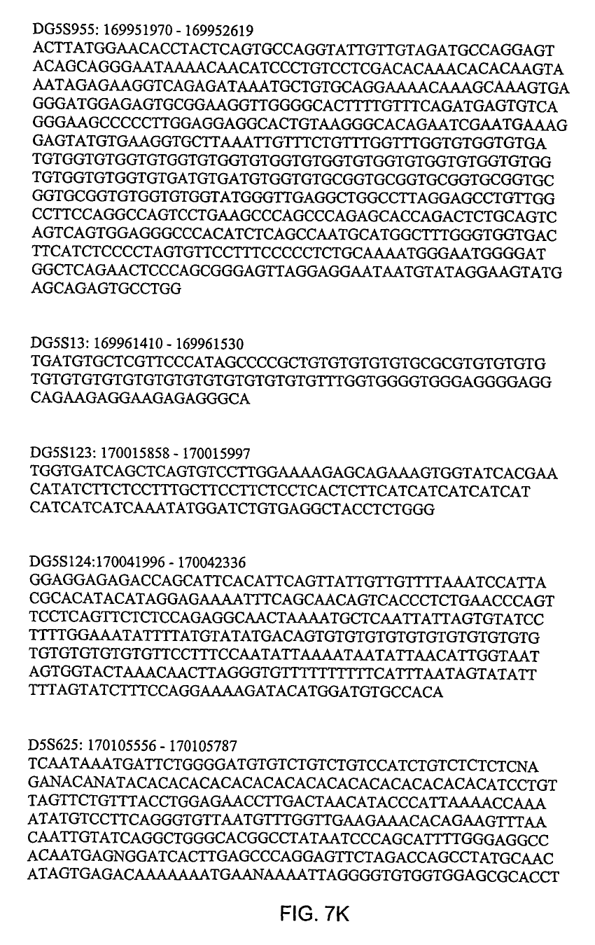

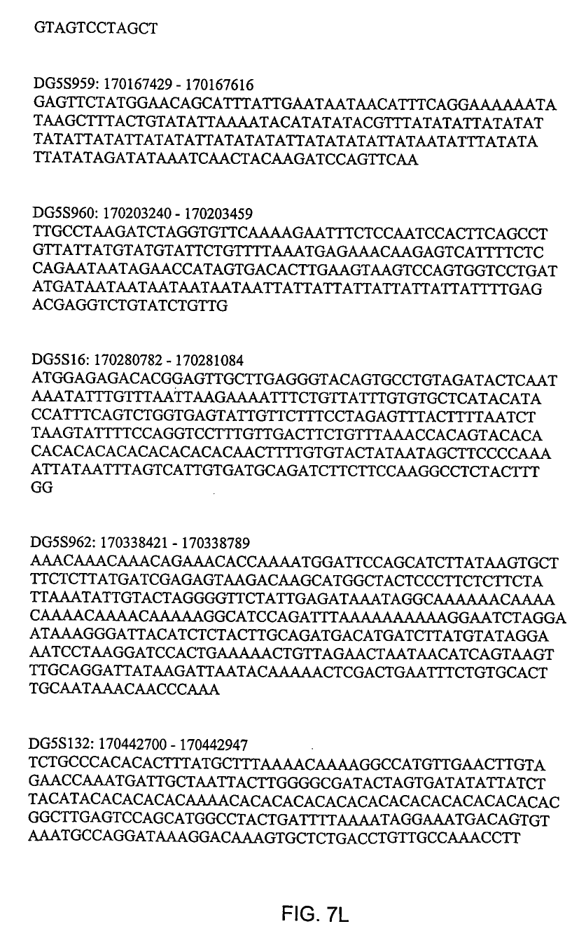

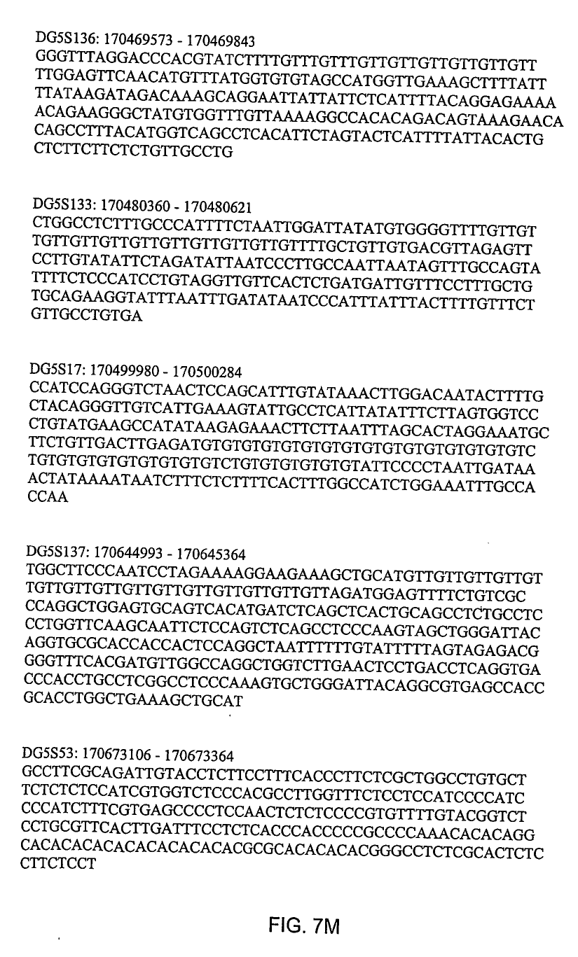

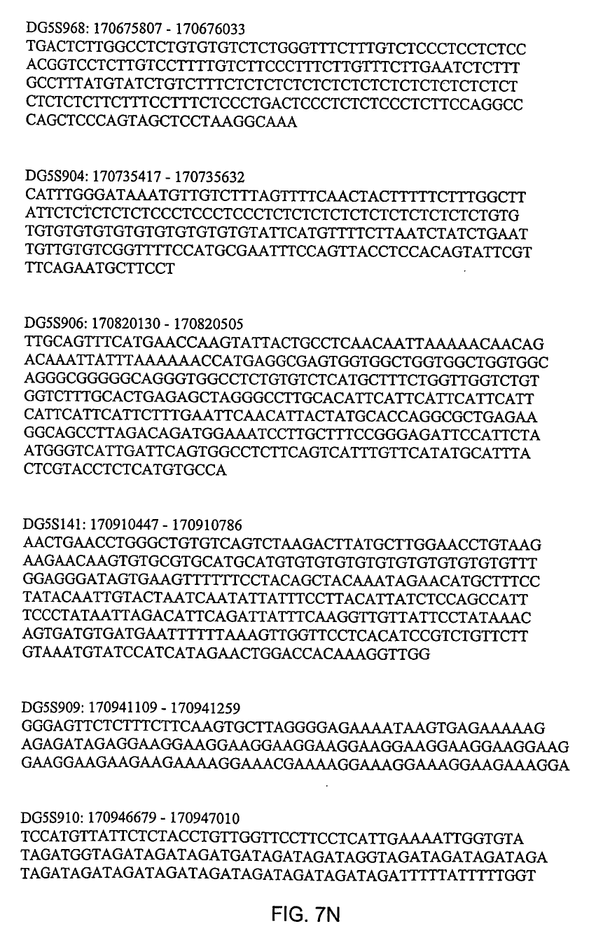

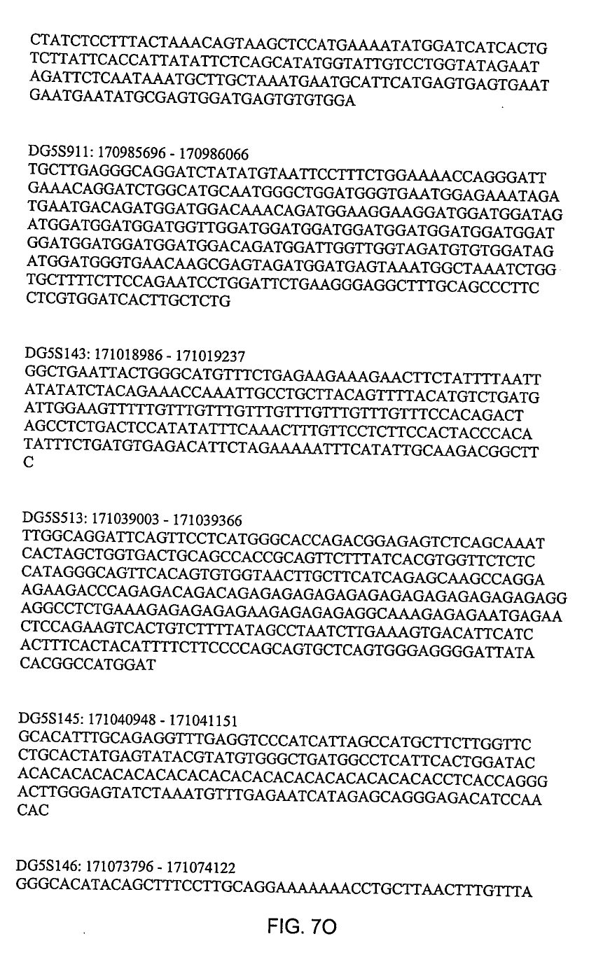

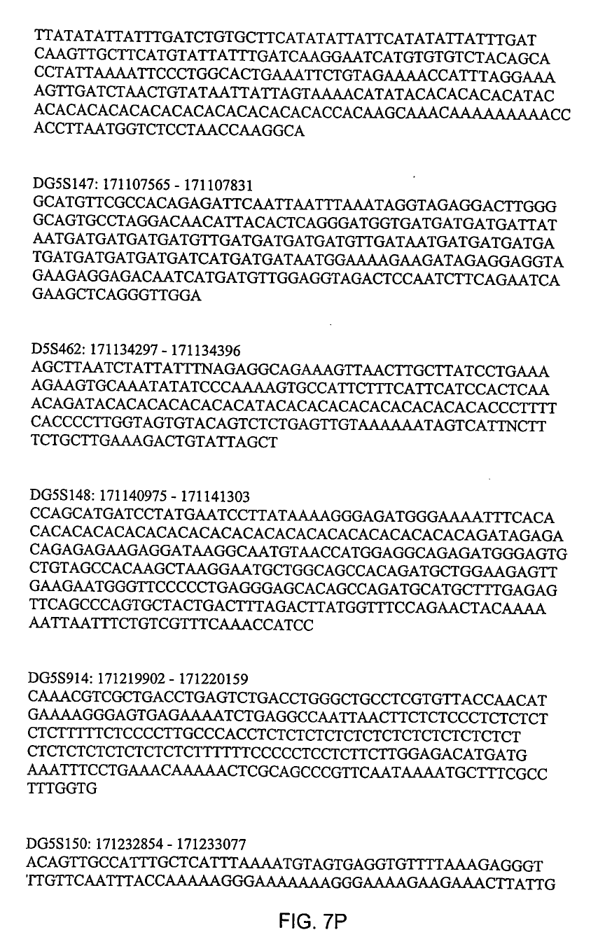

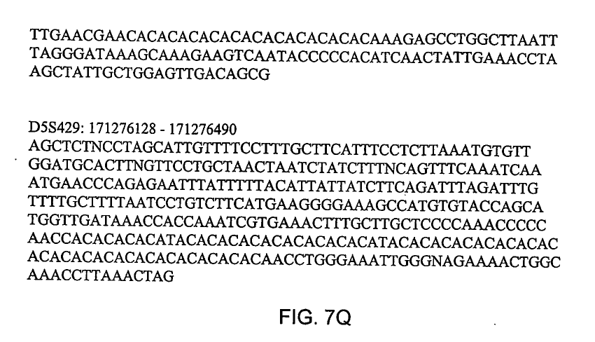

Sixty-seven markers were identified and typed in the 1-LOD-drop, in addition to the 17 markers already typed and used in the linkage analysis. The locus-wide association microsatellites are as shown in FIG. 7. The new polymorphic repeats (dinucleotide or trinucleotide repeats) were identified with the Sputnik program. The smaller allele of CEPH sample 1347-02 (CEPH genomics repository) was subtracted from the alleles of the microsatellites and used as a reference. Thus, a total of 84 markers were available for the association analysis, i.e., an average density of one marker every 42 kb or one marker every 0.107 cM. All those markers were typed for 590 non-obese diabetics and 477 unrelated controls.

Statistical Methods for Association and Haplotype Analysis

For single marker association to the disease, the Fisher exact test was used to calculate a two-sided P-value for each individual allele. When presenting the results, allelic frequencies were used rather than carrier frequencies for microsatellites, SNPs and haplotypes. Haplotype analyses were performed using a computer program developed at deCODE called NEMO (NEsted MOdels) (Gretarsdóttir, et al., Nat Genet. October;35(2):131-8 (2003)). NEMO was used both to study marker-marker association and to calculate linkage disequilibrium (LD) between markers, and for case-control haplotype analysis. With NEMO, haplotype frequencies were estimated by maximium likelihood and the differences between patients and controls were tested using a generalized likelihood ratio test. The maximum likelihood estimates, likelihood ratios and P-values were computed with the aid of the EM-algorithm directly for the observed data, and hence the loss of information due to the uncertainty with phase and missing genotypes was automatically captured by the likelihood ratios, and under most situations, large sample theory could be used to reliably determine statistical significance. The relative risk (RR) of an allele or a haplotype, i.e., the risk of all allele compared to all other alleles of the same marker, was calculated assuming the multiplicative model (Terwilliger, J. D. & Ott, J. A haplotype-based ‘haplotype relative risk’ approach to detecting allelic associations. Hum Hered 42, 337-46 (1992) and Falk, C. T. & Rubinstein, P. Haplotype relative risks: an easy reliable way to construct a proper control sample for risk calculations. Ann Hum Genet 51 (Pt 3), 227-33 (1987)), together with the population attributable risk (PAR).

In the haplotype analysis, it may be useful to group haplotypes together and test the group as a whole for association to the disease. This is possible to do with NEMO. A model is defined by a partition of the set of all possible haplotypes, where haplotypes in the same group are assumed to confer the same risk while haplotypes in different groups can confer different risks. A null hypothesis and an alternative hypothesis are said to be nested when the latter corresponds to a finer partition than the former. NEMO provides complete flexibility in the partition of the haplotype space. In this way, it is possible to test multiple haplotypes jointly for association and to test if different at-risk haplotypes confer different risk.

As a measure of LD, two standard definitions of LD, D′ and R² were used as they provide complementary information on the amount of LD (Lewontin, R. “The interaction of selection and linkage I. General considerations: Heterotic models.” Genetics, 1964. 49:49-67; Hill, W. G. and A. Robertson, “Linkage disequilibrium in finite populations.” Theor. Appl. Genet., 1968. 22:226-231). For the purpose of estimating D′ and R², the frequencies of all two-marker allele combinations were estimated using maximum likelihood methods and the deviation from linkage disequilibrium was evaluated using a likelihood ratio test. The standard definitions of D′ and R² were extended to include microsatellites by averaging over the values for all possible allele combinations of the two markers weighted by the marginal allele probabilities.

The number of possible haplotypes that could be constructed out of the dense set of markers genotyped in the 1-LOD-drop was very large, and even though the number of haplotypes that were actually observed in the patient and control cohort was much smaller, testing all those haplotypes for association to the disease was a formidable task. Note that the analysis was not restricted to haplotypes constructed from a set of consecutive markers, as some markers might be very mutable and might split up an otherwise well conserved haplotype constructed out of surrounding markers.

The approach taken to the problem of identifying those haplotypes in the candidate region that show strongest association to the disease was two-fold: First, the haplotypes tested were restricted to span a sub-region small enough that the included markers may be expected to be in substantial LD. In this study, only haplotypes that span less than 300 kb were considered. Second, an iterative procedure was applied, that gradually builds up the most significant haplotypes. Starting with haplotypes constructed out of 3 markers, those haplotypes that showed strong association to the disease were selected, other nearby markers were added to those haplotypes, and the association test was repeated. By iterating this procedure, those haplotypes that show strongest association to the disease were identified.

Results

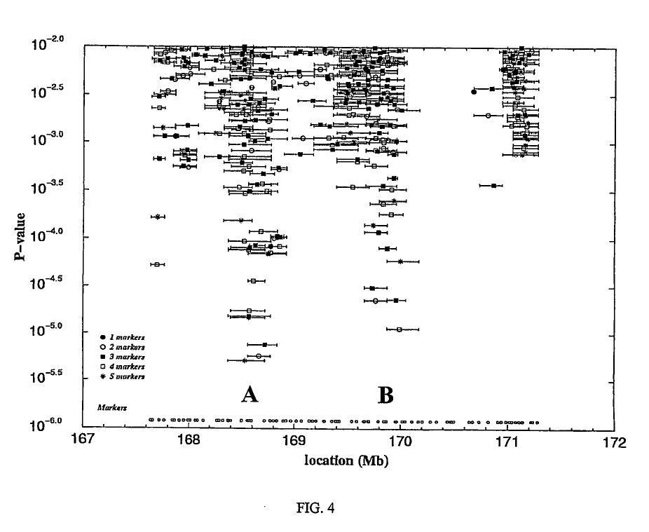

For the association analysis, 590 non-obese Icelandic Type II diabetes patients and 477 unrelated population controls were genotyped using a total of 84 microsatellite markers. These markers were distributed evenly across a region of approximately 3.5 Mb. The region was centered on the linkage peak and corresponded to the 1-LOD-drop. The procedure described above was then followed, and single-markers and haplotypes consisting of up to 5 markers that showed association to the disease were identified. The result is summarized in FIG. 4. In FIG. 4, the location of a marker or a haplotype is shown on the horizontal axis and the corresponding P-value from the associaton test on the vertical axis. This is shown for all haplotypes tested that have a P-value less than 0.01. The horizontal bars indicated the size of the corresponding haplotypes and the location of all markers is shown at the bottom of the figure. All locations are in Mb and refer to the NCBI Build33.

A series of correlated haplotypes were observed that show strong association for non-obese diabetics in two locations within the 1-LOD-drop. Those regions were denoted A (168.37-168.83 Mb) and B (169.70-170.17 Mb), and in Table 2 are listed the most significant haplotype in each of those regions. For each haplotype, the table includes a two-sided single-test P-value for association, calculated using NEMO, the corresponding relative risk, the estimated frequency of the haplotype in the patient and the control cohorts, the region the haplotpe spans, and the markers and alleles (in bold) that define the haplotype. Note, however, that some of the haplotypes listed within each of the two regions are very correlated and should be considered as a single observation of association to the disease. This is demonstrated for region A in Table 3, which lists the pairwise correlation, both D′ and R², between the haplotypes. Based on the correlation, we can split the A-haplotypes into two groups; group I includes A1, A4 and A6, and group II includes A2, A3 and A5. Haplotypes within each of the groups are very correlated, however, there is much less correlation between haplotypes in different groups. From Table 2 it is observed that group I can be defined by haplotype A6 alone as both haplotypes A1 and A4 are a subset of A6. Likewise, group II can be defined by A2 alone. As the correlation between A2 and A6 is weak, they constitute almost independent observations of association to non-obese diabetes in region A. Hence it is possible to test haplotypes A2 and A6 together as a group for association to non-obese diabetes. This test yields a P-value=2.9×10⁻⁹ with a corresponding relative risk of 4.2, a population atributable risk of 11.5%, and an allelic frequency of 0.078 and 0.020 in the patient and the control cohorts, respectively.

Investigation of Region A

Genes in Region A

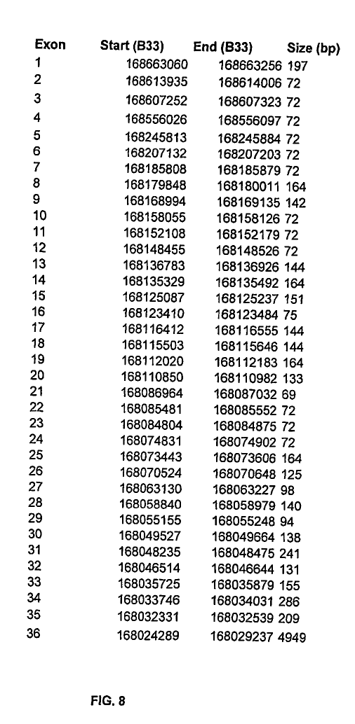

All genes in and around region A were identified (UCSC (University of California at Santa Cruz (http://www.cbse.ucsc.edu/Genome/; this is a human reference sequence based on NCBI Build 33, produced by the International Human Genome Sequencing Consortium). In the region defined by the six most significant haplotypes, 168.37-168.83 Mb, there is only one gene, SLIT3 (slit homolog 3 (Drosophila)). SLIT3 is a rather big gene that extends over 600 kb, from 168.03 to 168.66 Mb, and the at-risk haplotypes are localized in the 5′-end of the gene and include the first four exons. This is shown in FIG. 5, which shows the location of all microsatellites in the interval 167.6 to 169 Mb (filled circles), the locations of all the exons of SLIT3 (filled boxes) and the span of the at-risk haplotypes A1, . . . , A6 (grey horizontal bars). The figure also shows the location of four neighbouring genes ODZ2 (odd Oz/ten-m homolog 2), KIAA0869, RARS (arginyl-tRNA synthetase) and PANK3 (pantothenate kinase 3) (shaded boxes) that are located centromeric to SLIT3, i.e. 500 kb away from the observed association signal. Exons of SLIT3 are also shown in FIG. 8, which depicts the Build33 location of the exons.

Identification of SNPs and Microsatellites

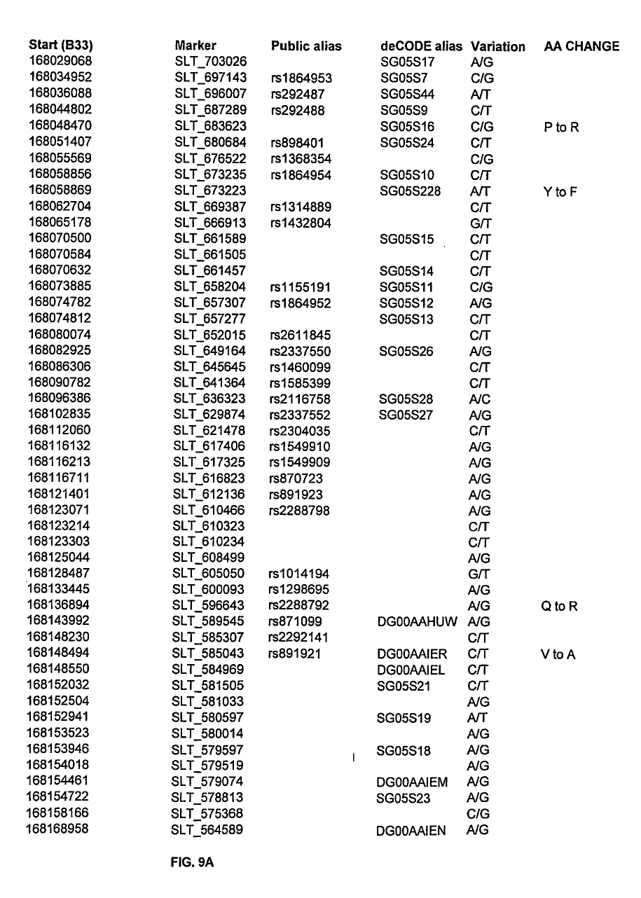

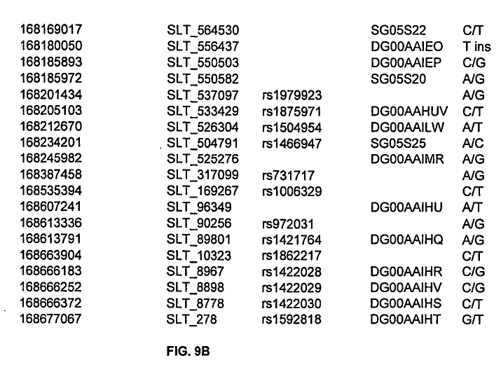

In order to identify SNPs across SLIT3, all 36 exons of SLIT3 and their flanking regions were sequenced on 94 non-obese diabetic patients. As a consequence, 68 SNPs were identified, and are shown in FIG. 9 (depicting the Build33 location of SNPs found across SLIT3 after sequencing of the exons and flanking sequences). They include four non-synonymous amino acid changes—SLT_{—}683623 (P to R), SLT_{—}673223 (Y to F), SLT_{—}596643 (Q to R) and SLT_{—}585043 (V to A). Two SNPs, SLT_{—}596643 and SLT_{—}585043, are SNPs that have been previously reported in the public domain as rs2288792 and rs891921, respectively. Additional SNPs were identified across the gene by selecting SNPs from the public domain (US National Center for Biotechnology Information's SNP database) and designing SNP assays for them. SNPs SG05S458 and SG05S459 were identified from spot sequencing the 5′ end of SLIT3 on 12 population-based DNA samples. See FIG. 10 for the DNA sequences of the SNPs identified across SLIT3; and FIG. 11 for the Build33 location of all SNPs and microsatellites identified as polymorphic across SLIT3.

SNPs on 470 non-obese diabetics and 658 population-based controls were genotyped using a method for detecting SNPs with fluorescent polarization template-directed dye-terminator incorporation (SNP-FP-TDI assay) (Chen, X., Zehnbauer, B., Gnirke, A. & Kwok, P. Y. Fluorescence energy transfer detection as a homogeneous DNA diagnostic method. Proc. Natl. Acad. Sci. USA 94, 10756-10761 (1997)).

Association Study of SLIT3

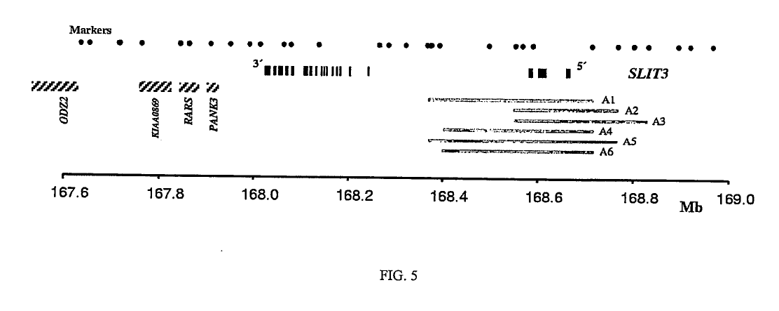

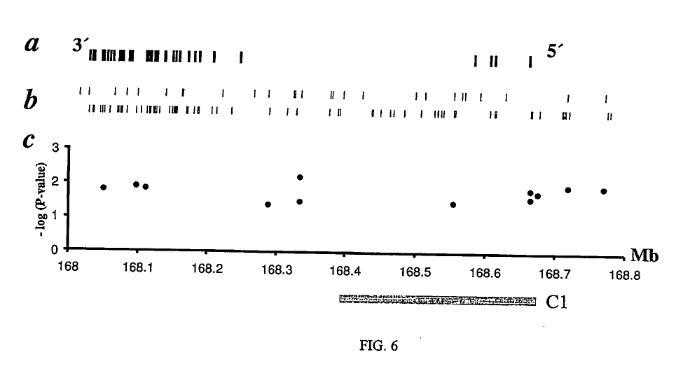

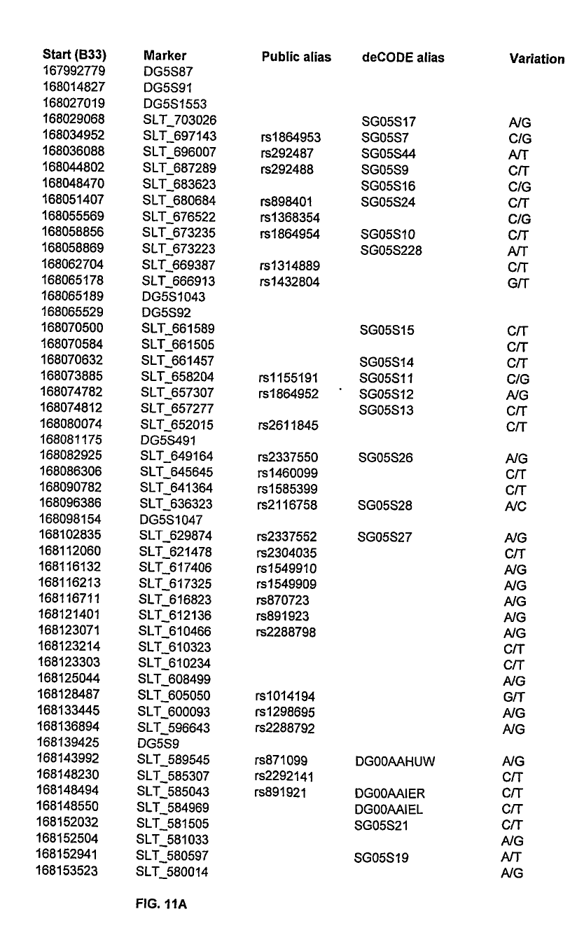

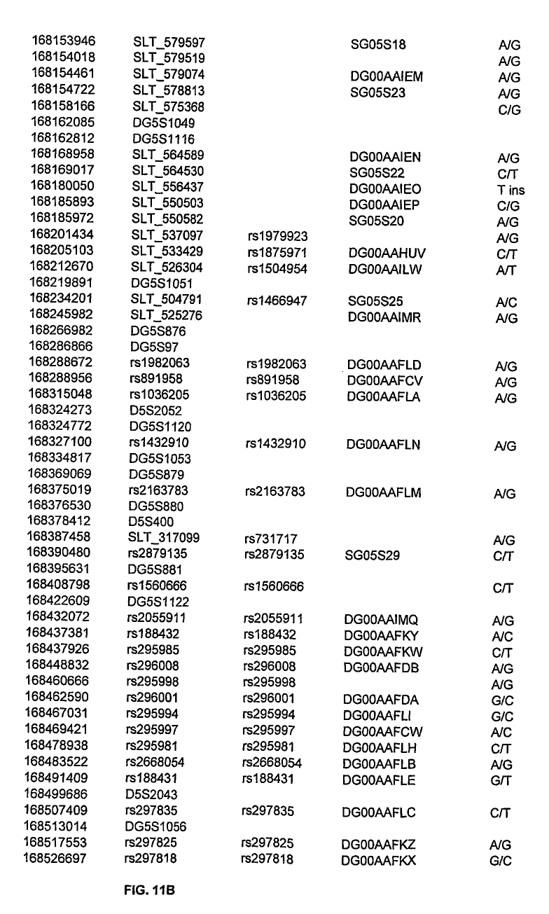

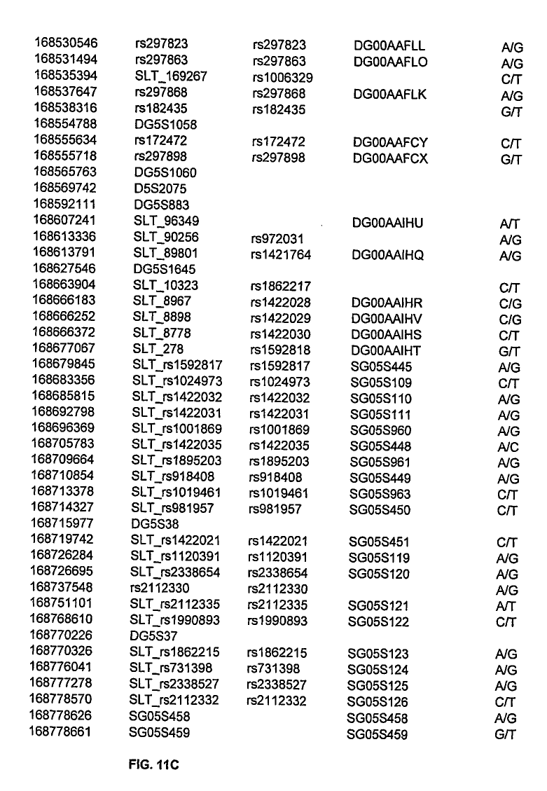

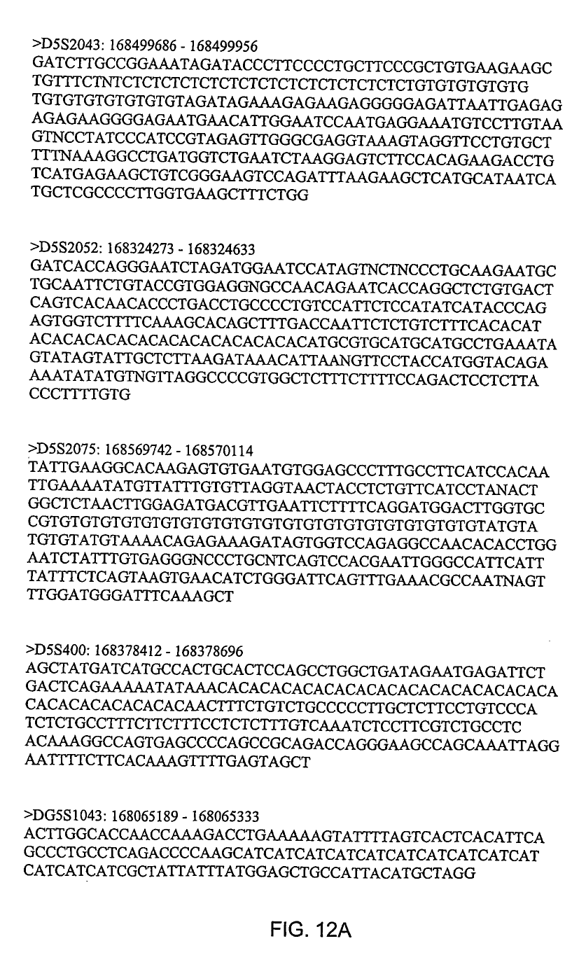

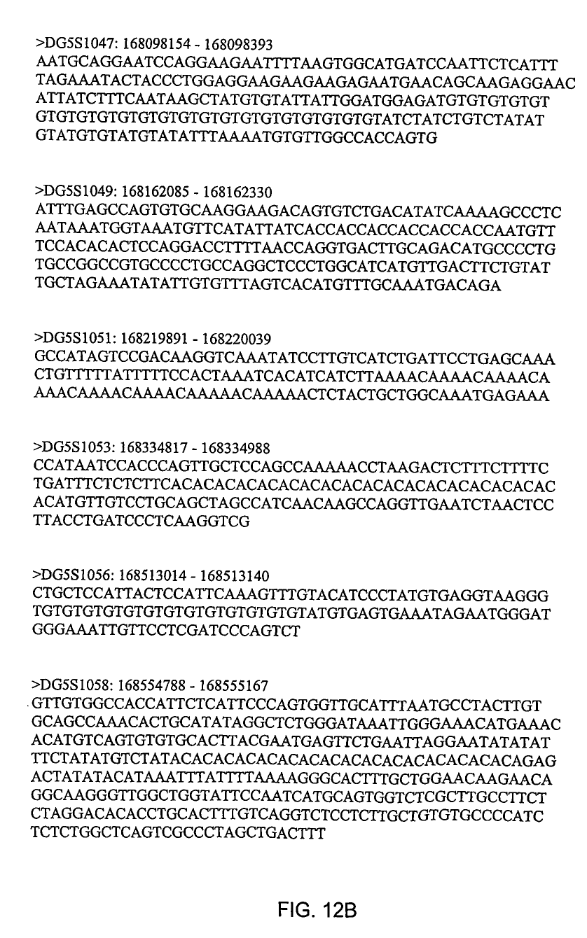

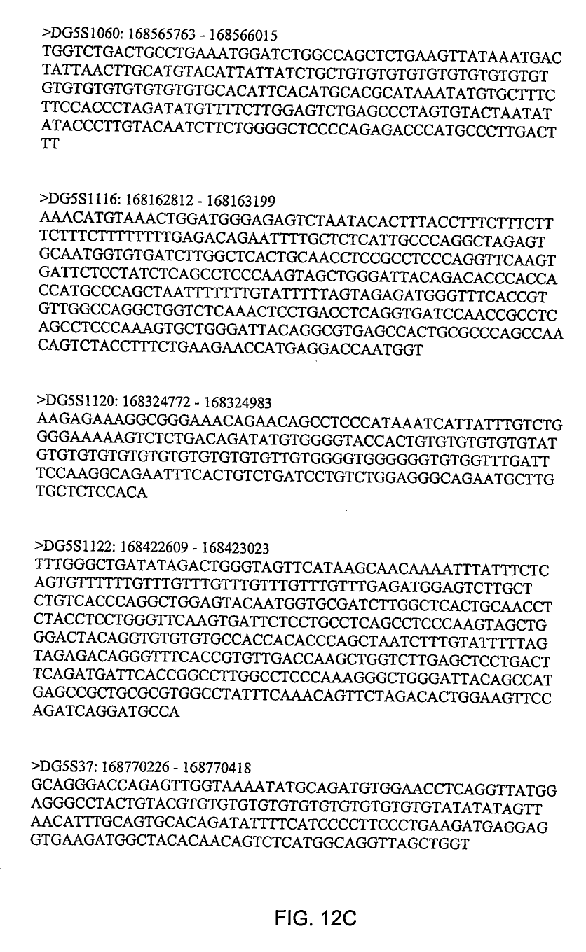

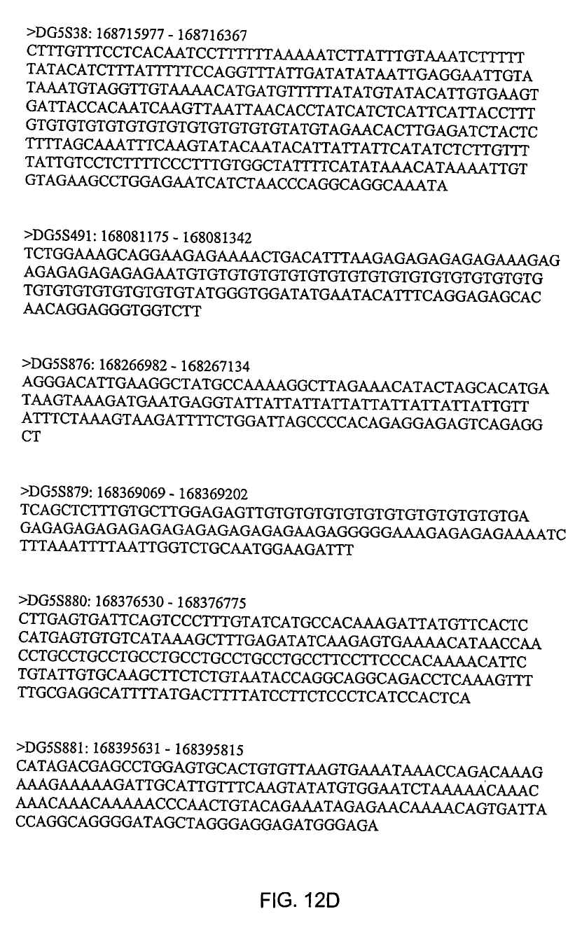

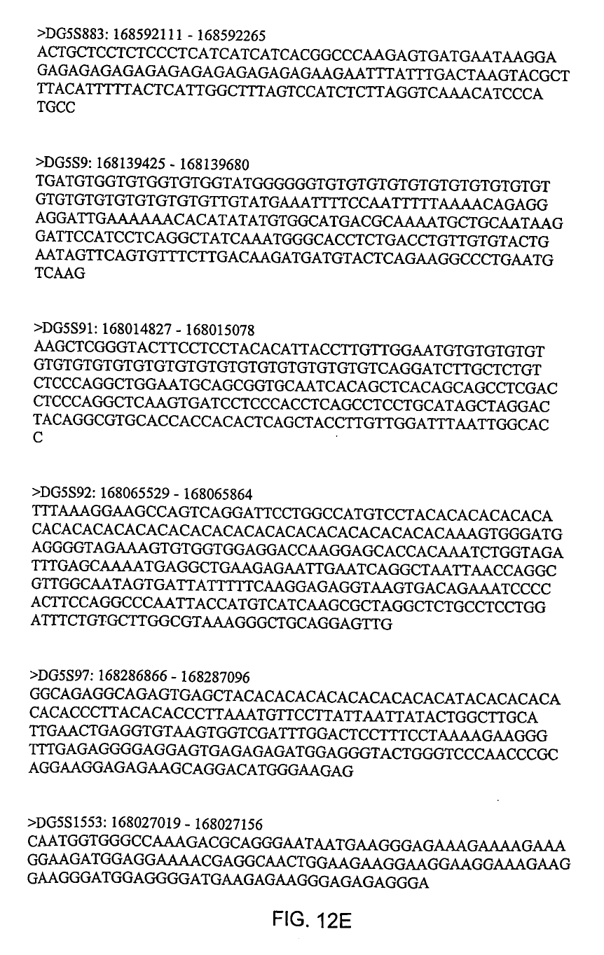

Twenty-nine microsatellite markers and 77 SNPs, located in and around SLIT3, were tested for single-marker association to non-obese diabetics. FIG. 12 shows the DNA sequences of the microsatellites employed for the association studies across SLIT3 (including Build33 locatrions); FIG. 13 shows the names of the SNPs and microsatellites employed for the association analysis across SLIT3. For this part of the association study, 523 non-obese diabetics and 323 unrelated population controls that had been typed for both the microsatellites and the SNPs were used. Thirteen markers had different allelic frequencies between patients and controls with a P-value less than 0.05. Those results are listed in Table 4. FIG. 6 shows the results of the single-marker association (FIG. 6c), together with the exonic structure of SLIT3 (FIG. 6a) and the location of the 106 microsatellites and SNPs (FIG. 6b).

Five of the 13 markers that show association were located in the 5′ end of SLIT3, close to, and downstream of, the first exon. The haplotype analysis was repeated, restricted to the 106 microsatellites and SNPs in SLIT3 and, as for the locus-wide association, only haplotypes shorter than 300 kb that included five or less, possibly non-consecutive, markers, were tested. Table 5 shows the five haplotypes that showed strongest association to non-obese diabetes, with P-values ranging from 2.3×10⁻⁸ to 6.9×10⁻⁸. Like the most significant haplotypes observed in the locus-wide association, these five haplotypes, which are strongly correlated to each other, span the first four exons in the 5′ end of SLIT3. The span of haplotype C1 is shown at the bottom of FIG. 6. Indeed, the key SNPs in defining those haplotypes are located very close to the first exon. The four most significant haplotypes, C1-C4, are very common, with allelic frequency of 0.28 in patients and 0.16 in controls, with relative risk 2.1 and population attribuatble risk of 27.5%.

Although haplotypes C1, . . . , C5 are localized in the same region as the most significant microsatellite haplotypes observed in the locus-wide association study, they constitute an independent observation of association to non-obese diabetes of the 5′-end of SLIT3. For example, the correlation coffecient R² between haplotype C1 and haplotypes A2 and A6 is 0 and 0.02 respectivly. Again, just as with A2 and A6, haplotypes C1, A2 and A6 can be tested together as a group for association to non-obese diabetes. This test yields a P-value=6.3×10⁻¹¹ and corresponding relative risk and population atributable risk of the haplotypes as a group is 2.2 and 33%. The frequency of the haplotype group is 0.33 in non-obese diabetics and 0.18 in the control cohort.

Association Study of Other Genes in the Region

In order to verify if any of the neighboring genes showed any association to diabetes, the exons of ODZ2, KIAA0869, RARS and PANK3 were sequenced, and the SNPs that were found were typed, together with a number of microsatellite marker and public SNPs, in the same cohort of non-obese diabetics and population controls. No association was observed across any of these genes.

Tables

TABLE 1
The number of patients and families that contribute to
the genome-wide linkage scan, both when all the patients
are used, and when the analysis is restricted to obese
or non-obese diabetic patients respectively.

| | | No. of families | No. of patients |
|---|---|---|---|
| | Total Number | contributing to | contributing to |
| Phenotype | of Patients | the analysis | the analysis |
| All diabetics | 763 | 227 | 763 |
| Obese | 296 | 92 | 219 |
| Non-obese | 467 | 154 | 413 |

TABLE 2
Haplotypes within the 1-LOD-drop that show the strongest association to non-obese diabetes. For each haplotype, we
show (i) a two-sided P-value for a single test of association to non-obese diabetes, (ii) the corresponding relative
risk (RR), (iii) the estimated allelic frequency of the haplotype in the patient and the control cohort, (iv) the span
of the haplotype (refering to NCBI33) and (v) the alleles (in bold) and markers that define the haplotype. The haplotypes
are separated into two groups, A and B, corresponding to two different regions within the 1-LOD-drop.

| | | P-value | RR | Aff. frq | Ctrl. frq | Span (Mb) | Haplotype |
|---|---|---|---|---|---|---|---|
| Region A | A1 | 0.000005 | >10 | 0.033 | 0.000 | 168.37-168.72 | 0 DG5S879 4 DG5S881 −4 C5S2075 0 DG5S883 4 DG5S38 |
| | A2 | 0.000006 | 3.81 | 0.053 | 0.015 | 168.55-168.77 | 4 DG5S1058 −6 DG5S37 |
| | A3 | 0.000008 | 3.64 | 0.054 | 0.015 | 168.55-168.83 | 4 DG5S1058 −6 DG5S37 0 DG5S101 |
| | A4 | 0.000015 | 6.18 | 0.046 | 0.008 | 168.40-168.72 | 4 DG5S881 4 DG5S1068 −4 DGS2075 0 DG5S883 4 DG5S38 |
| | A5 | 0.000015 | 4.42 | 0.047 | 0.011 | 168.37-168.77 | 0 DG5S879 4 DG5S1058 −6 DG5S37 |
| | A6 | 0.000018 | 6.94 | 0.045 | 0.007 | 168.40-168.72 | 4 DG5S881 −4 D5S2075 0 DG5S383 4 DG5S38 |
| Region B | B1 | 0.000011 | >10 | 0.039 | 0.000 | 169.87-170.17 | 0 DG5S953 0 DG5S955 0 DG5S13 5 DG5S959 |
| | B2 | 0.000023 | >10 | 0.034 | 0.000 | 169.65-169.87 | 27 DG5S888 0 DG5S953 |
| | B3 | 0.000023 | 5.26 | 0.049 | 0.010 | 169.87-170.04 | 0 DG5S953 0 DG5S955 4 DG5S124 |
| | B4 | 0.000031 | >10 | 0.034 | 0.000 | 169.65-169.87 | 27 DG5S888 0 DG5S44 0 DG5S953 |
| | B5 | 0.000060 | >10 | 0.034 | 0.000 | 169.87-170.17 | 0 DG5S953 0 DG5S955 0 DG5S13 0 DG5S123 6 DG5S959 |

TABLE 3
Pairwise correlation between the six haplotypes in the A-region
that show the strongest association to non-obese diabetes.
Estimates of D′ are shown in the upper right corner, and
estimates of R² are shown the the lower left corner. The haplotypes
are labelled A1, . . . , A6 as in Table 2.

D′

| | | A1 | A2 | A3 | A4 | A5 | A6 |
|---|---|---|---|---|---|---|---|
| R² | A1 | — | 0.72 | 0.85 | 1.00 | 0.72 | 1.00 |
| | A2 | 0.25 | — | 1.00 | 0.36 | 1.00 | 0.41 |
| | A3 | 0.31 | 1.00 | — | 0.35 | 1.00 | 0.41 |
| | A4 | 0.64 | 0.10 | 0.10 | — | 0.36 | 1.00 |
| | A5 | 0.31 | 0.86 | 0.86 | 0.10 | — | 0.44 |
| | A6 | 0.73 | 0.14 | 0.14 | 1.00 | 0.16 | — |

TABLE 4
The most significant single-marker allelic association results with SLIT3.
All results with a two-sided P-value <0.05 are shown, both for microsatellites
and SNPs. Included in the table is the corresponding relative risk (RR),
the number of non-obese diabetics and controls used in the test and the corresponding
frequency of the at-risk variant in both cohorts.

| Location | Marker | Allele | P-value | RR | #aff | Aff. frq | #ctrl | Ctr. frq |
|---|---|---|---|---|---|---|---|---|
| 168.334817 | DG5S1053 | 24 | 0.007 | 9.61 | 461 | 0.015 | 312 | 0.00 |
| 168.719742 | SG05S451 | C | 0.012 | 2.49 | 518 | 0.045 | 240 | 0.02 |
| 168.770226 | DG5S37 | −6 | 0.013 | 1.94 | 491 | 0.060 | 314 | 0.03 |
| 168.098154 | DG5S1047 | −12 | 0.013 | 1.35 | 468 | 0.277 | 313 | 0.22 |
| 168.666372 | SLT_8778 | A | 0.015 | 1.34 | 502 | 0.778 | 311 | 0.72 |
| 168.112080 | SLT_621478 | T | 0.015 | 1.42 | 476 | 0.563 | 124 | 0.48 |
| 168.051407 | SLT_680684 | C | 0.017 | 1.44 | 504 | 0.302 | 152 | 0.23 |
| 168.677067 | SLT_278 | G | 0.018 | 1.32 | 505 | 0.777 | 317 | 0.73 |
| 168.666183 | SLT_8967 | C | 0.026 | 1.31 | 492 | 0.323 | 267 | 0.27 |
| 167.992779 | DG5S87 | 0 | 0.033 | 1.27 | 434 | 0.460 | 279 | 0.40 |
| 168.334817 | DG5S1053 | 26 | 0.034 | 7.52 | 461 | 0.012 | 312 | 0.00 |
| 168.554788 | DG5S1058 | −2 | 0.038 | 4.35 | 461 | 0.014 | 305 | 0.00 |
| 168.288956 | rs891958 | G | 0.044 | 1.33 | 496 | 0.192 | 311 | 0.15 |

TABLE 5
Microsatellites and SNP haplotype association within SLIT3. The five haplotypes, that include 5
or less markers and are shorter than 300 kb, that show strongest association to non-obese diabetes.
The five haplotypes, that are strongly correlated, all span the 5′ end of the gene.

| | P-value | RR | Aff. frq | Ctrl. frq | Haplotype |
|---|---|---|---|---|---|
| C1 | 2.334E−08 | 2.12 | 0.286 | 0.159 | 4 DG5S831 G SLT_90256 G SLT_89801 C SLT_8967 G SLT_278 |
| C2 | 4.329E−08 | 2.09 | 0.283 | 0.159 | 4 DG5S881 G SLT_89801 0 DG5S1845 C SLT_8967 G SLT_278 |
| C3 | 4.553E−08 | 2.10 | 0.282 | 0.158 | 4 DG5S881 G SLT_89801 0 DG5S1645 C SLT_8967 T SLT_8778 |
| C4 | 5.503E−08 | 2.07 | 0.286 | 0.162 | 4 DG5S881 G SLT_90256 G SLT_89801 C SLT_8967 T SLT_8778 |
| C5 | 6.927E−08 | 2.25 | 0.244 | 0.125 | 4 DG5S881 T rs297898 G SLT_89801 0 DG5S1645 C SLT_8967 |

The teachings of all publications cited herein are incorporated herein by reference in their entirety. While this invention has been particularly shown and described with references to preferred embodiments thereof, it will be understood by those skilled in the art that various changes in form and details may be made therein without departing from the scope of the invention encompassed by the appended claims.

1. A method of diagnosing a susceptibility to Type II diabetes in an individual, comprising detecting a polymorphism in a SLIT-3 nucleic acid, wherein the presence of the polymorphism in the nucleic acid is indicative of a susceptibility to Type II diabetes.

2. A method of diagnosing a susceptibility to Type II diabetes comprising detecting an alteration in the expression or composition of a polypeptide encoded by SLIT-3 nucleic acid in a test sample, in comparison with the expression or composition of a polypeptide encoded by a SLIT-3 nucleic acid in a control sample, wherein the presence of an alteration in expression or composition of the polypeptide in the test sample is indicative of a susceptibility to Type II diabetes.

3. The method of claim 1, wherein the polymorphism in the SLIT-3 nucleic acid is indicated by detecting the presence of a least one of the polymorphisms indicated in FIG. 11.

4. An isolated nucleic acid molecule comprising a SLIT-3 nucleic acid, wherein the SLIT-3 nucleic acid has a nucleotide sequence selected from the group of nucleic acid sequences as shown in FIG. 10, or the complements of the group of nucleic acid sequences as shown in FIG. 10, wherein the nucleotide sequence contains a polymorphism.

5. An isolated nucleic acid molecule which hybridizes under high stringency conditions to a nucleotide sequence selected from the group of nucleic acid sequences as shown in FIG. 10, or the complements of the group of nucleic acid sequences as shown in FIG. 10, wherein the nucleotide sequence contains a polymorphism.

6. A method for assaying for the presence of a first nucleic acid molecule in a sample, comprising contacting said sample with a second nucleic acid molecule, where the second nucleic acid molecule comprises a nucleotide sequence selected from the group consisting of: nucleic acid sequences as shown in FIG. 10 and the complement of the nucleic acid sequences as shown in FIG. 10, wherein the nucleotide sequence contains a polymorphism and hybridizes to the first nucleic acid under high stringency conditions.

7. A vector comprising an isolated nucleic acid molecule selected from the group consisting of:
a) nucleic acid sequences as shown in FIG. 10; and
b) complement of one of the nucleic acid sequences are shown in FIG. 10; and
wherein the nucleic acid molecule contains a polymorphism and is operably linked to a regulatory sequence.

8. A recombinant host cell comprising the vector of claim 7.

9. A method for producing a polypeptide encoded by an isolated nucleic acid molecule having a polymorphism, comprising culturing the recombinant host cell of claim 8 under conditions suitable for expression of the nucleic acid molecule.

10. A method of assaying for the presence of a polypeptide encoded by an isolated nucleic acid molecule according to claim 4 in a sample, the method comprising contacting the sample with an antibody which specifically binds to the encoded polypeptide.

11. A method of identifying an agent that alters expression of a SLIT-3 nucleic acid, comprising:
a) contacting a solution containing a nucleic acid comprising the promoter region of the SLIT-3 nucleic acid operably linked to a reporter gene with an agent to be tested;
b) assessing the level of expression of the reporter gene; and
c) comparing the level of expression with a level of expression of the reporter gene in the absence of the agent; wherein if the level of expression of the reporter gene in the presence of the agent differs, by an amount that is statistically significant, from the level of expression in the absence of the agent, then the agent is an agent that alters expression of the SLIT-3 nucleic acid.

12. An agent that alters expression of the SLIT-3 nucleic acid, identifiable according to the method of claim 11.

13. A method of identifying an agent that alters expression of a SLIT-3 nucleic acid, comprising:
a) contacting a solution containing a nucleic acid of claim 4 or a derivative or fragment thereof with an agent to be tested;
b) comparing expression with expression of the nucleic acid, derivative or fragment in the absence of the agent;
wherein if expression of the nucleotide, derivative or fragment in the presence of the agent differs, by an amount that is statistically significant, from the expression in the absence of the agent, then the agent is an agent that alters expression of the SLIT-3 nucleic acid.

14. The method of claim 13, wherein the expression of the nucleotide, derivative or fragment in the presence of the agent comprises expression of one or more splicing variant(s) that differ in kind or in quantity from the expression of one or more splicing variant(s) the absence of the agent.

15. An agent that alters expression of a SLIT-3 nucleic acid, identifiable according to the method of claim 14.

16. An agent that alters expression of a SLIT-3 nucleic acid, selected from the group consisting of: antisense nucleic acid to a SLIT-3 nucleic acid; a SLIT-3 polypeptide; a SLIT-3 nucleic acid receptor; a SLIT-3 binding agent; a peptidomimetic; a fusion protein; a prodrug thereof; an antibody; and a ribozyme.

17. A method of altering expression of a SLIT-3 nucleic acid, comprising contacting a cell containing a SLIT-3 nucleic acid with an agent of claim 16.

18. A method of identifying a polypeptide which interacts with a SLIT-3 polypeptide comprising a polymorphism indicated in FIG. 11, comprising employing a yeast two-hybrid system using a first vector which comprises a nucleic acid encoding a DNA binding domain and a SLIT-3 polypeptide, splicing variant, or a fragment or derivative thereof, and a second vector which comprises a nucleic acid encoding a transcription activation domain and a nucleic acid encoding a test polypeptide, wherein if transcriptional activation occurs in the yeast two-hybrid system, the test polypeptide is a polypeptide which interacts with a SLIT-3 polypeptide.

19. A Type II diabetes therapeutic agent selected from the group consisting of: a SLIT-3 nucleic acid or fragment or derivative thereof; a member of the Robo family nucleic acid or fragment or derivative thereof; a polypeptide encoded by a SLIT-3 nucleic acid; a polypeptide encoded by a member of the Robo family nucleic acid; a SLIT-3 receptor; receptor for a member of the Robo family; a SLIT-3 nucleic acid binding agent; a Robo family member nucleic acid binding agent; a peptidomimetic; a fusion protein; a prodrug; an antibody; an agent that alters SLIT-3 nucleic acid expression; an agent that alters a Robo family member nucleic acid expression; an agent that alters activity of a polypeptide encoded by a SLIT-3 nucleic acid; an agent that alters activity of a polypeptide encoded nucleic acid of a Robo family member; an agent that alters posttranscriptional processing of a polypeptide encoded by a SLIT-3 nucleic acid; an agent that alters posttranscriptional processing of a polypeptide encoded by a nucleic acid of a Robo family member; an agent that alters interaction of a SLIT-3 nucleic acid with a SLIT-3 binding agent; an agent that alters interaction of a nucleic acid of a member of the Robo family with a Robo family binding agent; an agent that alters interaction of a SLIT-3 nucleic acid with a Robo family member; an agent that alters transcription of splicing variants encoded by a SLIT-3 nucleic acid; an agent that alters transcription of splicing variants encoded by a nucleic acid of a Robo family member; and a ribozyme.

20. A pharmaceutical composition comprising a Type II diabetes therapeutic agent of claim 19.

21. The pharmaceutical composition of claim 20, wherein the Type II diabetes therapeutic agent is selected from one or more of the group consisting of an isolated nucleic acid molecule comprising a SLIT-3 nucleic acid or fragment or derivative thereof, and a polypeptide encoded by the SLIT-3 nucleic acid.

22. (canceled)

23. A method of treating a disease or condition associated with SLIT-3 in an individual, comprising administering a Type II diabetes therapeutic agent to the individual, in a therapeutically effective amount.

24. The method of claim 23, wherein the Type II diabetes therapeutic agent is a SLIT-3 nucleic acid agonist or a SLIT-3 nucleic acid antagonist.

25. (canceled)

26. (canceled)

27. A method for assaying a sample for the presence of a SLIT-3 nucleic acid, comprising:
a) contacting said sample with a nucleic acid comprising a contiguous nucleotide sequence which is at least partially complementary to a part of the sequence of said SLIT-3 gene under conditions appropriate for hybridization, and
b) assessing whether hybridization has occurred between a SLIT-3 gene nucleic acid and said nucleic acid comprising a contiguous nucleotide sequence which is at least partially complementary to a part of the sequence of said SLIT-3 nucleic acid;
wherein if hybridization has occurred, a SLIT-3 nucleic acid is present in the nucleic acid.

28. The method of claim 27, wherein said nucleic acid comprising a contiguous nucleotide sequence is completely complementary to a part of the sequence of said SLIT-3 nucleic acid.

29. The method of claim 27, further comprising amplification of at least part of said SLIT-3 nucleic acid.

30. The method of claim 27, wherein said contiguous nucleotide sequence is 100 or fewer nucleotides in length and is either: a) at least 80% identical to a contiguous sequence of nucleotides in one of the nucleic acid sequences as shown in FIG. 10; b) at least 80% identical to the complement of a contiguous sequence of nucleotides in one of the nucleic acid sequences as shown in FIG. 10; or c) capable of selectively hybridizing to said SLIT-3 nucleic acid.

31. A reagent for assaying a sample for the presence of a SLIT-3 nucleic acid, said reagent comprising a nucleic acid comprising a contiguous nucleotide sequence which is at least partially complementary to a part of the nucleotide sequence of said SLIT-3 nucleic acid.

32. The reagent of claim 31, wherein the nucleic acid comprises a contiguous nucleotide sequence, which is completely complementary to a part of the nucleotide sequence of said SLIT-3 nucleic acid.

33. A reagent kit for assaying a sample for the presence of a SLIT-3 nucleic acid, comprising in separate containers:
a) one or more labeled nucleic acids comprising a contiguous nucleotide sequence which is at least partially complementary to a part of the nucleotide sequence of said SLIT-3 nucleic acid, and
b) reagents for detection of said label.

34. The reagent kit of claim 33, wherein the labeled nucleic acid comprises a contiguous nucleotide sequences which is completely complementary to a part of the nucleotide sequence of said SLIT-3 nucleic acid.

35. A reagent kit for assaying a sample for the presence of a SLIT-3 nucleic acid, comprising one or more nucleic acids comprising a contiguous nucleic acid sequence which is at least partially complementary to a part of the nucleic acid sequence of said SLIT-3 nucleic acid, and which is capable of acting as a primer for said SLIT-3 nucleic acid when maintained under conditions for primer extension.

36. The use of a nucleic acid which is 100 or fewer nucleotides in length and which is either:
a) at least 80% identical to a contiguous sequence of nucleotides in one of the nucleic acid sequences as shown in FIG. 10;
b) at least 80% identical to the complement of a contiguous sequence of nucleotides in one of the nucleic acid sequences as shown in FIG. 10; or
c) capable of selectively hybridizing to said SLIT-3 nucleic acid, for assaying a sample for the presence of a SLIT-3 nucleic acid.

37. The use of claim 36, wherein the SLIT-3 nucleic acid of (c) has at least one nucleotide difference from the first nucleic acid.

38. The use of a nucleic acid which is 100 or fewer nucleotides in length and which is either:
a) at least 80% identical to a contiguous sequence of nucleotides in one of the nucleic acid sequences as shown in FIG. 10;
b) at least 80% identical to the complement of a contiguous sequence of nucleotides in one of the nucleic acid sequences as shown in FIG. 10; or
c) capable of selectively hybridizing to said SLIT-3 nucleic acid; for diagnosing a susceptibility to a disease or condition associated with a SLIT-3.

39. A method of diagnosing a susceptibility to Type II diabetes in an individual, comprising determining the presence or absence in the individual of a haplotype shown in Table 2 or a haplotype shown in Table 5, at the 5q35 loci, wherein the presence of the haplotype is diagnostic of susceptibility to Type II diabetes.

40. The method of claim 39, wherein determining the presence or absence of the haplotype comprises enzymatic amplification of nucleic acid from the individual.

41. The method of claim 40, wherein determining the presence or absence of the haplotype further comprises electrophoretic analysis.

42. The method of claim 39, wherein determining the presence or absence of the haplotype further comprises an analysis selected from one or more of the group consisting of restriction fragment length polymorphism analysis and sequence analysis.

43. (canceled)

44. A method of diagnosing a susceptibility to Type II diabetes in an individual, comprising:
a) obtaining a nucleic acid sample from said individual; and
b) analyzing the nucleic acid sample for the presence or absence of a haplotype shown in Table 2 or shown in Table 5, at the 5q35 loci comprising a SLIT-3 gene, wherein the presence of the haplotype is diagnostic for a susceptibility to Type II diabetes.

45. A method of diagnosing a susceptibility to Type II diabetes in an individual, comprising determining the presence or absence in the individual of a haplotype comprising one or more markers and/or single nucleotide polymorphisms as shown in FIG. 11, in the locus on chromosome 5q35, wherein the presence of the haplotype is diagnostic of a susceptibility to Type II diabetes.

46. A method for the diagnosis and identification of a susceptibility to Type II diabetes in an individual, comprising: screening for an at-risk haplotype in the SLIT-3 nucleic acid that is more frequently present in an individual susceptible to Type II diabetes compared to an individual who is not susceptible to Type II diabetes wherein the at-risk haplotype increases the risk significantly.

47. The method of claim 46 wherein the significant increase is at least about 20%.

48. The method of claim 46 wherein the significant increase is identified as an odds ratio of at least about 1.2.

49. Use of a Type II diabetes therapeutic agent for the manufacture of a medicament for the treatment of a disease or condition associated with SLIT-3 in an individual.

50. The use of claim 49, wherein the Type II diabetes therapeutic agent is selected from one or more of the group consisting of a SLIT-3 nucleic acid agonist and a SLIT-3 nucleic acid antagonist.

51. (canceled)
